(19) United States
(12) Patent Application Publication
(10) Pub. No.: US 2026/0355205 A1
(43) Pub. Date: Nov. 12, 2026

(54) HERPESVIRUS-INDUCED GENE OR PROTEIN EXPRESSION AND METHODS FOR TREATING NEUROLOGICAL DISORDERS

(71) Applicant: UNIVERSITY OF MASSACHUSETTS, Westborough, MA (US)

(72) Inventors: Elaine Teng-Ting LIM, Newton, MA (US); Yingleong Rigel CHAN, Newton, MA (US)

(21) Appl. No.: 19/122,478

(22) PCT Filed: Oct. 19, 2023
(86) PCT No.: PCT/US2023/077265

§ 371 Date: Apr. 18, 2025

Related U.S. Application Data
(60) Provisional application No. 63/380,098, filed on Oct. 19, 2022.

(51) Int. Cl. A61K 31/522; A61K 31/197; A61K 31/35; A61K 31/381; A61K 31/4015; A61K 31/4045; A61K 31/428; A61K 31/445; A61K 31/53; A61K 31/55; A61K 31/5513; A61P 25/28; A61P 31/22; C12Q 1/6883

(52) U.S. Cl. CPC A61K 31/522; A61K 31/197; A61K 31/35; A61K 31/381; A61K 31/4015; A61K 31/4045; A61K 31/428; A61K 31/445; A61K 31/53; A61K 31/55; A61K 31/5513; A61P 25/28; A61P 31/22; C12Q 1/6883; C12Q 2600/158

(57) ABSTRACT

The present disclosure relates to compositions and methods for the treatment of neurological disorders.

### CROSS REFERENCE TO RELATED APPLICATIONS

This PCT application claims priority to, and the benefit of, U.S. Provisional Patent Application No. 63/380,098, filed Oct. 19, 2022, entitled “MODULATORS OF HERPESVIRUS-INDUCED GENE OR PROTEIN EXPRESSION FOR TREATING NEUROLOGICAL DISORDERS,” which is incorporated by reference herein in its entirety.

### STATEMENT REGARDING FEDERALLY SPONSORED RESEARCH

This invention was made with government support under Grant No. U01 AG061835 awarded by National Institutes of Health. The government has certain rights in the invention.

### FIELD

The present disclosure relates to compositions and methods for the treatment of neurological disorders.

### BACKGROUND

Neurological disorders are among the most significant public health challenges in today's society having a multifactorial etiology including genetic and environmental causes. Some evidence indicates that some neurological disorders, such as Alzheimer's disease, also have infectious etiologies, wherein the presence of a pathogen, such as a virus, can induce the expression of genes and/or proteins associated with neurological disorders. In addition, current challenges in treating neurological disorders include lack of reliable biomarkers for early diagnosis, and ineffective preventative strategies and therapies. Given limitations of current therapeutic options for treating neurological disorders, there is a need to address the problems mentioned above by developing compositions and methods for treating neurological disorders. The methods and kits disclosed herein address these and other needs.

### SUMMARY

Disclosed herein are methods of treating, preventing, detecting, and/or diagnosing a neurological disorder. Also disclosed are kits and methods of use thereof.

In one aspect, disclosed herein is a method of treating a neurological disorder in a subject in need thereof, the method comprising collecting a biological sample from the subject, detecting a gene expression signature from the biological sample, and administering a therapeutically effective amount of an anti-viral agent to the subject with the gene expression signature, wherein the gene expression signature comprises at least one gene affected by the neurological disorder and a herpes simplex virus 1 (HSV-1).

In some embodiments, the at least one gene is selected from a first gene set comprising AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, or YAP1.

In some embodiments, the at least one gene is selected from a second gene set comprising ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, or WNT7B.

In some embodiments, the first gene set is combined with the second gene set.

In some embodiments, the method detects an increase or decrease in expression of AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, YAP1, ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, WNT7B, or a combination thereof, relative to a healthy control subject.

In some embodiments, the neurological disorder comprises Alzheimer's disease (AD), compulsive disorder (OCD), attention-deficit/hyperactivity disorder (ADHD), schizophrenia, or amyotrophic lateral sclerosis (ALS).

In some embodiments, the anti-viral agent comprises acyclovir or valacyclovir. In some embodiments, the anti-viral agent is administered with a therapeutic agent. In some embodiments, the therapeutic agent comprises a neurological therapeutic agent. In some embodiments, the neurological therapeutic agent comprises levetiracetam, gabapentin, topiramate, lamotrigine, carbidopa, levodopa, donepezil HCl, sumatriptan succinate, oxcarbazepine, amitriptyline HCl, memantine HCl, divalprox sodium, pregabalin, pramipexole, ropinirole, rotigotine, clozapine, quetiapine, olanzapine, galantamine, rivastigmine, trifluoperazine, methylphenidate, atomoxetine, amphetamine, dextroamphetamine, pemoline, perphenazine, aducanumab, lecanemab, suvorexant, brexpiprazole, or a combination thereof.

In some embodiments, the method decreases amyloid-β (AB-42) or phosphorylated Tau (pTau-212) concentrations in the subject relative to an untreated subject with the neurological disorder. In some embodiments, the method prevents progression of the neurological disorder.

In some embodiments, the biological sample comprises a cerebrospinal fluid (CSF), blood, serum, or tissue biopsy. In some embodiments, the nucleic acid comprises an RNA molecule.

In one aspect, disclosed herein is a kit for detecting a panel of genes affected by a neurological disorder and a herpes simplex virus (HSV), the kit comprising a primer composition for binding at least one gene of said panel, and an enzymatic composition for amplifying the at least one gene, wherein the at least one gene comprises any one gene selected from AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, YAP1, ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, WNT7B, or fragments thereof.

In some embodiments, the primer composition comprises one, two, or more oligonucleotides. In some embodiments, the primer composition comprises a forward primer or a reverse primer. In some embodiments, the primer composition comprises a forward primer and a reverse primer.

In some embodiments, the oligonucleotides target a nucleotide sequence within the at least one gene. In some embodiments, the enzymatic composition comprises a polymerase and a buffer.

In some embodiments, the neurological disorder comprises Alzheimer's disease (AD), dementia, multiple sclerosis (MS), Parkinson's disease, Huntington's disease, obsessive compulsive disorder (OCD), attention-deficit/hyperactivity disorder (ADHD), schizophrenia, or amyotrophic lateral sclerosis (ALS).

In some embodiments, the HSV comprises an HSV-1. In some embodiments, the method further comprises a reagent for extracting a nucleic acid. In some embodiments, the method further comprises a reagent for detecting the at least one gene. In some embodiments, the nucleic acid comprises an RNA molecule.

### BRIEF DESCRIPTION OF FIGURES

The accompanying figures, which are incorporated in and constitute a part of this specification, illustrate several aspects described below.

FIGS. 1A, 1B, 1C, 1D, 1E, and 1F show the experimental setup and overview of the results from the RNA sequence datasets. FIG. 1A shows the five sets of experimental conditions generated by RNA-seq data on (Inf-vs-Ctrl cOrgs, Inf-vs-ACV cOrgs, Inf-vs-UV cOrgs, Inf-vs-Ctrl hiPSCs, Inf-vs-ACV hiPSCs). FIG. 1B shows the fluorescence images (20× magnification) of the cells across the experimental conditions. FIG. 1C shows the 3D PCA plot of the human transcripts (in FPKM), with the colored tints in the legend representing the experimental batches. Each number (1-9) represents an independent biological sample.

FIG. 1D shows the 3D PCA plot of the HSV-1 transcripts (in FPKM), with the colored tints in the legend representing the experimental batches. Each number (1-9) represents an independent biological sample. The percentages indicate the percentages of total transcripts that mapped to the HSV-1 transcriptome for each group of samples. The two sets of ACV-treated organoids are circled in purple and the infected organoids are circled in blue. FIG. 1E shows the intra-condition and inter-condition correlation plots showing pairwise FPKM values and Pearson's r for Inf-vs-Ctrl cOrgs 1 (Infected_Organoids4-6 versus Uninfected_Organoids1-3). FIG. 1F shows the volcano plot of differentially expressed human genes for Inf-vs-Ctrl cOrgs1 (Infected_Organoids4-6 versus Uninfected_Organoids1-3), with the log 2 fold change calculated as the expression of the transcript from HSV-1 infected cOrg cells versus uninfected cOrg cells, and −log₁₀ P-values were calculated using the Benjamini-Hochberg adjusted P-values of the differential expression.

FIGS. 2A, 2B, 2C, and 2D show the heatmaps depicting the expressions of immune transcripts. FIG. 2A shows the heatmap depicting human transcript expression in HSV-1 infected or uninfected cOrg samples. FIG. 2B shows the heatmap depicting human transcript expression from HSV-1 infected or ACV-treated cOrg samples. FIG. 2C shows the heatmap depicting human transcript expression from cOrg samples with UV-inactivated HSV-1 or HSV-1 infected cOrg samples. FIG. 2D shows the heatmap depicting human transcript expression from IAV infected or uninfected cOrg samples. FIGS. 2A, 2B, 2C, and 2D show the green bars on the right of the heatmaps annotate transcripts that were significantly differentially expressed in both datasets (adjusted P≤0.05). Red bars on the right of the heatmaps annotate transcripts that were significantly differentially expressed in cOrgs1 (adjusted P<0.05) but not in cOrgs2 (adjusted P>0.05). Gray bars on the right of the heatmaps annotate transcripts that were significantly differentially expressed in cOrgs2 but not in cOrgs1.

Black bars on the right of the heatmaps annotate transcripts that were not significantly differentially expressed in both datasets (adjusted P>0.05). Red vertical lines in the heatmaps separate both sets of discovery and replication datasets.

FIGS. 3A, 3B, 3C, 3D, 3E, 3F, 3G, and 3H show the rescue or exacerbation in HSV-1-infected and ACV-treated samples. FIG. 3A shows the −log₁₀ (Nominal P-value) from GSEA (y-axis) of the differentially expressed genes from datasets with GWAS-associated genes in common diseases and traits (x-axis). FIG. 3B shows the −log₁₀ (Nominal P-value) from GSEA (y-axis) of the differentially expressed genes from datasets with GWAS-associated genes in common diseases and traits (x-axis). FIG. 3C shows the scatter plot for the discovery dataset showing the log 2 fold change in infected versus ACV-treated cOrg samples, Inf-vs-ACV cOrgs1 (y-axis) and infected versus uninfected cOrg samples, Inf-vs-Ctrl cOrgs1 (x-axis), with the Pearson's r correlations of the log 2 fold changes of transcripts in each category shown on the bottom right. FIG. 3D shows the scatter plot for the replication dataset showing the log 2 fold change in infected versus ACV-treated cOrg samples, Inf-vs-ACV cOrgs2 (y-axis) and infected versus uninfected cOrg samples, Inf-vs-Ctrl cOrgs2 (x-axis), with the Pearson's correlations (r) of the log₂ fold changes of transcripts in each category shown on the bottom right. FIG. 4E shows the venn diagram showing the numbers and percentages of genes that were rescued or not rescued in both discovery and replication datasets (cOrgs1 and cOrgs2). FIG. 4F shows the venn diagram showing the numbers and percentages of genes that were in the exacerbated 1 or exacerbated 2 categories in both discovery and replication datasets (cOrgs1 and cOrgs2). FIG. 4G shows the concentrations of Aβ42/40/38 in pg/mL, Aβ42/40 ratios and Aβ42/38 ratios detected from conditioned media that had undergone heat inactivation for uninfected (control) cOrg cells, HSV-1 infected (HSV-1+) cOrg cells, HSV-1 infected and ACV-treated (Treated) cOrg cells, and cOrg cells with UV-inactivated HSV-1 (UV-HSV-1). P-values shown were calculated using 1-sided Wilcoxon ranked sum test with comparison to control uninfected cOrg cells. FIG. 4H shows the concentrations of Aβ42/40/38 in pg/mL, β42/40 ratios and Aβ42/38 ratios detected from conditioned media that had undergone heat inactivation for uninfected (control) cOrg cells and IAV infected (IAV+) cOrg cells. P-values shown were calculated using 1-sided Wilcoxon ranked sum test with comparison to control uninfected cOrg cells.

FIGS. 4A, 4B, 4C, 4D, 4E, 4F, 4G, and 4H show the cell type specific contribution to AD neuropathology markers. FIG. 4A shows the schematic of the flow cytometry experiments and computational analyses. FIG. 4B shows the UMAP plots showing the normalized fluorophore intensities for Aβ42 (red), HSV-1 (green) and Zombie fluorophore (blue) across uninfected, HSV-1 infected and ACV-treated cells. FIG. 4C shows the UMAP plots showing the normalized fluorophore intensities for pTau-212 (red), HSV-1 (green) and Zombie fluorophore (blue) across uninfected, HSV-1 infected and ACV-treated cells. FIG. 4D shows the pairwise adjusted correlation heatmaps using the intensities for HSV-1, Zombie fluorophore and Aβ42 or pTau-212 across uninfected, HSV-1 infected and ACV-treated cells. The adjusted correlations for 2 sets of replicates (Replicate 1 and Replicate 2) were shown. FIG. 4E shows the UMAP plots showing the normalized fluorophore intensities for Aβ42 (red), IAV (green) and Zombie fluorophore (blue) across uninfected and IAV infected cells. FIG. 4F shows the UMAP plots showing the normalized fluorophore intensities for pTau-212 (red), IAV (green) and Zombie fluorophore (blue) across uninfected and IAV infected cells. FIG. 4G shows the pairwise adjusted correlation heatmaps using the intensities for IAV, Zombie fluorophore and Aβ42 or pTau-212 across uninfected and IAV-infected cells. The adjusted correlations for 2 sets of replicates (Replicate 1 and Replicate 2) were shown. FIG. 4H shows the pairwise adjusted correlation heatmaps using the intensities for HSV-1, Zombie fluorophore and cell type markers (Iba1-microglia, GFAP-astrocytes, OLIG1-oligodendrocyte progenitor cells or NeuN-neurons).

FIGS. 5A, 5B, 5C, 5D, 5E, 5F, 5G, and 5H show the cell type specific analyses of HSV-1 infection and rescue by ACV. FIG. 5A shows the CellScores calculated using Orgo-Seq method and CellScores>1 indicate that there is an enrichment, whereas CellScores<1 indicate that there is a depletion, of (5A) astrocytes, (5B) excitatory neurons, (5C) inhibitory neurons, (5D) microglia, (5E) oligodendrocytes and (5F) oligodendrocyte progenitor cells. FIG. 5G shows the ORs calculated for HSV-1 infected live cOrg cells versus with uninfected live cOrg cells, or ACV-treated live cOrgs cells versus uninfected live cOrg cells, using 14 cell type markers. 2 sets of replicate experiments were performed for each marker (depicted by green and purple points). Shaded points represent significant odds ratios with P<0.05 and open points represent non-significant odds ratios with P>0.05. The yellow bars represent average ORs<1 across both replicates and the blue bars represent average ORs>1 across both replicates. FIG. 5H shows the ORs calculated for HSV-1 infected dead cOrg cells versus with uninfected dead cOrg cells, or ACV-treated dead cOrg cells versus uninfected dead cOrg cells, using 14 cell type markers. 2 sets of replicate experiments were performed for each marker (depicted by green and purple points). Shaded points represent significant odds ratios with P<0.05 and open points represent non-significant odds ratios with P>0.05. The yellow bars represent average ORs<1 across both replicates and the blue bars represent average ORs>1 across both replicates.

FIGS. 6A, 6B, and 6C show heatmaps comparing AD subtypes using post-mortem brains with cOrg samples. FIG. 6A shows the heatmap comparing the average log₂ fold changes from human post-mortem brains across the AD subtypes with the average log₂ fold changes from cOrg samples on the same scale. FIG. 6B shows the heatmap comparing the average log₂ fold changes from human post-mortem brains across the AD subtypes with the average log₂ fold changes from cOrg samples on different scales. FIG. 6C shows the enrichment of differential expression between human post-mortem brains and cOrgs across the 5 subtypes (A, B1, B2, C1 and C2). cOrgs1 are the results using Inf-vs-Ctrl cOrg1 and cOrgs2 are the results using Inf-vs-Ctrl cOrgs2.

FIGS. 7A, 7B, 7C, 7D, 7E, 7F, 7G, 7H, 7I, 7J, 7K, 7L, 7M, and 7N show the PCA, volcano and odds ratios plots of human and HSV-1 transcripts. FIG. 7A shows the PCA plot of human transcripts, showing that PC2 separates the cerebral organoid samples from hiPSC samples. FIG. 7B shows the PCA plot of viral transcripts, showing that PC2 separates the infected cOrg samples from the ACV-treated cOrg samples and PC3 separates the cOrg samples from hiPSC samples. FIG. 7C shows the Inf-vs-Ctrl cOrgs2 (Infected_Organoids7-9 versus Uninfected_Organoids4-6), with the log 2 fold change calculated using the expression of human transcripts in infected cOrgs over uninfected cOrgs. FIG. 7D shows the Inf-vs-UV cOrgs2 (Infected_Organoids7-9 versus UV-HSV-1_Organoids1-3), with the log₂ fold change calculated as the expression of human transcripts in infected cOrgs over cOrgs with UV-inactivated virus. FIG. 7E shows the Inf-vs-Ctrl hiPSCs (Infected_hiPSCs1-3 versus Uninfected_hiPSCs1-3), with the log₂ fold change calculated as the expression of human transcripts in infected hiPSCs over uninfected hiPSCs. FIG. 7F shows the Inf-vs-ACV cOrgs1 (Infected_Organoids4-6 versus Treated_Organoids1-3), with the log₂ fold change calculated using expression of human transcripts in infected cOrgs over ACV-treated cOrgs.

FIG. 7G shows the Inf-vs-ACV cOrgs2 (Infected_Organoids7-9 versus Treated_Organoids4-6), with the log₂ fold change calculated using the expression of human transcripts in infected cOrgs over infected and ACV-treated cOrgs. FIG. 7H shows the ACV-vs-Ctrl cOrgs2

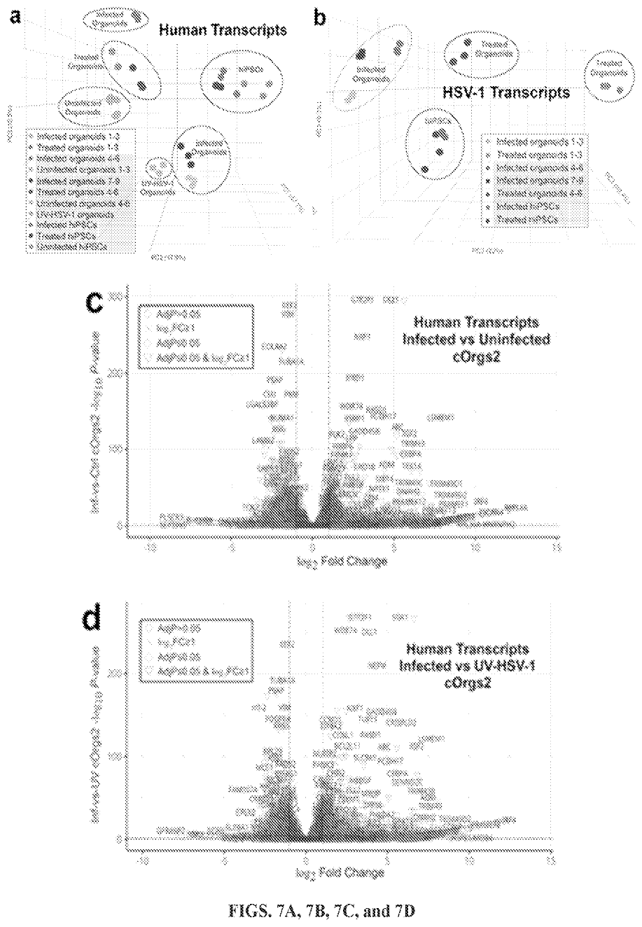

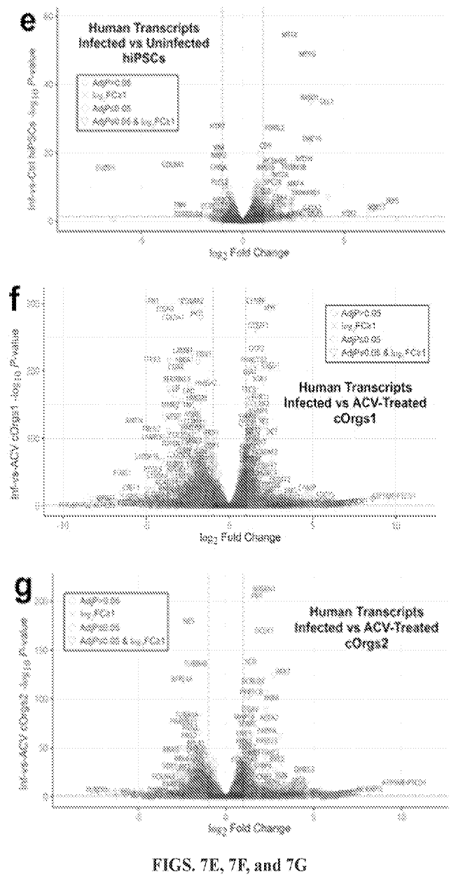

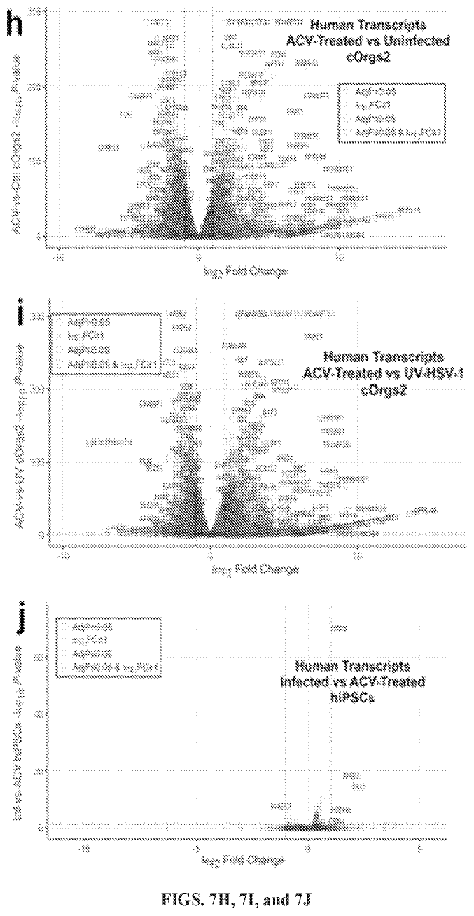

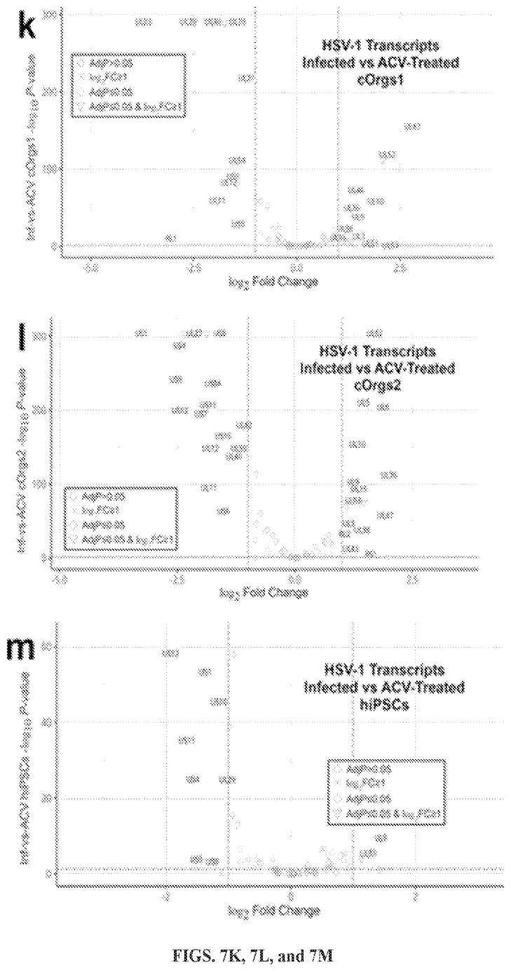

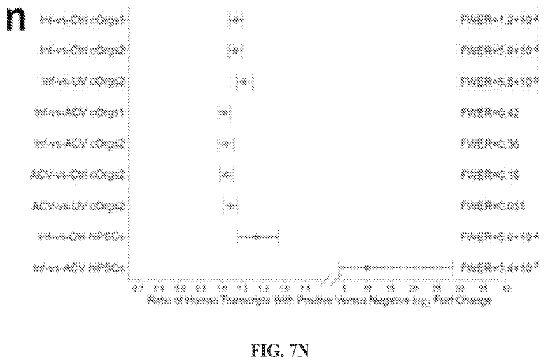

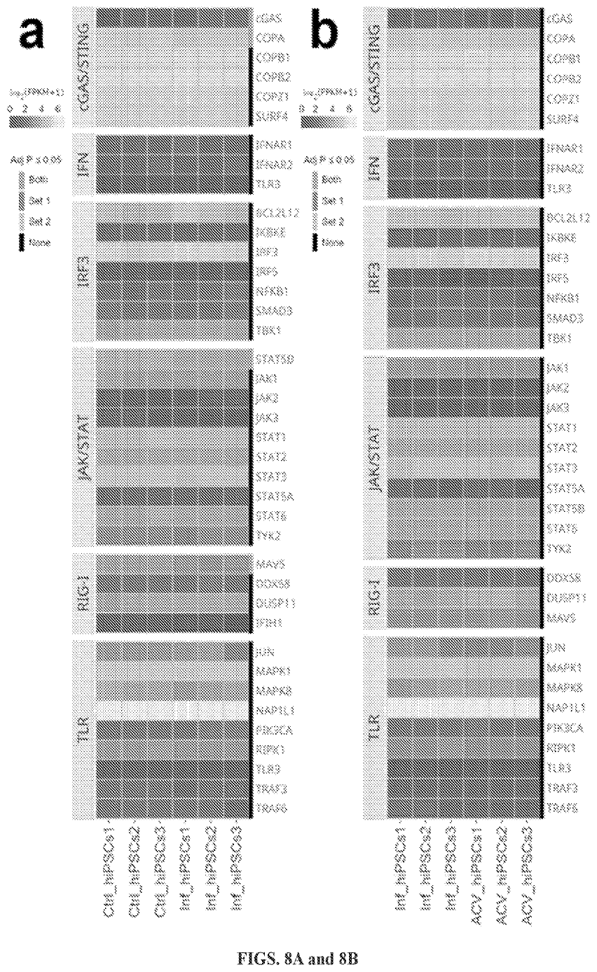

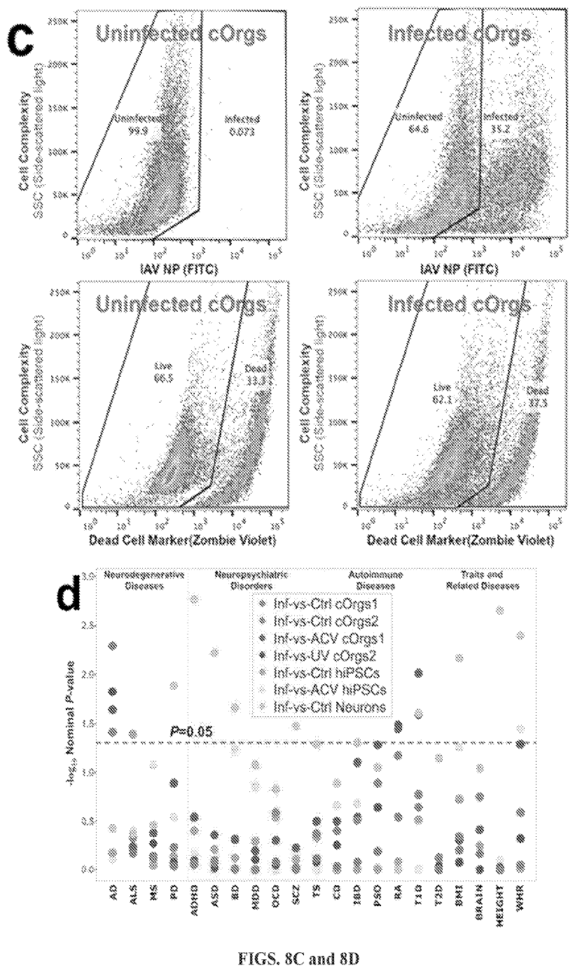

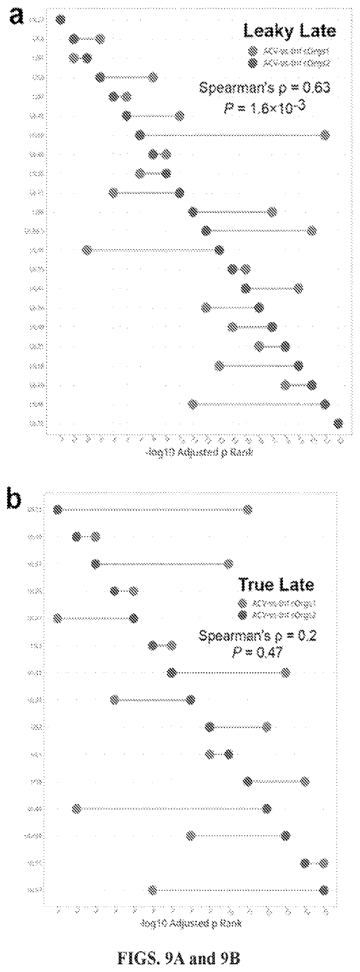

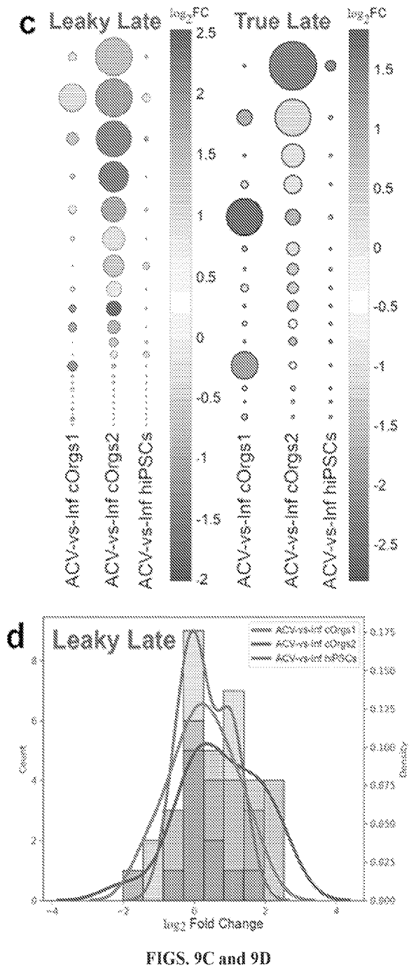

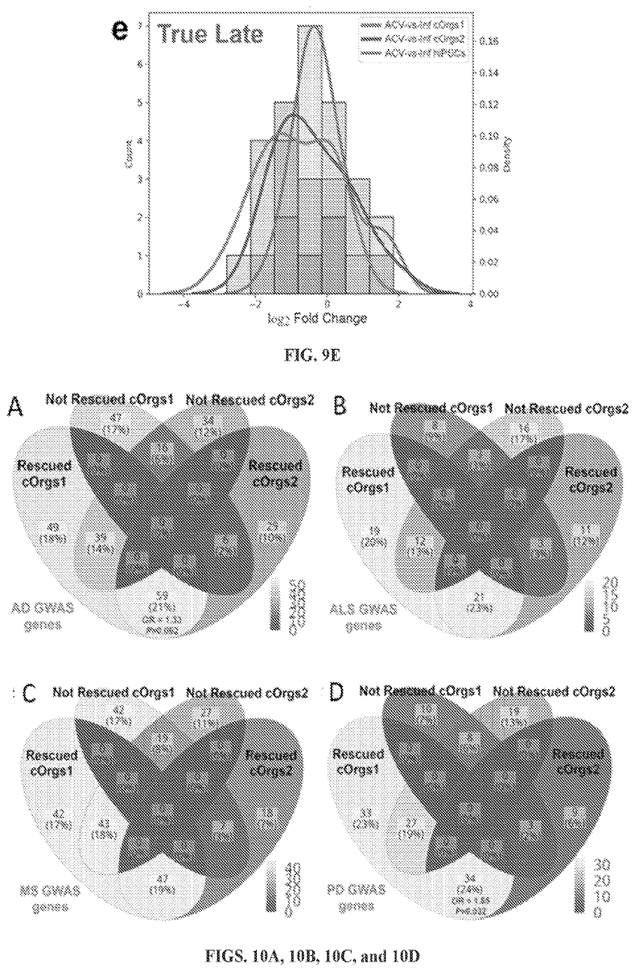

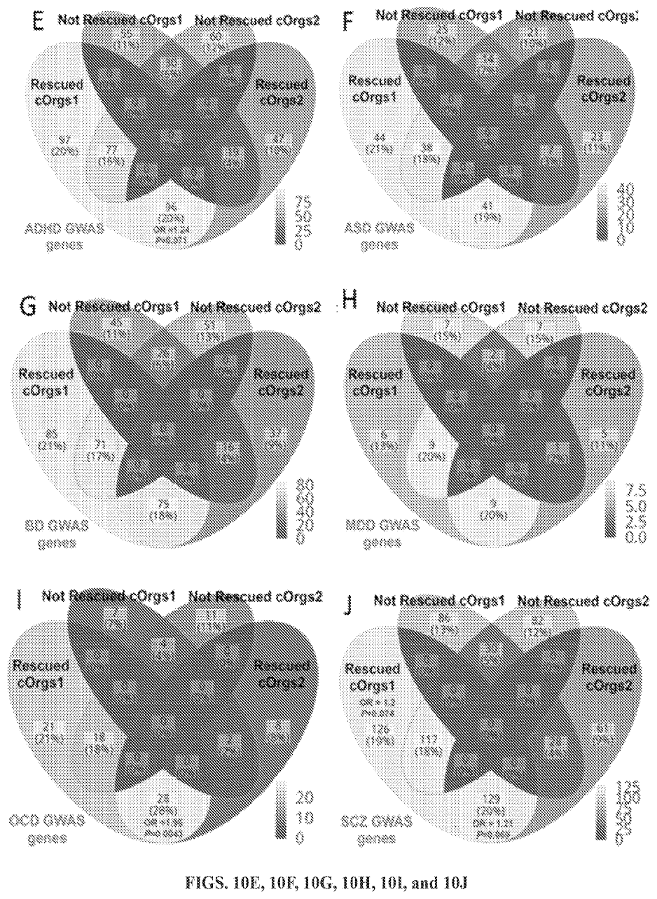

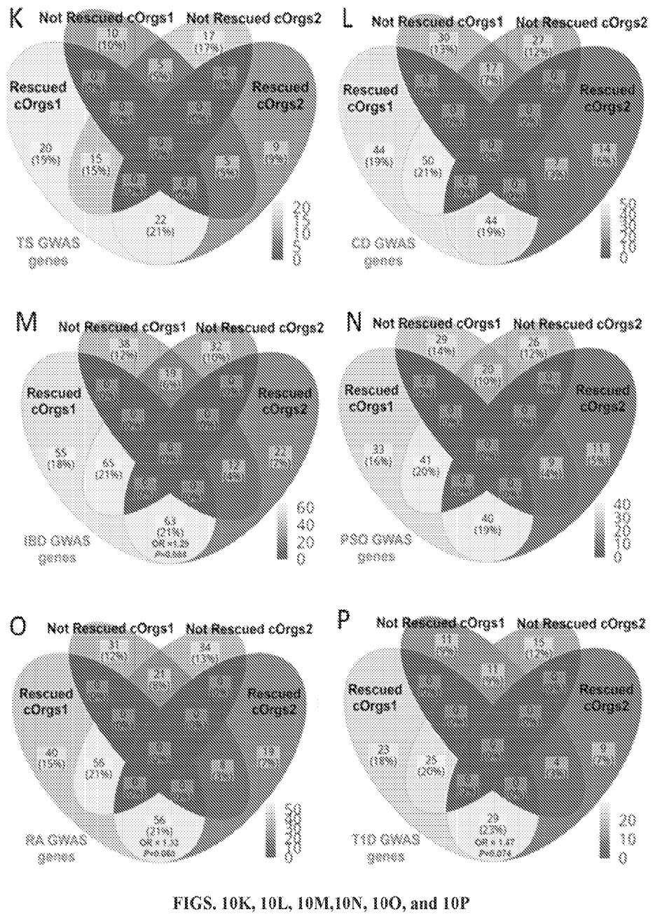

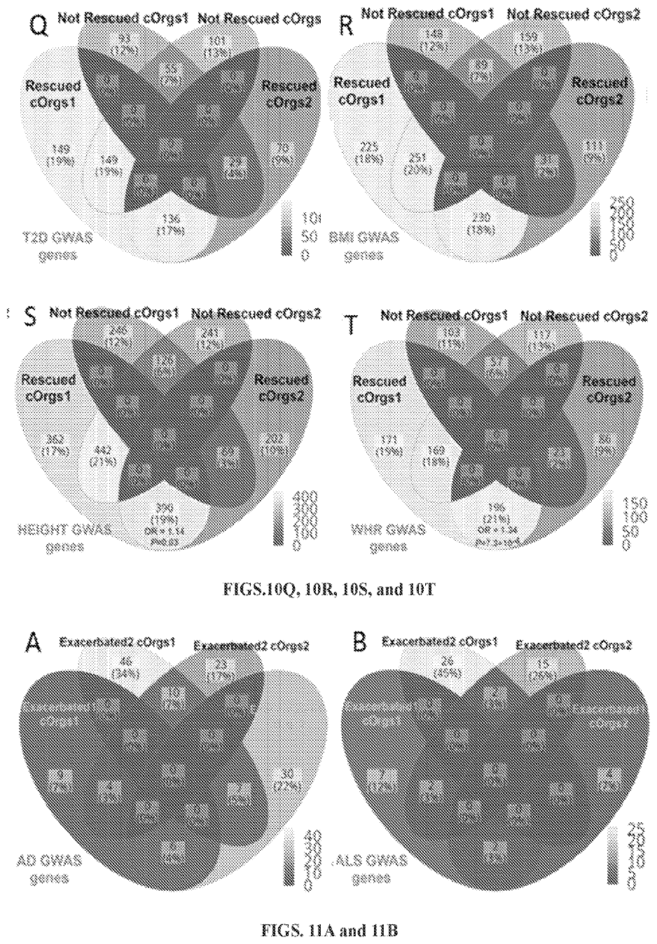

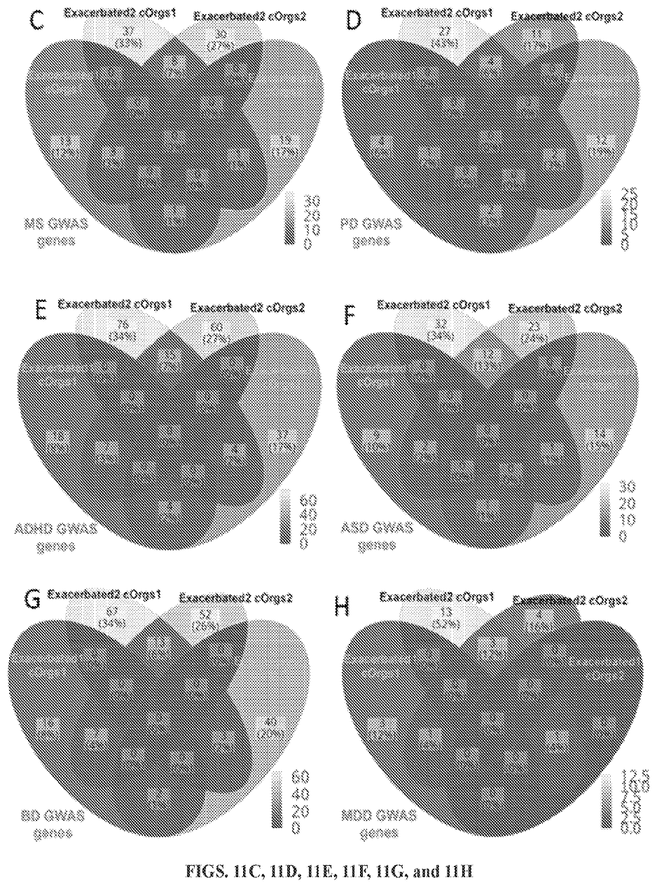

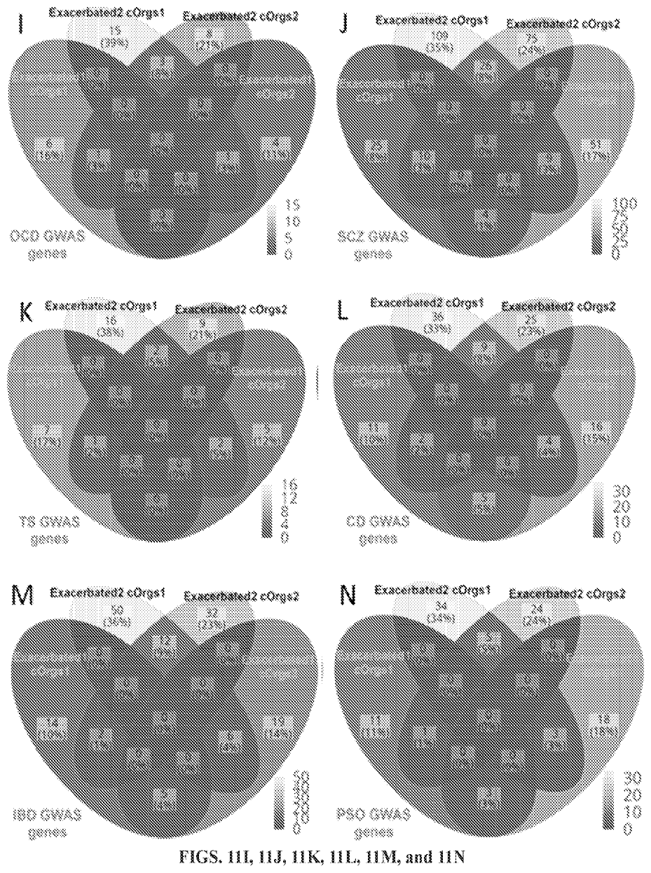

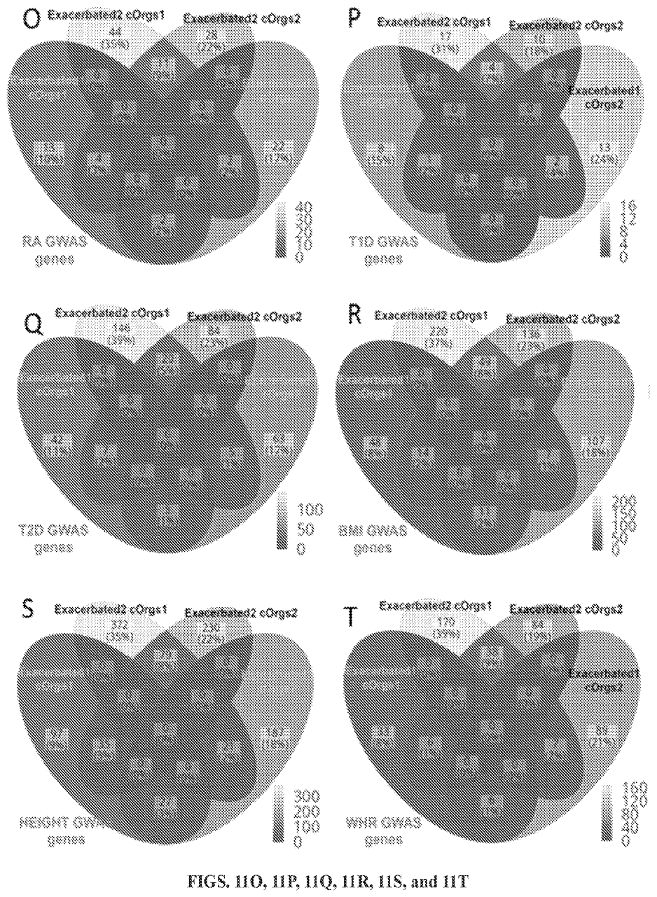

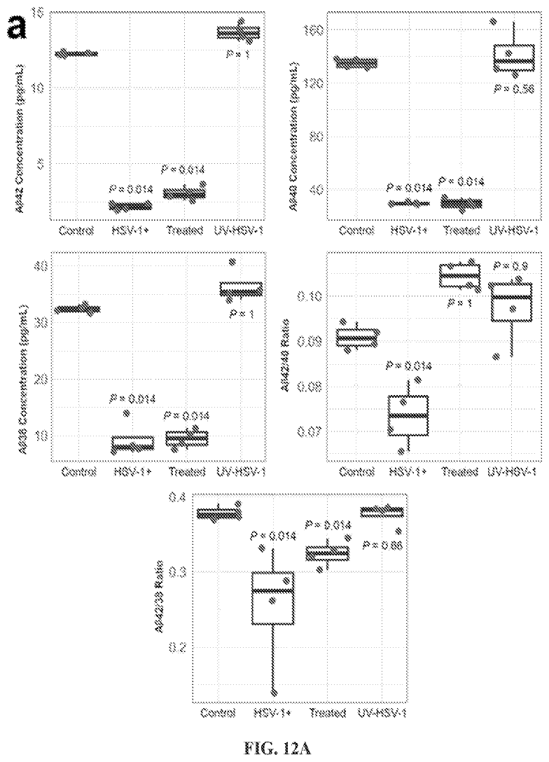

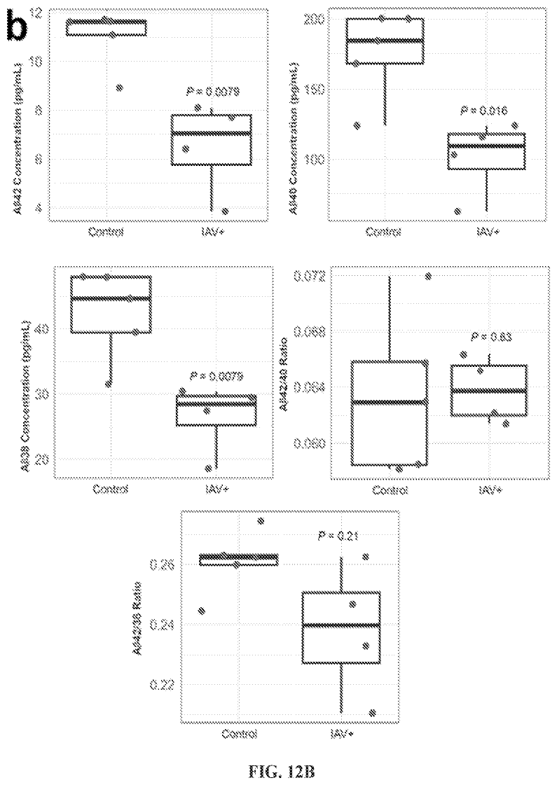

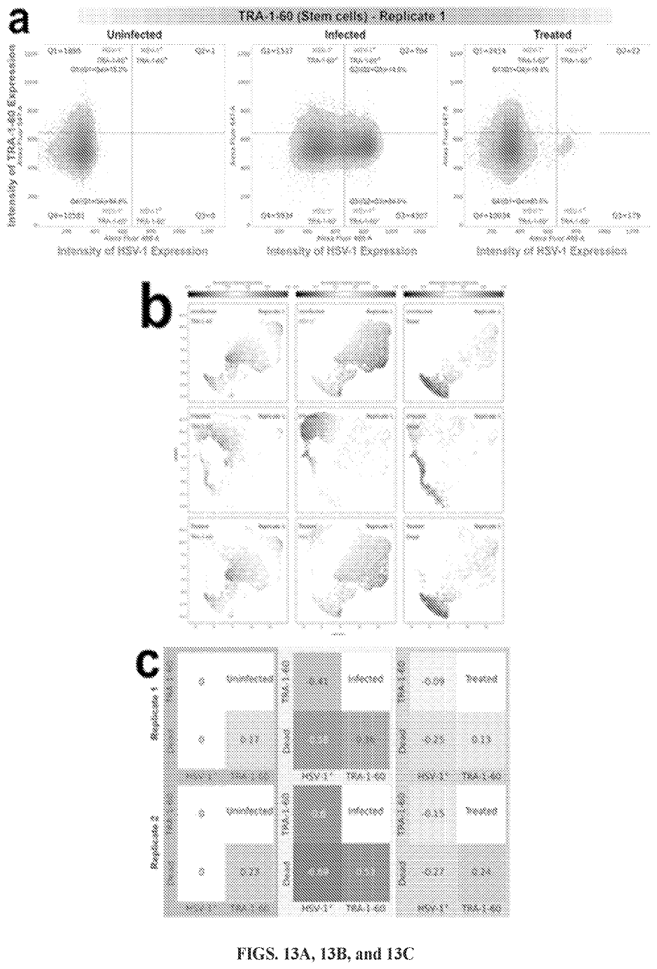

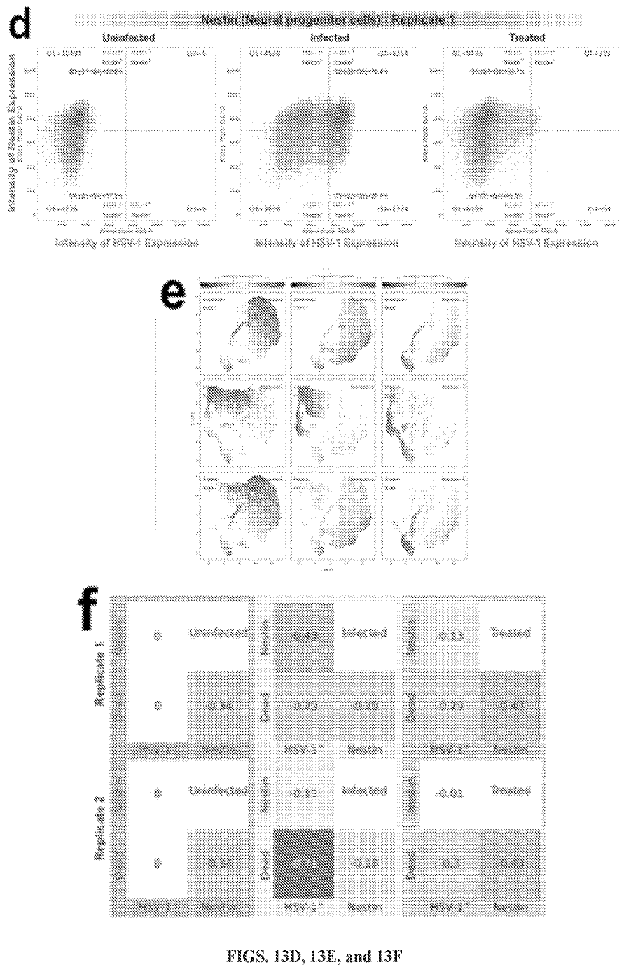

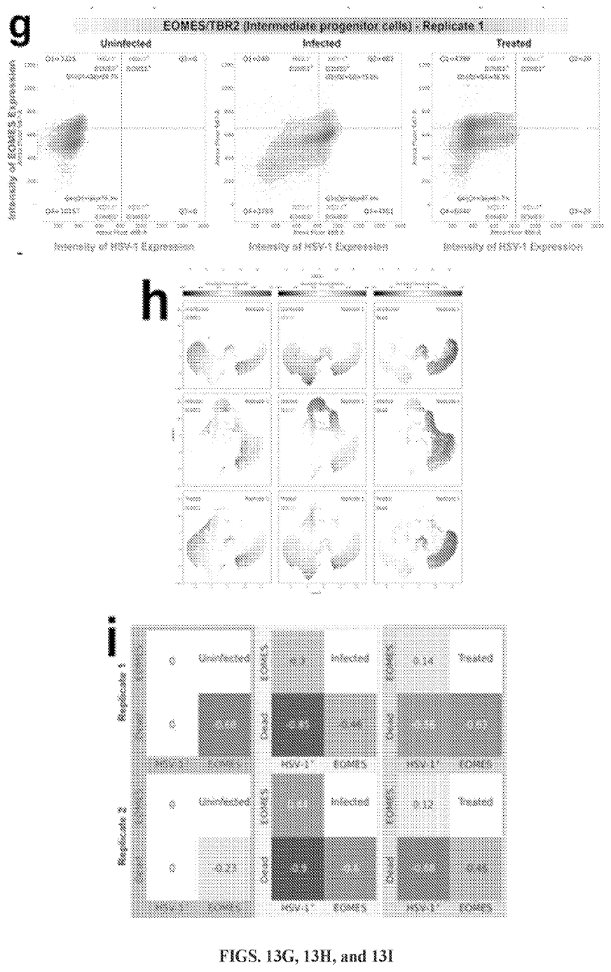

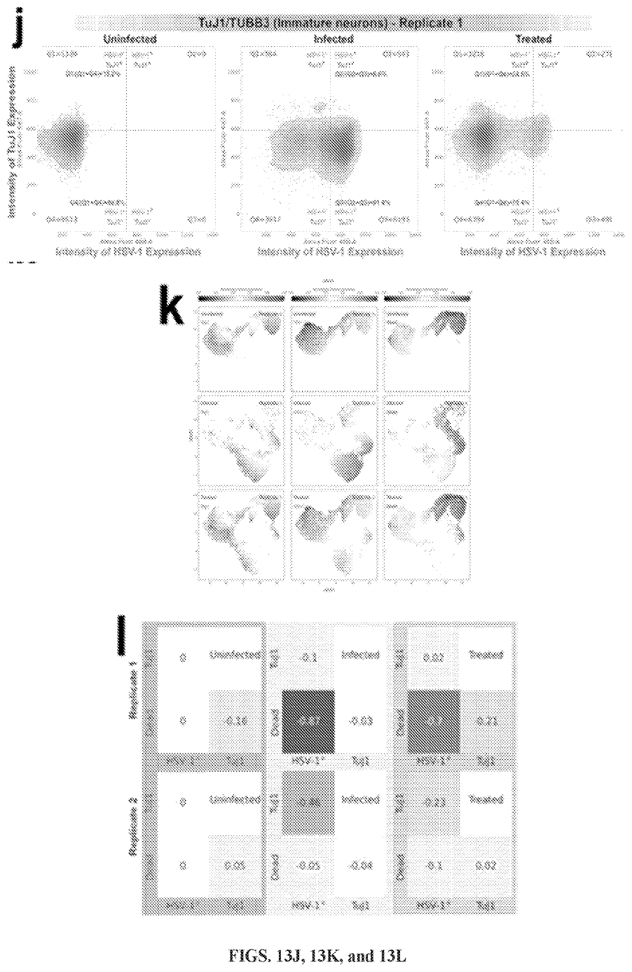

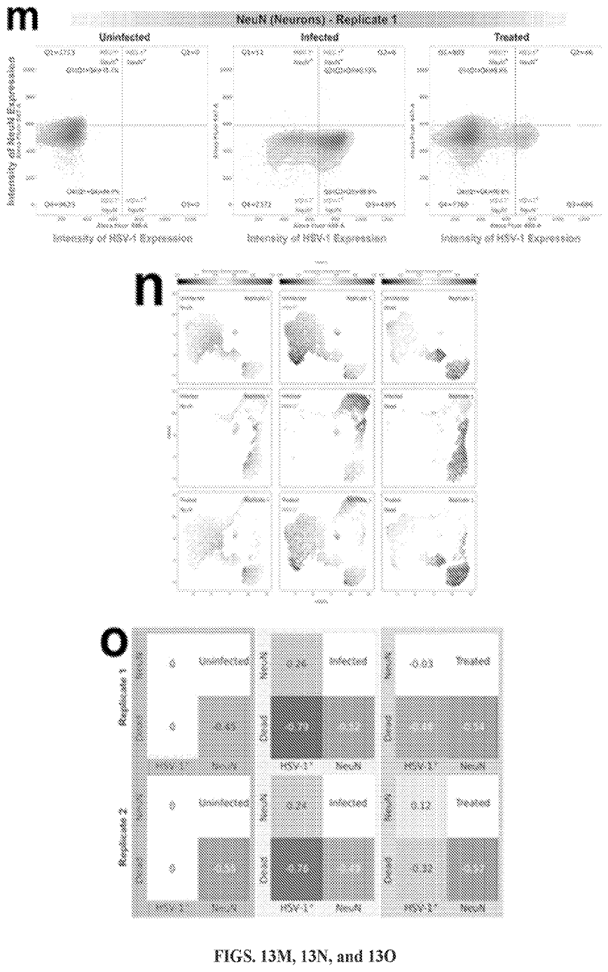

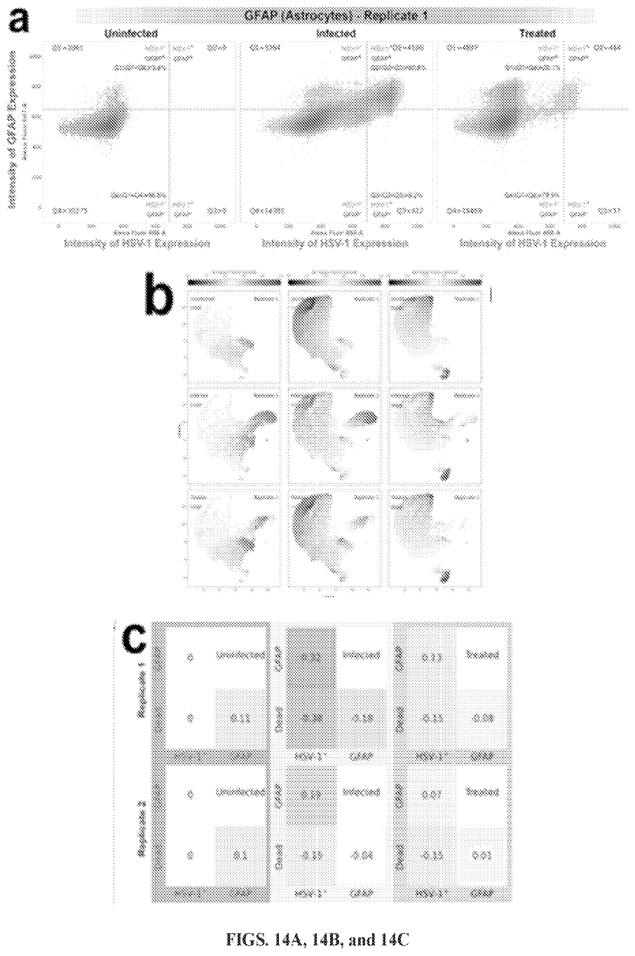

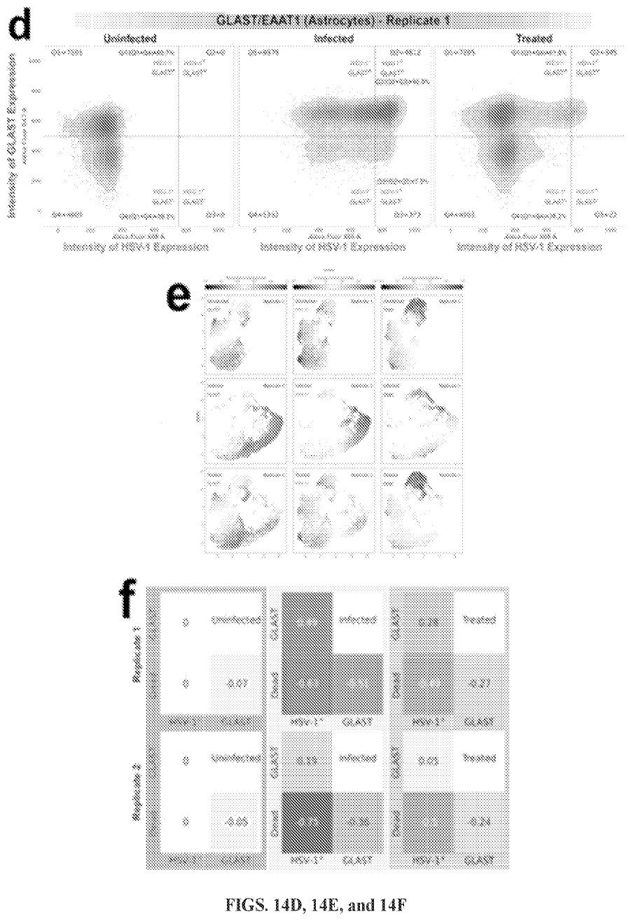

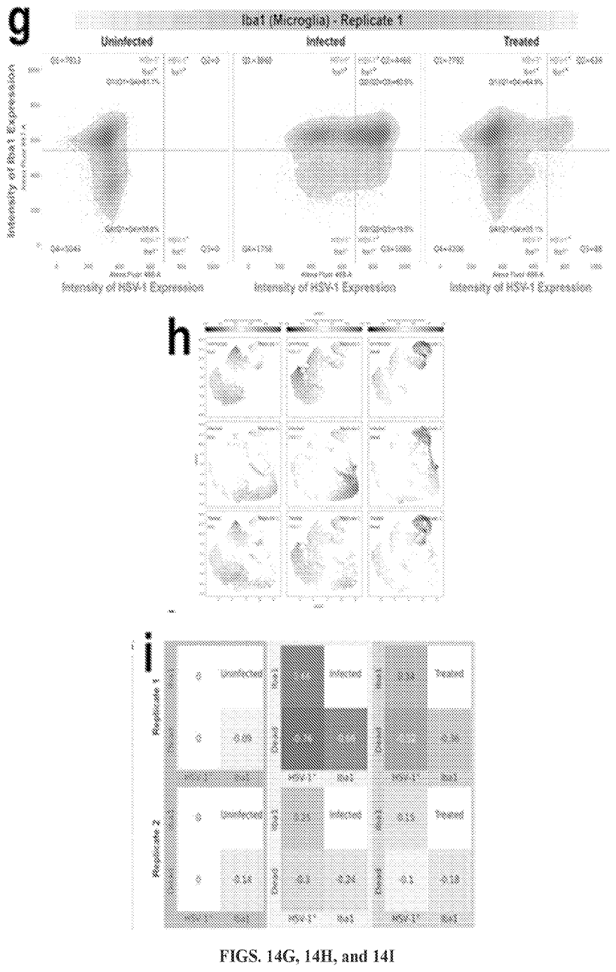

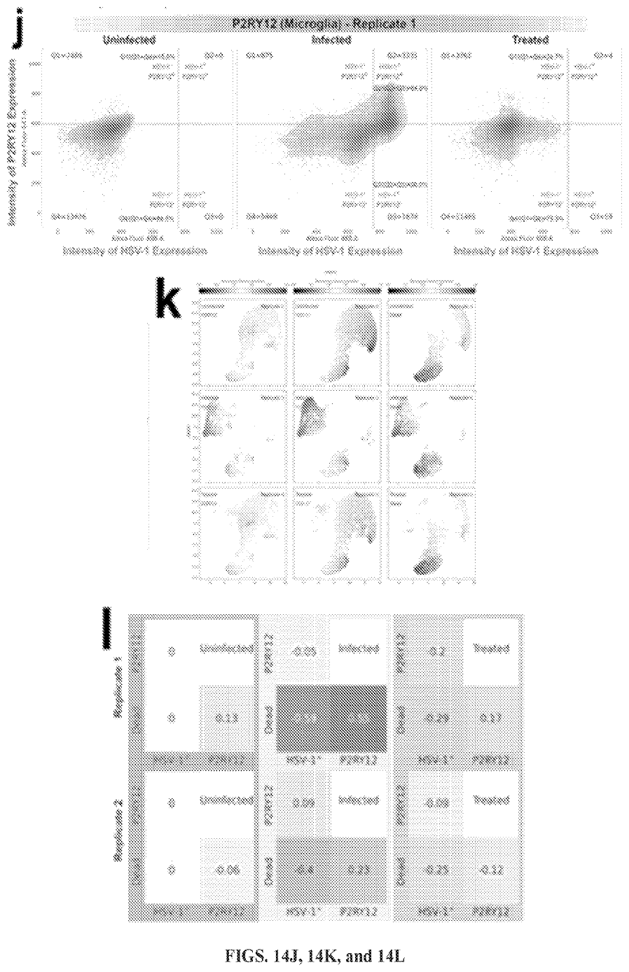

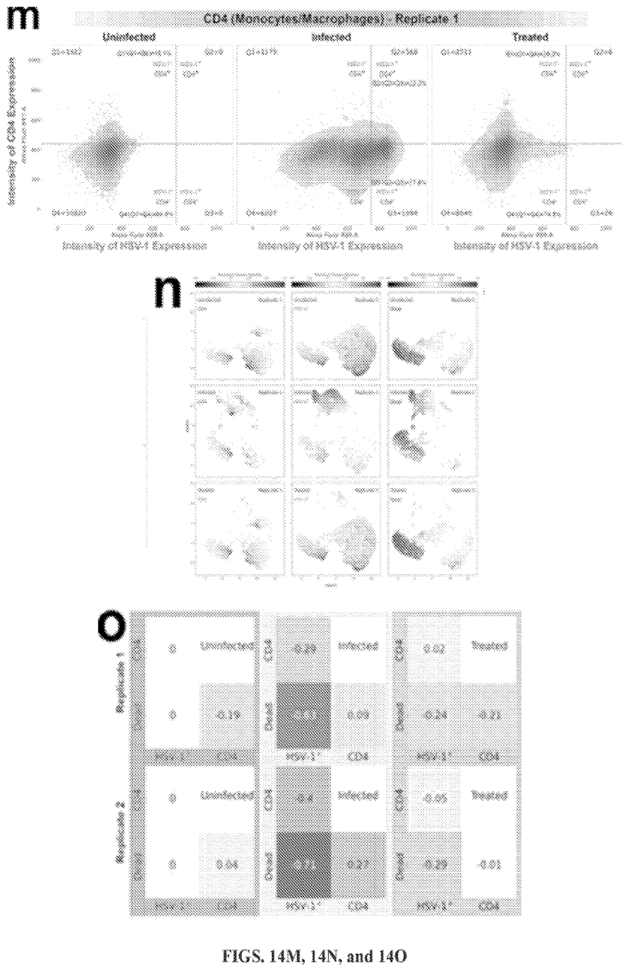

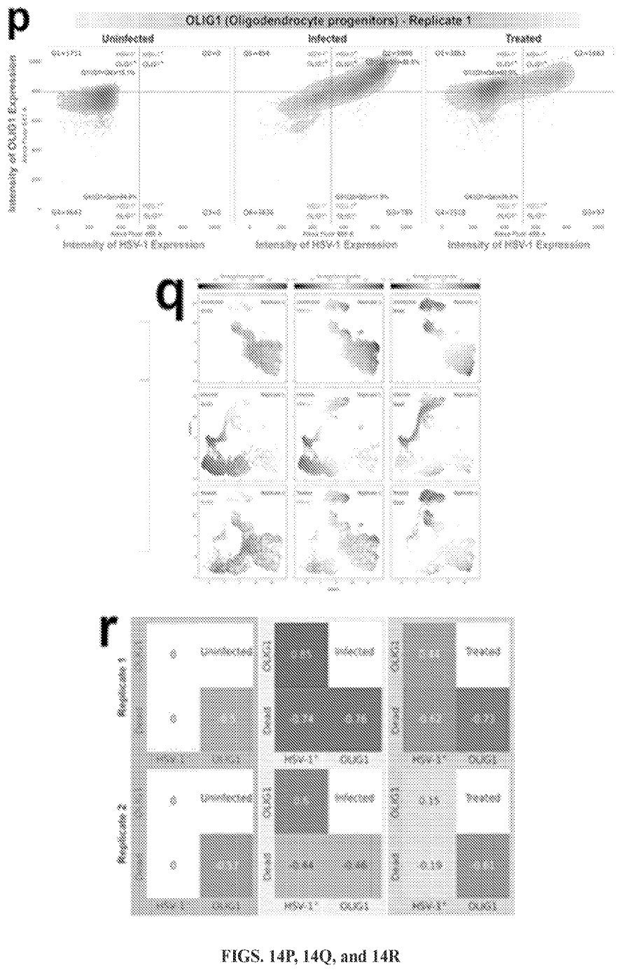

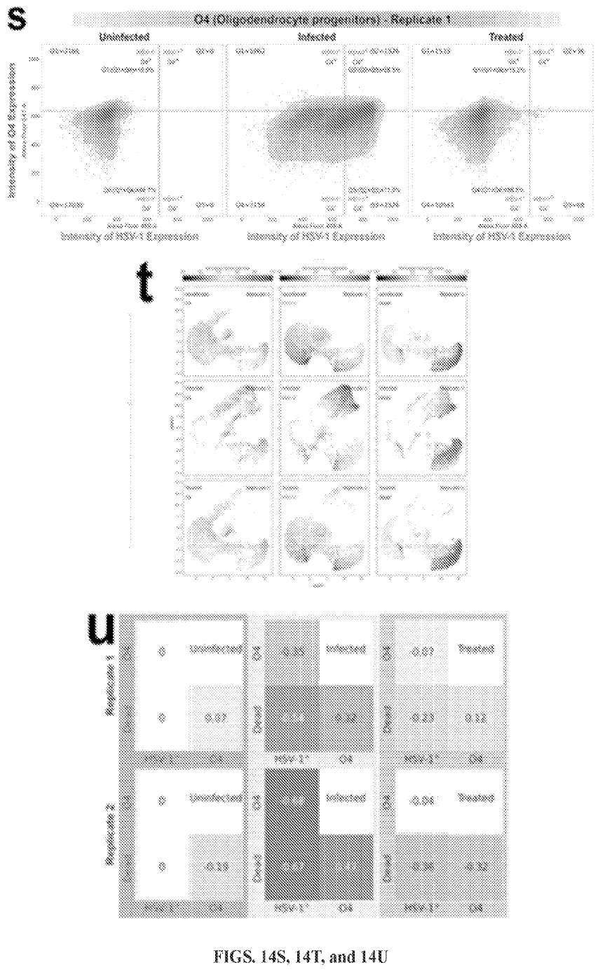

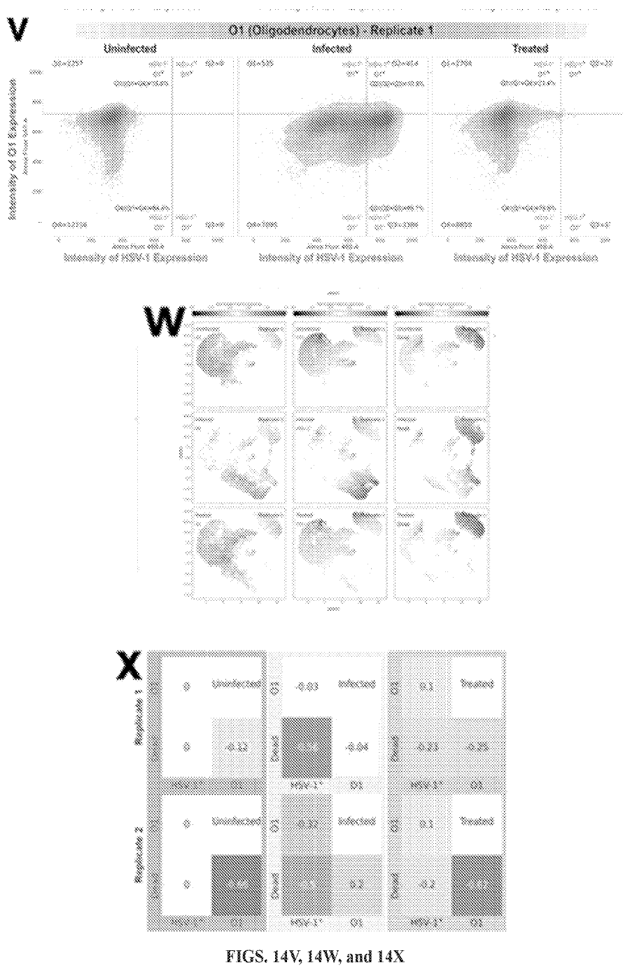

(Treated_Organoids4-6 versus Uninfected_Organoids4-6), with the log 2 fold change calculated as the expression of human transcripts in ACV-treated cOrgs over uninfected cOrgs. FIG. 7I shows the ACV-vs-UV cOrgs2 (Treated_Organoids4-6 versus UV-HSV-1_Organoids1-3), with the log₂ fold change calculated as the expression of human transcripts in ACV-treated cOrgs over cOrgs with UV-inactivated virus. FIG. 7J shows the Inf-vs-ACV hiPSCs

(Infected_hiPSCs1-3 versus Treated_hiPSCs1-3), with the log 2 fold change calculated as the expression of human transcripts in infected hiPSCs over ACV-treated hiPSCs. FIG. 7K shows the Inf-vs-ACV cOrgs1 (Infected_Organoids4-6 versus Treated_Organoids1-3), with the Log 2 fold change (or Log 2FC) calculated as the expression of HSV-1 transcripts in infected cOrgs over ACV-treated cOrgs. FIG. 7L shows the Inf-vs-ACV cOrgs2

(Infected_Organoids7-9 versus Treated_Organoids4-6), with the Log 2 fold change (or Log 2FC) calculated as the expression of HSV-1 transcripts in infected cOrgs over ACV-treated cOrgs. FIG. 7M shows the Inf-vs-ACV hiPSCs (Infected hiPSCs1-3 versus Treated hiPSCs1-3), with the Log 2 fold change (or Log 2FC) calculated as the expression of HSV-1 transcripts in infected hiPSCs over ACV-treated hiPSCs. FIG. 7N shows the ratios of human transcripts that were up-regulated with positive log₂ fold changes versus down-regulated with negative log₂ fold changes across the datasets. The ratios for the infected cOrgs versus uninfected control cOrgs or cOrgs with UV-inactivated virus, are tinted in pink. The ratios for the comparisons with ACV-treated cOrgs are tinted in blue. The ratios for the hiPSCs are not tinted (in white). −log₁₀ P-values in all plots were calculated using the adjusted P-values from the differential expression.

FIGS. 8A, 8B, 8C, and 8D show the negative control experiments in hiPSCs, IAV and SH-SY5Y-differentiated neurons. FIG. 8A shows the heatmap depicting the expression of human transcripts in uninfected hiPSCs (Ctrl_hiPSCs1-3) and HSV-1 infected hiPSCs (Inf_hiPSCs1-3). FIG. 8B shows the heatmap depicting the expression of human transcripts in HSV-1 infected hiPSCs (hiPSCs1-3) and ACV-treated hiPSCs (ACV_hiPSCs1-3). FIG. 8C shows the flow cytometry plots showing that there are 35.2% of IAV-infected cOrg cells using a protocol and there are similar percentages of dead cOrg cells among the uninfected sample (33.3%) and infected sample (37.5%). FIG. 8D shows the gene set enrichment plot including HSV-1 infected versus uninfected (Inf-vs-Ctrl) SH-SY5Y-differentiated neurons. −log₁₀ Nominal P-value in the plot was calculated using the nominal P-value from Gene Set Enrichment Analysis (GSEA). The diseases/traits are: Alzheimer's disease (AD), amyotrophic lateral sclerosis (ALS), multiple sclerosis (MS), Parkinson's disease (PD), attention-deficit/hyperactivity disorder (ADHD), autism spectrum disorders (ASD), bipolar disorder (BD), major depressive disorder (MDD), obsessive-compulsive disorder (OCD), schizophrenia (SCZ), Tourette syndrome (TS), Crohn's disease (CD), inflammatory bowel disease (IBD), psoriasis (PSO), rheumatoid arthritis (RA), Type 1 diabetes (TID), Type 2 diabetes (T2D), body mass index (BMI), functional MRI measures of brain anatomy (BRAIN), height (HEIGHT) and waist-to-hip ratio (WHR).

FIGS. 9A, 9B, 9C, 9D, and 9E show the HSV-1 differential viral gene expression in ACV-vs-Inf samples. FIG. 9A shows the UpSet plot for the ranks of the adjusted P-values for differential expression of leaky late genes (γ₁), with the red points indicating the ranks in the discovery dataset (cOrgs1) and the blue points indicating the ranks in the replication dataset (cOrgs2). FIG. 9B shows the UpSet plot for the ranks of the adjusted P-values for differential expression of true late genes (12), with the red points indicating the ranks in the discovery dataset (cOrgs1) and the blue points indicating the ranks in the replication dataset (cOrgs2). FIG. 9C shows the differential viral transcript expression for leaky late genes and true late genes comparing ACV-treated cOrgs and hiPSCs with HSV-1 infected cOrgs and hiPSCs, with the sizes of the bubbles representing-logo (adjusted P-values) and the colors of the bubbles representing the log 2 (Fold Change) or log 2FC of each viral gene. FIG. 9D shows the histogram and density plots of the differential viral transcript expression for leaky late genes. FIG. 9E shows the histogram and density plots of the differential viral transcript expression for true late genes.

FIGS. 10A, 10B, 10C, 10D, 10E, 10F, 10G, 10H, 10I, 10J, 10K, 10L, 10M, 10N, 100, 10P, 10Q, 10R, 10S, and 10T show the venn diagrams representing the numbers of GWAS-associated genes that were rescued or not rescued across both datasets. Venn diagrams showing the numbers of genes that were found to be rescued or not rescued in only cOrgs1, only cOrgs2 or in both cOrgs1 and cOrgs2, for each set of disease-associated genes identified from GWAS. The legends show the color scale in increasing numbers of genes (blue to yellow). Odds ratios (ORs) and P-values were calculated using 2-tailed Fisher's Exact Test to evaluate if the number of transcripts that were rescued in both cOrgs was enriched for the disease compared to the overall Venn diagram. ORs with P<0.1 were shown on the diagrams.

FIGS. 11A, 11B, 11C, 11D, 11E, 11F, 11G, 11H, 11I, 11J, 11K, 11L, 11M, 11N, 110, 11P, 11Q, 11R, 11S, and 11T show the venn diagrams representing the numbers of GWAS-associated genes that were exacerbated across both datasets. Venn diagrams showing the numbers of genes that were found to be in only the Exacerbated1 category (significantly opposite fold changes in Inf-vs-Ctrl cOrgs versus Inf-vs-ACV cOrgs) or only the Exacerbated2 category (adjusted P>0.05 in Inf-vs-Ctrl cOrgs and adjusted P≤0.05 in Inf-vs-ACV cOrgs), or both Exacerbated1 and Exacerbated2. The legends show the color scale in increasing numbers of genes (blue to yellow). FBRAIN results are missing from this figure because there are no genes in common with the exacerbated genes.

FIGS. 12A and 12B show the AB ELISA assays using conditioned media that were UV-inactivated. FIG. 12A shows the concentrations of Aβ42/40/38 in pg/mL, Aβ42/40 ratios and Aβ42/38 ratios detected from conditioned media that had undergone UV-inactivation for uninfected (control) cOrg cells, HSV-1 infected (HSV-1+) cOrg cells, HSV-1 infected and ACV-treated (Treated) cOrg cells, and cOrg cells with UV-inactivated HSV-1 (UV-HSV-1). FIG. 12B shows the concentrations of Aβ42/40/38 in pg/mL, Aβ42/40 ratios and Aβ42/38 ratios detected from conditioned media that had undergone UV-inactivation for uninfected (control) cOrg cells and IAV infected (IAV+) cOrg cells. P-values shown were calculated using 1-sided Wilcoxon ranked sum test with comparison to control samples.

FIGS. 13A, 13B, 13C, 13D, 13E, 13F, 13G, 13H, 13I, 13J, 13K, 13L, 13M, 13N, and 130 show the flow cytometry results for stem cells, neural progenitors and neurons. FIG. 13A shows the gated 2-channel intensity plots of the first replicate experiments using TRA-1-60 for stem cells, with Alexa647/APC (TRA-1-60) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for TRA-1-60 are shown as Q1/(Q1+Q4) or Q2/(Q2+Q3). FIG. 13B shows the UMAP plot of the first replicate experiments using TRA-1-60 (red: Alexa647/APC, green: HSV-1, blue: Violet Zombie) for uninfected, HSV-1 infected and ACV-treated cOrgs flow cytometry results. FIG. 13C shows the correlations in the intensities of HSV-1, TRA-1-60 and Zombie (dead) marker for both replicates (top and bottom) across uninfected (green background), HSV-1 infected (yellow background) and ACV-treated (pink background) cOrg cells. FIG. 13D shows the gated2-channel intensity plots of the first replicate experiments using Nestin for neural progenitor cells, with Alexa647/APC (Nestin) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for Nestin are shown as Q1/(Q1+Q4) or Q2/(Q2+Q3). FIG. 13E shows the UMAP plot of the first replicate experiments using Nestin (red: Alexa647/APC, green: HSV-1, blue: Violet Zombie) for uninfected, HSV-1 infected and ACV-treated cOrgs flow cytometry results. FIG. 13F shows the correlations in the intensities of HSV-1, Nestin and Zombie (dead) marker for both replicates (top and bottom) across uninfected (green background), HSV-1 infected (yellow background) and ACV-treated (pink background) cOrg cells. FIG. 13G shows the gated 2-channel intensity plots of the first replicate experiments using EOMES for intermediate progenitor cells, with Alexa647/APC (EOMES) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for EOMES are shown as Q1/(Q1+Q4) or Q2/(Q2+Q3). FIG. 13H shows the UMAP plot of the first replicate experiments using EOMES (red: Alexa647/APC, green: HSV-1, blue: Violet Zombie) for uninfected, HSV-1 infected and ACV-treated cOrgs flow cytometry results. FIG. 13I shows the correlations in the intensities of HSV-1, EOMES and Zombie (dead) marker for both replicates (top and bottom) across uninfected (green background), HSV-1 infected (yellow background) and ACV-treated (pink background) cOrg cells. FIG. 13J shows the gated 2-channel intensity plots of the first replicate experiments using TuJ1 for immature neurons, with Alexa647/APC (TuJ1) intensities on the y-axis and GFP (HSV-1) intensities on the x-axis) across uninfected, HSV-1 infected and ACV-treated cOrg cells. The numbers of cells for each quadrant (Q1-Q4) are shown in each plot; the percentages of cells positive for TuJ1 are shown as Q1/(Q1+Q4) or Q2/(Q2+Q3). FIG. 13K shows the UMAP plot of the first replicate experiments using TuJ1 (red: Alexa647/APC, green: HSV-1, blue: Violet Zombie) for uninfected, HSV-1 infected and ACV-treated cOrgs flow cytometry results. FIG. 13L shows correlations in the intensities of HSV-1, TuJ1 and Zombie (dead) marker for both replicates (top and bottom) across uninfected (green background), HSV-1 infected (yellow background) and ACV-treated (pink background) cOrg cells. FIG. 13M shows the gated 2-channel intensity plots of the first replicate experiments using NeuN for neurons, with Alexa647/APC (NeuN) intensities on the y-axis and GFP (HSV-1) intensities on the x-axis) across uninfected, HSV-1 infected and ACV-treated cOrg cells. The numbers of cells for each quadrant (Q1-Q4) are shown in each plot; the percentages of cells positive for NeuN are shown as Q1/(Q1+Q4) or Q2/(Q2+Q3). FIG. 13N shows the UMAP plot of the first replicate experiments using NeuN (red: Alexa647/APC, green: HSV-1, blue: Violet Zombie) for uninfected, HSV-1 infected and ACV-treated cOrgs flow cytometry results. FIG. 130 shows the correlations in the intensities of HSV-1, NeuN and Zombie (dead) marker for both replicates (top and bottom) across uninfected (green background), HSV-1 infected (yellow background) and ACV-treated (pink background) cOrg cells.

FIGS. 14A, 14B, 14C, 14D, 14E, 14F, 14G, 14H, 14I, 14J, 14K, 14L, 14M, 14N, 140, 14P, 14Q, 14R, 14S, 14T, 14U, 14V, 14W, and 14X shows the flow cytometry results for glia cells and glia progenitors. FIG. 14A shows the gated 2-channel intensity plots of the first replicate experiments using GFAP for astrocytes, with Alexa647/APC (GFAP) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for GFAP are shown as Q1/(Q1+Q4) or Q2/(Q2+Q3). FIG. 14B shows the UMAP plot of the first replicate experiments using GFAP (red: Alexa647/APC, green: HSV-1, blue: Violet Zombie) for uninfected, HSV-1 infected and ACV-treated cOrgs flow cytometry results. FIG. 14C shows the correlations in the intensities of HSV-1, GFAP and Zombie (dead) marker for both replicates (top and bottom) across uninfected (green background), HSV-1 infected (yellow background) and ACV-treated (pink background) cOrg cells. FIG. 14D shows the gated 2-channel intensity plots of the first replicate experiments using GLAST for astrocytes, with Alexa647/APC (GLAST) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for GLAST are shown as Q1/(Q1+Q4) or Q2/(Q2+Q3). FIG. 14E shows the UMAP plot of the first replicate experiments using GLAST (red: Alexa647/APC, green: HSV-1, blue: Violet Zombie) for uninfected, HSV-1 infected and ACV-treated cOrgs flow cytometry results. FIG. 14F shows the correlations in the intensities of HSV-1, GLAST and Zombie (dead) marker for both replicates (top and bottom) across uninfected (green background), HSV-1 infected (yellow background) and ACV-treated (pink background) cOrg cells. FIG. 14G shows the gated 2-channel intensity plots of the first replicate experiments using Iba1 for microglia, with Alexa647/APC (Iba1) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for Iba1 are shown as Q1/(Q1+Q4) or Q2/(Q2+Q3). FIG. 14H shows the UMAP plot of the first replicate experiments using Iba1 (red: Alexa647/APC, green: HSV-1, blue: Violet Zombie) for uninfected, HSV-1 infected and ACV-treated cOrgs flow cytometry results. FIG. 14I shows the correlations in the intensities of HSV-1, Iba1 and Zombie (dead) marker for both replicates (top and bottom) across uninfected (green background), HSV-1 infected (yellow background) and ACV-treated (pink background) cOrg cells. FIG. 14J shows the gated 2-channel intensity plots of the first replicate experiments using P2RY12 for microglia, with Alexa647/APC (P2RY12) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for P2RY12 are shown as Q1/(Q1+Q4) or Q2/(Q2+Q3). FIG. 14K shows the UMAP plot of the first replicate experiments using P2RY12 (red: Alexa647/APC, green: HSV-1, blue: Violet Zombie) for uninfected, HSV-1 infected and ACV-treated cOrgs flow cytometry results. FIG. 14L shows the correlations in the intensities of HSV-1, P2RY12 and Zombie (dead) marker for both replicates (top and bottom) across uninfected (green background), HSV-1 infected (yellow background) and ACV-treated (pink background) cOrg cells. FIG. 14M shows the gated 2-channel intensity plots of the first replicate experiments using CD4 for monocytes/macrophages, with Alexa647/APC (CD4) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for CD4 are shown as Q1/(Q1+Q4) or Q2/(Q2+Q3). FIG. 14N shows the UMAP plot of the first replicate experiments using CD4 (red: Alexa647/APC, green: HSV-1, blue: Violet Zombie) for uninfected, HSV-1 infected and ACV-treated cOrgs flow cytometry results. FIG. 14O shows the correlations in the intensities of HSV-1, CD4 and Zombie (dead) marker for both replicates (top and bottom) across uninfected (green background), HSV-1 infected (yellow background) and ACV-treated (pink background) cOrg cells. FIG. 14P shows the gated 2-channel intensity plots of the first replicate experiments using OLIG1 for oligodendrocyte progenitor cells, with Alexa647/APC (OLIG1) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for OLIG1 are shown as Q1/(Q1+Q4) or Q2/(Q2+Q3). FIG. 14Q shows the UMAP plot of the first replicate experiments using OLIG1 (red: Alexa647/APC, green: HSV-1, blue: Violet Zombie) for uninfected, HSV-1 infected and ACV-treated cOrgs flow cytometry results. FIG. 14R shows the correlations in the intensities of HSV-1, OLIG1 and Zombie (dead) marker for both replicates (top and bottom) across uninfected (green background), HSV-1 infected (yellow background) and ACV-treated (pink background) cOrg cells. FIG. 14S shows the gated 2-channel intensity plots of the first replicate experiments using 04 for oligodendrocyte progenitor cells, with Alexa647/APC (04) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for 04 are shown as Q1/(Q1+Q4) or Q2/(Q2+Q3). FIG. 14T shows the UMAP plot of the first replicate experiments using 04 (red: Alexa647/APC, green: HSV-1, blue: Violet Zombie) for uninfected, HSV-1 infected and ACV-treated cOrgs flow cytometry results. FIG. 14U shows the correlations in the intensities of HSV-1, 04 and Zombie (dead) marker for both replicates (top and bottom) across uninfected (green background), HSV-1 infected (yellow background) and ACV-treated (pink background) cOrg cells. FIG. 14V shows the gated 2-channel intensity plots of the first replicate experiments using O1 for oligodendrocytes, with Alexa647/APC (01) intensities on the y-axis and GFP (HSV-1) intensities on the x-axis) across uninfected, HSV-1 infected and ACV-treated cOrg cells. The numbers of cells for each quadrant (Q1−Q4) are shown in each plot; the percentages of cells positive for 041 are shown as Q1/(Q1+Q4) or Q2/(Q2+Q3). FIG. 14W shows the UMAP plot of the first replicate experiments using 01 (red: Alexa647/APC, green: HSV-1, blue: Violet Zombie) for uninfected, HSV-1 infected and ACV-treated cOrgs flow cytometry results. FIG. 14X shows the correlations in the intensities of HSV-1, 01 and Zombie (dead) marker for both replicates (top and bottom) across uninfected (green background), HSV-1 infected (yellow background) and ACV-treated (pink background) cOrg cells.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### TERMINOLOGY

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of neurodegeneration), during early onset (e.g., upon initial signs and symptoms of neurological disorders), or after an established development of the neurological disorder.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

As used herein, “diagnose”, “diagnosed”, “diagnosing”, and any grammatical variations thereof as used herein, refers to the act of process of identifying the nature of an illness, disease, disorder, or condition in a subject by examination or monitoring of symptoms.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

As used herein, the term “polymerase” refers to an enzyme that synthesizes long chains of polymers or nucleic acids. DNA polymerase and RNA polymerase are used to assemble DNA and RNA molecules, respectively, by copying a DNA template strand using base-pairing interactions.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing of physical phenomenon. An event or change in environment is sensed and signal output released in the form of light, color change, heat, or a combination thereof.

A “primer” is a short polynucleotide, generally with a free 3′—OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

The term “mRNA” refers to messenger ribonucleic acid, or single stranded molecule of RNA that corresponds to the genetic sequence of a gene, and is translated by a ribosome in the process of synthesizing a protein. mRNA is created during the process of transcription, where a gene is converted into a primary transcript mRNA (or pre-mRNA). The primary transcript is further processed through RNA splicing to only contain regions that will encode protein. mRNA can also be targeted for epigenetic modifications, such as methylation, to impact mRNA translation, nuclear retention, nuclear export, processing, and splicing.

A “neurodegenerative disease” is caused, for example, by the progressive loss of structure or function of neurons or glial cells, which make up the nervous system. These diseases include but are not limited to amyotrophic lateral sclerosis (ALS), multiple sclerosis, Parkinson's disease, Alzheimer's disease, Huntington's disease, and prion diseases. Neurodegenerative diseases can lead to cognitive and physical impairments, neuroinflammation (inflammation of the brain and spinal cord), and deterioration of brain and spinal cord tissues.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

### Methods

The present disclosure provides methods of treating, preventing, decreasing, reducing, detecting, and/or diagnosing a neurological disorder.

In one aspect, disclosed herein is a method of treating a neurological disorder in a subject in need thereof, the method comprising collecting a biological sample from the subject, detecting a gene expression signature in a nucleic acid isolated from the biological sample, and administering a therapeutically effective amount of an anti-viral agent to the subject with the gene expression signature, wherein the gene expression signature comprises at least one gene affected by the neurological disorder and a herpes simplex virus 1 (HSV-1).

In some embodiments, the at least one gene is selected from a first gene set comprising AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, or YAP1.

In some embodiments, the at least one gene is selected from a second gene set comprising ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, or WNT7B.

In some embodiments, the first gene set is combined with the second gene set. In some embodiments, the at least one gene comprises AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, YAP1, ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, WNT7B, or a combination thereof.

In some embodiments, the method detects an increase or decrease in expression of AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, YAP1, ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, WNT7B, or a combination thereof, relative to a healthy control subject.

In some embodiments, the neurological disorder comprises Alzheimer's disease (AD), dementia, multiple sclerosis (MS), Parkinson's disease, Huntington'disease, obsessive compulsive disorder (OCD), attention-deficit/hyperactivity disorder (ADHD), schizophrenia, or amyotrophic lateral sclerosis (ALS). In some embodiments, the neurological disorder comprises Alzheimer's disease (AD).

In some embodiments, the anti-viral agent comprises acyclovir, valacyclovir, or variants thereof. In some embodiments, the anti-viral agent is administered with a therapeutic agent. In some embodiments, the therapeutic agent comprises a neurological therapeutic agent. In some embodiments, the neurological therapeutic agent comprises levetiracetam, gabapentin, topiramate, lamotrigine, carbidopa, levodopa, donepezil HCl, sumatriptan succinate, oxcarbazepine, amitriptyline HCl, memantine HCl, divalprox sodium, pregabalin, pramipexole, ropinirole, rotigotine, clozapine, quetiapine, olanzapine, galantamine, rivastigmine, trifluoperazine, methylphenidate, atomoxetine, amphetamine, dextroamphetamine, pemoline, perphenazine, aducanumab, lecanemab, suvorexant, brexpiprazole, or a combination thereof.

In some embodiments, the anti-viral agent comprises acyclovir. In some embodiments, the anti-viral agent comprises valacyclovir.

In some embodiments, the method decreases amyloid-β (Aβ-42) or phosphorylated Tau (pTau-212) concentrations in the subject relative to an untreated subject with the neurological disorder. In some embodiments, the method prevents progression of the neurological disorder.

In some embodiments, the biological sample comprises a cerebrospinal fluid (CSF), blood, serum, or tissue biopsy. In some embodiments, the nucleic acid comprises an RNA molecule.

In some embodiments, the neurological disorder is a neurodegenerative disease. As used herein, the term “neurodegenerative disease” refers to a varied assortment of central nervous system disorders characterized by gradual and progressive loss of neural tissue and/or neural tissue function. A neurodegenerative disease is a class of neurological disorder or disease, and where the neurological disease is characterized by a gradual and progressive loss of neural tissue, and/or altered neurological function, typically reduced neurological function as a result of a gradual and progressive loss of neural tissue. Examples of neurodegenerative diseases include for example, but are not limited to, Alzheimer's disease (AD), Parkinson's disease (PD), Huntington's Disease, Amyotrophic Lateral Sclerosis (ALS, also termed Lou Gehrig's disease) and Multiple Sclerosis (MS), polyglutamine expansion disorders (e.g., HD, dentatorubropallidoluysian atrophy, Kennedy's disease (also referred to as spinobulbar muscular atrophy), spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple system atrophy, Pelizaeus-Merzbacher disease, Pick's disease, primary lateral sclerosis, Refsum's disease, Sandhoff disease, Schilder's disease, spinal cord injury, spinal muscular atrophy (SMA), Steele-Richardson-Olszewski disease, Tabes dorsalis, and the like. In some embodiments, the neurodegenerative disease is Parkinson's disease.

In some aspects, disclosed here is a method of a neurological disorder in a subject in need thereof, the method comprising collecting a biological sample from the subject, detecting a gene expression signature in a nucleic acid isolated from the biological sample, and administering a therapeutically effective amount of an anti-viral agent to the subject with the gene expression signature, wherein the gene expression signature comprises a change in expression of one or more genes affected by Alzheimer's disease (AD), Attention-deficit

Hyperactivity Disorder (ADHD), Amyotrophic Lateral Sclerosis (ALS), Autism Spectrum Disorder (ASD), Bipolar Disorder (BD), Body-Mass Index (BMI), Inflammatory bowel disease (IBD), Major Depressive disorder (MDD), Multiple Sclerosis (MS), Obsessive compulsive disorder (OCD), Parkinson's disease (PD), Progressive supranuclear palsy (PSP), Rheumatoid arthritis (RA), schizophrenia (SCZ), Tourette syndrome (TS), Type I Diabetes mellitus, Type II Diabetes mellitus, WHR, additional brain disorders, or disorders affecting height.

In some embodiments, the one or more genes affected by AD comprise FBXL7, CACNA2D3, TOMM40, HBEGF, PFDN1, ECHDC3, USP6NL-AS1, TSPOAP1-AS1, NECTIN2, APOC1, APOE, NFIC, BCAM, APOCIP1, CBLC, KIR3DL2, CEACAM16-AS1, BCL3, RNU6-560P, LINC02695, NIFKP9, BIN1, CR1, APOC4, MARK4, PPPIR37, TRAPPC6A, LINC02705, MS4A2, CD33, SIGLEC22P, ANKRD31, SORL1, ARL17B, CLU, EPHA1-AS1, SLC24A4, MEF2C-AS1, ZCWPW1, GPR141, EPDR1, CELF1, HLA-DRB1, HLA-DQA1, PTK2B, CD2AP, ABCA7, NDUFAF6, SCIMP, ZNF594-DT, CASS4, B3GNTL1P2, CD2AP-DT, INPP5D, MS4A6A, PILRA, LINC02360, DSG2, MIR4432HG, LINGO2, MIR7154, CHST1, CATSPERB, ATP5PF, GRIN2B, C12orf40, SLC2A13, LINC02098, PCNX1, MPZL1, DSTNP5, FRMD4B, LMOD3, ATXN7L1, MAILR, SLC25A6P5, LUZP2, CDON, FOXN3, NDUFA12, HPRT1P2, MS4A3, FANCD2OS, FANCD2, TREM2, CELF2, AIDAP3, CNTNAP2, SUDS3, DAPL1, PDE1A, CADM2, FBXO40, LINC00290, LINC02500, OFCC1, PEX6, PICALM, SORD, LINC01684, KCNN3, HSPA8P9, CLDN18, RN7SL193P, IL21-AS1, SERINC5, KRT18P45, CDC42SE2, RAPGEF6, FNIP1, MEIKIN, ACSL6, SH2D4B, MS4A4A, LINC02210-CRHR1, WNT3, CDR2L, HID1-AS1, PPIAP17, PAX2, LINC01725, VDACIP12, PCDH11X, LRAT, GAβ2, EDAR, MTHFDIL, SLC4A1AP, HAS2-AS1, PROX1-AS1, ZFYVE9P2, RBMS3, POLN, HAUS3, STK24, ATP8A2P3, RNF6, SCARA3, C5orf64, MMP12, MMP3, DHFRP3, PPP1R3B, RN7SKP199, NFUIP1, MAPREIP2, NRXN3, TLN2, HIGDIAP3, SUMO2P6, ZNF225, ZNF224, PCEDIB, PPIAP45, TSNAX-DISC1, DISC1, FRMD4A, PRRC2C, FMN2, CTNNA2, LIMS2, MOBP, STK32B, AFF1, ANKRD55, PGAM5P1, CAMK4, DMXL1, MEGF10, LINC01184, SAP30L, NKAIN2, PDE7B, PLEKHG1, BZW2, CYCS, ELMO1, EXOC4, CSMD1, ST18, NCS1, PLPP4, SPON1, ARHGAP20, SLC4A8, CRADD, ANO4, GPC6, MYO16, CLMN, GABRG3, LIPC-AS1, LIPC, ALDH1A2, LINC01567, TNRC6A, VATIL, SP6, CACNAIG, BCAS3, PPIAP59, TGM6, PARVB, HECW1, FERMT2, SQSTM1, TREML2, AP2A2, ADAMTS20, EEF1A1P17, IGHV3-71, IGHV2-70, SPPL2A, TRIP4, RELN, LINCR-0001, BMPER, SLC8A1-AS1, MRC1, APOC4-APOC2, PLCG2, RN7SL354P, APP-DT, APP, USP50, PLEC, IL34, IGHG1, IGHEP1, CHRNE, C17orf107, MROHI, SPATCI, PRKD3, RN7SL553P, VRK3, RN7SL299P, WDR70, HNRNPKP3, ACER3, TSKU, RERGL, PIK3C2G, LINC02254, LINC02152, ROBO2P1, EPOP, SRCIN1, RPL13AP7, RNA5SP489, CENPM, LINC00634, Y_RNA, TNXB, LHX1-DT, SFT2D2, MIR155HG, MRPL39, SERPINE1, TRIM56, CCDCl71, C9orf92, OR7E109P, DIRAS2, MAPK7, FBXL13, ANTXR1, PAX5, RPL32P21, LNC-LBCS, LINC02414, TMCC1-DT, TRH, MIR302F, MAP4K4, SCARB2, FAM47E, BANK1, LINC00899, WNT7B, MTSS2, CDCP1, CLEC3B, EFL1, NTM, OR2B2, OR2W6P, CIS, TSPAN14, RPSAP24, LINC01960, MED12L, CDHR3, SYPL1, DVL2, MGME1, OVOL2, ZAP70, PUS1, ABCA1, MAP2K5, ANGPT4, ARIH1, LINC01098, LINC01099, MSX2, BCL10, DDAH1, FAM83E, PER3P1, RNU4ATAC8P, C4BPAP2, CD55, SALL4P1, GPBP1, CNTNAP5, TENT5A, IL19, NCKAP5, RN7SKP93, CCDC85C, SDR9C7, NARS2, ALDH4A1, PKNOX2, CRY2, SLC35C1, OR4C3, OR4S1, TCF7L2, MADD, CLPTM1, APOB, LIPG, UBE2L3, PVR, EPHA1, SCARB1, LILRA5, MIR4752, AGFG2, AP4M1, CASTOR3, STAG3L5P, STAG3L5P-PVRIG2P-PILRB, CHRNA2, EPHX2, GULOP, PSMC3, RAPSN, SPI1, ADAM10, SNORD3P1, BCKDK, ZNF232, PPIAP55, CYB561, CNN2, CEACAM20, CEACAM19, CEACAM22P, RELB, EXOC3L2, CSTF1, ZNF652, PHB, NKPD1, BLOC1S3, CLASRP, GEMIN7-AS1, IGSF23, HLA-DRA, HLA-DRB9, GEMIN7, STAG3, RPS16P9, CKM, HESX1, IL17RD, UNC5CL, LRFN2, ALPK2, MEIOSIN, APHIB, ADAMTS4, KAT8, APOC2, MS4A6E, CRIL, ADGRF2, ZFP3, ACE, TRIM51CP, GTF2IP11, AHNAK, CCDC89, LINC02763, LINC02762, DDX25, HSPA8P5, GAU1, NDUFA9, AICDA, OVCH1-AS1, PA2G4, ERBB3, SNORA70, RNU7-106P, OTOGL, BRWD1P2, RNU6-148P, TMEM132C, MRPS31P4, LINC00345, RABEPKP1, ARF4P4, LINC00377, FARP1, GGACT, LINC00343, FGF10, F2R, F2RL1, SGK1, SNX9, TULP4, PHF14, LINCO2214, RPS17P2, ZCCHC10, RNU7-156P, LARS1, G3BP1, HAND1, CIR1P1, TMED9, FAM193B-DT, CADPS2, IQUB, ZNF862, INSIG1, EN2, BLACE, NKAIN3, COL12A1, FZD6, CTHRC1, LINC01151, SMILR, LINC00861, PDCDILG2, MTATP6P30, ATP5MFP3, RFC5P1, LINC00476, FOXE1, TRMO, MTND3P4, PNPLA7, WAC, JCAD, RN7SL825P, RPL34P19, C10orf71, MRPL50P4, SLC16A9, LINC02651, RPL5P26, LINC02653, HTR7, SORCS3, LINC02752, ZBED5-AS1, MICAL2, LINC02699, HSD17B12, MIR129-2, LINC02518, EYA4, WDR41, FILNC1, KCNN2, NPM1P11, CHN2, NEUROD6, MTDHP1, ZNF117, EEF1A1P28, PQLC1P1, DLX5, AP1S2P1, KDMIB, CDKAL1, CDC5L, DST, KHDRBS2-OT1, C8orf44-SGK3, SGK3, PPPIR42, LINC02532, SOBP, SCML4, L3MBTL4, LAMA1, NFE2L3P1, PMAIP1, RAB3D, TSPAN16, LINC01801, ZNF599, ZNF813, CST8, CST13P, CSTP2, CST1, RPS2P1, ASIP, LINC01271, RN7SL636P, U6, SYNJ1, PDE9A, LINC01671, LINC01679, SIK1, COL18A1, SEPTIN5, TEX33, TST, LINC01117, OSBPL6, F13A1, RNU6-261P, CCDC88C, PPP4R3A, FAM181A-AS1, FAM181A, RPL15P2, LINC02325, HERC2, TRPM1, THSD4, AGBL1, PDPK1, PDPK2P, TMC5, SDR42E2, NCOA5LP, RPL34P29, CDH13, FENDRR, LINC00917, ALOX12-AS1, COX10-AS1, MPP3, CD300LG, DCAF7, STRADA, TCAMIP, ABCA8, RPL12P37, RPTOR, LAMP1, RBFOX1, ARVCF, HSPG2, UBXN11, RPS29P6, RNA5SP43, HYI, SZT2, PLPPR4, AHCYL1, NMNAT1P2, TGFB2, RNU4-77P, RNU6-649P, ITSN2, OTOF, MSH2, PPPIR21-DT, FOXN2, CCDCl2P1, LINC01185, RABIA, SNORA74, SPRED2, LINC01868, LINC01965, DDX18, RN7SKP141, CTAGE14P, B3GALT1-AS1, LRRC2P1, FAM228B, ZNF804A, COL4A4, RANP7, SALL4P5, HMGB3P12, CFAP20DC, PTPRG, LINC00877, LINC02008, INHCAP, RNU6-637P, TERC, SNRPCP13, ENPP7P11, GABRA2, RAC1P2, NDUFS5P4, RNU7-149P, SETD7, RN7SL152P, TBC1D9, NR3C2, ANXA10, SH3RF1, LINC02268, MRPS36P2, FAT1, RNU6-381P, RNU6-362P, MRPL58, PAK2, SPATA48, ADCY8, CTAGE12P, OTX2P1, LINC00423, PDS5B, OBI1-AS1, RORA-AS1, RORA, SLC44A5, NME9, RNU6-952P, YAP1, ZNF292, CDC42EP3, UTS2B, IRF6, RBBP4P4, GAPDHP15, PMS2CL, TMEM106B, EPC2, RNU2-9P, CCDCl34, GMNC, GLIS3, NCR2, HMGB3P23, MTCO3P30, SLC2A9, RNU1-80P, TNRC6C, VSNL1, NEPRO, LINC02044, RIMBP2, MPP7, MFSD4BP1, RPL3P11, ATP5PDP3, H3P28, ARID1B, CASC18, CCDC66, RN7SL782P, RN7SKP122, ADAMTS9-AS2, DCHS2, PUM3, HRK, SLC28A1, LINC01016, MLN, OACYLP, SEC11C, RBM19, DYNLL1P4, ACMSD, CCNT2-AS1, DOCK4, DOC2A, ADAMTS1, NCK2, TREML5P, OARD1, AGT, RPL21P119, ERBB4, FAM163A, BLOC1S4, KIAA0232, PFKP-DT, LINC02645, MIR633, TRMT112P3, LINC01712, LINC02815, NIT2, AARS2, SPATS1, RN7SKP211, SLC14A2, NRXN1, LINC01793, HDAC9, BMP1, NKX2-6, HNF4G, PKMP4, RPS19P6, LINC02746, SNRPGP16, LINC01081, TXNL1, ST6GAL1, RAB20, SPSB1, BDH1, ADARB2, TOP1, LIN28B, C2orf83, TIAM2, IL6R, CCRL2, KRBOX1, CYP8B1, ACKR2, or combinations thereof.

In some embodiments, the one or more genes affected by ADHD comprise AATK, BAIAP2, ABHD17C, ABHD5, RNU6-367P, ABI1, ACTGIP22, ACTGIP23, E124P1, ACTN2, ACTR5, PPPIR16B, ADAM15, ADAMTS2, ADCY2, ADCYAP1R1, NEUROD6, ADGRB2, COL16A1, ADGRL2, LINC01362, ADGRL3, ADGRL3-AS1, ADGRL4, AFF3, AHCYL1, AK8, AKAP6, AKAP8P1, IMP3P1, ALMS1, AMBRA1, AMD1P4, HIVEP1, ANK3, ANKS1B, ANO3, ANO4, ANO5, SLC17A6, ANXA2R, ZNF131, ANXA8, ZNF488, AP1S1, ARHGAP15, ARIDIB, ARID4A, ARID5B, ARPP21, ARSB, ASCC2, ASCC3, ASNS, TACI, ASPSCRI, ASTN2, ATF1P1, COPS5P1, ATP2A2, ATP2C2, ATP8B1, ATXN2, AUTS2, BAG5, RNU7-160P, BANK1, SLC39A8, BCL11A, BCL11B, BDNF, BDNF-AS, BEND7, BIRC6, BMPRIB, BMPR2, RN7SL40P, BNC1, BNC2, RN7SL720P, BNIP3P1, LINC00645, BORCS7-ASMT, AS3MT, BPTF, BRAF, CCT4P1, BRINP1, BRWD1-AS1, BRWD1, BTAF1, C2CD5, LRRC34P1, C8orf86, C9orf62, SOCS5P2, CA10, CABP1, CACNAIC, CACNAID, CACNAII, CACNA2D3, CACNB2, CADM2, CADPS2, FEZF1, CALNI, CAMKID, CCDC3, CAMTA1, CARMIP1, LINC01231, CBX8, CCDCl92, CCDC68, LINC01929, CCDC73, CCDC88B, PRDX5, CCDC91, CCL1, C17orf102, CCNJP1, RNU6-1335P, CD276, INSYN1, CD40, CDH22, CDH13, CDH23, CDH8, CDK10, SPATA33, CDK14, CELF2, CELF2-AS2, CEMIP2, CEP112, CEP85L, CFAP221, CFAP43, CHD7, IFITM3P8, CHFR, GOLGA3, CHMP7, CHRNA3, CILKI, CLDN17, GRIK1, CLEC17A, CLYBL, ZIC5, CNBD1, CNKSR2, RNU6-133P, CNNM2, CNTLN, CNTN3, NIPA2P2, CNTN4, CNTNAP5, COPB2-DT, COX6CP4, LINC02853, CPLX2, CPNE4, CPSF6, CPXM2, CRBN, SUMF1, CREB5, CRPP1, CRP, CRYGC, CSMD1, CSMD2, CSRNP3, CTBP1, CTNNA2, ANKRD11P1, CTNNA3, CTNND1, CTTNBP2, CULIP1, CUL3, CCDCl95, CUX1, CWC15, ST13P11, CYCSP6, RNU6-827P, CYP24A1, PFDN4, DAB1, DACH1, DCC, DCDC1, DENNDIA, DENNDIB, Clorf53, DGKI, DIRC3, CACYBPP2, DISC1FP1, DLEU1, DLEU7, DLG2, DLX6-AS1, DLX6, DNAJAIP1, HMGN2P39, DNAJC1, DNAJC11, DNM1, DNMT3B, DOCK10, NYAP2, DPP10, DPP6, DRD2, DSTNP5, DUSP6, POCIB, EBLN3P, ECM1P2, RNU6-578P, EEF1A1P11, RN7SL831P, EFCAB8, EFNA5, EIF3M, WT1-AS, ELAVL2, IZUMO3, ELAVL4, ELL, FKBP8, ELMO1, ELMO1-AS1, ELOAP1, PSMD14P1, ELOVL3, NOLC1, ELOVL6, ELP1, EMP2, ENOX1, EPCAM-DT, EPHB1, EPHX2, GULOP, EPM2A, UTRN, ERAP2, ERBB4, ERC1, ERC2, ERCC4, TMF1P1, EREG, EPGN, ESR1, ESRRB, ESRRG, ETV3, FADS2, FAM135B, FAM13A, FAM149B1P1, FAM168A, PLEKHB1, FBN3, FBXL16, FBXL17, FBXO11, MSH6, FERMT3, FGF3, ANO1, FGFR1, FGFR2, FHIT, FHL2, C2orf49, FLII, FLJ40194, FMOD, LARP7P1, FOLHIB, FOXP1, FOXP2, FPGT-TNNI3K, TNNI3K, FRMD1, FSTL5, FTCDNL1, RN7SL717P, FTLP18, GRIK3, FTLP4, RPS26P2, FURIN, FUT2, FXR1, GAβ4, GABRA2, GABRB1, GABRB2, GALNT13, RNA5SP107, GAPVD1, Metazoa_SRP, GGACT, GGNBP1, ZBTB9, GID4, GIGYF2, GKAP1, GLT8D1, GMIP, GNPATP, RPS27AP16, GPC6, GPR135, GPR139, GRIA4, GRID1-AS1, GRIK4, GRIN2A, GRM3, GRM3-AS1, GRM5, GRM8, H4C8, H3C9P, HACE1, NPMIP10, HAUS6P1, POM121L12, HCN1, MRPS30, HDAC4, HELZ, HERC1, HHLA2, HINT1, HLA-DMA, HLA-DMB, HLA-DPB2, HLA-DRB5, EIF4EBP2P3, HMGB1P45, LINC02808, HMGB1P47, RNA5SP182, HMGB3P23, RNA5SP281, HNRNPA1P1, ZNF184, HNRNPA1P46, HNRNPA1P64, HMGB1P18, HNRNPK, C9orf64, HOOK1, CYP2J2, HPCAL4, HPRT1P2, RN7SKP207, RPL19P11, HPS5, HRAT17, HS6ST3, HSD17B12, HSPA12A, HSPD1P6, LINC02033, HSPE1-MOB4, MOB4, HULC, HYI-AS1, PTPRF, IFITM8P, RN7SKP135, IFNAR2, LINC01548, IGBP1P5, LINC02261, IGFIR, IGSF11, LINC02024, IGSF21, IGSF9B, IKZF2, IL19, ILIR1, ILIR2, IL21, IMMP2L, INPP4B, IQCJ-SCHIP1, IQCJ, ITGA1, ITGA11, ITGAE, ITIH3, ITPR3, JADE2, JCAD, JPH2, JPTIP1, CDCA7, KC6, NPM1P1, RPL17P45, KCNB1, KCNC2, KCNG2, CTDP1, KCNH3, KCNJ3, KCNJ6, KCTD3, KDELR2, DAGLB, KDM3A, U8, KDM3B, KDM4A-AS1, KDM4A, KDM4B, KIAA1328, KIAA2012, KIAA2012-AS1, KIF21B, KIF5C, KIF6, KIRREL3, KIZ-AS1, KIZ, KLC1, KLHL29, KMT2A, KRT8P37, L3MBTL2, L3MBTL2-AS1, LAMA2, LIG4, LIN28AP1, LINC00222, FOXO3, LINC00424, LINC00456, MBNL2, LINC00461, LINC00470, AIDAP3, LINC00494, RNU7-144P, LINC00499, NOCT, LINC00534, CALB1, LINC00644, LINC00700, MIR6072, LINC00707, LINC00907, LINC00917, LINC00927, ARNT2, LINC01068, LINC01038, LINC01122, LINC01208, MTND5P15, LINC01239, LINC01288, LINC01320, LINC01360, RN7SKP19, LINC01392, POLR2DP2, LINC01412, TEX41, LINC01414, LINC01416, LINC01445, LINC01470, LINC01479, LINC02421, LINC01485, CPEB4, LINC01505, LINC01515, OLIG1, LINC01572, LINC01645, LINC01647, DISP3, LINC01683, LINC02573, LINC01707, LINC01735, PLXNA2, LINC01737, LINC01748, LINC02778, LINC01787, UBE2WP1, LINC01811, LINC01822, RN7SL117P, LINC01830, LINC01833, LINC01837, LINC01871, LINC00298, LINC01875, TMEM18, LINC01876, LINC01905, LINC01934, LINC01950, PSMC1P5, LINC01965, LINCO2010, PLSCR5, LINC02027, GBE1, LINC02057, SMIM15-AS1, LINC02127, LINC02128, LINC02141, NPAPIL, LINC02163, RNU6-334P, LINC02191, ZKSCAN2-DT, LINC02236, LINC02196, LINC02240, LINC02253, RN7SKP181, LINC02273, LINC02288, LINC02295, LINC01550, LINC02313, ARHGAP5-AS1, LINC02326, LINC02374, LINC02387, DENND5B, LINC02429, Y_RNA, LINC02466, ZSWIM5P3, LINC02500, LINC00290, LINC02511, TERFIP3, LINC02520, LINC02554, MN1, LINC02588, LINC02294, LINC02607, LINC02609, BARHL2, LINC02612, FABP5P10, LINC02625, LINC02626, ATRNL1, TRUB1, LINC02663, LINC02670, LINC02668, LINC02676, LINC02682, LINC02751, LINC02694, LINC02712, KIRREL3-AS3, LINC02758, METTL15, LINC02774, AKT3, LINC02789, LINC02797, LINGO1, LMAN2L, CNNM4, LMCD1-AS1, LNC-LBCS, LRFN2, LRFN5, LRGUK, LRPPRC, RNU6-566P, LRRC4C, LINC02741, LRRC7, LRRN2, LRRTM4, LUZP2, MACROD2, MAD1L1, ELFN1, MAGI2, MAIP1, SPATS2L, MAML3, MAN2A2, MANBA, MAPIB, MAP2K5, MAP3K20, MAP3K7, MAPK14, MAPK13, MAPRE1, MAPT, MARK2P11, LINC02227, MBIP, RN7SKP21, MCHR2, MCTP1, MCTP2, H3P40, MDGA2, ME2P1, MIR873, MED27, MEF2C-AS1, MEF2C, MET, EED, NUCKS1, YWHAZ, MFSD4B, REV3L, MGAT4C, MGAT5, RN7SKP93, MICC, UBQLNIP1, MINDY1, MINK1, MIR137HG, MIR203A, KIF26A, MIR2113, MIR3976HG, MIR4432HG, RNA5SP94, MIR4643, MIR5007, HNF4GP1, MIR646HG, LINC01718, MIR8084, C8orf87, MIR924HG, MIR99AHG, MIRLET7BHG, MLIP-AS1, MLIP-IT1, MLIP, MLN, LINC01016, MMP24, MMP24OS, MOBP, MOCS2, MORC1, MPHOSPH9, MPP6, MRM2, MROH5, MIR1302-7, MSC-AS1, MSL2, MTA3, MTCO2P4, LINC01181, MTCO3P1, HLA-DQB3, MTCYBP27, RNU6-976P, MTND2P8, KRT18P24, PSAT1, MTND5P17, LINC02630, MTUS2, MYO18A, MYRFL, PRANCR, MYTIL, NAA11, LINC01088, NAβ2, NALCN-AS1, NAPRT, NAV2, NBEA, NCAM1, NCAM2, PPIAP1, NCKAP5-AS2, NCKAP5, NCL, NCOA5, NDUFAF2, NEGR1, RPL31P12, NEURL1, NFASC, NFAT5, NFIA, NFIB, CDCA4P1, NGEF, NGF-AS1, LINC01765, NINL, NKILA, NLGN1, NMRAL2P, NOL4L-DT, NPAS3, NR2F2-AS1, NRAP, NRG3, RPA2P2, NRGN, NRXN1, NRXN2, NRXN3, FXNP1, NSUN3, ARMC10P1, NT5C2, NT5DC3, NTM, NTRK3, LINC00052, NUFIP2, OBI1-AS1, OCA2, OLFM1, OLFM4, OR2J4P, OR5AZIP, OR5BAIP, OR5H3P, OR5H5P, OSBPL3, GSDME, OTX1, RPL27P5, OTX2, PACRG-AS1, CAHM, PACSIN3, DDB2, PALB2, PARD3B, PARPBP, PAUPAR, PAWR, PBX1, PBX2P1, SLC9A9, PCCA-DT, PCDH15, PCDH7, LINC02497, PCLO, PDE11A, PDE1A, DNAJC10, PDEIC, PDE4B, PDE7A, MTFR1, PFKFB2, PHACTR1, PHC2, PHF20L1, TMEM71, PHF21A, PI4K2B, PITPNM3, PIWIL4, PJA1, PKD1L2, PKD1L3, PKN2-AS1, PLCL1, PLCL2, PLEKHA5, PLUT, PLXNA4, PMFBP1, POLG-DT, MIR9-3HG, POLRIH, POSTN, TRPC4, POU3F2, PPIAP21, RNU6-743P, PPMIF-AS1, PPPIR13B, PPPIR21-DT, FOXN2, PPP2R2B, PRKDC, PRKG1, PRPF3, PRPF40B, FAM186B, PRPSIP1, FIGN, PRRC2B, PSMD11, PTK2, ERICD, PTPRD, PTPRG, PTPRK, PTPRN2, PWRN4, RNU6-741P, PXDNL, RAB19, RAB3GAP2, RAB7A, RABGAPIL-DT, RAI1, RANBP17, RANBP2, RAP1BP2, ALCAM, RAPGEF4-AS1, RARB, RNA5SP125, RASGRF2, RASL11A, RNU6-70P, RBFOX1, RBM19, RCOR2, NAA40, REEP5, RERE, RERE-AS1, REST, RFPL4AP3, GBA3, RFT1, PRKCD, RGL1, RGS6, RHEBL1, DHH, RHOT2, RIMS1, RIT2, RMI1, SLC28A3, KRT8P21, RN7SKP190, RN7SKP231, PRR13P7, RN7SKP279, DNAJB6P1, RN7SL101P, MIR4275, RN7SL167P, SNX19, RN7SL178P, RN7SL553P, GRM7-AS3, RN7SL592P, SMARCA2, RN7SL618P, ZNF646P1, RN7SL675P, RN7SL795P, RPS2P6, RNA5SP169, RNA5SP173, NDUFB5P1, RNA5SP199, KIF4B, RNA5SP202, ECI2-DT, RNA5SP214, RNA5SP299, RNA5SP404, RNA5SP403, RNA5SP472, LINC01782, RNA5SP87, RNF13, ANKUB1, RNF217-AS1, NKAIN2, RNF227, KCNAβ3, RNU1-130P, RNU1-15P, ASSIP11, RNU1-29P, KCND2, RNU1-84P, NPAS4, RNU6-1088P, PLA2G1B, RNU6-1270P, PDZRN3, RNU6-168P, MAT2B, RNU6-170P, CIDEA, RNU6-256P, SORL1, RNU6-276P, RNU6-281P, RNU6-289P, CCDC34P1, RNU6-293P, RSPH3, RNU6-297P, LINC00648, RNU6-439P, RPL7P13, RNU6-440P, FZD7, RNU6-463P, SORCS3, RNU6-508P, LINC01793, RNU6-573P, ARAP2, RNU6-655P, RNU6-699P, RNU1-63P, RNU6-917P, SIRPB2, RNU7-147P, RNU7-66P, NUFIP1P1, ROBO2, RPL10AP3, PPPIR3B, RPL12P34, RN7SL761P, RPL17P35, CYP2C61P, RPL36P1, TSHZ2, RPL38P2, LINC01851, RPL6P5, METAP2P1, RPL6P8, ARL14, RPL7L1, BICRAL, RPL7P20, LINC01947, RN7SKP60, RPL7P47, NPIPA8, RPL7P6, CAPZA3, RPP40P2, RNU7-88P, RPS19BP1, RPS26, ERBB3, RPS29P33, RPS2P48, KIF2B, RPS3AP1, RPS3AP17, RPS6KL1, RPTOR, RSRC1, RTN1, RUNXIT1, FLJ46284, RXRG, RYR2, SAP18P1, BIN2P2, SCAI, SCMH1, FOX06, SCN2A, SDCCAG8, SDK1, SEM1, SEMA3A, SEMA3F-AS1, SEMA6D, SEPHS1P2, SETD5, SFTA2, MUCL3, SGIP1, SGMS1, SGO1-AS1, SGSM2, SHC3, CKS2, SHISA9, SHMT2, NXPH4, SHTN1, SIPAIL2, SKINTIL, SLC12A5, SLC14A2, SETBP1, SLC1A5, SLC24A3, SLC30A9, SLC35F1, SLC35F2, SLC35F4, SLC4A10, SLC6A6P1, SLC6A9, SLC8A1-AS1, SLIT2, LINC02438, SMARCE1P4, KCNU1, SMG6, SMIM19, CHRNB3, SMIM31, APELA, SMIM7P1, MIR548AD, SMYD2, SNAP91, SND1, SNORC, SNRK, POMGNT2, SNTG1, SOCS5P5, FCF1P10, SOX2-OT, SOX5, SPA17, SPAG16, SPATA2P1, RN7SKP6, SPATA7, SPCS2P2, ERLIN1, SPDYE4, MFSD6L, SPG7, SPOCK1, SPPL3, ACADS, SRPK2, SRRM1P2, CYP51A1P1, LINC00971, SRXN1, TCF15, SSR4P1, PICSAR, ST3GAL3-AS1, ST3GAL3, STAG1, STIM1, STIMATE, STIMATE-MUSTN1, STIP1, STK32C, STPG2, COX7A2P2, STT3A, FEZ1, STX17-AS1, SULF2, SULT1A2, SUMO1P1, BCAS1, SUPT3H, SYNE1, SYNE2, ESR2, SYT14, SYT4, RNA5SP455, TACC2, TAF5, PCGF6, TAF9BP2, TAFA5, TANGO6, HAS3, TANK-AS1, TANK, TBCID5, TCAIM, TCF20, TCF4, LINC01415, RNA5SP459, TDRKH, TEAD1, TENM2, TENM4, RNU6-544P, TERF2IP, TET2, TET3, TFAP2B, TFAP2D, TFEB, NPM1P51, THBS4, THOC7, THSD7B, TLL2, TMC3-AS1, TMEM114, TMEM132B, TMEM161B-AS1, TMEM163, TMEM182, CRLF3P1, TMEM219, TMX3, MTL3P, TNR, TNRC6A, TOX, TPTIP1, SCAF4, TPTIP9, TLR4, TRAF3, TRAF3IP2-AS1, TRAF3IP2, TRAIP, UBA7, TRHDE, TRIM26, TRIM51, TRIM71, RPL23AP43, TRIM8, ARL3, TRMT9B, LINC00681, TRPS1, TRPT1, TSHZ3, LINC01791, TSNARE1, TTC28, TTC29, TULP4, TYR, TYW3, LHX8, U3, LINCO2869, U6, ARID2, UBAP2L, UBBP1, UBE2D3, UBE2E1, UBE2V2, UGT1A10, UGT1A8, UGT1A9, UNC5B, LINC02622, UNC79, UPF1, COMP, USH2A, USP28, VINACIP, TTL, VLDLR-AS1, VPS45, OTUD7B, VRK2, VTI1A, WDPCP, WDR27, WNT3, WSCD2, ASCL4, WSPAR, XKR6, XXYLT1, MIR1263, XYLT1, CARSIP2, LINC02549, RCC2P3, YAE1, POU6F2, ZBTB16, ZBTB20, ZCCHC7, ZFHX3, ZFPM2, ZIC4, ZKSCAN5, ZMIZ1, ZNF385D, ZNF407, ZNF423, ZNF471, ZNF518A, BLNK, ZNF521, ZNF536, ZNF544, ZNF8-DT, ZNF584, ZNF615, ZNF654, CGGBP1, ZNF75A, ZNF804A, ZP3P2, ZSCAN12, ZSCAN31, or combinations thereof.

In some embodiments, the one or more genes affected by ALS comprise DACH1, LINC01685, HOXD10, MRAS, ABCG1, LINC00351, RGS6, LINC02215, ABCC12, ASSIP10, KC6, NEDD4L, TBXAS1, TUBB4BP4, LINC02756, NUDT4, LINC00540, IQCF5-AS1, IQCF1, CTNND2, PDZPHIP, OPCML, LINC00111, PCSEAT, MYOM2, TSPAN9, CALML3, LASTR, TFAP2A, ARIDIA, RNU7-29P, LINC01741, LRRC8C, RPS16P2, LAPTM4A-DT, STON1, STON1-GTF2AIL, SLC25A12, PTH2R, TCL6, LINCO2318, GSE1, ASIC2, NSFP1, C17orf67, MACROD2, PROCR, ITGA9, CHODL, ZFYVE26, RN7SKP252, C22orf34, MEG8, WSPAR, TAF8, LINC01747, KRT18P3, NFATC2, UNC13A, C9orf72, KRT18P55, ANKRD29, RPS10P27, EPB41, RN7SKP156, NFASC, CASC11, PVT1, CDRT15P5, ADAMTSL1, LINC00474, ANK3, WAPL, GRID1, LDHC, DNAJB6P1, RBM19, TMEM132B, ADGRD1, OLFM4, ERBB4, HSPA9P1, RPL23AP28, PLXNA1, KCNMB2, KCNMB2-AS1, MASP1, CCSER1, LEF1, HADH, INPP4B, LINC01320, METTL21A, TRPM8, SPP2, ANXA3, PDLIM5, BMPRIB-DT, ETNPPL, OSTC, SYNPO2, LAMA2, CALNI, PDGFRL, CLVS1, RN7SL592P, KLF6, ATP2B2, ST6GALNAC3, TPIIP1, SARMI, MOBP, IDE, CAMTA1, PSD3, SEC61GP1, SLC9A9, KCNS3, FHDC1, LINC02055, ZFP64, SUSD1, DPP6, STK36, VIL1, CTDSP1, CPNE4, FKBP5, LINC02531, LINC02712, SMIM2-AS1, RNU6-745P, ANKRD34C-AS1, KIAA0513, PIGL, CENPV, PPY2P, LAMA3, REEP2, EGR1, LINC02408, MTND5P1, LINC02575, LINC01182, CREB5, TBC1D1, KRT18P24, LINC01937, PPP2R2D, LINC02713, RPL7P40, ARL5AP5, PBDC1, ITPR2, TIAM1, KCNG2, CTDP1, FOLHIB, OR52K3P, AFF1, C4orf36, CAMKIG, SUSD2, ZBTB40, PPIAP34, KALRN, APTX, LINC02697, MIR1297, RPL13AP25, LINC02582, SQLE, NME9, DISC1, NT5CIA, EFEMP1, PNPT1, B4GALT6, RN7SKP44, SLC39A11, ATXN1, Clorf112, SELL, RNU1-98P, MIR99AHG, KIFAP3, MORN2, ZNF746, ZNF767P, COMMD10, SCN7A, RBMS1, CREB1, MOB3B, LIPC-AS1, LIPC, ALDH1A2, CLCN3, ERICH6B, PTPRN2, SOD1, G2E3, MTCO3P1, HLA-DQB1, KIF5A, CFAP410, TNIP1, SLC9A8, TBK1, ERGICI, SCFD1, ATXN3, LINC02096, RPS18P12, ACSL5, TYW3, RNU6-1069P, LINC02422, NPAPIP6, MAPK1, YPEL1, LINC01287, ARHGEF2, PTPRF, KDM4A, CCDCl92, SLC18A1, RPL30P9, CNOT2, NRXN3, ST3GAL3, ARAP2, IFRD1, PCSK5, ANKSIB, WASHC5, NPEPPS, or combinations thereof.

In some embodiments, the one or more genes affected by ASD comprise CTBP2P4, RNF111, SLTM, NTM, RASGRP4, FHIT, SNHG18, TAS2R1, ZBED3-AS1, MSNP1, PARD3B, EYA1, TAFIC, MACROD2, PPP2R5C, CSDE1, TRIM33, BCAS2, AMELY, TBL1Y, DDX3Y, UTY, KDM5D, EIF1AY, SLC52A2, FBXL6, MYO9B, NFIA, NFIA-AS2, ESRRG, SYNPR, SYNPR-AS1, TENM2, RN7SKP159, MCPHI, RPL34P23, BSX, MIR4495, PAFAHIB2P2, KCTD12, RN7SL571P, NABIP1, NAALADL2, LINC01151, OR2M4, SGSM2, LINC00907, INHCAP, CUEDC2, SMIM21, FGFR1, SSUH2, NLRP4, POLR2E, CDK4P1, SORBS1, LUZP2, MIR7154, CHST1, LINC00376, HSP90Aβ6P, HS6ST3, LINC00922, MPPED1, GPR156, LRRC58, PBX2P1, SLC9A9, AACSP1, ZNF354A, SPTLC1P2, RPL10P19, RNU6-682P, LINC02827, LINC02404, RP1L1, SOX7, RN7SKP32, RNU6-819P, ASAP1, MROH5, RSU1, EGR2, MRPL35P2, GUCY1A2, PCDH9, ESRRB, SERPINA2, SERPINA1, LINC02210-CRHR1, MAPT, KANSLI, GALNT1, NEDD4L, ZNF568, KIZ, XRN2, ZNF877P, LINC02790, RN7SL831P, EEF1A1P11, SNX7, FTLP17, CAMSAP2, PKP4, SGO1-AS1, CADPS, RSRC1, RN7SKP222, FGF12, LINC00461, LINC02058, LINC02163, RNU6-334P, KCNN2, GALNT10, ADTRP, GABBR1, EIF4EBP2P3, KMT2E, RNU6-1103P, SORCS3, RBFOX1, TLK1, GALNT12, THSD4, CITED1, DCAF12L2, ARHGEF6, RPL7P56, NEGR1, ITIH3, GPR89P, RSL24D1P1, RNU7-160P, BAG5, EXT1, HLA-A, NRGN, ITIH4, SRPK2, MIR137HG, ZKSCAN3, BORCS7, PTGES3P4, TRIM10, SUMO2P1, MOG, TSBP1-AS1, HLA-DQA1, HFE, OR2J3, OR2J2, ACTGIP22, HLA-DRB5, HLA-DRB9, NOTCH4, LINC02033, HSPD1P6, OR5V1, HLA-W, GRM3-AS1, GRM3, FURIN, VRK2, SLC39A8, LINC01416, LINC01415, MUC22, HCG20, ACTR5, PPP1R16B, LINC01929, ZSWIM6, C6orf15, RNU6-1133P, HLA-DRB1, HLA-DQB1, HLA-DOA, BRD2, DDR1, FOXP1, LINC02797, RN7SL167P, SNX19, NGEF, MTCO3P1, LINC02571, HLA-B, NOP56P1, RPSAP2, HLA-G, HCP5B, C2, HLA-DRA, MUC21, NAPGP2, LINC00243, HLA-DRB6, HMGN4, HCG11, ZBED9, TSBP1, BANK1, DDX39BP2, HCG9, PBX2, KLC1, COA8, PRRC2A, H2BC15, H2BC16P, ZSCAN9, MAD1L1, IMMP2L, TNXB, MICA, HLA-S, SFTA2, MUCL3, IGSF9B, LINC02730, MEF2C-AS1, PPPIR13B, GRIN2A, LINC02057, GRAMDIB, ETF1, RGS6, H2AC10P, DPYD, CHRNA3, TUBB, PSMB9, PPPIR2P1, ADAMTSL4-AS2, FALEC, ZNF804A, SATB2, RNU7-147P, CUL3, GIGYF2, STAG1, FXR1, IGBP1P5, LINC02261, CISD2, CALNI, DMTF1, RN7SL178P, MSRA, TSNARE1, HMGB3P24, RNF38, SLC25A6P5, TMEM38B, AS3MT, BORCS7-ASMT, ARL14EP, FSHB, DRD2, CACNAIC, GLTP, TCHP, ATP2A2, MPHOSPH9, LINC02326, UNGP3, DYNLLIP1, NDRG4, SETD6, GID4, WNT3, TCF4, ZNF536, CACNAII, RNU1-61P, MSH5-SAPCD1, MSH5, HLA-C, HCG27, NXPH4, SHMT2, BTN2A2, PTPRF, KDM4A, TMEM219, BRINP2, LINC01645, SLC38A7, CNOT1, DHFRP2, RN7SKP19, LINC01360, CYP2D7, ZFP57, ZDHHC20P1, MTND4LP18, PCNX1, HLA-V, IFITM4P, RNU6-1029P, SF3B1, RPL10P2, PRSS16, CAPZA1P4, HLA-DMB, HLA-Z, EPHX2, GULOP, HLA-DQB3, BTN3A1, TOB2P1, ZNF192P2, GGNBP1, HCG4B, MCCD1P1, TRIM8, ARL3, CLCN3, EP300-AS1, HLA-H, POM121L2, LINC02829, HLA-U, OR11A1, LINC01470, KDM3B, SYNGAP1, ALPK3, ZNF592, VNIR10P, ZNF204P, HLA-DMA, DGKI, PRKCD, RFT1, SMIM15-AS1, TRIM26, PSORSIC1, HLA-T, ATAT1, HLA-DQA2, SRR, RERE, RERE-AS1, MOB4, HSPE1-MOB4, BTN2A1, TWF2, PPDPF, EEF1A2, HCP5, GUSBP2, MICB-DT, LINC00533, FKBPL, PRRT1, MIR3143, MICD, LINC01648, EHMT2, DDX10P2, ATP2B2, GRIA1, BTN3A2, H3C9P, ZSCAN16-AS1, ZDHHC20P2, FGFR3P1, UBQLNIP1, MICC, MDK, MAIP1, CNTN4, LINC01149, HCN1, LINC00240, LYPLA2P1, MYL12BP3, MMP16, TMX2, MTCYBP27, SNAP91, CHRNA5, ZNF823, PRKG1, RIMS1, TCF20, KRT18P42, MAN2A1-DT, C2-AS1, ZNF318, ABCC10, F2, RPL8P1, LINC01012, CARMIL1, CMAHP, TRIM31-AS1, TRIM31, NT5C2, BEND4, OLFM4, PAUPAR, PKN2-AS1, MIR2113, FSTL5, CACNAIC-IT3, DEF8, TUBB3, BCL10, DDAH1, IMPA2, LINC02172, FBXW12, RHOJ, DRAXIN, LRRC20, FOXB1, RPLPOP1, PTH2R, UFL1-AS1, CNNM2, SNORC, ZMIZ1, PPPIR3B, RPL10AP3, FOXN2, PPPIR21-DT, ANK3, TCF15, SRXN1, LINC00707, RNA5SP403, RNA5SP404, CLEC17A, ZP3P2, SMARCA2, RN7SL592P, POLRIH, ANKSIB, ZNF615, SEMA3A, MRM2, GRIK1, CSMD1, FEZ1, STT3A, KMT2A, SLC17A6, ANO5, PRPF3, KIF5C, LINC01239, CACNB2, PALB2, MPP6, RNA5SP459, GLT8D1, ADGRL4, NTRK3, SPATA33, CDK10, ILIR2, ILIR1, NEURL1, SLC35F2, HINT1, RCC2P3, PPP2R2B, RNA5SP169, KIF21B, HDAC4, ZFPM2, SYNE1, ZSCAN12, PCLO, METTL15, LINC02758, OR5BAIP, OR5AZIP, GRM5, TYR, DCC, CCDC68, CTDP1, KCNG2, KIZ-AS1, SLC12A5, RPL31P12, HNRNPA1P46, LINC01830, RNA5SP87, NCOA5, MIRLET7BHG, CNKSR2, PJA1, ZCCHC7, CNNM4, LMAN2L, TCAIM, RPS19BP1, L3MBTL2-AS1, L3MBTL2, KCNB1, SHISA9, RPS6KL1, SPPL3, AKAP6, CAMTA1, RNU1-130P, BCL11B, PDE4B, HYI-AS1, PMFBP1, PXDNL, SOX5, RPTOR, SPAG16, CCT4P1, BRAF, NAA40, RCOR2, CTNND1, FTCDNL1, RN7SL717P, RTN1, LINC02694, ZSCAN31, SLC30A9, NPAS3, ACADS, AMBRA1, MYO18A, GSDME, OSBPL3, LINC02240, FOLHIB, OTUD7B, VPS45, MOCS2, FADS2, TMEM161B-AS1, ENOX1, MIR1302-7, THOC7, GMIP, ESR2, SYNE2, GRIK3, FTLP18, LINC01876, LINC01088, NAA11, PLCL1, LRFN5, or combinations thereof.

In some embodiments, the one or more genes affected by BD comprise SLIT3, ADGRE1, OXCT1, NCAN, KRTAP5-11, KRTAP5-10, HTR6, NUTF2P8, CNNM4, LMAN2L, GPR35, CAPN10, ATP2B2, LINC02033, HSPD1P6, PBRM1, CADM2, KIAA1109, ADCY2, Y_RNA, KDM3B, VTRNA1-3, PCDHA1, H2BC9, H3C8, EIF4EBP2P3, RPS6KA2, MAD1L1, THSD7A, SP4, MSRA, PLEC, TUBBP5, CACNB2, ANK3, XPNPEP1, FADS2, PACS1, PC, SHANK2, CACNAIC, IFT81, ALOX5AP, B3GLCT, LINC02694, STARD9, INSYN1-AS1, TBC1D21, ZNF592, PIGW, MYO19, HDAC5, ILF3, STK4, RBPJL, KCNB1, RNU6-751P, MIR522, CUBN, ERI1, RPL10P19, LINC01378, WDR49, MACROD2, LINC01221, ANKHD1-EIF4EBP3, ANKHD1, MYO16, CACNA2D2, ASTN2, RPSAP74, SYNE1, EEF1A2, PPDPF, FSTL5, CACNAIC-IT3, HLA-DRB5, BABAM2, MRPL33, SLC25A17, FADS1, RNU4-74P, TMEM178B, KCNG1, LINC01748, LRRC3C, IL21, LINC02280, TMEM179, ACTGIP22, RRS1-AS1, RPSAP12, LINC02384, ATP2A2, MESTP3, LINC02364, PARP10, AK5, TENM4, CDANI, TTBK2, ITIH4, LINC01392, BRINP1, MAPK8IP1, ANKRD23, CNNM3, PRSS36, ANKS1B, GRIN2A, DHH, RHEBL1, MYO1G, PURB, TWF2, PPPIR3B, RPL10AP3, GFIIB, NGFR, LINC02075, ATP8B3, CRACDL, ITIH1, MCTP1, RNU6-745P, TLE3, CTNNA2, KIT, LINC02260, CTSF, ZNF385D, GARNL3, SIAE, LINC00596, SEPTIN7P14, PDE5A, UBE2R2-AS1, LINC00927, ARNT2, LINC01307, SUMF1, UBAP2, SVEP1, SPRED1, ZMIZ1, COLEC12, THOC1-DT, CMKLR1, LINC01498, LINC01354, TARBP1, DLC1, PDE10A, LINC02779, PRELID3BP1, RPL21P17, SGO1-AS1, NPAS3, CMTM8, DGKH, RNPEPL1, PALB2, SLC35F4, TDRD9, BICRAL, LAMP3, CDC25B, DPY19L3, DHFRP2, HLA-B, RN7SKP94, CCDCl82, MIR3681HG, TCF23, AHCYP3, DPP10, CAPN10-DT, FAM126A, SMARCE1P4, TLE4, WHRN, ADAMTS14, HSP90AA2P, KIF18A, LINC01146, PHBP21, ZNF490, MGAT4A, HMGAIP6, HNRNPC, INSYN1, CD276, TPIIP1, ST6GALNAC3, CDKAL1, LYRM2, ANKRD6, LINC00508, LINC02441, LINC00375, PRSS35, NUMB, LINC00620, ATP6VIH, GNL3, WSCD1, RNU6-1264P, LINC01648, MYOM3, RCC2P3, KIF5B, BASP1-AS1, BASP1, PIM1, TMEM217, INPP4A, CRYBB2P1, C15orf32, SP8, RPL23P8, TRANK1, GRIK5, PARD6B, BCAS4, CTSH, CACNB3, RPS15AP15, RN7SL635P, FOX06, RASIP1, SLC45A4, C11orf80, LINC02611, ITIH3, NFIA, AUTS2, TTC39B, RPL7P33, PPMIK-DT, FAM189A2, MIR4675, CNTN1, TMEM63C, KRTDAP, FFAR2, KCNS1, OR14J1, OR2U2P, GGNBP1, SNAP91, GSDME, GRM3-AS1, GRM3, BRAF, CCT4P1, FGFR1, TSNARE1, BORCS7-ASMT, U6, MXI1, SNRPGP12, SPTBN2, DRD2, PRKD1, RGS6, PPPIR13B, CHRNA3, UBE2Q2P1, FURIN, TMEM219, KCTD13, MCHR1, TCF20, BTN3A2, H3C9P, FTCDNL1, RN7SL717P, NGEF, CNTN4, SLC39A8, ZSWIM6, JADE2, LINC01470, CMAHP, CARMIL1, BTN3A1, H2BC15, H4C12, LINC01830, MIR548AE1, ZNF804A, RNU6-1029P, SF3B1, VRK2, TLCD4-RWDD3, TLCD4, RN7SL480P, PLEKHO1, PAX5, C16orf72, RPL21P119, MOCS3, RNA5SP63, U3, DOCK2, INSYN2B, SEC61GP1, DNAJC17P1, SAMMSON, GSDMA, MPP6, FKBP2, RPA1, RTN4RL1, THRA, PCGEM1, SLC44A3P1, ALDH7A1P4, ADO, EBLN3P, BCL11B, VENTXP5, PUS7, SRPK2, CUL4A, RPL13, CPNE7, OSBPL2, BTN2A1, CERS6, MIR2113, SCN2A, HOMER2, MIR124-1HG, ACTN3, SSBP2, PLXNA4, LINC00636, CD47, ZSCAN2, CLASP1, RPS17P7, DCBLD1, SLAIN2, SLC10A4, KCNN3, PMVK, AKAP6, COX6A1, RPS27P25, HUWE1, PPIAP48, CFAP221, KRT8P2, LINC02149, KIF15, PDE3B, HLF, STXBP4, RPL29P25, PPPICC, NPTX1, RPL32P31, PSMD3, CSF3, VIPR1, RPL35AP8, MAPK1, YPEL1, BICRA, RN7SL322P, TSBP1-AS1, TSBP1, LGMNP1, STARP1, ASAP2, IFITM3P9, RN7SL361P, MYRF-AS1, LAMA4-AS1, WFDC12, WFDC5, ZCCHC2, MACROD1, ZCCHC7, RBKS, IQCH, IQCH-AS1, RIMS1, ARIH2P1, SMYD3, LINC01505, FGF12, RNU6-239P, HAX1, KCNN2, RNA5SP149, PEX5L, TMEM131, OFCC1, IL9RP1, MVB12B, LYARP1, SLC8A3, PABPCIL2B, PABPC1L2A, DLGAP2, SEMG1, SEMG2, LINC01203, C12orf76, LAYN, TNKS, SLAMF9, LINC01133, HIVEP2, FYN, VAV3-AS1, NRF1, KPNA4, LINC00970, WNK2, RNA5SP272, MMP16, SLC4A10, KCTD2, ATP5PD, RN7SL573P, MIR378C, CDH13, SLC44A2, IL12A-AS1, LINC02096, CDRT7, ADGRL4, AIFM1, LINC00641, RNU6-133P, CNKSR2, WSCD2, RPTOR, LINC02264, CAMKID, GNA14, GNAQ, LINC02751, SLC12A9, SLC12A9-AS1, PRKCA, U2, LETM2, HDAC7, NPHP3-AS1, LINC00933, CNTNAP5, PLCL1, SPATS2L, RNU6-334P, PCLO, SUFU, TRIM8, NT5C2, SNX19, RN7SL167P, KLC1, COA8, TAOK2, MAU2, RN7SKP19, LINC01360, DDN, ERBB2, PGAP3, RBMS3, ME1, CMTR2, CALB2, LINC02513, PHAX, PEBP4, LINC01754, ZMYND8, TUBGCP5, SERGEF, MKLN1, ADD3, NFIX, RASAL2, LINC00698, UBL5P3, ZBED9, GPX5, SLIT1, RPS27P18, IGSF9B, CPNE8, ARHGEF15, LINC01643, ZNF204P, ZNF391, ZNF184, HNRNPAIP1, KMT2D, GPR1-AS, HLA-U, HLA-K, SPTLC1, LINC00243, KCNMB2-AS1, KCNMB2, NLRC5, PDZD8, EMX2OS, COMMD10, PIK3C2A, GPR89P, RSL24D1P1, TTLL6, ALDOAP1, POCIA, CREB5, GULOP, SUMO2P2, LINC01239, ANAPC7, KLF16, STAU1, MIR137HG, LINC02776, RPL30P1, LINC00879, PPP2R3A, FAT4, DMTF1, DLEU1, CGNL1, PRKAG2, CADM3, RYR2, FXR1, SOX2-OT, LINC00440, NALCN, SLC35F1, RXRG, BCAS1, SUMO1P1, TRUB1, LINC02626, TAF9BP2, CEP85L, NLGN1, ASCC2, VLDLR-AS1, PTPRG, ATRNL1, TRIM42, NAALADL2, TSHZ2, RIN2, FBXL17, PDZD2, SLC25A36, DSEL-AS1, IGKV1OR2-118, IGKV3D-7, RINT1, MTFR2, PDE7B, RN7SL87P, Metazoa_SRP, EFCAB10, JPH3, TAFA5, CLSTN2, CDH4, TCF7L2, BRI3BP, AACS, ALOX12-AS1, ALOX12P2, CFAP57, EBNA1BP2, ZNF577, SH3PXD2B, LINC02058, GOLGA8S, HERC2P6, CLHC1, ADAM19, SEPHS1P2, WWOX, TMEM132B, CDH23, APOB, LINC02850, ROR2, NFIL3, ARSG, LINC01121, ANTXR2, CLTA, LINC00533, RPSAP2, ACADS, SPPL3, ENOX1, RPL31P12, SORCS3, TUBGCP6, MYH15, LINC01645, CNNM2, RSRC1, GRIK3, FTLP18, RNA5SP87, TMEM258, RBFOX1, HMGB1P18, TMEM106B, AREL1, RNU7-160P, BAG5, RPS29P5, COP1, LINC02240, SLC30A9, LRFN5, SHISA9, SOX5, GRM5, SCAMP1, TENM2, MIR924HG, GRIK2, ASCC3, LINC02853, SOX6, SETBP1, RNU6-695P, PSMCIP5, LIN28B-AS1, LINC01613, ESR2, SYNE2, MARK2, CNTN5, OLFM4, LINC02163, L3MBTL2-AS1, L3MBTL2, LINC01572, PMFBP1, OSBPL3, DCC, MEF2C-AS2, PIPOX, PAUPAR, FAM172A, NOL4, ASXL3, MTND2P8, LINC01876, CCDC73, DENNDIA, KDM2B, NFAMI, ZNF536, RAB27B, NTRK2, RTN1, MIR548AI, BDH2P1, RNU4ATAC8P, RNA5SP50, Clorf53, DENNDIB, ATP13A4, LINC00534, TLR4, TPTIP9, EMILIN3, TCAIM, SPCS3, PXDNL, CARMIP1, ARHGAP15, RERE, RERE-AS1, CACNAIE, WASF3, LINC00336, DGKG, ETV5, ATP2A1, SH2B1, CLN3, LINGO2, NEGR1, EEF1A1P11, UBE2WP1, MAP2K5, FTHIP5, RPGRIP1L, RN7SL831P, DTX2P1-UPK3BP1-PMS2P11, IFITM3P3, ILRUN, RN7SL299P, SIPAIL2, SLC10A7, CTTNBP2, PTPRC, ATP6VIG3, RN7SKP120, TUSC1, FHL2, NKAIN2, ADGRB1, TACRI, CWC27, LINC00676, LINC00399, CLCN3P1, FGGY-DT, LINC01358, EGFR, PTS, SESTD1, TCF3, TBC1D3P2, EFCAB3, LINC01789, LINC01885, RPSAP72, RPL17P25, SLC25A26, PSMC1P8, RPRD2, SDCCAG8, CSMD1, E2F7, CSRP2, ADAMTS2, ZNF354C, DCHS2, FTO, TEX2, PCSK5, NCBP3, METTL9, MDFIC, FOXP2, ADAMTS19, CCBE1, TFEC, RPL31P57, RNU1-146P, FAM178B, LINC02488, HLA-V, IFITM4P, HLA-DMA, ADCY1, FAM177A1, LINC00461, GRAMDIB, MYO1H, EFLIP1, DNM1P41, AMPD3, IFT88, ALDH1A1, ANXA1, MYO18B, AMIGO1, GPR61, MINDY2, LINC02188, LINC02181, ZNF385C, FTH1, LINC02733, LYZL4, GNA12, CARD11, NBAS, LRATD1, MKNK2P1, C6orf89, PI16, RNU6-461P, ZPLD1, RCL1, ECMIP1, SORT1, MOCOS, KLF9, NCMAP, TMEM229B, PLPP4, RPL21P16, THSD4, FAAH, NSUN4, FGGY, CASP3P1, LINC01788, MGC27382, GSTM4, RPL7P8, CAMKMT, PSMC1, KCNK13, RANP7, ZBTB20, EIF4E2P2, PDPR, TARSI, TOMM40P3, LINC01622, DGKB, HLCS, CHCHD3P1, KRT8P37, PSAP, CHST3, SHC4, ADAM12, PGLYRP3, LORICRIN, LRPIB, LINC02761, SDCBP2, SDCBP2-AS1, SLTM, RNF111, DNAJC7, LINC02329, RNU6-976P, IDO2, LINC02866, FBXO11, PPIAP62, ZFAT, MTCO1P49, TYRL, TRIM51FP, UBE2G1, RNU6-1271P, FBXO28, SLC30A3, UBE2HP1, PAG1, LINC01006, MYL12BP2, RN7SL28P, LINC02200, HMGB3P16, TRIM26, LINC01929, SNORC, FOXN2, PPPIR21-DT, TCF15, SRXN1, LINC00707, RNA5SP403, RNA5SP404, CLEC17A, ZP3P2, SMARCA2, RN7SL592P, POLRIH, ZNF615, MUCL3, SFTA2, SEMA3A, MRM2, GRIK1, FEZ1, STT3A, KMT2A, SLC17A6, ANO5, PRPF3, KIF5C, HLA-DQB3, MTCO3P1, TCF4, RNA5SP459, GLT8D1, NTRK3, SPATA33, CDK10, ILIR2, ILIR1, AS3MT, NEURL1, SLC35F2, HINT1, PPP2R2B, RNA5SP169, KIF21B, HDAC4, HLA-DMB, BANK1, ZFPM2, ZSCAN12, METTL15, LINC02758, OR5BAIP, OR5AZIP, TYR, CCDC68, CTDP1, KCNG2, KIZ, KIZ-AS1, SLC12A5, PTPRF, KDM4A, LINC02797, HNRNPA1P46, NCOA5, MIRLET7BHG, PJA1, OLFM3, FHIT, LINC02500, LINC00290, PAK5, ACTRT2, DSCR9, SLC6A6, ATCAY, WT1-AS, EIF3M, LINC02057, CACNAII, RPS19BP1, RPS6KL1, PPPIR16B, ACTR5, CAMTA1, RNU1-130P, PDE4B, HYI-AS1, ARL3, STAG1, DGKI, SPAG16, MOB4, HSPE1-MOB4, NAA40, RCOR2, CTNND1, GID4, NXPH4, SHMT2, ZSCAN31, IMMP2L, MEF2C-AS1, NRGN, AMBRA1, MYO18A, FOLHIB, OTUD7B, VPS45, LINC01416, EPHX2, MOCS2, TMEM161B-AS1, MROH5, MIR1302-7, THOC7, GMIP, LINC01088, NAA11, HCN1, MPHOSPH9, or combinations thereof.

In some embodiments, the one or more genes affected by IBD comprise ABI1, FAM238C, ACO2, ACTA2, ADAD1, ADAM30, NOTCH2, ADAMTS6, ADCY3, ADCY7, ADGRL2, ADO, ALDH7A1P4, AFAPIL1, ABLIM3, AHR, AIMP1P2, ALDH5A1, LINC02929, ANK2, ANKRD55, ANTXR2, APEH, ASAP2, ASCL2, MIR4686, ATG16L1, SCARNA5, ATG5, PRDM1, ATP6VIG3, PTPRC, ATXN2, ATXN2L, B3GALT6, C1QTNF12, BACH2, BANK1, BOK-AS1, BPIFA2, BPIFB4, BRD2, BSN, BTBD8, BTF3L4P3, LINC02539, BTF3P2, LINC02300, BTNL2, TSBP1-AS1, BZW2, C10orf67, OTUD1, C16orf96, C17orf67, Clorf141, IL23R, C3orf84, C5orf66, C6orf47, C6orf47-AS1, CACNA2D1, CAMK2A, CAPZB, CARD9, SNAPC4, CAVINI, CBLC, CBX3P1, HSP90AA2P, CCDC26, CCDC85B, FIBP, CCDC88B, CCHCR1, CCL2, CCL7, CCL20, CCL21, FAM205A, CCND3, CCNY, CCR2, UQCRC2P1, CCR5AS, CCR6, CEP43, CCT5P2, CD226, CD244, ITLN1, CD28, CD40, RPL13P2, CD6, CDC37, CDC42SE2, CDH13, CDH3, CDHR3, SYPL1, CDK4P1, LINC01676, CDKAL1, CDKN2A-DT, MTAP, CDYL2, CEBPA, SLC7A10, CELSR3, CEP72, MIR4456, CFAP45, TAGLN2, CFB, CHCHD2P3, LSM14A, CHD5, CHP1, CIITA, CIT, CLCA2, CLEC16A, CLN3, CMC1, CNTNAP2, COG2, COL5A1, COX19, ADAP1, COX6B1P6, RNU6-925P, CPEB4, CPXM2, CRB1, CREM, CRTC3, CRTC3-AS1, CSF2RB, NCF4, CSMD1, CTH, CHORDCIP5, CTIF, SMAD7, CUL1, CUL2, LINC02635, CXCL5, PPBP, CXCR2, CXCR1, CYCSP42, CYLD-AS1, NOD2, CYTH1, CYTL1, RN7SKP113, DAD1, DAG1, DAP, DAP3, DAPK2, DCTD, TENM3, DELEC1, TNFSF15, DENND1B, DLD, PIGCP2, DNMT3A, DNMT3B, COMMD7, DOCK7, DPH5, DPH6-DT, DSE, CBX3P9, DTNB, DUSP16, DUSP22, IRF4, DUSP29, DUSP5-DT, EEF1AKMT2, EIF2S2P3, HHEX, ELF1, EMSY, LINC02757, EPHB4, EPO, POP7, ERAP1, ERAP2, ERGICI, ERN1, ERRFI1, ETS1, EXOC6, Y_RNA, F5, FABP5P10, RBM43, FADS1, FADS2, FAM118A, FAM171B, FAP, IFIH1, FCAR, RNU6-222P, FCGR2A, FCHSD2, FEN1, FERMT1, FGFRIOP2P1, RPS21P8, FLJ31356, FNBP1, FNDC3A, FOSL2, FOXP1, FOXP2, FUT2, MAMSTR, FXNP1, NRXN3, GABRA5, GABRB3, GAL3ST2, GALC, GALNTL6, GATD3A, GBAP1, GCKR, GLYAT, GNA12, AMZ1, GNPDA1, NDFIP1, GOT1, LINC01475, GPBAR1, ARPC2, GPR18, UBAC2, GPR35, AQP12B, GPR65, GRAMDIB, GRB7, IKZF3, GRID2IP, GRP, SEC11C, GSAP, CCDCl46, GSDMA, LRRC3C, GSDMB, ZPBP2, HCG27, HLA-C, HDAC11-AS1, NUP210, HDAC7, HDAC9, HGFAC, HIF3A, PPP5C, HIGDIAP10, SYVNI, HIPK1, HLA-B, LINCO2571, HLA-DQA1, HLA-DRB1, HLA-DQB1, HLA-DQB1-AS1, MTCO3P1, HLA-DRA, HLA-DRB6, HLA-DRB9, HNRNPA1P41, JAK2, HNRNPCP4, HORMAD2, LIF-AS1, HOXA13, HOXA11-AS, HUNK, IDI1P2, IFITM4P, HLA-V, IFNG-AS1, IFNGR2, IGLV3-21, IKZF1, SPATA48, IL10, IL12B, LINC01845, IL12RB2, DNAJB6P4, IL15RA, IL2RA, IL17REL, IL18R1, IL18RAP, SLC9A4, IL19, ILIR1, ILIR2, ILIRL1, IL2, IL21-AS1, IL21, RNU4ATAC4P, IL26, IL27, NUPR1, IL3, CSF2, IL6R, IL7R, IMPG2, INAVA, INKA2, INS-IGF2, IGF2-AS, INSL4, INSL6, INTS11, IPMK, IRF1-AS1, IRF1, LINC02863, SLC22A5, IRF5, IRF6, IRGM, SMIM3, ITGA4, LINC01934, ITGAL, ITLN2, JAZF1, KAT2A, KCP, KEAP1, KIAA1109, KIF21B, MROH3P, KIR3DL2, KLF6, LINC02639, KPNA7, SMURF1, KPNB1-DT, NPEPPS, KRT18P39, KSR1, LACC1, NRAD1, LAMB1, LCAT, PSMB10, LCE3A, LCE3B, LDHD, ZFP1, LINC00243, LINC02570, LINC00448, LINC00484, LINC00491, LINC00492, LINC00581, LINC00598, LINC00604, LINC00824, LINC00861, LINC00892, RNU6-320P, LINC00993, LINC01082, LINC02132, LINC01147, LINC01185, LINC01239, LINC01248, LINC01250, LINC01271, RN7SL636P, LINC01438, MIR297, LINC01485, LINC01620, LINC01700, RPL23AP12, LINC01756, LINC01648, LINC01806, PSMD14, LINC01882, PTPN2, LINC01898, LINC01953, LINC01958, RPLPOP7, LINC01989, LINC02009, CCRL2, LINC02042, CD200R1L-AS1, LINC02065, LINC02229, LINC02098, LINC02128, LINC02163, NUDT12, LINC02213, LINC02230, TMEM174, LINC02278, KLF3-AS1, LINC02341, AKAP11, LINC02357, RBPJ, LINC02360, LINC02380, LINC02513, LINC02537, VEGFA, SLC17A1, LINC02625, LINC02723, RPS6KA4, LINC02800, IFNLR1, LINC02814, LINC02888, LITAF, LNC-LBCS, LPP, LPXN, LRRC58, FSTL1, LRRK2, LINC02471, LRRK2-DT, LRRTM3, CTNNA3, LSAMP, LSP1, LTA, TNF, LTBR, LURAPIL, LURAPIL-AS1, LUZP2, LY75, LY75-CD302, LYRM9, NOS2, MAD1L1, EEF1A1P27, MAGI1, MAGI3, MAML2, MAP3K8, MFSD13A, ACTRIA, MHENCR, STMN3, MICB-DT, MICD, HLA-W, MIR3936HG, SLC22A4, MIR4425, RUNX3, MIR4679-2, MLN, MST1, HLA-DQB3, MIND4P6, ST7, MTRNR2L13, TRMT112P1, MUC19, MYRF, TMEM258, MYTIL, NCF4-AS1, NEDD4L, NELL1, NFATC1, NFKB1, NFKBIZ, NIFKP1, CCND3P1, NKD1, SNX20, NKX2-3, NLRP1, IL23R; Clorf141; IL23R; IL23R-RNU4ATAC4P; IL23R; IL23R; IL23R-RNU4ATAC4P, NOTCH1, NOTCH4, NPM1P17, NR5A2, NRP1, NXPE1, NXPE2P1, NXPE4, OPCML, OR5B21, OSMR, OTUD3, RNF186, P2RY2, P4HA2, PDLIM4, PARK7, PARPIP1, ANKRD10, PCSK5, PDE4A, PDGFB, RPL3, PER3, PHACTR2, PHTF1, RSBN1, PIK3R1, PKIG, PLA2G2D, PLA2G5, PLA2G4A, PLA2R1, PLAU, C10orf55, CAMK2G, PLCG2, PLCL1, PLCXD2, PHLDB2, PNKD, TMBIM1, POFUT1, KIF3B, POU5F1, PSORSIC3, PPIAP34, ZBTB40, PPMIG, NRBP1, PPP2R3C, FAM177A1, RN7SKP211, PRDM16, PRDX5, PRKCB, PRKCQ, PROCR, MMP24OS, PRXL2B, TNFRSF14, PSMA2P1, PSMA6, NFKBIA, PTGIR, CALM3, PTK2B, PTPN22, PTPRD, PTPRK, THEMIS, PUS10, PYGO2, SHC1, RAD51AP2, PSMC1P10, RAPIA, RARB, RASGRP1, LINC02694, RAVER1, RBMS3, U3, RDX, RELA, RFT1, RFX6, RGS14, RIC8B, RIPK2, RIT1, RLN2, RMI2, RN7SKP181, RN7SKP226, PVT1, RN7SL18P, RN7SL51P, RN7SL72P, RPL32P17, RNASET2, MIR3939, RNF220, RNFT1P2, GIPC2, RNU1-150P, TTC33, RNU4ATAC14P, U2, RNU6-119P, RNU6-144P, RNU6-474P, CTLA4, RNU6-793P, PPIAP9, RNU6-794P, RNU6-921P, ZFP36L1, RNU7-147P, RORC, SYNGR1, RPL35P9, LINC00460, RPL5P26, COL13A1, RPL7P47, RPS23P10, HSPA6, RPS6KB1, RPS6P12, RPSAP64, RSPO3, RTEL1-TNFRSF6B, TNFRSF6B, RTEL1, RTF1, SATB1-AS1, SATB2, SBK1, NPIPB6, SBNO2, SBSPON, SCAMP3, SDK1, SEMA6D, SERPINB6, SETDIA, SFMBT1, SH2B1, SH2B3, SIAH3, ZC3H13, SKAP2, SLAIN2, SLAMF8, SLC22A23, SLC25A28, SLC2A13, LINC02555, SLC39A11, LINC00511, SLC43A3, RNA5SP341, SLC9A2, SLC9A8, SLIT1, SMAD3, SNN, SNORD116, SNRPGP8, SOX5, SP140, SPDEF, SPRED2, DNAJB12P1, SRP14P1, SRRM1P3, RPL22P23, STAT3, STAT4, STK11, SUFU, SULF1, SUOX, SYNDIGIL, TAβ2, TAβ2-AS1, TAGAP-AS1, TAP2, PSMB8, TBC1D1, TC2N, TCERGIL, TET2, TEX41, THADA, TLR4, TM9SF4, TMC04, TMEM106B, TMEM117, TWF1, TNFRSF1A, TNIP1, TNRC18, TNXB, TOM1, TRAF3IP2-AS1, TRAF3IP2, TRIB1, TRIM15, TRPM3, TSHZ2, RPL36P1, TSPAN14, TUBD1, TYK2, FOS, CCR12P, GPR183, UBASH3A, UBE2L3, UBE3D, UBR4, IFFO2, UQCR10, UQCRHP1, NCR3, USP1, KANK4, VSTM2B, VSX2, CXCR5, RBX1, YBX1P5, PTCD2, ZBTB38, ZBTB9, GGNBP1, ZDHHC23, ZEB1-AS1, MAGOH3P, ZFP90, ZGPAT, ZMIZ1, ZNF300, ZNF300P1, ZNF831, ZNRD1ASP, ZYXP1, or combinations thereof.

In some embodiments, the one or more genes affected by MDD comprise RN7SKP19, LINC01360, LINC02163, RNU6-334P, BTN3A2, VNIR11P, VNIR13P, MIR3143, RPL10P2, MCFD2P1, ZNF184, H3C11, H1-5, OR2W4P, IQCB2P, PGBD1, ZSCAN12, RPSAP2, LINC00533, HCG14, ZNF90P2, OR14J1, OR2U2P, OR5V1, LINC01015, LINC02829, ZFP57, ZDHHC20P1, POLRIH, TRIM26, ATAT1, FLOT1, SFTA2, HCG21, MUCL3, LRFN5, LINC02315, RPL31P12, OR5BAIP, OR5AZIP, FURIN, LINC02441, LINC02369, GRIK3, AGBL4, ELAVL4, NRDC, GAPDHP24, COP1, HNRNPA1P46, RNA5SP87, LINC01830, VRK2, MYOSLID, ERBB4, RBMS3, ZKSCAN7-AS1, SOCS5P3, BSN, FHIT, RSRC1, FAM172A, CCDCl62P, ADH5P4, HIVEP2, THSD7A, TMEM106B, FAM183BP, KRT8P20, PCLO, FOXP2, AKAP8P1, JKAMPP1, RN7SL720P, ZCCHC7, FAM120A, ASTN2, CNNM2, SORCS3, METTL15, LINC02758, DAGLA, SPTBN2, GRM5, NCAM1, DRD2, ACVR1B, B3GLCT, HS6ST3, ESR2, AREL1, TRAF3, RNU7-160P, BAG5, SEMA6D, MEGF11, MRPL46, METTL9, MIR4318, CELF4, KC6, NPMIP1, DCC, LINC01929, TCF4-AS1, TCF4, LINC01415, LINC01416, CTDP1, KCNG2, NCOA5, MIRLET7BHG, MARK3, ACTL8, TM4SF4, ENPP2, LINC02571, SETDIB, RN7SKP254, PGAMIP12, LINC01582, ZC2HCIA, PKIA, PVT1, MROH9, TAF4, RNU1-89P, LZTS2, ACTR5, CACNAIC, OR4E2, LINC02665, ZSCAN9, TOB2P1, ZNF192P2, ZNF603P, ZSCAN16-AS1, SMIM15P2, ZSCAN31, ZSCAN26, ZNF192P1, NKAPL, ZKSCAN4, ZKSCAN3, PDE4B, ZKSCAN8, ZSCAN23, RNU2-45P, KRT18P1, LINC01556, OR2W2P, TYR, KLC1, CCDC68, KIZ, KIZ-AS1, SLC12A5, PTPRF, KDM4A, NEGR1, LINC02797, ACTG1P22, ITIH3, CNKSR2, PJA1, or combinations thereof.

In some embodiments, the one or more genes affected by MS comprise HLA-DQA1, HLA-DRB1, TNXB, CD58, HLA-F-AS1, HLA-F, IL12A-AS1, IL2RA, TNFRSFIA, HNRNPKP2, CLEC16A, IL7R, LINC02132, CD6, TTC33, MPHOSPH9, ZMIZ1, MGAT5, RPL5, DIPKIA, METTL1, CYP27B1, RPL13P2, CD40, USP31, ASAP1, KIAA2026, MLANA, LINC02580, HLA-DRA, HLA-DRB9, STAT3, LINC01845, IL12B, TBKBP1, KPNB1, Y_RNA, CD86, TAGAP, TAGAP-AS1, RMI2, DLEU1, LINC01967, HLA-W, HLA-A, EPS15L1, PLEK, WDR4P2, SLC30A7, HNRNPAIP68, SSTR5-AS1, SOX8, OR7E108P, SYK, TNFSF14, ZFP36L1, RNU6-921P, ZNF767P, PFDN4, CYP24A1, FCRL3, IL12A, RPL32P23, MAF, LINC01229, RNA5SP443, MAP3K14, MPV17L2, MYNN, SESN3, FAM76B, LINC02085, ZPLD1, PTPRK, THEMIS, RGS1, SAE1, SP140, TSPAN31, AGAP2, AHI1, ALPK2, BACH2, BATF, INAVA, TIMMDC1, CD5, CDC37, CHST12, CLECL1, CXCR5, DDAH1, BCL10, DKKLI, EOMES, LINC01980, EVI5, EXTL2, LINC01147, IL22RA2, IL20RA, VWA8, RGCC, MALT1, FUT2, MAMSTR, MANBA, MAPK1, MERTK, TTC34, MYB, HBSIL, PVT1, LINC02539, NCOA5, NDFIP1, KLHDC7B-DT, ODF3B, PKIA-AS1, PLCL2, CEACAM16-AS1, LMAN2, RGS14, RPS6KB1, RREB1, TCF7, SLC15A2, ZBTB46, LINC02664, ZNF438, SPEF2, LINC01762, TSBP1-AS1, BRINP1, IGF2R, UGT1A12P, UGT1A1IP, MET, NLRP11, ZNF627, CIGALT1, BUD13, LINC02702, ZFHX3, LEKR1, RN7SL332P, PDZRN4, RPL23AP41, GPC5, CSMD1, ADAMTSL1, C20orf202, RAD21L1, RPL30P3, ASAP2, LINC02032, SVIL, KCNIP1, FOXO3, MXI1, BICD1, CPAMD8, LINC02313, NUBPL, KIFIB, CLSTN2, BTNL2, LINC02633, RASGEFIA, VAV2, ZNF433, ZNF433-AS1, CBLB, TSBP1, NDUFA5P8, MRGPRE, ARHGEF10, KBTBD11-OT1, LINC00298, ARL11, RCBTB1, WTAPP1, HACE1, VTI1A, SAMD12, NCKAP5, TLL1, RNASEL, FAM47B, DSC1, EGFL6, ATXN1, CASC19, PCATI, VANGL2, STAT4, TOX2, NPM1P33, KRT18P39, DOCK10, LRRC34, LRRIQ4, TRIM14, RHOH, LINC02265, ALDHIL1, BCL2, VDACIP9, TENT4A, LINC02102, IFNGR2, FLVCR2, JADE2, CXCR4, CBFA2T3, ACSF3, IFNAR1, IL10RB, OS9, SYPL1, CDHR3, TGFBR3, RNA5SP431, SLC2A4RG, GTDC1, JAK1, TYK2, RBM17, IRF1-AS1, IRF1, FAM167A, FAM167A-AS1, JAZF1, PLAU, CAMK2G, SPRED2, DNAJB12P1, PRDX5, CCDC88B, TERT, LINC01934, CRIL, NCF4, NCF4-AS1, CRTAP, PLXNC1, WWOX, SLAMF7, CD48, SPATA48, NLRC5, ALDH7A1P4, IFITM3P8, NASPP1, LBH, RN7SKP211, SLC9B1, ANKRD55, IQCB1, PUS10, RRAS2, COPB1, TTC28, FOXP1, WAKMAR2, CARD11, RASGRF1, CTSH, SLAMF1, SETP9, KPNB1-DT, ZHX3, BTF3L4P3, BHLHE40-AS1, ITPR1, VMP1, KEAP1, PDE4A, IL6, MTCYBP42, ZC3HAVI, SLC7A10, CEBPA, CD27-AS1, CD27, JAK2, HNRNPA1P41, ETV7, FPR1, POU2AF1, FLI1, ETS1, LINC02660, LINC02639, UCK2, HOXA1, HOTAIRMI, DMRTA1, CDKN2B-AS1, ADCY3, SETDIA, SYDE2, Clorf52, LZTFL1, MEF2B, BORCS8-MEF2B, FTH1P22, GNG2, MIR3681HG, PRICKLE1, TBX6, GALC, ETV3, ETV3L, KCP, IRF5, NCOR2, ZNHIT3, IFI30, PHGDH, FAM241A, LINC00271, ATP5PBP6, TSGA10IP, DRAP1, HTR3A, GFI1, ELMO1, SLC2A4, DLL4, ISCA1P4, SCAMP2, GABARAPL3, GOLGA2P8, UBASH3B, GLULP3, PRXL2B, RUNX3, SMG6, HIC1, TXK, PHACTR2, MAPK10, RN7SL519P, ATF1, BCL2L14, ETV6, N4BP2, RN7SL203P, TRAM1, PITPNM2, LINC02390, NR1D1, BATF3, NSL1, MEMO1P2, SATB1-AS1, IKZF3, LEF1, ADIIP1, LPIN3, TRAF3, RCOR1, HORMAD1, CTSS, HDGFL2, UBXN6, MARCKS, DNMT3A, PRR5L, GATA3, SLC6A16, CD37, TNIP3, MTHFR, MED6P1, RNA5SP158, KLF3, PHKG1, MYBPC3, LINC01624, LTBR, SRP14P1, CD226, BCAS1, MCTP1, AFF1, LINC02732, PPA2, RN7SL89P, APOA4, APOA5, ERG, ZNF652, FLJ40194, ESPN, ITK, FAM71B, TNFAIP8, PHLDB1, IFITM3, L3MBTL3, MAZ, TOP3A, TNFRSF14, RNU1-150P, PLEKHG5, AGBL2, FCRL1, CENPO, LINC01185, CORO1A, MAPK3, TET2, PPIAP9, RNU6-793P, PXT1, NPEPPS, MRPL45P2, SKAP2, TREHP1, RNU6-376P, HNRNPCP4, TM9SF2, CCR12P, RNU6-415P, IQGAP1, CCR4, GLB1, CDH3, GRB7, SLC44A2, SLC9A8, IKZF1, MMEL1, CD69, TSFM, CSF2RB, TEF, TOMM22P3, LINC00423, COG6, JDP2, RASGRP1, RPL23AP91, CCL22, SLAMF6, MIR181A1HG, CD28, ZBTB38, LINC01100, LPP, SH3BP2, FNDC1, NCOA2, VCAM1, ZFP36L2, CNRIP1, LINC02084, NFKB1, TNFAIP3, LINC02528, PKIA, ZC2HCIA, CSGALNACT2, LINC-ROR, BODIL2, RELN, KCNB2, RNA5SP271, SGCD, PCSK5, AXDND1, SNHG28, CHROMR, PRKRA, HLA-DRB6, HLA-DQB3, MTCO3P1, HLA-DQB1, TNPO2, GGNBP1, ITPR3, IP6K3, RN7SL824P, RPAP2, SIPRI, ARHGEF3, WNT9B, NUTF2P8, ARNTL, TM2D3, SGO1-AS1, UBBP1, PTGFR, BBX, LINC01990, PADI1, LINC02616, NCOA4P1, FUT8, CUEDC1, SUMF1, LINC01551, YWHAG, TRIM2, HSPB1, BVES-AS1, POPDC3, ENTHD1, GRAP2, EXOC2, CASP8, UNK, SMARCA4, NRROS, LINC01063, ANKRD36BP2, TMEM244, RNU6-919P, RPS6KA5, RPL23AP12, NCKAP5L, SCAF11, TRIM71, ARHGAP26, NR3C1, LIPG, SMUGIP1, ARHGAP27, MPO, TSPOAP1, PTPN11, SERBP1P3, RFT1, SGSM2, FAM221B, TMEM8B, LCN6, RORA, MEF2C, MEF2C-AS2, PDE4B, TNIP2, MIR4435-2HG, SLA, TG, LINC01136, LINC01353, MINK1, AKAP13, LINC02644, MYO5B, LINC01953, IKZF2, NAβ1, PLEC, EPPK1, ATXN2, SH2B3, SIDT1, GRB2, LIMK2, MARCHF1, GIMAP2, GIMAP6, LINC02163, GCG, EIF3EP2, or combinations thereof.

In some embodiments, the one or more genes affected by OCD comprise RNU6-765P, YWHABP1, ZCCHC7, ZFPM2, PXDNL, CSMD1, FGFR1, EPHX2, GULOP, TUSC3, PPM1AP1, TSNARE1, MROH5, MIR1302-7, ZNF804B, FLNC, GRM3, GRM3-AS1, PCLO, RPL31P35, SEC61G, OSBPL3, GSDME, MAD1L1, BRAF, CCT4P1, DGKI, IMMP2L, SRPK2, HLA-DRB5, EIF4EBP2P3, UFL1-AS1, SNAP91, SNORA70, LINC01526, LCA5, SH3BGRL2, RIMS1, ZSCAN31, CARMIL1, NPMIP10, R3HDM2P2, MEF2C-AS1, LINC00461, TMEM161B-AS1, LINC02057, PDE4D, LINC02101, MOCS2, HCN1, TENM2, LINC01470, PPP2R2B, KDM3B, LINC02240, LINC02163, RNU6-334P, GRID2, NAA11, LINC01088, LINC02260, KIT, SLC30A9, DMP1, LINC02172, MANBA, BANK1, SLC39A8, POUIF1, THOC7, CACNA2D3, ITIH3, FBXW12, TCAIM, HSPDIP6, LINC02033, FECHP1, CNTN4, RPL21P17, RSRC1, STAG1, LSAMP, TCF20, L3MBTL2, L3MBTL2-AS1, CACNAII, RPS19BP1, NFATC2, ATP9A, KCNB1, ACTR5, PPPIR16B, KIZ-AS1, KIZ, Y_RNA, RPLPOP1, LMAN2L, CNNM4, LINC01122, VRK2, ACTG1P22, NGEF, SP110, SLC16A14, LINC01830, RNA5SP87, SPAG16, PTH2R, LAPTM4A-DT, RPS16P2, RN7SL717P, FTCDNL1, PLCL1, HSPE1-MOB4, MOB4, LINC01876, CD320, CHCHD2P3, LSM14A, GMIP, WDR7, LINC01416, TCF4, LINC01929, DCC, KC6, IMPA2, RPTOR, LINC01987, MYO18A, GID4, GRIN2A, TUBB3, DEF8, PMFBP1, RBFOX1, TMEM219, SHISA9, H3P40, LINC01581, FURIN, CHRNA3, FOXB1, LINC02694, TUBGCP5, BCL11B, ADCK1, RPS6KL1, RGS6, SYNE2, ESR2, RHOJ, RTN1, GPR135, LRFN5, NPAS3, AKAP6, NUBPL, PPP1R13B, RNU7-160P, BAG5, ACOD1, RPL7P44, LM07, OLFM4, ENOX1, TRPC4, ANKS1B, TAFA2, SHMT2, NXPH4, LINC02395, CACNAIC, SOX5, SLCO1C1, SLCO1B3, MPHOSPH9, HCAR2, HCAR3, SPPL3, ACADS, ATP2A2, FOLHIB, RCOR2, NAA40, FADS2, CTNND1, AMBRA1, PAUPAR, IGSF9B, RN7SL167P, SNX19, NRGN, NECTIN1, DRD2, LRRC20, LINC00702, CACNB2, ADRB1, NHLRC2, SORCS3, NT5C2, TRIM8, ARL3, EEF1A1P11, RN7SL831P, RNU1-130P, CAMTA1, PKN2-AS1, DDAH1, BCL10, RERE, RERE-AS1, LINC01360, RN7SKP19, RPL31P12, PDE4B, PTPRF, HYI-AS1, FTLP18, GRIK3, ATP6V1G3, PTPRC, LINC01645, S100A7, RN7SL44P, VPS45, OTUD7B, DRAXIN, or combinations thereof.

In some embodiments, the one or more genes affected by PD comprise TMEM175, BST1, LRRK2, LAMTOR2, RAB25, CCNT2-AS1, MCCC1, SNCA, STK39, CCDC62, LINC02210, LINC02210-CRHR1, POLR3B, RFX4, ISM1, TASP1, FAM47E-STBD1, FAM47E, ITGA8, NSF, NUCKS1, CYP17A1, WBPIL, MMRN1, SNCA-AS1, LINC02331, GAK, HLA-DRA, STAP1, DLG2, SEMASA, SLC2A13, SLC41A1, RAB29, PLEKHM1, TAS1R2, PAX7, BRINP1, DGKQ, RNU6-525P, GFPT2, SPPL2C, MAPT-AS1, RIT2, SREBF1, RNU6-1326P, CYCSP42, PRDM15, CNKSR3, TSBP1-AS1, SLC50A1, CTSB, FDFT1, SH3GL2, ANO5, LINC02349, PRSS53, ZNF646, WNT3, TCIM, LMNB1, MARCHF3, CLRN3, ODAPH, CTC1, WNT9A, CICP26, COL5A2, IGSF11, LINC02224, MDGA2, RPA2P1, ZNF396, INO80C, GCH1, TMEM229B, BCKDK, MAPT, TMPRSS9, DDRGK1, LZTS3, RN7SL474P, SIPA1L2, HLA-DQB1, MTCO3P1, GPNMB, INPP5F, IGSF9B, SPATA19, LRRK2-DT, CNTN1, KANSLI, KCNN3, PMVK, UNC13B, CCDC82, TMC3-AS1, TMC3, COL13A1, LINC01307, MCCC1-AS1, ITPKB, ITPKB-IT1, FYN, SV2C, CHL1, CHL1-AS1, SP1, LINC01500, CYP4Z1, GABRG2, TMC5, NKAIN2, RNF217-AS1, FANCF, LINC01495, RNU6-161P, TPIIP1, RN7SKP190, MYLK2, DYRKIA, TRPM2, PRAG1, LINC01012, RERE, RERE-AS1, MROH3P, LINC01127, ILIR2, NGEF, TBC1D5, PARP9, ASSIP14, SPTSSB, MTNDIP24, LINC02400, GPR65, TPM1, LACTB, ELOVL7, HLA-DQA1, HLA-DRB1, LINC02527, KLHL7, ZDHHC2, CACNAIB, BAG3, BICD1, RESF1, MED12L, CD38, SLC39A8, CAMK2D, STX1B, LINC02240, ZNF608, MED13, Y_RNA, SRSF10P1, SMAD4, CHRNB1, MUC19, SETDIA, SCARB2, CLCN3, EIF3KP1, PAM, C5orf24, TXNDC15, RSL24D1P1, GPR89P, TRIM40, LINC00326, HMGB1P13, SAPCD2P3, GS1-124K5.4, BIN3, CYRIB, UBAP2, GBA, RNU6-481P, FCGR2A, VAMP4, EEF1AKNMT, KCNS3, KCNIP3, MAP4K4, TMEM163, RBMS3, IP6K2, KPNA1, LCORL, GXYLT1, RNA5SP443, ARHGAP27, RETREG3, UBTF, FAM171A2, BRIP1, DNAH17, ASXL3, SPPL2B, CRLS1, SLC25A44, SEMA4A, SLC44A4, EHMT2-AS1, DNMIL, FGD4, GBF1, RNF141, NCKAP5L, SCAF11, HIPIR, FBRSL1, CAβ39L, MBNL2, LINC00456, MIPOL1, RPS6KL1, RNU6-835P, SYT17, RABEP2, CD19, NOD2, CASC16, PHBP21, DCUNID1, KLHL7-DT, FAM126A, RADIP1, TIALI, OR5AZIP, OR5BD1P, LINC02451, NDUFAF2, CA8, HSD17B1P1, NAGLU, ING1, ITGAE, HASPIN, ZSCAN16-AS1, ZNF165, RPL13AP3, KTN1, SCN2A, AGAP1, LTK, ITGA2B, ITIH1, DSG3, OCA2, ATF6, PRRG4, QSER1, AAK1, COL3A1, DIRC1, TCEANC2, MX2, ZP3, TRAPPC2L, PABPNIL, KCNIP4, MIR663AHG, HTR2A-AS1, HTR2A, ZNF474-AS1, PTPRD, MCTP2, KLHL29, RNU6-121P, AQP10, ANO2, GPR19, THSD4, APOE, SLCO1B3, NR1D2, DENND2B, CRISPLD1, LINC02885, NTRK2, RNU7-137P, AACS, ASSIP13, DRAP1, BCCIP, LINC02629, FAM184A, LINC02114, ZNF789, SULT1C2, GPR32P1, GPR32, SQOR, PRKN, PLPPR1, PWRN4, LHFPL2, TRPS1, KLHDC1, LINC00476, or combinations thereof.

In some embodiments, the one or more genes affected by RA comprise TYK2, PTPN22, STAT4, IL2RA, ANKRD55, TAP2, HLA-DRB1, HLA-DQA1, MTCO3P1, HLA-DQB3, HLA-DQB1, HLA-DOB, HLA-DRB9, HLA-DRA, TSBP1-AS1, FLI1, ETS1, DRAIC, KCP, IRF5, MACIR, RAD51B, RASGRP1, CDK6, FAM76B, SESN3, LINC02357, SYNGR1, DUSP22, IRF4, TNFSF4, TTC34, SPRED2, COG6, C5, TRAF1, TNFRSF14-AS1, WDFY4, REL, RUNX1, TAGAP-AS1, IL6R, CBFA2T3, ACSF3, CCR6, RGS22, GSDMB, SH2B3, ATXN2, CLNK, LINC02498, ICOS, CTLA4, LINC02664, ZNF438, FLT3, LINC02528, TNFAIP3, ILF3, ACOXL, MIR4435-2HG, FCGR2A, PHTF1, RSBN1, MECP2, ARID5B, CXCR5, Y_RNA, ATG5, LINC02539, BTF3L4P3, LINC02132, LINC01082, CD40, BLK, FAM167A, ICOSLG, NONOP2, LINC01185, IL12B, LINC01845, USP8P1, HLA-C, TRAF3IP2-AS1, TRAF3IP2, LINC02085, NFKBIZ, RPL23AP96, GS1-24F4.2, CTNNA3, INSL6, Clorf141, STAT2, FBXL19-AS1, CTF2P, NOS2, IL23R, RN7SL51P, GYPC, TNS1, RPL7LIP9, TNIP1, GABBR1, HLA-F-AS1, HLA-F, HLA-G, HCP5B, MICD, RNF39, TRIM31, TRIM26, HCG17, HCG18, MICC, MRPS18B, PPP1R10, HCG20, DDR1, MUC22, POU5F1, PSORSIC3, LINCO2571, MICA, RPL15P4, BAG6, HSPAIB, HSPAIA, SKIV2L, TNXB, NOTCH4, HLA-DMA, SLC16A10, REV3L, MFSD4B, ATP5MC2P2, EDIL3, ZP3P1, ANO6, AKAP6, NPAS3, CSF2RB, CCDCl62P, ILIR1, ILIR2, RPL23AP12, ERAP1, RNU6-283P, DHFRP2, LINC01934, MALRD1, ANTXR2, HLA-S, SRP14P1, LTBR, TTC33, CARD9, SNAPC4, KPNB1, TBKBP1, KIF21B, LINC01147, LYRM9, CDC37, GATD3A, HHAT, LINC01980, EOMES, IL7R, CAPSL, POT1, C7orf77, UBE2L3, FGFR3P1, HCG9, MIR4425, RUNX3, INAVA, GPSM1, TNFRSF1A, MRPL45P2, NPEPPS, ERAP2, GPR35, BACH2, ZMIZ1, LINC01475, GOT1, DNAJB6P4, IL12RB2, ERAP1 x RNU6-283P-FGFR3P1, ERAP1-ERAP2 x RNU6-283P-FGFR3P1, HS3ST1, USP8, ADAM28, PCSK5, LINC02096, CHSY1, MYOM2, KRT8P20, FAM183BP, PTTGIIP, FAS, RASSF8-AS1, MIR4302, MEFV, HLA-B, NUP88, RABEP1, RTKN2, LINC02621, LINC02356, OS9, PRKCH, CD83, LINC02786, LINC01343, FLACCI, ZC3H7A, TXNDC11, TPD52, PPIL4, RCAN1, IFNGR2, IFNAR1, LINC00824, NEFHP1, GAPDHP64, CSF2, P4HA2-AS1, SFTPD, GRHL2, RN7SL563P, RNU6-481P, CD226, B3GNT2, TEC, YDJC, JAZF1, PLD4, GPC5, RBFOX1, PADI4, TMEM151B, NFKBIE, PTPN2, ANXA3, EXOC2, LINC02676, LINC00709, CUL5, FADS2, FADS1, IL2, VPS37C, PRR5L, TRAF6, PADI2, FCGR2B, TNFRSF9, UTS2, LINC02649, GATA3, MTF1, UBASH3A, PRDX6-AS1, ETV7-AS1, HTD2, RPP14, SUOX, LINC01104, KRT18P39, CD28, CDKN2AIPNLP3, MMEL1, PLCL2, ZPBP2, SLC17A1, RBPJ, PHF19, CCL21, FAM205A, STAG1, H3P5, LBH, FCRL3, VDACIP9, NEUROD2, CDK12, BOLL, CCR2, CCR5AS, VDAC1, TCF7, IQGAP1, AFF3, TMEM187, SLC9A9, DDX6, RNU6-376P, IL2RB, DNASEIL3, RPL32P23, GATA3-AS1, ELMO1, RPL13P2, RPS12P4, CCDC201, HLA-DRB5, RNU6-420P, GMCL2, RNU1-61P, DPP4, CDK5RAP2, LINC02656, KIF5A, RNU6-474P, AIRE, PDE2A, CEP170B, GCH1, GABARAPL3, ZNF774, PRKCB, ARL15, APOM, FAM205BP, CD247, JDP2, BATF, TSBP1, FAM107A, TNPO3, KIAA1109, CASTOR1, ANO8, DDA1, TCTE1, GPR174, P2RY10BP, CXCL13, IRAK1, RNU6-921P, ZFP36L1, VANGL2, SLAMF6, GTF2IRD1, GTF2I, SWAP70, NFKBIA, PSMA6, LINC00158, LINC02789, PHRF1, GRM5, GUCY1B2, RNASEH2B, CLYBL, LINC01898, SETP16, PTPRC, MIR181A1HG, SMTNL2, CD200R1, CD200R1L, RAVER1, NAβ1, PRDM16, CDC42EP3, PLCL1, CFAP20DC-AS1, CFAP20DC, LINC01088, U6, ALPK1, ZNF366, FSTL4, CPA4, ZNF679, LNCPRESS1, CYRIB, DENNDIA, RNA5SP287, RNU6-1035P, SH3PXD2A, AIFM2, COL2A1, SENP1, MSI1, TMEM179, NECAβ2, PTX4, TELO2, TMEM235, CTIF, POLR3GP2, RPL7AP14, SRC, PHACTR3, SDF2L1, GP1BB, TBX1, LINC02098, KNOP1P1, RN7SL38P, RPS26, ERBB3, ZNF689, ABHD16B, C20orf181, SMC1B, NPM1P33, RBM17, LINC02865, SSBP3, PTGS2, DGUOK-AS1, BAD, GPR137, TPCN2, ARAP1, DAP, PRKCQ, LEF1, P2RY10, MIR4328, PRXL2B, TNFRSF14, MAPK14, IKZF1, NFATC1, DNMT3L, PRDX5, CCDC88B, RNF19A, RIN3, LINC00323, ANAPC4, NDUFB9P2, EEF1B2P6, ARHGEF3, TRHDE, CASP10, CELF2, FBXW8, TGFA, MIR3142HG, WAKMAR2, ICAM3, RN7SL335P, HECTD4, SMG7, NCF2, CCR3, SNORA72, PPMIL, PTPRM, ATG16L1, SCARNA5, BSN, IRF1, IRF1-AS1, IRGM, ALDH7A1P4, TSPAN14, SLC25A28, NKX2-3, EMSY, LINC02757, NOD2, HORMAD2, RPL3, MAGI3, AIMP1P2, RNF186, OTUD3, IL10, UQCRC2P1, APEH, HNRNPA1P41, JAK2, IFNG-AS1, KSR1, LINC01989, CCL2, STAT3, KPNB1-DT, C17orf67, RPS6KB1, ERN1, SLC39A11, CHCHD2P3, LSM14A, SBNO2, STK11, KEAP1, HIF3A, PPP5C, FUT2, POFUT1, KIF3B, DNMT3B, COMMD7, LINC01620, PKIG, SLC9A8, ZNF831, RTEL1-TNFRSF6B, TNFRSF6B, CYCSP42, UQCR10, PDGFB, FAM118A, ZEB1-AS1, DENNDIB, LINC02800, IFNLR1, B3GALT6, C1QTNF12, PARK7, TMCO4, HIPK1, RORC, LCE3A, LCE3B, TEX41, FAP, IFIH1, RPS23P10, HSPA6, PPIAP34, ZBTB40, NR5A2, IL19, ASAP2, ADCY3, DNMT3A, GCKR, FLJ31356, THADA, PUS10, DNAJB12P1, IL18R1, GBAP1, CD244, ITLN1, RNU7-147P, CXCR2, CXCR1, SNRPGP8, CCL20, SP140, AQP12B, GAL3ST2, SATB1-AS1, CMC1, MST1, NPM1P17, BANK1, NFKB1, IL21, IL21-AS1, LINC02213, OSMR, LINC00604, YBX1P5, PTCD2, LINC02230, TMEM174, LINC02863, NDFIP1, CPEB4, RNU6-793P, CDKAL1, LINC00581, DELEC1, TNFSF15, TLR4, NOTCH1, IL15RA, NIFKP1, CCND3P1, CREM, PLAU, ACTA2, HHEX, EIF2S2P3, PRDM1, THEMIS, PTPRK, CEP43, GNA12, AHR, SKAP2, SPATA48, KPNA7, SMURF1, PIGCP2, DLD, RN7SL72P, RPL32P17, COX6B1P6, RNU6-925P, TRIB1, RN7SKP226, PVT1, ASCL2, MIR4686, GLYAT, OR5B21, LPXN, CD6, TMEM258, MYRF, LINC02723, RPS6KA4, FIBP, RDX, NXPE1, LRRK2, HDAC7, RIC8B, RPS21P8, FGFRIOP2P1, LINC00598, NRAD1, FNDC3A, UBAC2, CCR12P, PPP2R3C, FAM177A1, U2, FOS, LINC02694, SMAD3, RMI2, HNRNPCP4, CLEC16A, SNN, LITAF, IL27, ITGAL, SETDIA, NKD1, SNX20, CYLD-AS1, AP4B1-AS1, RPL6P8, ARL14, DGKQ, LIMK1, EIF4H, SCT, DRD4, PTPN11, PRR12, CCDCl16, SBK1, NPIPB6, ATXN2L, PDE4A, MHENCR, STMN3, ADGRL2, LINC01250, CYTL1, RN7SKP113, DCTD, TENM3, RNU6-144P, LURAPIL, LURAPIL-AS1, FNBP1, LINC00993, RPL35P9, LINC00460, ADCY7, LINC00892, RNU6-320P, SRRM1P3, RPL22P23, CRB1, IL18RAP, SLC9A4, DAG1, RNU1-150P, INS-IGF2, IGF2-AS, or combinations thereof.

In some embodiments, the one or more genes affected by SCZ comprise SLAMF1, SETP9, NFKB1, GRB10, RPL14P5, SHISA9, MYBPC3, LINC02742, ZNF804A, FTLP18, GRIK3, NOTCH4, NKAIN2, TCF4, CLC, LEUTX, FEZ1, PRRC2A, CNNM2, BORCS7-ASMT, BRD1, LINC01741, FBXO11, ARHGAP31, POM121L2, MIR3143, RPL10P2, HLA-DQA1, AGAP1, CACNAIC, NT5C2, SNX19, RN7SL167P, NFUIP2, RPL7P9, NDUFS5P2, MAD1L1, ZSWIM6, ANK3, CSMD1, GPR89P, RSL24D1P1, TMEM245, BCL9, Metazoa_SRP, ST3GAL1-DT, NEBL, IRF4, EXOC2, PPFIA2, LINC01016, CNTNAP5, TENM3, TMTC1, LINC01478, SGCZ, PTBP2, RN7SKP270, VRK2, LINC00492, SLCO6A1, PRSS16, CCDC60, ACSM1, ACSM3, PLAA, LSMI, TRIM26, RPS19BP1, CACNAII, DMRT1, PLA2G4A, DOCK1, RAB6C, ARHGAP42P2, RUFY1, U4, MCHR1, SLC25A17, DENND2B, ZFP57, ZDHHC20P1, LINC01378, HS3ST4, COMT, SEPTIN14P21, MIR873, MKNK1, KIT, ADCY7, TENT4B, LINC02645, LINC00701, NOM1, LMBR1, LINC02304, TBX1, CNTN4, LINC01370, LINC02238, MGAT5, RNU6-1029P, SF3B1, SRPK2, LINC00571, DNAJA3, MEMO1P1, SETD4, HMGB1P36, RNU6-281P, TRAF3IP2-AS1, STAG1, BRINP2, LINC01645, LINC00862, SATB2, ZFAND2B, NAβ1, RNU6-1001P, CBX3P6, PPPIR13B, NEU1, PCNX1, LINC00606, TBC1D19, STIM2, LINC01470, LINC01416, LINC01415, ZNF823, SOX5, SP4, MSRA, IMMP2L, GULOP, CMAHP, HLA-DMA, HLA-DMB, CUX1, FOXP1, HLA-DOB, SCNIA-AS1, IGSF9B, EIF4E2P2, HAX1, RNU6-239P, MIR29B2CHG, PROX1-AS1, HFE, INSIG1-DT, RN7SKP280, MMP16, MSR1, PRDM14, SUMO2P20, EBLN3P, CACNB2, LINC02144, MIND4LP18, OR8Q1P, OR8A1, GLG1, LINC01539, LINC01905, ZCCHC14, PHACTR3, NPYIR, MCL1, KIF21B, PCDH15, SCN9A, HNRNPA1P47, NEGR1, TRAPPC3, CAPN2, LRRN2, ZCCHC17, RNA5SP87, SPHKAP, LINC02033, HSPD1P6, BCL11A, ATOHI, LPP, SATB1-AS1, RNU6-367P, EMB, EPC2, TMEM178B, PPP2R3A, LINC01394, FOXF2-DT, FRMD4B, RN7SL18P, KLHL29, NOTO, RAB11FIP5, NRBF2, RNU6-543P, LINCO2195, SKAP1, RTKN2, SUMO2P2, MINDY2, CRYGFP, MEAF6P1, LEP, MIR129-1, MTCYBP27, GTF2A1, DYNLLIP1, TMCO5B, ZNF365, CHRNA3, NFATC3, RBFOX1, RPTOR, SPECC1, GID4, PPIAP14, PCBP3, THOC7, MICC, UBQLNIP1, CASP12, TSBP1-AS1, TSBP1, FGF8, FBXW4, CRB1, NEURL1, CFAP58, LINC02706, MTUS2, NCOA4P1, KCNS3, MSGN1, ACTGIP22, CUL3, RPL21P38, DYNC112, FUT9, KRT18P50, MGLL, PPMIM, WDR82, HS6ST3, GALNT10, LINC00301, GRIN2B, LPCAT4, ACTGIP15, IQANK1, JRK, ARC, RALGPS1, GALNT2, HTR3B, HTR3A, LINC01929, RNU11-5P, PRKD1, DMTF1, PMVK, KCNN3, EBNA1BP2, PDE4B, LINC01776, ELAVL4, SLC45A1, LINC02549, PRSS35, ME1, CCDCl92, Y_RNA, C11orf87, TRPC4, LINC02334, RTN1, NXPH4, SHMT2, KLF12, AKAP6, KLC1, COA8, SEC11A, NMB, LIPC, ALDH1A2, LIPC-AS1, MSI2, ZEB2, RMND5A, LINC01876, PNPP1, TDRD15, FXR1, SLC39A8, SLAIN2, SLC10A4, BANK1, VTRNA1-3, PCDHA1, GRIA1, RANBP3L, SLC17A3, ZSCAN31, CARMIL1, ABCB1, MAILR, LINC00457, GLIS3, SPATA2P1, AKT3, RIMS1, PCGEM1, MED30, FHIT, SPATS2L, NRBF2P3, OTUD7B, VPS45, ARNILA, DTNB, SH3GL3, SHC4, HLA-V, IFITM4P, OR14J1, OR2U2P, RBX1, RNU7-147P, NTRK3, LINC02264, RALGAPA1, TMEM132D, CIR1, LINC01648, HCN1, RBMS3, GRM7, ADCY1, NR3C2, LINC02710, FBLIM1P2, IL20RB, GABBR2, CNTN2, ZFHX3, POP7, EPO, VNIR18P, ARL14EP, RPL12P30, DST, ZDHHC2, KLF6, RPSAP52, SOX2-OT, MEF2C-AS2, PLCL1, EFR3B, ERCC8, XYLB, GRID1, DLG2, HLA-DRB9, CALNI, NOL4, MOSMO, ZDHHC8, LINC02194, PRKCB, RAB8B, SIPAIL1, PIK3C2A, LINC02165, SMG6, KDM4C, TNKS, ZNF536, LINC02438, FAM184B, TTLL1, TTLL1-AS1, EMX1, GGNBP1, MPHOSPH9, RFPL4AP3, SHISA6, CTB-178M22.2, TENM2, TBC1D29P, LINC02812, SLC25A3P1, PCLO, ARHGAP40, INHBA, ZBTB7B, ADGRV1, TRA2A, CLK2P1, NOS1, DOPIB, DNAH1, RGS6, CACNAID, LINC01761, LIMK2, KIFBP, TBC1D5, DRD2, ITIH3, ITIH4, TMEM182, CHRNA2, SFMBT1, HLA-B, LINC02571, MIR137HG, BCL11B, LILRP2, MOCS2, TLE1, RNU6-1035P, EEFSEC, MAIP1, EXOC4, ST13P7, RPL32P31, NPTX1, MEF2C-AS1, MIR217HG, MAU2, GATAD2A, CXXC4-AS1, SLC4A10, DPP4, OPCML, FRMD5, ALGIL13P, FAM86B3P, SCAPER, TRMT9B, LINC00681, KYAT1, PTPRF, RERE-AS1, RERE, ATXN8OS, PSORSIC1, LIN28B, CRYAA, U6, DNAJC11, NRGN, TSPAN2, ARHGEF26, ALMS1, PAK6, KCNG2, CTDP1, NOSIP, PRRG2, YPEL1, GNG7, ZBED4, ARL2, BATF2, PCNX3, TCF20, ZMYM4, KCNB1, MRPS11P1, RN7SL607P, STK4, ARFGEF2, PREX1, CLIP1, LRRTM4, H2AC10P, RPL30P1, LINC02776, PRKG1, SORCS3, GUCY1A2, KRT19P2, NDUFA12, CALB2, RHBDL3, PIK3R1, GUSBP2, TEK, DPYD, SLC9B2, DNAJB8-AS1, RPL35AP15, SDCCAG8, RN7SKP60, PTPRK, GRAMDIB, CADPS2, FEZF1, SNORC, AZUIP1, CDKN2AIPNLP3, POU6F2, CAPZA1P4, FMN2, TTYH3, CHD2, LINC01122, ANKRD23, CNNM3, FER, ALOX15P2, SEC61GP1, B3GAT1-DT, HLA-DQB1, SCAMP1-AS1, THRB, ZDHHC20, PCDH9, SH3RF3, SEPTIN10, EEF1B2P5, LGSN, RCN1P1, ZNF292, FOXO3, AIG1, SELENOH, SYNGAP1, ADAMTSL3, RBM26, RNU7-152P, GPC6, LINC00390, SMIM2-AS1, EP300-AS1, RANGAP1, ZC3H7B, TAβ1, MGAT3, ANKRD36, SNAPC3, SUFU, HECW2, ERLIN1, SPCS2P2, EIF3FP3, LINC01795, ATP2B2, PLCL2, ZBTB20, ZBTB20-AS5, ELAC2, YWHAE, GRIN2A, TMF1P1, LINC01149, HCP5, ITPR3, JAM3, DLX2-DT, ADIIP2, KLF10, HMGB3P24, RNF38, LINC02326, FOX06, CENPM, RN7SL100P, BICC1, DDB2, PACSIN3, TCPIP3, KCNA1, KCNA5, TMEM233, GPM6A, EFHD1, GIGYF2, RPL23AP68, DDX10P2, ZNF318, ABCC10, PRORP, JKAMP, BORCS7, PTGES3P4, LINC02698, RPL12P46, MARK2, GSDME, MPP6, LETM2, U3, AGO4, ASPG, TDRD9, LINC02828, LINCO2240, STK31, HDAC2-AS2, ILDR2, TADA1, PEPD, BNIP3L, PAK2, CLCN3, ANKS1B, POCIB, LINC02406, TACC2, LRP4, C12orf76, SMAD7, DYM, C12orf42, RPL7P43, ZDHHC17, GGTA2P, RAB11AP2, LINC01360, LINC02762, LINC02763, CREB1, EIF5, HMGB3P26, CNOT1, SLC38A7, FANCA, ZNF385B, LINC02050, RN7SL825P, KIF5B, MIR583HG, ATXN7L1, FAM120A, MIR9-3HG, POLG-DT, SGCD, DLGAP2, ASH2L, TMEM219, KCTD13, CWC22, GRM3-AS1, GRM3, LINC01725, KRT8P21, RN7SKP19, QPCT, SNAP91, RASA3, CFAP97D2, ETF1, RIT1, NT5ELP, BTN3A1, TNXB, STUM, RNU6-526P, RNU6-1151P, LINC02404, LINC02219, PPDPF, PTK6, EPN2, MAN2A1, LINC02057, LIMA1, DOPIA, SRR, LINC02267, NAβ2, ZNF664, RFLNA, KDM4B, UHRF1, UBLCP1, LINC01932, NLGN1, H2BC15, H4C12, SPG7, CLDN10, CLDN10-AS1, RNF14P1, SPOPL, RNU6-236P, CXXC5, MIRLET7BHG, ROBO2, EML6, SPTBN1, NTM, LINC01911, LINC02797, NPAS3, ASCL1, LHFPL3, PPP1R3B, DGKI, FAM53C, CDC25C, CYP26B1, MRPS30, DCC, PLCH2, LINC01814, SNRPEP5, FURIN, LEMD2, MAGI2, ASAP1, PTPRD, APBAI, ATP2A2, FAM214A, RUSC2, OSBPL10, LINC01476, RNU1-130P, LINC01787, TTC12, KALRN, RPL4P2, FMO10P, TRPM6, MTHFDIL, OPRD1, DDX56, NPCIL1, MECR, RNU6-63P, LNX2, AGBL1, AGTRAP, DRAXIN, PTGS2, SSBP3, PTPRG, PLCB2, STT3A, CCDC39, ADAMTS6, PCDH20, LINC02881, CXCL12, SALL4P1, ZFYVE28, BMP7, LINC01255, RORA, HLA-DRB1, TBX5, LINC02459, KIF26B, NLRC5, HHAT, NKAPL, TSPAN18, RPL23P11, LINC00838, CEACAM21, C3orf49, NLGN4X, CNKSR2, PJA1, LINC02498, KDM4A, NOP56P1, RPSAP2, SINHCAFP3, RELN, RENBP, OR2J2, OR2J4P, CUL9, EYS, IGF2BP3, TP53TG1, TMEM243, SGCE, LINC02686, LINC02696, SYT13, ARHGAP1, RAB3IL1, TENM4, LINC02551, LINC02730, R3HDM2, WSCD2, P2RX4, P2RX7, MLXIP, CCDC62, HIPIR, PSD3, RPL10AP3, RP1, MIR124-2HG, TSNARE1, RN7SL591P, LINC02621, C10orf95-AS1, AS3MT, ATP6VOB, DPH2, RASSF1, DOCK3, PTN, FGFR1, WBP1L, PITPNM2, FRY, SLC66A2, COX7A2P2, SLC9B1, NEK1, KRT18P42, MAN2A1-DT, RPL7P19, PPP2R2B, LINC02338, LINC00933, ZSCAN2, CORO7-PAM16, CORO7, TAOK2, TNFRSF13C, IL1RAPL1, AGBL4, AGBL4-IT1, ANKRD45, KLHL20, AXDND1, RBKS, MRPL33, RN7SL361P, AHCYP3, ATPAF2, DRC3, SNORD3A, THAP8, SLC32A1, ACTR5, PTGIS, NRIP1, ENTHD1, NIPA2P1, FZD1, HSPE1-MOB4, HSPD1, TRANK1, MSTIR, MONIA, HYAL2, HYAL1, CACNA2D2, MAPKAPK3, PBRM1, CTNNA2, MIR924HG, MIR4719, PPP1R16B, L3MBTL2-AS1, L3MBTL2, BTN3A2, H3C9P, CDK2AP1, AMBRA1, LINC02715, RARG, TLCD4-RWDD3, TLCD4, LINC01830, CNNM4, LMAN2L, ZBED9, LINC00533, OR5V1, SUMO2P1, MOG, ZNF70P1, HTATSF1P1, LINC01004, BRAF, CCT4P1, RN7SL717P, FTCDNL1, NGEF, SH3RF1, TMEM161B, KDM3B, CTNNA1, HSPA9, H4C5, H2BC8, LINC02060, TMEM161B-AS1, H2BC6, H2AC11, H4C9, OR11A1, H1-12P, RETREG2, UBE2Q2P1, PES1, UTRN, PGM2, LINC01088, NAA11, GCFC2, SUCLA2P2, SAMD3, RNA5SP228, RN7SL417P, DNMIP51, MIR548AE1, PGBD1, MROH6, NAPRT, ZSCAN23, COQ10B, EFLIP1, DNMIP41, SLC7A6OS, LINC01579, BMPRIB, MACROD1, LINC02163, RNU6-334P, TRIM8, ARL3, SLC38A8, MBTPS1, SPPL3, ACADS, GTF2IP7, YWHAG, HSPB1, EEPD1, SIPAIL2, SPAG16, COL21A1, RASGEF1B, RPL35P8, SNORA70, ETS2, SNORA62, MIR4432HG, PPP3R1, LINC02494, LINC02232, RNU6-1296P, RNU6-480P, FYN, PPPIR3A, SNF8P1, LINC01807, ZFPM2, STAU2, ADAM10, FES, LINC02858, SLC7A6, ZSCAN16-AS1, KIAA1217, CLSTN3, NRXN3, RYR3, SLC38A3, GNAT1, HLA-DQB2, HLA-A, HLA-W, HLA-Z, HMGB1P38, ROBO1, RNU6-939P, RNU6-111P, RPSAP28, HYKK, FAM184A, GIGYF1, NGF-AS1, NGF, FALEC, ADAMTSL4-AS2, ALDH7A1P4, LINC01884, MRTFA, GAPDHP37, OR2UIP, OR2GIP, OR2ADIP, ZNF311, OR12D3, ZKSCAN4, MDK, SFXN5, STK19, LRRN3, MEF2C, TRIM27, LINC02829, H2AC14, COX11P1, SFTA2, MUCL3, HSPA1A, HSPA1B, BTN1A1P1, LINC00243, LINC02570, LINC00240, FLOT1, ABCF1, PPP1R10, MRPS5P3, SELENOTP2, NDST3, LINC01320, H2BP5, SLC17A4, NSUN6, RAIL, PLA2G15, DPYD-IT1, NDRG4, NSD3, TMX2, LINC00637, SCAFI, ATXN7, MTHFR, LTF, PRICKLE2, LINC01170, ZBTB34, PDGFD, CNTN1, RPL35AP34, SEC14L2, SEC14L6, GAL3ST1, OSBP2, LARGE1, Clorf167, PGAMIP1, RPL7P20, GABBR1, RN7SKP94, CCDCl82, C2orf88, DSG1-AS1, LINC01173, DSCAM, LINC02778, RNF111, SLTM, YWHABP1, FAM201A, LINC02105, NDUFS4, RNU7-183P, GS1-279B7.1, LINC02188, LINC02181, MEF2B, BORCS8-MEF2B, STARP1, PRKD3, RN7SKP224, TMEM17, GSX2, CDCA4P1, LINC01239, LINC02873, LINC02882, ENOX1, RCBTB1, ARL11, NIN, PPP2R5E, ZNF207, PSMD11, PRKG2, PCGF3, CYSTMI, IGIP, LINC01289, LINC01414, PHF2, KCNMA1, CERS5, TTC7B, LINC02210-CRHR1, FUT2, ANKFY1, UBE2G1, ZZEF1, ASAH2, SGMS1, PANK1, GTF2H4, DDR1, PHF2P2, EDIL3, LRP1B, ANKRD27, GPR1-AS, HLA-U, HLA-K, SPTLC1, KCNMB2-AS1, KCNMB2, PDZD8, EMX2OS, COMMD10, SLC30A8, LINC01583, EFNB2, RPL35P9, LINC00841, UNGP1, RN7SL183P, TTLL6, COL25A1, RAF1P1, HS3ST5, TBXAS1, PIK3C2G, CASC17, RNU7-155P, FARSB, SGK1, GALNT13, MOBP, RPSA, TCF7L2, ZKSCAN3, TRIM10, OR2J3, HLA-DRB5, MUC22, HCG20, C6orf15, RNU6-1133P, HLA-DOA, BRD2, MTCO3P1, HLA-G, HCP5B, C2, HLA-DRA, MUC21, NAPGP2, HLA-DRB6, HMGN4, HCG11, DDX39BP2, HCG9, PBX2, H2BC16P, ZSCAN9, MICA, HLA-S, TUBB, PSMB9, PPP1R2P1, MKNK2P1, TJAP1P1, RPS26P56, MOCOS, ZRANB2-AS2, LINC01812, PI16, KAZN, SPSB4, NAV3, GALNT5, ABCA6, ABCA9, IDO2, LINC02866, LINC02683, RNA5SP331, SYBU, IGFBP7, CD207, LINC01143, CCDCl2, RPS3AP40, OR7EIP, GNA12, RPL32P35, C6orf89, RPS26P54, CCBE1, UBE2WP1, MINDY1, PRKCD, RFT1, MSL2, UBE2D3, SMIM15-AS1, CREB5, GKAP1, ARID5B, PCGF6, INA, DGKZ, CADM1, ZNF592, LINC00320, IGBP1P5, LINC02261, CISD2, RN7SL178P, DUSP6, SEMA6D, SIRPB2, RNU6-917P, RPRD2, NPM1P46, ANKRD44, ANAPC7, KLF16, STAU1, PHF3, EIF4EBP2P3, MIR2113, CCDC92, DNAH10, DNAH10OS, GGNBP2, MYO19, YJEFN3, SLC25A6P5, TMEM38B, FSHB, GLTP, TCHP, UNGP3, SETD6, WNT3, LINC00879, BEND7, SEPHS1, TOMM22P6, COLEC12, RNU1-61P, MSH5-SAPCD1, MSH5, HLA-C, HCG27, BTN2A2, DHFRP2, CYP2D7, EPHX2, HLA-DQB3, TOB2P1, ZNF192P2, HCG4B, MCCDIP1, HLA-H, ALPK3, VNIR10P, ZNF204P, HLA-T, ATAT1, HLA-DQA2, MOB4, BTN2A1, TWF2, EEF1A2, MICB-DT, FKBPL, PRRT1, MICD, EHMT2, ZDHHC20P2, FGFR3P1, LYPLA2P1, MYL12BP3, CHRNA5, C2-AS1, F2, RPL8P1, LINC01012, TRIM31-AS1, TRIM31, PSORSIC2, MTCO3P40, RNU7-160P, BAG5, RNA5SP459, TRAF3, TNIK, LINC02077, GPC1, AMIGO1, GPR61, E2F7, CSRP2, ZNF385C, FTH1, LINC02733, RPL35AP8, LYZL4, CARD11, NBAS, LRATD1, RNU6-461P, ZPLD1, RCL1, ECMIP1, SORT1, KLF9, NCMAP, TMEM229B, PLPP4, RPL21P16, NCBP3, THSD4, FAAH, NSUN4, PDXDC1, RRN3, NDE1, MYH11, ATP2A1, SH2B1, IRX3, LINC02169, LINC00922, SMPD3, CDH13, GAS7, AP2B1, TAF15, SEHIL, NPC1, MIR4318, CELF4, KATNAL2, RNU6-655P, MED26, GPC5, MIR17HG, FARP1, STK24, RBM23, LGALS3, MAPKIIPIL, ESR2, MAP3K9, DENND4A, LINGO1, CPEB1, SNX29, FAT3, MAML2, NCAM1, MIR100HG, OR8B4, OR8X1P, ATF7IP, DENND5B, DDN, PRPF40B, ERBB3, RPS26, NRAD1, LACC1, PCDH17, BACE2, DENND6B, SHANK3, LINC02055, MROH5, CYHR1, RGS3, NFIB, TTLL11, BRD3, CAMKID, CTNNA3, BNIP3, JAKMIP3, TEAD1, CCDCl62P, GLCCI1, ICA1, SLC25A5P5, PDEIC, RN7SL7P, SAMD9, FOXP2, NUP205, CNOT4, PTPRN2, RNU6-1213P, MIR5708, NUDCD1, TRHR, SPRED2, CDC42, WNT4, BARHL2, LINC02609, RAPGEF4, SESTD1, CYB561D1, PLEKHO1, LINC02789, ARPC3P2, LINC00467, LINC01655, DUSP10, PDIA6, NRXN1, KNOP1P3, ZNF638, AFF3, ST3GAL3-AS1, ST3GAL3, TANK-AS1, GIPC2, DNAJB4, HAT1, SLC25A12, BCAR3, RAPIA, CADM3-AS1, CADM3, CRLF3P1, TGFBRAP1, ARHGAP15, KCNJ3, RNA5SP107, VWC2L, MAML3, PPID, FNIP2, LINC01098, RNU1-45P, NDUFAF2, LINC00461, MIR3660, EFNA5, FBXL17, RN7SKP122, KCNN2, LINC01957, RNU6-793P, ATXN1, IP6K3, LINC00691, NR1D2, MAP4, RBM6, DCAF1, CADM2, BBX, IGSF11, LINC01471, LINC02016, MSANTD1, EPHA5, MIR1269A, IFITM3P1, TET2, LINC02466, ADAM12, PGLYRP3, LORICRIN, LINC02761, SDCBP2, SDCBP2-AS1, SGO1-AS1, ADAMTS2, ZNF354C, DCHS2, FTO, TEX2, PCSK5, DNAJC7, LINC02329, RNU6-976P, LINC00676, LINC00399, PPIAP62, METTL9, MDFIC, ZFAT, MTCO1P49, TYRL, TRIM51FP, RNU6-1271P, TFEC, FBXO28, SLC30A3, ADAMTS19, RPL31P57, RNU1-146P, UBE2HP1, PAG1, LINC01006, MYL12BP2, RN7SL28P, LINC02200, HMGB3P16, RN7SKP120, TUSC1, FAM178B, LINC02488, FAM177A1, MYO1H, ZSCAN12, METTL15, LINC02758, OR5BAIP, OR5AZIP, GRM5, TYR, CCDC68, KIZ, KIZ-AS1, SLC12A5, RPL31P12, HNRNPA1P46, NCOA5, OLFM3, LINC02500, LINC00290, PAK5, ACTRT2, DSCR9, SLC6A6, ATCAY, WT1-AS, EIF3M, ARNTL, C12orf65, SLC45A4, ZCCHC7, TCAIM, RPS6KL1, CAMTA1, RN7SL831P, EEF1A1P11, HYI-AS1, PMFBP1, PXDNL, NAA40, RCOR2, CTNND1, LINC02694, SLC30A9, MYO18A, OSBPL3, FOLHIB, OLFM4, FADS2, MIR1302-7, GMIP, SYNE2, RSRC1, PAUPAR, LRFN5, ZMIZ1, FOXN2, PPPIR21-DT, TCF15, SRXN1, LINC00707, RNA5SP403, RNA5SP404, CLEC17A, ZP3P2, SMARCA2, RN7SL592P, POLRIH, ZNF615, SEMA3A, MRM2, GRIK1, KMT2A, SLC17A6, ANO5, PRPF3, KIF5C, PALB2, GLT8D1, ADGRL4, SPATA33, CDK10, IL1R2, ILIR1, SLC35F2, HINT1, RCC2P3, RNA5SP169, HDAC4, SYNE1, or combinations thereof.

In some embodiments, the one or more genes affected by TS comprise ACOD1, RPL7P44, ACTGIP22, ACTR5, PPPIR16B, AKAP6, AMBRA1, ANKSIB, ATP2A2, BANK1, SLC39A8, BCL11B, BOLA3P2, MUC16, BRAF, CCT4P1, C10orf67, OTUD1, CACNAIC, CACNAII, CACNB2, CAMTA1, CD276, INSYN1, CD47, CFAP99, CHRNA3, CNTN4, COL27A1, CSMD1, CSMD3, CTNND1, DCC, DGKI, DMP1, DNAH10OS, DNAH10, CCDC92, DRD2, EEF1A1P11, RN7SL831P, EFL1, EIF4EBP2P3, MIR2113, ENOX1, EPHX2, GULOP, ERC2, FADS2, FECHP1, FGFR1, FLT3, FOLHIB, FOXP1, FPR3, ZNF577, FTLP18, GRIK3, FURIN, FZD9, GID4, GMIP, GRIN2A, GRM3, GRM3-AS1, HCN1, HLA-DRB5, HSPD1P6, LINC02033, HSPE1-MOB4, MOB4, HYI-AS1, PTPRF, IGSF9B, IMMP2L, ITIH3, KCNB1, KCNH3, KDM3B, KIZ-AS1, KIZ, L3MBTL2, L3MBTL2-AS1, LINC00461, LINC00587, LINC01122, LINC01360, RN7SKP19, LINC01416, LINC01470, LINC01645, LINC01830, LINC01876, LINC01929, LINC02057, LINC02163, RNU6-334P, LINC02240, LINC02694, LMAN2L, CNNM4, LRFN5, LSAMP, MAD1L1, MEF2C-AS1, MGST1, LMO3, MOCS2, MPHOSPH9, MROH5, MIR1302-7, MYO18A, MYO19, GGNBP2, NAA11, LINC01088, NECTIN1, NFATC2, ATP9A, NGEF, NPAS3, NPM1P10, NRGN, NSD3, NT5C2, NUBPL, OLFM4, OSBPL3, GSDME, PAUPAR, PCLO, PDE4B, PHEX, PHF3, PICALM, RNU6-560P, PITPNM2, PLCL1, PMFBP1, POLR3B, POUIF1, PPPIR13B, PPP2R2B, PTPRU, PXDNL, RBFOX1, RCOR2, NAA40, RERE, RERE-AS1, RGS6, RIMS1, RN7SL167P, SNX19, RN7SL717P, FTCDNL1, RNA5SP172, RNA5SP87, RNU1-130P, RNU6-1060P, RNU6-1228P, RNU6-765P, YWHABP1, RNU7-160P, BAG5, RPL31P12, RPS19BP1, RPS6KL1, RPTOR, RSRC1, RTN1, SGPP2, SHISA9, SHMT2, NXPH4, SLC30A9, SNAP91, SNORC, SORCS3, SOX5, SPAG16, SPPL3, ACADS, SRPK2, STAG1, SYNE2, ESR2, TBC1D5, TCAIM, TCF20, TCF4, TENM2, TENM4, THOC7, TMEM161B-AS1, TMEM219, TRIM8, ARL3, TRPC4, TSHR, TSNARE1, TSPYL5, SNORD3H, VPS45, OTUD7B, VRK2, WDR7, XYLT1, YJEFN3, ZCCHC7, ZFPM2, ZNF536, ZSCAN31, or combinations thereof.

In some embodiments, the one or more genes affected by Type I Diabetes comprise HLA-DQB1, HLA-DQA1, RBM17, PFKFB3, BACH2, THADA, INS-IGF2, INS, PTPN22, SH2B3, ATXN2, LINC00320, STK39, LRPIB, TYK2, PGM1, BTN2A3P, GATA3, HLA-DQB1-AS1, RAB5B, PRKCQ, ERBB3, CTSH, PTPN2, UBASH3A, Y_RNA, IL10, CENPW, COBL, RNLS, CD69, LINC01550, CTRB1, CTRB2, SIRPG, LIF-AS1, HORMAD2, MIR3681HG, HLA-DRA, IFIH1, KIAA1109, GSDMB, ICOS, CTLA4, SKAP2, DNAH2, PRKD2, SMARCE1, CCR7, GAβ3, UMOD, GP2, RPL32P23, ASCL2, MIR4686, CLEC16A, C1QTNF6, LINC02357, GLIS3, MAGOH3P, ZFP36L1, NUPR1, IL27, MTCO3P1, IKZF4, IL2RA, CUX2, LINC02649, RMI2, PHTF1, RSBN1, NAA25, LINC01882, ITSN2, NCOA1, CD226, CAPSL, IL7R, LINC01104, IGF2-AS, RASGRP1, LPAR3, ADAD1, IL2RB, IL2, LINC02694, ZPBP2, EFR3B, LMO7, JAZF1-AS1, TNFRSF11B, RNU6-12P, PLB1, FOSL2, FGF3, FGF4, HPSE2, IL25, EFS, MEG3, RPS26, CCR12P, TM9SF2, CLN3, MAPT, FUT2, SIRPG-AS1, GATD3A, CAMSAP2, AFF3, ACOXL, FAP, LINC02009, CCRL2, LINC01179, MIR588, IKZF1, RNU6-474P, TH, ZFP1, H19, IGF2, TACC3, FGFR3, RUNX3, MIR4425, PSMB2, INPP5B, NOTCH2, PTPRC, ATP6VIG3, BATF3, LINC02773, MIR4432HG, SEPTIN2, LINC02362, CAMK4, IRF1-AS1, IRF4, ITGB8, CFTR, TOX, COLEC10, AGO2, GTF3C5, CEL, ADAMTS14, PRF1, KCNQ1DN, CDKNIC, LINC02757, EMSY, FLI1, KLRG1, LINC02341, ACAP1, PRR15L, PNPO, RDMIP3, ARHGAP27P2, AIRE, ARL8B, ACSL1, CENPU, STMND1, RN7SL366P, CBX3P1, BDNF, EIF4A1P4, HIGDIAP1, PI4KA, HLA-DRB9, NEMF, HLA-DRB5, PAX4, SND1, KCNQ1, ANKRD50, LINC02516, MNDA, OR10AAIP, RPAP2, ADAM30, IL6R, FASLG, SLC25A38P1, RGS1, TATDN3, STAT4, FARP2, DGKQ, KLF3-AS1, TTC33, ANKRD55, EXOC2, LINC02539, TAGAP-AS1, JAZF1, CTSB, OR7E158P, NRP1, IATPR, CD6, SLC15A3, FADS2, FADS1, RPS6KA4, M6PR, SPRYD3, RAD51B, EEF1A1P27, NR4A3, STX17-AS1, SLC1A2, FTO, RNA5SP94, DMRTA1, CDKN2B-AS1, TCF7L2, RGMA, SEPHS1P2, CLEC2D, LINC02390, FSTL4, RNASET2, MIR3939, PLEKHA1, PPIL2, MIR130B, TRIM31, TRIM31-AS1, LINC00243, MICA, HLA-S, NCR3, UQCRHP1, CFB, TSBP1-AS1, BTNL2, TAP2, MAGI3, AP4B1-AS1, BCL2L15, TUBB4BP5, QRFPR, GLRA3, PSD3, OR2PIP, OR2W1, HLA-U, HLA-K, HLA-W, HLA-A, RPP21, TRIM39-RPP21, HCG21, SFTA2, RNU6-1133P, C6orf15, POU5F1, RPL3P2, HCP5, LINC01149, TNF, NOTCH4, TSBP1, HLA-DRB1, HLA-DQB3, HLA-DPB2, COL11A2P1, KIF17, RPS23P10, FCGR3A, LINC02771, SLC4A10, PHLDB2, GABRA2, TLL1, LINC02225, EYS, AHR, HECW1, PHF20L1, PLGRKT, NFIB, PTGES, HS3ST2, USP31, IKZF3, GRB7, PXK, TTC34, RBFOX1, SMAD3, SBK1, NPIPB6, ATXN2L, NKD1, PDE4A, KEAP1, MHENCR, STMN3, RPL23AP12, ADGRL2, LINC01250, CYTL1, RN7SKP113, DCTD, TENM3, RNU6-144P, LURAPIL, LURAPIL-AS1, FNBP1, LINC00993, RPL35P9, LINC00460, ADCY7, LINC00892, RNU6-320P, SRRM1P3, RPL22P23, Clorf141, IL23R, AIMP1P2, CRB1, IL18RAP, SLC9A4, ATG16L1, GPR35, DAG1, IL21-AS1, RNU1-150P, ERAP2, IRF1, IL12B, LINC01845, HNRNPA1P41, JAK2, TNFSF15, CARD9, ALDH7A1P4, ZMIZ1, SLC25A28, NKX2-3, LRRK2, SUOX, or combinations thereof.

In some embodiments, the one or more genes affected by Type II Diabetes comprise KCNQ1, SLC30A8, PPARG, GPSM1, HNF4A, LINC01080, SPRY2, KCNJ11, NTRK2, TCF7L2, CDCl23, CDKAL1, IGF2BP2, CTBP1-DT, EIF2S2P3, RN7SL644P, MIR4686, ASCL2, SFI1, ZMIZ1, JAZF1, COBLL1, CCND2, CCND2-AS1, PCSK6, TMEM154, RPS3AP18, ARL15, C5orf67, RPL26P19, ZBED3-AS1, JADE2, SSR1, POU5F1, TSBP1-AS1, KCNK16, CENPW, MIR588, SLC22A3, SLC22A2, DGKB, GTF3AP5, BRAF, KCNU1, SMARCE1P4, ANK1, TP53INP1, NDUFAF6, GLIS3, CDKN2B-AS1, DMRTA1, CHCHD2P9, TLE1, RNU6-1035P, GATAD2A, APOCIP1, APOC1, HMG20A, MACF1, FAF1, PROX1-AS1, PROX1, TMEM18, LINC01875, GCKR, THADA, MIR4432HG, MIR4435-2HG, GRB14, MIR5702, NYAP2, UBE2E2, ADCY5, LINC01991, LPP-AS2, MAEA, LINC02495, WFS1, SMIM43, ANXA5, RN7SKP188, TBC1D4, ITGA1, LARGE1, LINC01643, NKX6-3, ESRP1, CPQ, TRHR, CASC11, LINC00824, BOP1, ARHGAP39, Metazoa_SRP, HAUS6, FOCAD, CDKNIB, SSPN, ITPR2, RN7SKP15, PTHLH, HMGA2, TSPAN8, USP44, RMST, WSCD2, RFC5, KSR2, WSB2, SPPL3, HNFIA-AS1, HNFIA, OASL2P, SNORA70, KDM2B, ABCB9, FBRSL1, RNF6, HMGB1, KL, TOMM22P3, DLEU1, DLEU7, SEC16B, LINC01741, DSTYK, SRGAP2, LYPLAL1-AS1, ZC3H11B, ABCB10, MTCYBP14, MTND6P14, LINC01865, LINC01874, CYRIA, GACAT3, DTNB, LINC01819, LINC02580, ZFP36L2, LINC01122, CEP68, RABIA, SPRED2, STARD7-AS1, TMEM127, MIR375, CRYBA2, PIK3R1, VWA8P1, LINC02227, RPL35P2, NUDT3, IFITM3P3, SPDEF, ATP5PBP6, LINC01625, FILNC1, PTEN, MED6P1, RPL19P16, LINC01153, METTL15, ZNF664, RFLNA, SHBG, ATP1B2, KDM6B, ZBTB46, TCEA2, PABPC4, PABPC4-AS1, PATJ, DENND2C, PTGFRN, NOTCH2, ARNT, BNIPL, PLXND1, TMCC1, LINC01214, TSC22D2, MBNL1, ATP5MGP5, EGFEMIP, SLC2A2, EEF1A1P8, ABCC5, DGKG, ST6GAL1, PDE6B, TMEM271, PCGF3, FGFRL1, TACC3, FGFR3, HTT, PPP2R2C, FAM184B, DCAF16, PRDX4P1, THAP12P9, MAP3K1, SETD9, POC5, DMGDH, RASA1, OR7H2P, PAM, GIN1, RN7SL554P, RREB1, HLA-DRB1, HLA-DQA1, LRFN2, VEGFA, LINC02537, TFAP2B, RNU7-66P, BEND3, RSPO3, RPS4XP9, LRRC66, DCUNID4, SCD5, FAM13A, SMARCADI-DT, LINC02428, CENPE, TERFIP3, RNU1-89P, LINC02272, PDGFC, ACSL1, OTULINL, ANKH, LINC02224, RN7SL383P, RPS17P11, MFSD4BP1, ITGA2, FST, GIP, IGF2BP1, CCDC47, CSH1, GH2, ERN1, PITPNC1, BPTF, RPL17P41, LAMA1, CCDC68, TCF4, LINC01415, LINC01416, WDR7, SEC11C, GRP, MC4R, RNU4-17P, PHLPP1, BCL2, UHRF1, PTPRS, INSR, MAP2K7, HOOK2, RTBDN, FARSA, SUGP1, Y_RNA, LINC01101, LINC01871, CYTIP, RBMS1, TSEN2, PTHIR, KIF9-AS1, RBM6, RFT1, CACNA2D3, ATXN7, RNU6-739P, PRICKLE2-DT, ADAMTS9-AS2, SHQ1, ROBO2, SLC12A8, LINGO2, RN7SKP114, DCAF12, MTND2P8, LINC02603, AOPEP, ABO, LINC01451, RN7SL198P, TMEM256P1, NEUROG3, MTATP6P23, LINCO2651, RPL5P26, PPIF, CPEB3, HHEX, EXOC6, PCDH17, RNY4P29, CTAGE16P, LINC00370, IRS2, SLC7A7, AKAP6, CLEC14A, KRT8P1, NRXN3, PPP4R3A, MARK3, RASGRP1, LINC02694, LTK, RPAP1, EHD4, RPSAP55, ONECUT1, WDR72, TCF12, NPM1P47, C2CD4B, USP3, USP3-AS1, MAP2K5, IMP3, LINGO1, ABCC8, WT1-AS, QSER1, TCP11L1, EHF, PDHX, HSD17B12, MAPK8IP1, CELF1, SCYL1, SMIM38, MYEOV, CCND1, ARAP1, MTNRIB, SLC36A4, DEUP1, LINC02725, ETS1, ETS1-AS1, FLI1, PARP11-AS1, HSPA8P5, PEPD, ZNF229, APOE, RN7SL836P, GIPR, SYMPK, ZC3H4, RN7SKP140, GSTM3P1, RALY, GDAPIL1, HNF4A-AS1, EYA2, PELATON, CEBPB, LINC01524, GNAS-AS1, GNAS, NELFCD, LIF-AS1, HORMAD2, YWHAH, EP300, PNPLA3, PIM3, PANX2, MOVIOL1, NHEG1, RPL35AP3, RN7SL366P, QKI, AGMO, IGF2BP3, RPS2P32, CRHR2, YKT6, CAMK2B, PRKRIP1, FBXL13, PSMC2, RELN, CTTNBP2, CPA1, KLF14, H4P1, AOC1, UBE3C, MSRA, XKR6, LPL, RPL30P9, PURG, PLEKHA1, FAM99B, IGF2, H19, TH, INS, CD81-AS1, KCNQ1OT1, SLC22A18AS, PDE3B, ARPIN-AP3S2, AP3S2, PRC1, PRC1-AS1, FAM234A, CLUAP1, ATP2A1, TLCD3B, ZNF771, RBL2, FTO, NFAT5, ZFP1, CTRB2, CHST6, CMIP, SPG7, ATP2A3, ZZEF1, GLP2R, RAI1, MIR4733HG, RNF135, HNF1B, PSMC3IP, RETREG3, INS-IGF2, UBE2Z, GCK, RPSAP52, OASL, PPIAP23, MIR17HG, CTRB1, SLC9B2, HLA-DQB1, NECTIN2, LINC02698, RPL12P46, DCHS2, LY86-AS1, LY86, EIF3IP1, STIMATE, STIMATE-MUSTN1, ATP8A1, RNU6ATAC8P, HSPEIP14, IL23R, AK5, FMO4, SHB, BECN1, MYTIL, RBMS3, RBMS3-AS3, PLK2, IPCEF1, OPRM1, TMEM132D, ZNF217, HDAC9, ADAMTS18, STARD10, RCCD1, SLC16A11, EIF2S2P7, SINHCAF, RPSAP17, ATP8B2, INAFM2, CCDC9B, C16orf74, ADH5P4, ZBTB20, TRMT9B, SALL4P5, RPL24P7, PSMD6, CTBP1, CACNA2D1, GCC1, FSCN3, LEP, MIR129-1, GRK5, CCNQ, DUSP9, KRT18P48, SRBD1, LINC01877, FTCDNL1, TRA2B, LGMNP1, RPS27AP11, LINC02541, RNU6-300P, MTHFDIL, LINC02461, TPT1, GTF2F2, LINC00348, PPIAP14, LINC01892, RNU6-66P, RNU4-10P, SNX29, CPPED1, COMMD7, LINC00624, BCL9, RPL5P16, MARCHF1, PCNX2, LPIN2, DCDC2C, HUNK, KIF11, PEX5L, CR2, SDHAF4, ACHE, TCERGIL, ZPLD1, RNU6-461P, ACP7, PLS1, LINC01918, GPR45, LINC01499, LINC02745, LINC01339, CCNT2-AS1, TMEM163, TGFBR3, PALM2AKAP2, SYK, SACS, SGCG, SGCD, RHOU, TG, CCN4, MRM3, NXN, RBM38, HMGB1P1, ZFAND6, FAH, SNRPGP16, SYN2, GSTM5P1, RNA5SP110, RNA5SP111, DCD, VDACIP5, PTPRD, SRR, F3, SLC44A3-AS1, LINC01395, RPS27P20, LINC02752, MTND5P21, HLA-DQB3, MTCO3P1, UBE2E2-AS1, SLC16A13, ZNF800, ZFAND3, FITM2, R³HDML, PRICKLE2-AS1, VPS26A, HECTD4, OAS1, LINC01405, CUX2, PAX4, GABRA4, LINC00523, DLK1, RGS7, CAPN13, GALNT14, FGFR3P1, ZDHHC20P2, HECW1, CPA6, INTS8, HMGA2-AS1, FOXB1, TMEM175, DNER, MIR876, VPS33B, ETV1, TXNDC15, PCBD2, MPHOSPH9, PRELIDIP1, RNU6-200P, LINC01141, IPP, MAST2, PGM1, COLGALT2, TSEN15, MDM4, PIK3C2B, NUP133, SIX3, KRTCAP2P1, SCTR, EPC2, RNU2-9P, PCED1CP, LINC01878, ATG16L1, EIF4E2P2, CASR, HACD2, KLF7P1, EIF5A2, LINC00885, TFRC, MOB1B, RNU6-774P, NKX6-1, RANBP3L, PARP8, HMGCR, ANKRD31, PCSK1, CAST, FGFR4, ZNF346, HCG22, SMIM29, GLPIR, NUS1, NEPNP, ENPP3, ECT2L, HIVEP2, RBMX2P4, MYL7, RNA5SP230, ZNF713, AUTS2, DPY19L2P4, STEAP2-AS1, CALCR, RASA4DP, FAM185A, GRM8, SND1, LINC02356, LINC01234, RPL12P33, LINC00540, NYNRIN, RN7SKP17, LINC01629, MEG3, TRAF3, RCOR1, HERC2, PLCB2, MYO5C, SIN3A, LINC00261, NFATC2, ZNRF3, WNT7B, ATXN10, ASAH1, LINC01605, C8orf86, FGFR1, KCNB2, JPH1, TRIB1, EFR3A, H3P29, LINC01230, PTCH1, ABCA1, PTFIA, C10orf67, ARID5B, JMJDIC, TSPAN15, LRMDA, TNKS2, XRCC6P1, RPL17P34, ARHGAP19, BBIP1, BDNF, FAIM2, PTPRR, IGFIR, SLX4, GP2, PKD1L3, ZFHX3, CLEC10A, NF1, SUMO2, NUP85, SNAPC2, ZNF799, ZNF708, ZNF738, ZNF208, ZNF43, MAP3K11, KCNK7, SUMO2P17, EYS, CACNAIG-AS1, NUFIP2, RPL35AP35, IL17REL, MCC, LINCO2615, GYS2, SLCO4C1, HSF1, SYT4, RIT2, MIR466, E2F7, PMP2, PKN2-AS1, LRP12, GRIP1, MACIR, PPIP5K2, RPS3AP49, TCF19, RBM33, CEP120, BCL2L11, ZNF236, TMEM106B, MFHAS1, ALKBH3, LINC02451, IFT52, HSD17B6, PRIMI, COMMD4, RAMP2, ZNF429, MTDHP3, USP48, BMP8A, SLC1A2, EMB, PELO, POLK, LINC00461, DOCK11, IL13RA1, RPS6P12, GALNT3, ZBTB26, IPO9, TEX15, RNU6-1299P, LINC00968, RPL37P6, TNFAIP6, NMI, MAML3, MED23, PKLR, NME9, MRAS, RNU6-1231P, WBPIL, DNAJC2, CWH43, TNS1, DGKD, PSMD6-AS1, RNU6-891P, NSD1, FADS2, SNTB1, PCSKIN, FAAH2, ZXDB, RAPGEF5, KCNK17, BTBD9, SETBP1, NLGN1, GRID1, OR5B17, OR5BIP, SPHKAP, RPS29P1, ACTN1-AS1, TSHZ3-AS1, ODAPH, CDKL2, RPL13, ERLIN1, LINC00558, ZNF646P1, SEPHSIP2, LINC02838, CRYBA1, TWF1P1, CBX1, KDM4B, BTD, BRD3OS, SBF2, JARID2, STK17B, DNAH7, GTF2IP11, PHKG1P3, OR4RIP, OR4C9P, MTOR, STK31, CDH7, NEGR1, RPL31P12, EHHADH-AS1, LMF1, LINC01829, ZFPM2, TBCE, LRRC74A, NHSLI, MIR6074, KLHL42, CAMKK2, GPAT4, HLA-DRB9, EHMT2, ROR2, OSBPLIA, LIMS2, DAAM1, No mapped genes; KCNQ1; No mapped genes; No mapped genes, LINC01752, MARCHF3, TCN2, TM4SF4, B3GAT3P1, TRIM59, C3orf70, SLIT2, OCIAD2, OCIAD1, SHROOM3, UNC5C, TET2, RNU6-351P, PGRMC2, LARPIB, HHIP, SORBS2, UGT3A2, LMBRD2, LINC02060, TMEM161B-AS1, RPL7P19, ETF1, LNC-LBCS, GGNBP1, BAK1, REV3L, MFSD4B, LINC02523, HEY2, LINC02828, RGS17, ANKDD1B, EBF1, LINC02571, FOXK1, SUGCT, FIGNL1, DDC, GTF2I, CFAP69, POP7, EPO, LONRF1, LINC00681, SGCZ, CCAR2, EBF2, TRPS1, EPB41L4B, COL27A1, STRBP, VAV2, BEND7, LINC00838, REEP3, TET1, PCBD1, LINCO2622, RPL12P27, PDCD4, TRIM66, OR5D18, OR5L2, PLCB3, TSKU, CHD4, LDHB, PDZRN4, ZNF641, SMUG1, EP400, ZNF268, DDX39AP1, SNORD36, LINC02337, UNC79, LINC02338, DEPDC5, ART3, WNT8A, NME5, RANBP17, SRP54-AS1, USP49, MED20, KCNH7, STK35, ENO3, VPS11, HMBS, SLC38A9, TCF3, NUDT21, AMFR, GLRA1, LINC01933, IL34, DGAT1, SCRTI, MIRLET7A1HG, NAGLU, HSD17B1P1, NFE2L1-DT, YPEL2, SNRPGP17, ACE, LRRC8E, RPS29P23, TM6SF2, KIF3C, NFIB, VPS53, MIR4776-1, IKZF2, GCNTIP5, GSAP, RDH14, CRTC1, HMGB3P15, HEATR5B, LINC00907, MNAT1, SOAT1, COX5BP8, LEPR, TMEM87B, ZHX3, LCORL, SLC9B1, CSPG4BP, KCNQ3, ZRANB3, STK11, TSHZ3, MYBL2, SLCO4A1, RFPL4AP6, AP1B1, PLA2G6, CDKN2C, AQP10, CRYZL2P-SEC16B, LINC01793, SMARCC1, SCAANTI, INKA2-AS1, INKA2, RNU6-709P, ADRB1, MY019, HOXC4, ZNF76, TAT-AS1, MARVELD3, RPTOR, LHFPL3, ZFAT, MIR30B, EYA1, DGAT2, RN7SL786P, LINC02140, IRX3, STAU2, EEF1B2P5, FAM227B, PARD3B, KCNS3, MAFF, ZNF767P, ZNF746, CUL1, ALDH1A2, LINC02853, RORA, HMGN2P47, LINC02206, SEPTIN9, LINC01979, LINC01978, LDLRAD4, PCNT, KLHL21, RPS6KA1, STX12, GMEB1, SGIP1, TENT5C, VPS25P1, SV2A, SF3B4, DNM3, PIGC, IPO9-AS1, NAV1, PM20D1, SLC41A1, LINC02831, AFF3, LINC01102, MERTK, ZEB2, LINC01305, SP9, TTN, SCHLAP1, BMPR2, MTCO1P17, PLEKHM3, ERBB4, SETD5, ANKRD28, ARPP21, EIF4E3, FOXP1, VGLL3, CPNE4, NDUFS6P1, ZBTB38, HERC1, PTPN9, PDILT, RABEP2, TMEM219, CYB5B, SREBF1, EVI2B, CAMK2G, COPB1, SLC35C1, CRY2, MREGP1, TSHZ2, MIR296, PIEZO1P2, C20orf181, L3MBTL2, EP300-AS1, ACP6, SUMF1, TRANK1, U3, MARK2P6, ACAD9, CCSER1, OARD1, MAD1L1, AMZ1, CSMD1, DENNDIA, SPOCK2, XRRA1, POLRID, PITPNM2, RNA5SP30, BMF, C6orf89, PI16, MAML2, HS6ST3, UBE20, ALG10B, KIF5A, RPS4XP15, GRB10, PTPRQ, RN7SL597P, MIR3168, MDGA2, ARID4A, DCAF5, NPC2, C15orf54, MYO5A, PML, FSD2, PDXDC1, GINS2, LINC02182, BANP, SLC25A11, GPIBA, ZNHIT3, CDK12, NEUROD2, SLC39A11, CYTH1, RMC1, FCGRT, RAB3C, RNU2-43P, LBX1-AS1, KDM3A, CNTNAP2, SLC39A8, MAGI2, SAMMSON, PSMA3, ARMH4, PSMA3-AS1, HSPA12A, HCP5B, HLA-G, PRDX5, CCDC88B, PNKD, KCTD8, LINC02465, MGAT1, SUPT3H, CAPZA1P4, PRKD1, RPL5P22, ELP3, NOL4, EVA1B, SH3D21, CHD1, LINC02144, RFX3, SYNC, KIAA1522, H2BC8, H4C5, AZIN1, GAPDHP30, GDF6, HAT1, SLC25A12, MPPED2-AS1, TMEM244, L3MBTL3, NELL1, PKHD1, RPL23AP95, DPY19L2P2, LINC00529, PHGR1, FBXW7, ZNF257, CCHCR1, PPPIR11P2, SPIN2A, PQBP1, CNKSR2, HCG27, HLA-C, RNU6-567P, PHF2, C1QTNF7-AS1, MIR5094, TSPAN3, LINC02106, CAMKID, MNX1, MLX, MYPOP, NANOS2, CPN1, TTLL6, C17orf58, MINDY1, SENP2, TPCN2, MRPS35, SBNO1, MTMR3, HORMAD2-AS1, KIF9, ASCC2, PTPN23, CALCOCO2, TNIK, NUDT6, YTHDC2, NFKBIL1, HLA-DQB1-AS1, HLA-DOB, MIR4421, LINC02576, LINC02245, SOCS2, CRADD, ANPEP, CAVINI, STAT3, OSBPL7, TEX14, IGBP1P2, TACO1, MAP3K3, SAMM50, UBBP1, HNRNPKP2, NIPSNAP2, PSPH, CFAP77, CCDC77, B4GALNT3, SFTA3, RNU7-93P, XYLT1, RPS17P5, ARG1, PRAG1, FAM86B3P, RNU6-1151P, RNU6-526P, PINX1, ZNF34, CDKN2B, UBAP2, PTPDC1, CHUK, MTNR1B x CRYBB2-IGLL3P, MTNR1B x MMP19, MTNR1B x LINC01038-LINC00382, MTNRIB x CMIP, MTNRIB x PRELID3A, MTNRIB x RPL7AP69-USP29, MTNR1B x FCGR3A-FCGR2C, MTNRIB x LINC01814, LINC00299, MTNRIB x MRPL50P1-N/A, MTNR1B x MIAP, MTNRIB x HECW2, MTNRIB x SLC19A3, MTNR1B x NAALADL2, MTNR1B x MYL12BP2-LINC02363, MTNR 1B x N/A-CSMD1, MTNR1B x YTHDF3, MTNR1B x RN7SL135P-N/A, MTNRIB x RIMS2, MTNRIB x MACROD2, MTNR1B x RNF6P1, MTNRIB x TCF7L2, RTN4RL1, LARP7P3, LINC01910, TFDP1P3, API5P1, AGTR2, TRPM1, ADARB1, MASP1, MTND5P17, MAGEC3, LINC02030, LINC02152, PALDI, OCA2, RNU11-5P, LINC02353, LINC02506, CCDC42, MYH10, TOMM22P6, LINC01548, IFNAR2, LINC01853, RN7SL283P, SDK1-AS1, CPED1, PTPN22, SH2B3, ATXN2, IGF2-AS, ERBB3, ICOS, CTLA4, CLEC16A, LINC01882, PTPN2, GCA, UBASH3A, RNLS, CTSH, BACH2, IFIH1, CLN3, IL2RA, IL10, RAVER1, RPL32P23, RBM17, COBL, CIQTNF6, AGPAT1, PPT2-EGFL8, HSD17B1, HSD17B1-AS1, SYCE2, GCDH, LTBP3, CCDC92, CDK2AP1, C12orf65, FRAT2, FRAT1, H1-7, OR10AD1, NUP160, MIR924HG, STAG1, STEAP2, JMY, ZNF10, WWTR1, RN7SL40P, LINC00910, PLEKHM2, ARVCF, ZNF487, ZNF239, CKMT1B, STRC, KRT18P1, LINC01556, BIN3, EMSY, TRIM63, PDIKIL, APIP, TEX41, GBA2, ASTN2, ZFPM1, SLC7A5, KLHDC4, BDNF-AS, CNTN2, PDE3A, HPSE2, YTHDF2, MLIP, LRRC1, ACVRIC, CICP11, SUMO2P3, LIN7A, ANO6, ARNTL, EML6, LINC02306, OR7K1P, AOAH, SEC23IP, LINC02641, NACAD, CCM2, KCNJ12, LINC02790, MED27, LAMC1, KDF1, TRPV5, FIBCD1, WDR82P2, HSP90B3P, PRDM5, FXYD2, FXYD6-FXYD2, SIAH3, ZC3H13, RNY4P30, KPNA3, SIDT1, NOS1, TOM1, SYNDIGIL, ZNF654, CGGBP1, ADAMTSL3, CCNQP1, LINC00862, UBE2L5, GUCY1B1, RNU6-243P, TREML2, TREML3P, TGFB1, SH2B2, ELFN1, RNU4-41P, SPTB, NPM1P10, RNU6-267P, TCAF2, LINC02032, HNRNPA1P20, TAF12, OR7A3P, OR7A11P, VTI1A, DAOA-AS1, ANO4, PPARA, GUCY2EP, LRRC32, APCDDIL-DT, FYB2, ST18, DEPDC1-AS1, LINC00385, KATNAL1, RPL12P23, AP3B1, RPL32P14, DLG2, PRELID3BP3, MIR633, LMCD1-AS1, PRDM6, LINC01450, RN7SL178P, CFAP61, CHDIL, PXK, SLC25A26, DPPA3P3, LINC00377, THBS1, LINC02915, CDK5RAP1, ITM2B, POLR2KP2, ARHGEF39, ATP6APIL, SCAMP4, CSNK1G2, SNHG17, LBP, SLC66A2, EPHB2, C1QB, PAX2, UNC13C, MIR3681HG, TMEM132B, RNU7-74P, RBM47, RBFOX1, RHBDL2, ABCC1, TLE4, CPS1, RPS27P10, ZNF503-AS2, RNU6-54P, MTCLIP1, TUBA4A, SOCAR, FGFBP2, FGFBP1, MSH2, EPCAM, SERPINF2, EMC8, ABCG1, UMODL1, RNU6-1060P, CASC15, NEK4P1, RNU1-98P, LINC02755, TTC7B, CBLN2, LINC01899, LINC01247, SILC1, CCDCl72, TMEM212, FNDC3B, CRTAC1, EXOC6B, RPS26, RPL23AP44, RNU4-69P, LINC00316, LINC00315, LINC02508, HSP90AA4P, SAG, DLC1, LINC01638, DCBLD1, ASXL3, TSPAN2, BBS4, PCEDIB, ARHGAP24, RN7SL77P, TTC9, RNU6-481P, FCGR2A, OR51E2, OR51CIP, DLGAP1, DLGAP1-AS4, UNC5CL, CAPN14, IMMP2L, RPL3P8, SKP1P2, LMO3, MMADHC-DT, LINC01931, GRN, SLC13A3, CHST1, LINC02687, MED30, ADAMTS6, MYOM2, MORC4, EEF1A1P40, SLIRPP1, MIR514A3, KRT8P17, MYCLP2, SLC35F1, CYCSP55, KRT18P9, RN7SKP167, LDLRAD2, C2orf16, PDCD4-AS1, RBM20, RNU7-165P, PPIAP39, HABP2, WDR11, MTAP, KRT18P24, RPS20P25, RPS19P6, MYO3A, SNORD28B, H3P28, HLA-DQA2, FILIP1, TUBG1, ATP5MGP7, MAPT, PKIG, PRELID3B, MRPS16P2, ZBTB46-AS1, UQCR10, FMO5, ERO1B, GPR137B, PPIEL, CERS6, GAD1, ERICH2, RN7SL117P, RNA5SP87, LINC00926, LINC01413, DRAIC, RGMA, OSGIN1, MLYCD, CRELD2, ZCCHC12, DMD, GRIPAP1, HUWE1, CFLIP1, KLLN, GALNT18, CERT1, CAMKMT, CFAP44, LINC02743, OPCML, OR4A1P, TRIM51DP, EHBP1L1, FAM89B, GAβ2, SOX5, DNAJB12P1, GLI2, KCNH7-AS1, SEMA3F-AS1, P2RY1, RPL26P30, LINC02098, FGF6, FGF23, LINC02438, UBE2D3, DNMT3A, ADGRG6, BCAM, LINC01427, TYRL, GRM5P1, RBBP5, TOMIL2, DDX52, USP36, LRRN2, RNA5SP74, SYT10, ASSIP14, NLRC3, CT66, CCT4P1, NUDT12, LINC02163, BTNL2, DNAH8, PPP3CA, KIF14, ZNF281, ZNF251, BRAP, RPH3A, HIGDIC, ZNRD2-AS1, MTND3P22, C1GALT1, DOCK4, UBA7, TRAIP, SNUPN, AKTIP, CYB5D2, ANKFY1, EML2, CHMP4B, POCIA, ALDOAP1, KCNG1, GFI1, RN7SL824P, RMI2, MACROD2, MACROD2-AS1, PALLD, ISCA2, PXYLP1, ELMO1, TNRC6A, AGR3, AGR2, MUSK, SVEP1, SULT4A1, LINC02240, ZNF608, RAB7B, SLC26A9, UMOD, PTDSS1, CELSR2, PSRC1, LINC02398, KAZN, C8orf87, MIR8084, NBPF13P, PRKAβ2, SVIL2P, RN7SKP178, NRP2, LPP, FSTL5, U2, KCNVI, GRAMD2B, GRHL1, GDF7, LDAH, ZNF512, EEFSEC, RN7SL541P, LINC01843, RFX6, LINC01162, LMTK2, DPY19L4, PCATI, CASC8, POU5F1B, NUDT5, SEC61A2, RAPSN, HTR3B, KNL1, ARFRP1, TNRC6B, NUCB2, NCR3LG1, IDE, RN7SL51P, TH2LCRR, IL13, TNIP1, IL12B, HLA-B, TRAF3IP2, FYN, TNFAIP3, TMC1, RDX, COQ10A, ANKRD52, PSMA6, NFKBIA, STX4, STX1B, TYK2, IFNLR1, RUNX3, LCE3A, LCE3B, FAP, LINC01845, ADO, ALDH7A1P4, or combinations thereof.

In some embodiments, the one or more genes affected by BMI comprise ADCY3, FTO, NRXN3, MESTP3, LINC02306, LINC01579, LINC01581, TFAP2D, LINC01541, RPL31P12, RNU4-17P, MC4R, LINC01875, TMEM18, SEC16B, DNAJC27, FAIM2, LINC01865, LINC01874, SNRPC, SLC39A8, RPS17P5, TFAP2B, PIK3C3, SEMA3B, HIPK3, KIAA1549L, CEP120, SULT1A1, SLC7A14-AS1, SLC7A14, LEPROT, LEPR, PCSK1, PRDX4P1, THAP12P9, DLG2, KCNS3, SPRY4, COLEC10, SH3PXD2B, LINC01944, LINC01878, RBM47, RNU7-74P, NOS1AP, CACNAIE, DOCK4, NECAβ2, ZNF257, ZNF208, CNTNAP2, LINC02292, SYNE3, CEP170P1, CALCR, SEPHSIP2, ADGRE4P, WNT7B, PAPPA, PIK3C2G, NUFIP1, LINC00330, DOCK1, P2RY1, EVC2, EVC, PTPRD, PMFBP1, CST2, CST5, LINCO2302, ENPP3, ASSIP14, PKP2, PSD3, SLC35F4, CNTN4, LINC00598, JAKMIP2, ST13P19, HHAT, SLX4IP, KSR2, MIGA2, SH3GLB2, TAS2R1, SNHG18, ADAMTS6, MYO10, OGFRL1, RNU6-411P, PRELID3BP6, PRELID3BP7, Y_RNA, EBF1, TMBIM7P, TJP2, TMEM161B, SLC29A4, PAK6, C15orf56, BUB1B-PAK6, PHLDA3, TNNII, TTN, MRPS31P1, LINC02112, FSTL4, C12orf56, ATP5F1AP4, KCNH5, ADCY9, SRL, KIF18B, GFAP, SLC14A2, SEMA6B, TFDP2, LINC02032, GYG1, DHX36, CFAP20DC, TIPARP-AS1, KRT18P34, RSRC1, LINC02043, EGFEMIP, SLC2A2, TNIK, NLGN1, SOX2-OT, LINC01995, RPL7LIP8, MAP6D1, EEF1AKMT4-ECE2, EEF1AKMT4, DGKG, LPP, XXYLT1, RNF168, UBXN7-AS1, RGS12, LDB2, LCORL, LINC02438, ANAPC4, PCDH7, KLF3, KCTD8, SPATA18, LNX1, LINC02283, LINC02260, KIT, LINC02358, RNU6-699P, ART3, PCAT4, LINC02469, ABCG2, CCSER1, GRID2, UNC5C, ADHIB, PPP3CA, NSD1, NRN1, PKMP5, HULC, OFCC1, HIVEP1, PHACTR1, RNF144B, MIR548A1HG, E2F3, CASC15, SPTLC1P2, LINC02828, MLN, PACSIN1, RPS10-NUDT3, ILRUN-AS1, ANKSIA, BTBD9, LRFN2, OARD1, FRS3, PRPH2, MAD2L1BP, LINC02537, VEGFA, RCAN2, FBX021, OLFM1, LDLRAD4, SHTN1, GRID1, VPS11, SLC10A7, RTN4RL1, ASIC2, AATK, BAIAP2, SPATA16, LINC02628, SGCZ, DLEU1, SPARC, PAX2, Metazoa_SRP, SLC16A3, POLD2P1, TRIM66, MYH15, ADARB1, BORCS7, PTGES3P4, NPY, CRTC1, GIPR, PMS2P3, LINC00114, ETS2, PTK2B, ZKSCAN5, SLC25A37, GOLGA3, LINGO1, AXIN1, NTRK2, SETBP1, SAMMSON, FSTL5, LINC02860, SKAP2, MRAS, EFR3A, KIAA1522, AK5, RPL7P6, PRKD1, MTND5P40, SSBP3, ERBB4, ZNF131, FBXL18, ZCCHC7, EPB41L4B, FRRSIL, DMXL2, MTND4LP25, MIR100HG, MACF1, NXPH4, GPR151, ZC3HAVI, TTC26, DNALI1, SOX5, WNK1, MAP2K1, LINC02853, EIF2AK4, LINC00923, DYNLL2-DT, SRSF1, B4GALNT2P1, IGF2BP1, DPYD, SNX19, TCF7L2, APC, CCDCl71, RPA1, ALPK1, JAKMIP3, TRAF3, UTP4, LINC01741, CRTAC1, MAML3, CCND1, TCF4, TNNI3K, LRRC53, FPGT-TNNI3K, PCDH9-AS2, PCDH9, PDS5B, NRXN1, CCK, SALL4P6, ACTL11P, MSTIR, LINC01650, LINC02607, RN7SL831P, EEF1A1P11, PTPRC, PRMT6, NTNG1, GPR61, NRBF2P3, KCND3, NRAS, LINC01649, RNA5SP56, PSMC1P12, TBX15, WARS2, BNIPL, ZBTB7B, MEX3A, MIR9-1HG, PRRC2C, RABGAPIL, ENTRIP2, MRPS14, Clorf21, EDEM3, BRINP3, SYT14, PDE4B, HMGB1P18, ARHGAP15, TRIP12, RFTN2, LSAMP, RNA5SP125, EIF5A2, KLF7P1, CCNL1, LINC00881, RNU6-1252P, NECTIN2, LINC02240, PURPL, CDH9, CTNNA2, UNC79, NEK6, COL16A1, ADGRB2, AKT3, RNU1-131P, RNU6-27P, RGS17, POMGNT2, RNU6-367P, C3orf86, TCAIM, U3, LARS2, SMARCC1, DOCK3, ITIH4, CACNAID, FHIT, PTPRG, CADPS, LINC00698, SLC25A26, MITF, RNU6-281P, ROBO2, GBE1, RPTOR, PDILT, BDNF, BDNF-AS, SPHKAP, YEATS4, KNTC1, C12orf42, RPS3AP49, GPN3, PANTR1, POM121C, LINC01505, POCIB, DUSP6, ELOVL3, NOLC1, MLLT10, KLC1, CELF1, CCDC92, DNAH10, HYOU1, SLC37A4, LINC01122, MIR1471, COPS7B, AUTS2, ERI1, RPL10P19, SGO1-AS1, CADM1, DDC, ETV5, MEF2C, MEF2C-AS2, TTC19, NCOR1, REEP3, ERBB3, PRKG1, LRMDA, UBASH3B, METAPID, SSBP2, CALNI, LINC02687, SNRPEP3, GP2, PDZRN4, MIR138-1, DOC2A, LINC01915, AFF3, ARNTL, CYB5B, NFAT5, LINC00838, PARD3, SGMS1, PCDH15, ANK3, JMJDIC, ANXA2P3, LINC02671, ADK, KAT6B, KCNMA1, LINC00856, PCGF5, CPEB3, SORBS1, ARHGAP19, ARHGAP19-SLIT1, SLF2, GBF1, C10orf95-AS1, SDCCAG8, AGBL4, PATJ, COBLL1, LINC02706, LINC02714, LINC00558, ZNF646P1, RNA5SP30, GPRC5B, BPTF, CCNE1, C19orf12, SILC1, TNRC6B, ABHD17C, CEMIP, BMPRIB, CLVS1, RBMS1, PPP1R3A, RNF103-CHMP3, RPS6KA5, RASA2, LINGO2, LINC00907, AKAP6, LINC01524, NPC1, RMC1, RPL35AP28, BCDIN3D, MEG9, LINC02285, CPS1, TNRC6A, ANKFN1, LINC00470, AIDAP3, ZBTB7C, PEPD, ZC3H4, TMEM160, STON1-GTF2A1L, GTF2A1L, LHCGR, FLT3, LINC01067, BNC2, IFT57, HHLA2, LOXL1-AS1, TBC1D21, PGPEP1, CADM2, CXXC5, RNU6-236P, PRKN, KCNJ12, MAP2K3, SEMA4D, GADD45G, MIR4719, EHBP1, RSL24D1P2, RN7SKP140, NKX2-4, FIGN, POLR2DP1, PARD3B, RIT2, SYT4, CTBP2, AS3MT, BORCS7-ASMT, LINC02627, VWA2, ENO4, RPL21P16, LINC02641, ZRANB1, NPMIP31, C10orf143, EBF3, TCERGIL, ADGRA1-AS1, CHID1, BRSK2, ASCL2, MIR4686, SLC22A18AS, MIR8070, KCNJ11, LIN7C, LINC02742, MPPED2, HS6ST3, RPL35AP22, ME2P1, CHCHD2P8, MTIF3, RNU6-63P, HIVEP2, CMIP, RANBP17, GLYR1, POC5, SLC25A5P9, OLA1, SPATA19, NCAM1, MEF2D, RELN, MYO19, SUPT3H, MAST4, LINC01248, MIR3681HG, TRIB2, DTNB, KCNK3, PPMIG, NRBP1, BABAM2, PPP1CB, SMIM7P1, RN7SL602P, RPL21P36, CRIM1-DT, FEZ2, VIT, SLC8A1-AS1, LDHAP3, HNRNPA1P57, CAMKMT, LINC01118, ASB3, RTN4, ACTGIP22, NUDT21, TTC12, CDH13, SMG6, KRTAP4-6, KRTAP4-12, GRP, SEC11C, TAF9P3, EXT1, BBS4, ERC2, KLHL2P1, GTF2IP12, CWC27, RMDN1, NAMA, MICALL1, KLF7, PSMC1P5, LINC02894, VIRMA, CASZ1, MTOR, MTOR-AS1, NPPB, SBF1P2, RNU1-1, LINC01783, ATP13A2, MICOS10-NBL1, NBL1, MICOS10, LACTBL1, TEX46, A3GALT2, CSMD2, PABPC4-AS1, PABPC4, RLF, HIVEP3, IPP, PDZKIIP1, TAL1, ADH5P2, ADGRL2, TTLL7, DDAH1, LINC02609, BARHL2, EVI5, GALNT13, ITGB6, LINC02478, SLC4A10, DPP4, GRB14, SCN2A, SCNIA-AS1, SCNIA, XIRP2, SP5, EIF2S2P4, DYNC112, LRRC2P1, HNRNPA1P39, SCHLAP1, SAP18P2, RNU6ATAC19P, TMEFF2, PLCL1, FTCDNL1, RN7SL717P, NBEALI, CYP20A1, DSTNP5, ZDBF2, LINC00523, DLK1, LPCAT1, FOX03, ASCC3, NTM, TSKU, GUCY2EP, UBE3B, MMAβ, ZCCHC8, GNAI2P1, RPL30P11, LINC02349, VPS13C, DTX2P1, DTX2P1-UPK3BP1-PMS2P11, ZMIZ2, AHR, TMEM60, LINC01019, NFIB, CDCA4P1, KDM4C, ARAP1, SP1, TAFA5, GALC, DPH6, C3orf38, ABCF2P1, NDUFA5P5, NSUN3, ARMC10P1, BBX, ZBTB20, GSK3B, ADCY5, HACD2, DNAJB8-AS1, CPNE4, EPHB1, HMGNIP10, STAG1, LINC01793, RNU6-508P, RNA5SP94, BCL11A, RN7SL361P, LINC02831, NFUI, RNA5SP99, CHMP3, ACTRIB, REVI, LONRF2, LINC01965, FHL2, PAX8, PAX8-AS1, NCKAP5, LRP1B, TEX41, LINC01911, RNU6-692P, LINC02612, SQOR, NLRC3, ADAMTS18, VN2R10P, BCL2, DPYSL5, CDKN2AIPNLP2, CPSF6, CIGALTIP1, SUGP1, TCEA2, ANKS1B, LINC01774, FGFR4, LINC00458, DPF3, DNAJC11, OR5BP1P, LRRC55, ELOCP3, MRM1, PLCD4, ZNF142, PTPRN, DNER, AGAP1, GPC1, LMCD1-AS1, SETD5, CIDECP1, VGLL4, PPARG, GSTM5P1, LINC02022, NUP210, ANKRD28, SATB1-AS1, RARB, ARPP21, ACVR2B-AS1, ACVR2B, ULK4, UCHL5, ZBTB41, IPO9, DAB1, NEGR1, DNAJB4, GIPC2, ARL8A, OPTC, ATP2B4, LINC01698, LINC01696, LYPLAL1-AS1, DNAH14, ALKAL2, SNTG2, ATP6VIG1P7, LINC01309, ACER2P1, CADM2-AS2, SSR3, MIR4527HG, SKOR2, ADGRB3, ADGRG6, FTHIP5, TBKBP1, TBX21, HIP1, SUFU, TRIM8-DT, MTCH2, MAP2K5, NPM1P42, ADPGK-AS1, SBK1, SH2B1, LINC00678, GPR139, CRYZL2P-SEC16B, CDYL, NPHP3-AS1, GNGT1, RFX3, PRR5-ARHGAP8, ARHGAP8, LRP1, ECE1, KIAA1328, MIR1302-7, CACNAIB, ZNF578, ADAM19, ZC3H12C, RDX, BANK1, LINC02748, LINC01517, PTFIA, YWHAZP3, DNAJC3, TAFA2, FXNP1, PDE10A, FOXN3, LINC02228, LINC01304, DCDC2C, LINC01808, MRPS5P4, ANGPT1, RNU6-502P, BTN1A1, SLC7A6, NMRAL2P, PRKCE, LINC01822, LINC00882, POLR2M, KRT85, KRT89P, RN7SKP211, TRAK1, STK39, SNX10, KIAA0586, MAP2, MICAL3, GRXCR1, MYOID, TMEM98, NIFKP3, APOBEC1, DNAJB6, SUCLG2, KLF14, TSGA13, IL3, ACSL6, HSPE1P24, CD2, KAZN, STX18-AS1, TLL1, LINC01803, RPL6P21, SYDE2, Clorf52, COL6A5, COX10-AS1, ATXN2L, NPIPB9, ERICH3, RNFTIP2, MGC27382, LINC01720, TUFM, KCTD15, RN7SL150P, UHRF1BP1, TDRG1, MAST2, PIK3R3, ZZZ3, GNAT2, LMOD1, TMEM161B-AS1, FBXL17, MMS22L, PRORP, HMGB3P15, PAFAH1B2, SIK3, ZSWIM6, L3MBTL3, MOBIAP1, RAC1, DGKI, NRG1, GOLGA2, RSU1, LINC01872, TKTL2, RPL35AP12, MDGA2, LINC00461, LINC02382, PPARGCIA, TEX29, TTLL4, INKA2, MCTP1, RNA5SP272, MTND2P8, JAZF1, CSMD1, ANKRD6, LYRM2, RNU6-144P, ZYXP1, RNU6-1213P, MAFB, MACROD2, LINC02421, MAGI2, NKAIN2, UBN1, FKBPIA, NSFLIC, MASP2, SRM, LINC01713, CAMTA1, CADPS2, LINC01485, CPEB4, ASXL3, CHP2, PRKCB, LINC00643, ELP3, HCK, RNA5SP404, RNU6-1, RALYL, TPM3P3, RCN1, POU6F2, MED23, ARG1, PRMT7, DIAPH3, RBBP6, LINC02465, SMIM30, CDH22, ALKBH3, HSD17B12, LINC02163, RNU6-334P, BTD, CACNAIC, DCP1B, LINC02177, MGA, OTUD7A, SLIT2, GCNTIP5, GSAP, SLCO3A1, TTC34, YWHAQ, TUBA4A, MFAP2, SCARB2, LINC02288, DYRKIA, SALL4, LINC02273, KLHL32, EMC3, STK24, HSPA8P17, PRSS27, SRRM2-AS1, NREP-AS1, NREP, MCC, LINC01957, COMMD10, TUBAP15, RNU6-718P, JADE2, HBEGF, DIAPH1, GLRA1, LINC01470, GRIA1, MFAP3, MIR3142HG, ATP10B, TENM2, WWC1, RBFOX1, KLF1, CSNK1G2, EBF2, OPCML, CACNG3, TRPM3, PDEIC, UBE2W, STAU2, SYT16, BAG6, PRRC2A, IQSEC1, SMCO4, LINC00469, PTP4A1, CAPZBP1, G6PC1, AARSDI, BCL11B, PGPEPIL, MAST3, TLE1, CFAP74, BACH1, EIF4BP4, LYZL4, DSCAM, LINC01938, MIR129-2, RORA, AOC1, KCNH2, RN7SKP106, RPS3AP23, ASCC1, LINC00861, TRIB1, UBE2WP1, YWHAZ, LINC02062, RORA-AS1, UBXN7, TUSC3, FGFR1, C8orf86, PRUNE1, FIBCD1, TRIM47, PKHD1, GFRAL, HCRTR2, BEND6, EEF1B2P5, LMBRD1, HLA-DRA, KCNQ5, KHDCIP1, LCA5, SH3BGRL2, TENT5A, KHDRBS2, TBX18, RRAGD, NACAP7, ATF1P1, MIR2113, PRDX2P4, SIM1, KLF16, FAM160A2, ECE2, CELA2B, TRPS1, NFIL3, CAST, RABEP1, NUP88, RPSAP64, LINC01700, CRB1, LINC00967, TSNARE1, MIR4432HG, GNAQ, LINC01876, DDX43P2, VWC2, PHLPP1, SSH2, HMGB1, AP1S1, HMGB3P19, OPRM1, ZNF536, RNU6-837P, EEF1A1P16, PHF2, IGHMBP2, MRGPRD, LINC01234, TRDN-AS1, TRDN, HEY2, NCOA7, RNU7-152P, SNORD28B, SLC22A3, QKI, RN7SL366P, GET4, SUN1, ZFAND2A-DT, UNCX, MAD1L1, CARD11, RNF216, ZNF815P, DGKB, TMEM196, SP4, LINC02523, CACNB2, LINC01977, LINC02337, GALNT16, SEMA6D, ASSIP10, LINC01414, LINC01289, LINC00583, MIR873, CCDC85A, ZFPM2, LINC00358, S100B, PRMT2, SNORD3P1, MINDY2, KCNB2, GALNTL6, TERFIP3, RNU1-89P, INPP4B, GAB1, ANAPC10, RNA5SP169, MARCHF1, BASP1, RN7SKP133, NNT, MRPL49P1, RGS7BP, AP3B1, PPIAP79, RBBP4P6, RNU6-727P, LINC02060, MEF2C-AS1, LINC02161, LINC00491, PAM, LINC01950, EFNA5, INO80, CIBARIP1, MAPKBP1, TTBK2, ONECUT1, RPSAP55, LINC02490, ZNF280D, TCF12, MYO1E, NPMIP47, C2CD4A, LINC02568, USP3, MEGF11, DIS3L, HMGN2P47, LINC02206, NEO1, STOML1, TAOK2, STX1B, RPGRIPIL, PPIAP48, SNTB2, AARS1, LINC01572, ZFHX3, LINC01568, LINC02125, RN7SKP190, ACSF3, CBFA2T3, MINK1, DLG4, KDM6B, MYO18A, SLC6A4, ACACA, COASY, BRCA1, ATXN7L3-AS1, KANSLI, SKAP1, SKAP1-AS1, ZNF264, DUXA, ANKRD34C-AS1, RASGRF1, AP3B2, MIR9-3HG, RHCG, IGFIR, ADAMTS17, MRPL28, NTHL1, CLUAP1, PPL, LINC02152, RPL21P119, C16orf72, BFAR, HOXB3, HOXB-AS3, HOXB5, TOMIL1, RN7SKP14, MSI2, DCAF7, UNC13D, PTPRM, CEP192, AQP4-AS1, DCC, RAB27B, LINC01415, RNU6-567P, LINC01898, SALL3, ABHD17A, SF3A2, ZBTB7A, CHAFIA, SLC35E2B, CDK11B, P2RX4, DUX4L52, PDXDC1, HTRIF, MAPK3, COROIA, RPSAP1, PCCB, MSL2, IGLON5, PRXL2B, TNFRSF14, HSD17B1, HSD17B1-AS1, HEYL, TBXAS1, HIFIAN, RNU7-133P, TTC36-AS1, KMT2A, LINC02744, JHY, KIRREL3, RN7SL167P, IGSF9B, B3GAT1-DT, CCND2, CCND2-AS1, MGST1, SLC15A5, PLEKHA5, WSCD2, RN7SKP250, MVK, RPH3A, HAUS8P1, RBM19, UBA52P7, RN7SL865P, RPS27P25, COX6A1, CLIP1, PITPNM2, CHFR, ZMYM2, LINC00423, NBEA, PCDH8, BOLL, VENTXP5, RPS26, ITGAX, PIAS4, LINC01812, GLTP, GCKR, ULK1, PUS1, RASSF10, RFC5, THEM7P, CTBP2P6, TTC17, ALKBH3-AS1, PHF21A, C11orf49, BAD, GPR137, NRXN2, EFEMP2, SF3B2, RNU1-84P, NPAS4, NAALAD2, DYNC2H1, ZBTB16, TAPBP, ARL14EP, CDH7, TOMM40, GLPIR, LINC02618, ILRUN, RN7SL618P, MIR1297, PCDH17, DNAJAIP1, LGMNP1, SLAIN1, CCT5P2, NIPA2P5, LINC02336, FAM155A, ARHGEF7, OR7KIP, RNU11-5P, LINC02326, ATP2A1, CHORDC1, SEMA3F, SKP1P2, R³HCCIL, PURG, RNU6-537P, RNY1P1, GTF3A, ENTPD4, ANKDD1B, PREX1, ARFGEF2, LMX1B, KRTAP4-11, KRTAP4-9, GFPT1, CTAGE16P, XKR6, POU6F1, AMHR2, PRR13, CBX5, SCAT2, PA2G4, RPL41, ATP23, CTDSP2, DYRK2, LYZ, RAB21, LINC02426, BRWDIP2, RNU6-148P, LINC02392, RMST, BTBD11, LRFN5, YWHAQP1, SOS2, FUT8, LINC02301, MIR377, MIR541, PPP2R5C, DYNCIHI, MOK, CKB, GABRB3, RNU4ATAC7P, RNU6-743P, RALY-AS1, PIGPP3, PTPRA, SYT3, MARK4, EXOC3L2, RNU6-797P, KDM7A-DT, PRDX5, CCDC88B, MAP3K3, TNRC6C, ENTPD6, BRWD1, MLIP, PTPN7, RNA5SP271, HNF4G, RNU2-54P, LINC01109, SLC2A3P4, FLJ46284, RUNX1T1, RN7SL685P, MROH5, FREM1, VAV2, KRT18P36, RPS26P2, UBAP2, TLE4, TUT7, ROR2, DNM1, MAP2K2, KRT8P11, TEX10, GRIN3A, DELEC1, ASTN2, RNU6-710P, TPTIP9, LINC01613, TTLL11, CRB2, STRBP, MED27, EHMT1, MIR4675, ACBD5, RNU6-1103P, POU6F2-AS1, OGDH, FIGNL1, POM121, GTF2I, RSBNIL, GRM3, RNA5SP89, KCTD13, LINC01992, PPFIA3, RPLPOP1, KIF16B, BACE2, TGFBRAP1, RPS24P8, TMEM158, LINC01440, LINC02409, KDMIA, MALATI, SNRPGP19, LINC01239, SUMO2P2, UBE2R2, GGNBP2, LINC02822, RERE, LINC02406, FOXP2, GRM8, AGAP3, PTPRN2, HAS2-AS1, EIF4EBP2P3, SCYL1, RPL7P25, IGHV4-28, GNB1, CALML6, HAPLN4, STK35, PLCB1, PLCB4, MIR663AHG, VSTM2L, YWHAB, SULF2, PTK6, PPDPF, C21orf62-AS1, BID, RPL10P2, MIR3143, NOTCH4, MIR124-1HG, RFLNA, ZNF664, RABEP2, COMT, CACNG2, TEF, TOB2, PKDREJ, PLXNB2, SPI1, ZC3H11B, IGF2BP2, NUDT3, CDKAL1, CNNM2, KCNQ1, RSL24D1P4, SEPTIN14P21, PRSS37, OR9A3P, CERS3, CERS3-AS1, CYP2E1, NKAIN1, SNRNP40, ADGRE1, RNA5SP182, CYP7B1, RNU6-839P, SDC1, DRGIP1, LINC02541, HOTTIP, EVX1-AS, GCK, MED30, SLC30A8, CERCAM, MIR219A2, CTSC, GAPDHP70, MAP3K12, HNF1B, MTPN, NID2, SUZ12P1, ZNF133, TAF4, DMD, VTRNA3-1P, HUWE1, IL13RA1, DUSP9, KRT18P48, NEK4, KRT18P9, CYCSP55, FAM185BP, RPL5P22, CDKN2B-AS1, DMRTA1, MYEOV, LINC02747, MIR924HG, GPR101, HLA-DRB1, HLA-DQA1, HMGA1, MDFIC, RPL10AP3, ZNF169, ANKS6, GAPVD1, RN7SL198P, CDCl23, STK33, LMO1, ALDH2, MAPKAPK5-AS1, IL27, NUPR1, NUCKS1, RAB29, HECTD4, BTBD7, RAIL, ATAD5, HLA-DRB9, FGR, LINC02574, GON4L, QPCT, RNU6-939P, LINC02613, HNRNPLL, PSME4, LINC01153, RPL19P16, ELP4, PAX6, GAβ2, PRDM6, EYS, RN7SKP135, NUTM2F, SWI5, B3GALNT2, ITIH3, HERC4, RRP12, FRAT2, SINHCAF, LINC02128, LINC02127, FNBP4, NADK, SYT11, U4, RBM6, MONIA, CD200R1L-AS1, FEZ1, NOP56P1, LINC01623, PRAMEF1, LINC01784, FHDC1, LINC02763, ADAMTSL1, IGDCC3, RNU5B-1, BIN2P2, RNU7-165P, PPIAP39, AMBRA1, USF3, RN7SL767P, KCTD19, LINC01605, RNU6-607P, SUCLA2P2, PNPP1, LINC01082, SPOCK1, PRPF6, ACOX1, RASGEF1B, HNRNPA3P13, MUSK, R3HDM2, RAPGEF4-AS1, TTC28, TTC28-AS1, MTHFD2P6, ETF1, SNPH, NAV1, IPO9-AS1, ELAVL4, QPCTL, ATP6VIG3, SH3RF1, COL19A1, OR5V1, GK5, TMEM52, PLCH2, C2orf50, RPL6P4, CEP162, HNRNPA1P51, CBX3P1, RNU6-1246P, FAM120A, TMEM163, NYAP2, MIR5702, COL4A4, LINC00237, ZNF101, ADAMTS9-AS2, SLC22A2, ITIH1, FLRT1, MACROD1, ITGB3, MYL4, GABPB2, SEMA6C, LINC02237, SLC66AIL, TLK1, METTL8, INO80E, NPBWR2, MYT1, KLF3-AS1, USP37, MCM6, ASB4, DDX42, RAB3GAP1, ACAP2, TRAFD1, ZNF12, ZNF316, KCNG3, RNU6-526P, RNU6-1151P, PTPN2, HLA-B, TCF20, CHASERR, TCTN1, FAM120AOS, KAT8, RNH1, ATXN2-AS, BRAP, DHH, NT5C2, GALNT9, SCAMP4, CNTNAP5, RNU6-1051P, HHIP, EEF1A1P8, HTR3D, CD47, LINC01215, MTCO3P1, HLA-DQB3, PACRG-AS1, CAHM, LEMD2, IP6K3, LINC02338, RAD51AP1P1, NCK1, LINC01405, CUX2, SMC5, KLF9, SLC24A5, COL25A1, RNU6-989P, RN7SKP42, OR4C15, OR4C14P, ABLIM3, EIF3H, HDAC2-AS2, OR4A5, OR4A6P, FGF2, OR5M11, OR5M5P, BCL2L2-PABPN1, SLC22A17, LINC02174, MIR148A, DENNDIA, TSHZ2, RPL36P1, LINC02086, HOXB-AS4, EFR3B, LPIN2, CHORDCIP4, STARD13-AS, STARD13, PRKCQ, SLC24A2, P2RY2, FCHSD2, NDUFB9P2, ARHGAP18, LAMA2, ADARB2, LINC01583, MEX3B, RNF223, AGRN, SIGLECL1, LLPHP3, AP3D1, MGAT1, OR2Y1, GRIK1, ROPNIL, MARCHF6, LINC01509, RAD23B, LINC01987, LINC01739, KCNE4, ZNF804B, HDAC9, ITGBL1, MIR4679-2, FAS, LDHBP3, RPL15P3, CRYL1, ABLIMI, PLA2G2D, PLA2G2F, CCDC91, PAX7, LINC01621, SERPINA3, ADIPOR1P2, TMEM212-AS1, IL34, NFE2L1-DT, No mapped genes x SMC5-KLF9, AKR1C6P, PALM2AKAP2, ENTRIP1, RN7SL342P, APOL5, LRRC7, ATP6VOE1, LINC02136, PRIMA1, ZBTB10, TMEM219, MIR3144, UPK3BP1, APOCIP1, APOC1, NUCB2, NCR3LG1, TPCN1, PANK4, COP1, ROBO1, ISCAIPI, HTRIA, LINC02550, PLEKHG5, RN7SKP182, RPL12P40, FSHB, ALK, PCATI, CASC19, NFIX, UBE3C, TCERG1P2, NPIPA7, YIPF7, RBFOX3, LINC01749, LINC01362, PCNX2, SPAG16, CCDC39, EPHA3, GAPDHP50, ADAMTS7, CHRNB4, PAUPAR, TTLL8, EIF3C, INTS10, LPL, ZSCAN32, SLIT3, PTGFR, IQGAP3, RPS15AP7, L1TD1, RN7SL854P, LINC01787, CRYZL2P, PHC2-AS1, PHC2, BEND5, MIGA1, PIGK, CSDE1, ANGPTL7, ANKRD45, PRDX6-AS1, SLC25A44, ASHIL, RUSC1, PSMA5, ZNF436, RPL7P9, NDUFS5P2, RNU1-130P, NDUFS5, DNAJB2, LMNA, SEMA4A, PTBP2, AP4B1-AS1, LINC01347, INKA2-AS1, HMGB1P45, LINC02808, LINC02765, ENAH, ACTRT2, PKN2-AS1, DTHD1, IQCK, DDX20, NCOA1, CASC20, RN7SKP33, EYA2, HSPA12A, DNAJC1, RNU6-543P, WAPL, LINC02629, MYBPC3, C1QTNF4, MIR4487, CKAP5, MIR670, ARL2, BATF2, PACS1, LGR4-AS1, LGR4, CFL1, OVOL1, FOSL1, SLC22A12, OR5M3, FGF19, LTO1, PTPRJ, NUP160, TSPAN4, LINC01488, BCL7A, MLXIP, SBNO1, HCAR1, DENR, DNAH10OS, HCAR2, KDM2B, CFAP251, MGAT4C, RAD52, ATXNIL, IST1, THRA, EBLN3P, MIR4291, PAX5, DAPK1, BARX1, NEBL, C10orf55, VCL, BBOX1-AS1, PPPIR10P1, LINC02551, METTL15, MIR4299, LINC02755, ESPL1, HCAR3, ANKRD52, PRKAG1, DDN-AS1, RNA5SP375, CDK2AP1, ATP6VOA2, PI15, CRISPLD1, SLC22A11, ZNF45, ARL15, RCCIL, ELFN1, CNGB3, ESRP1, LINC00536, LINC01111, RNU4-71P, SH2B3, ATXN2, BMP8A, PSIP1, RN7SL98P, DCAF12, PNPLA7, LINC00474, PES1P2, MTND5P41, MIR1915HG, RN7SL592P, SMARCA2, ANKRD26, ADAM10, DENND4A, RAB11A, EIF2S2, RALY, CCDC34, SPRY3, AAK1, MTA3, TMEM26, PBRM1, PXK, PNPLA2, DISC1FP1, EHBPIL1, TRIM49B, TRIM64C, GRM5, HSP90AA2P, LINC02743, PPP6R3, NOX4, DGLUCY, DCAF4, GPR65, RNU6-835P, VRK1, LINC00871, ZFYVE1, IQCH-AS1, IQCH, PIAS1, SCG3, MAPK6, LEO1, HCN4, RPL4, ZWILCH, LYSMD2, LINC01169, SKOR1, HNRNPA1P5, TLCD3B, ZNF19, PKD1L3, PHLPP2, SPNS1, NPIPB6, BCL7C, CCP110, CALB2, CMTR2, SEZ6L2, PML, REC114, WDR72, SMG1P7, MIR1275, MIR7159, MIR129-1, LEP, OPALIN, SERPINB1, LUZP1, UBQLN4, ATG4B, REXO1, USP53, PJA2, LOXL4, CRACR2B, SYPL2, CPNE7, AKAP10, C17orf58, TRIM65, SLC35G2, NCK1-DT, SIGLEC9, PJA1, ICAIL, HMGB3P11, TMEM18-DT, LINC01115, LINC01119, POMC, SH3YL1, ZAP70, DNAJC27-AS1, CENPO, LINC01104, NPM1P46, ANKRD44, CIR1, CRIM1, CRIPT, DNMT3A, LINC01795, MRPS9-AS2, SH2D6, RPSAP24, HMGB1P47, MIR583HG, MXD3, PRELID1, VRK2, PIK3C2B, LINC02360, ALG8, NDUFC2-KCTD14, H2BC5, MANIA1, FABP5P5, FAM114A2, LINC01861, LINC02106, RNU6-480P, PPPIR10, SKIV2L, RPL27AP5, FAM169A, LINC02144, ANKRD31, HMGCR, IRX1, NR2F1-AS1, TEAD3, SCUBE3, TCP11, ZNF90P2, LINC00533, RPSAP2, CYB5R4, LINC02857, MYL12BP3, SMIM40, GGNBP1, AFGIL, TULP1, ZNRDIASP, SLC16A10, FKBP5, CCDCl46, SPDYE18, TRIM73, LINC01392, LINC01393, ZBTB38, KRT18P35, SCAP, THUMPD3, THUMPD3-AS1, RPL35AP8, RNU6-1129P, RNU6-488P, COX6CP6, MAP4, HRH1, CGGBP1, RFT1, ZNF35, ZKSCAN7-AS1, KIF15, LINC00971, LINCR-0003, ACTBL2, MTNDIP24, LINC02400, SMUG1, RNA5SP360, UGGT2, STK24-AS1, NUS1P4, WBPIL, TRAP1, DNASE1, CD19, ZNF646, RPAIN, ZNHIT3, CSPG5, DUSP13, SPOUT1, RAPGEF3, ZFR2, ACHE, HIPIR, ZNF502, LINC01079, N4BP2L2, N4BP2L1, LINC01065, OLFM4, SLITRK6, LINC00373, LINC01075, LINC00374, MIR4704, AMN, MARK3, RPSAP5, LIMD1, CLSTN2, PPP2R3A, NDUFS6P1, PRSS43P, PRSS44P, ADGRL4, HSPA5, NATD1, PLD2, TTLL6, CALCOCO2, KIFIC, FASN, KRT17P3, TBC1D29P, GIP, MAPT, CTBP2P3, SUGP2, RN7SL70P, MAU2, CCDC61, SINHCAFP2, HMGN1P31, CHST8, CCDC9, BBC3, PPIAP58, NDRG3, SLA2, NINL, RNU6ATAC17P, DNAJC5, UCKL1, CST7, ACSS1, NCOA6, HM13, HMGB3P2, NORAD, ARIH2, ARIH2OS, SLC25A20, LINC02585, NCKIPSD, RN7SL664P, CDC25A, PFKFB4, CACNA2D2, MAPKAPK3, LINC02019, HEMK1, CISH, SENP2, LINC02702, LINCO2151, GRIN2A, AKTIP, ABHD15-AS1, LINC01916, TMEM241, RRN3P4, GRM2, IQCF6, TWF2, TRAIP, BSN, CAMKV, CDHR4, LAMB2P1, ANO10, LINC02006, GPR149, SSR4P1, ADSL, BCL2L13, MRTFA, MAPK11, ACTN3, PSMD3, RPL9P29, USP36, MIR4435-2HG, ACOXL, TBL1XR1, PLAGLI, EEF1A1P27, MROH8, ZFP64, PCSK2, PICSAR, FBXO46, DNASE2, EML2, GDF15, LRRC25, PDE4C, JUND, NPAS1, PTPRS, BCAM, SAE1, COG4, GDPD3, ZNF771, SEPHS2, ATP5F1AP3, NFATC2IP, PKD1, VAC14, GNAI3, GPR1, LINC02758, NEDD4L, PPARD, RMI1, HSPDIP15, ADAMTSL3, TMEM14DP, TET1, GRM4, HMGA2, MIR6074, PEX5L, RNA5SP149, LUZP2, POMK, SP2-DT, SP6, UGGT1, ZBED3-AS1, RPL38P3, RPL21P44, SNX18P23, CWH43, POLK, CERT1, GCNT4, ZCCHC4, PPMIK, ADH7, REEP5, TDH, PRPF38AP1, GATA3, CABP5, ADAMTS16, FUT9, LINC02652, NCKAP5L, SNX11, NPTX1, RPL32P31, SGSM2, B4GALNT2, CDK5RAP3, COPZ2, SEPSECS, ANTXR2, MIR4275, FRMD4B, LINC02008, PTPRG-AS1, HSPE1P19, FEZF2, CSNKA2IP, TRPM7, DRAIC, TLE3, APOBR, ANKRD13B, DZANK1, ZCCHC12, SERPINC1, NBAS, RN7SL813P, CWC22, ACYP2, PTMAP12, BTRC, HHEX, PSMC3, KCNRG, DLEU2, TRIM13, RN7SKP6, SPATA2P1, TRA2B, KCNH8, STAβ1, VGLL2, RNA5SP214, GCC1, ZNF800, PSCA, JRK, SRRM2, ANKRD27, LEKR1, LINC00880, ESR1, GPSM1, EIF2S2P3, ACADVL, PTCH1, PLCE1, SH3GL3, BANF2, RNU6-192P, NR5A2, KRT18P51, GYPA, KCNK5, SAYSDI, UBE3D, TPBG, MLXIPL, H4P1, LINC02398, TCP1P3, MIR4303, RNU4-41P, PDCL2P1, PTCHD1, AHNAK, SCGB1A1, EFCAB8, NCOR2, KIAA0895L, DNAJC6, EPHA4, MIR4268, SYNE2, ESR2, PRRC2B, MEIOSIN, EPB41L3, CRYGEP, RPL12P17, OR4A7P, OR4A8, LINC02854, LINC01445, ROCK2, CEP19P1, SH3BP4, BRINP3-DT, RNA5SP350, TRIM48, FMNL1-DT, HEXIM2, PAEP, LINC01502, RALGAPA1, SNX10-AS1, KIAA0087, WDR11, FAM184A, RASSF9, LINC02820, NTPCR, LINC01810, TBX5-AS1, RN7SKP216, LANCL1-AS1, LANCL1, POLN, HAUS3, IGBP1P5, RN7SL101P, MSANTD1, LINC02232, MTCO3P28, RNU1-63P, SPRY4-AS1, LINC02227, MIR4280HG, LINC02058, GRIK2, NPM1P10, HACE1, GCNT2, MARCKS, HNRNPA1P58, UBR2, POLH, GTPBP2, MLIP-AS1, LGSN, RPL17P25, RPSAP72, MTCYBP36, EPHA7, GATA4, TBC1D31, SNORA72, LZTS1, TMEM97P2, CDCA2, LINC01288, RNA5SP267, SLC2A13P1, RRS1-AS1, RPSAP74, RNU6-1200P, NR1I2, HLTF-AS1, ARHGEF26, TOMM22P6, OTOL1, LINC01323, MIR1263, RNU1-70P, KLHL24, KLHL6, RNU6-243P, ABHD5, STIMATE, STIMATE-MUSTN1, MRPS17P3, RN7SKP61, CLNK, ARSJ, UGT8, RPS14P7, TMOD3, MNS1, RPL36AP45, TMEM266, LINC01197, LINC02852, RRN3, SLC9A3R2, EGLN3, KTN1-AS1, NDN, RNU6-741P, BBS2, OGFOD1, UBE2FP2, RNU6-21P, SMPD3, DHRS11, SLC39A11, POLR3KP2, WBP2, LINC01894, LINC01899, LINC01893, LRRC30, RNA5SP472, ZNF507, RPL7P2, LINC01597, LINC01370, PPIAP21, ZMYND8, MAP3K7CL, KCNJ4, TPTEP2, M6PRP1, TDGFIP3, RNU6-133P, RN7SKP183, PHEX, ZFX, PTP4A1P5, ABCB7, NEXMIF, DACH2, RPL35AP21, OSGIN1, MLYCD, WDR48, LINC00927, CTXND1, RPL23AP28, PAX3, ETV5-AS1, TBX6, NFE2L1, ARRDC3-AS1, RPL35P2, SOCS5, ERLEC1, EIF2S2P7, LINC01247, ALCAM, RAP1BP2, BCL2L11, WDR33, LIMS2, R³HDM1, PKP4, PCGEM1, SLC44A3P1, LINC01802, CREB1, RESP18, PTMA, PDE6D, TSPAN2, RNU6-983P, LINC01724, MTCYBP14, MTND6P14, TMEM54, RNF19B, TMEM69, THAP3, HNRNPA1P64, MIR137HG, BUB3P1, EIF3IP1, EXOC4, ZDHHC4, GRID2IP, ATP5MF-PTCD1, PTCD1, ZNF618, JKAMPP1, AKAP8P1, SLC25A6P2, RN7SKP114, KRT18P24, SLC28A3, ASPN, CENPP, CACUL1, C10orf90, DPYSL4, BEND7, KIAA1217, MYPN, HMBS, DNAJC24, OR5W2, OR511, CTSW, USP35, FAT3, ASCL4, ANAPC7, SYT10, RPL36AP41, SRGAP1, UBE2L5, LINC00332, STARP1, SMAD7, CLN3, IFITM3P3, RNU6-1120P, AGO3, ITPR3, RREB1, RPL24P7, UBE2E2-AS1, LY86, BTF3P7, GRAMD2B, PPIAP66, MRPS22, LRATD1, LINC01811, RNU6-1230P, DCLK2, SUCLA2, PCDHA8, PCDHA6, PCDHA12, PCDHA13, PCDHA1, PCDHA4, PCDHA10, PCDHAC1, PCDHA5, PCDHA3, PCDHA7, PCDHA11, PCDHAC2, PCDHA9, PCDHA2, FAM180A, SNX29, IMMP1LP2, RPS15AP32, MICA, SPEF1, C20orf27, MPZ, PCP4L1, LINC01991, TRMT112P5, RNA5SP433, KCNG4, RNA5SP396, FAM81A, CIGALT1, COL28A1, SLC7A10, GPATCH2, LARGE1, MAD2L2, TAF4B, RHBG, RN7SKP32, MAPK6P4, TP73, LINC01824, MIR7515HG, TNFRSF19, MIPEP, TRAMIL1, ADAMTS2, LRP8, SPTLC3, LINC02279, TMEM252-DT, LINC00520, KCNE2, SGK1, GLIS3, IGF1, ZNF965P, CYP4F34P, PDGFC, LINC01986, CHL1-AS2, TAF11, SYN2, SULF1, LINC01080, LINC00707, PTCHD3, LINC02800, GRHL3-AS1, MRPL33, LINC00261, FOXA2, PLUT, OR4S1, OR4C3, GRB10, RIC3-DT, G6PC2, SPC25, MTNRIB, GTF3AP5, CRY2, MADD, C2CD4B, FADS1, FADS2, PROX1-AS1, PROX1, BTBD7P2, TRIM42, NAALADL2, AACS, BRI3BP, RIN2, PDZD2, SLC25A36, IGKV3D-7, IGKV1OR2-118, PUS7, RINT1, PDE7B, MTFR2, DSEL-AS1, WWOX, TMEM132B, CDH23, CDH4, ALOX12P2, ALOX12-AS1, EBNA1BP2, CFAP57, ZNF577, HERC2P6, GOLGA8S, CLHC1, RPSAP12, RN7SL87P, EFCAB10, JPH3, CRLF3P1, NELL1, CDH12, TOX, ARHGAP40, FGF9, RN7SL766P, LINC02885, AGBL1, CYP24A1, PFDN4, NPAS3, MAP3K1, FTLP19, ERO1B, EDARADD, RN7SL218P, CXCL5, ANKH, LINC02720, ARL6IP1P3, RNF6, RFTN1, ERAP2, ERAP1, TRIM9, TNFRSF1B, RPA3P1, RNU6-5P, SPAAR, LINC01801, RNU6-716P, RNU6-778P, DOCK2, LINC00343, SALLI, UNGP1, KEAP1, FERD3L, POLRIF, CELA3A, LINC01635, LINC00624, BCL9, CACNAIS, TPRA1, AKRIB15, PHOSPHO1, LCP2, UFM1, LINC00437, GNGT2, EFHC2, LINC02468, PDE3A, INHBC, STK36, IHH, DIS3L2, VTRNA1-3, LINCO2456, CSK, FAF1, POU2F1, PTPRT, MIR365B, RNU6-1134P, LGII, FRA10AC1, INSYN2A, RNA5SP164, CMYA5, LINC01455, OTX2P2, PABPCIP2, ATP13A5, ATP13A4, CRNDE, SPDYA, COL24A1, ZPR1, ATP2B1, ALDH1A2, LIPC-AS1, LIPC, CETP, HERPUD1, THEMIS3P, AKRIB1P6, CEACAM16-AS1, BCL3, CELSR2, TDRD15, APOB, SLC30A3, BAZIB, VPS37D, PLCG1, ZHX3, ADIIP1, SNRNP48, BMP6, EFNB2, ZNF787, LINC01864, CCDCl90, RAB3GAP2, MSRA, RSPO2, GRHL1, RNU7-2P, ABCA12, SEMA5B, LINC01438, MIR297, RPL34P23, BSX, TEAD4, TSPAN9, ITPK1, MX1, FAM238C, or combinations thereof. In some embodiments, the one or more genes affected by additional brain disorders include, but are not limited to CCDCl92, TRAF3, TNIK, LINC02077, GPC1, U3, IGKV1OR2-108, PAX8, LINC01374, LINC01375, KDM4F, SRSF8, POLD2P1, NR2F1-AS1, PLCE1, PLCE1-AS1, LINC01165, LINC02870, FHL5, TRPC6, UFL1-AS1, DGKB, ANO1, APOE, B9D1, CELA3B, HSPG2, EPHA3, IL17RD, MIR3912, RPSAP71, LINC02915, THBS1, DPP4, SMIM21, TSHZ1, MSRB3, PKDCC, VRK2, RN7SL101P, MIR4275, LINC02058, ACTG1P22, IRAG1, Y_RNA, RN7SKP224, VNIR18P, LINC02443, NTF3, FOXP2, MDFIC, ZIC4, LINC01394, FOXF2-DT, PPIAP62, TBC1D5, RNU6-488P, HSPE1P19, FAM172A, MIR100HG, RPL9P5, DACT1, FURIN, SEZ6, PIPOX, TBC1D22A, KRT18P32, LINC01090, OXTR, CAV3, CNTN6, CHL1, BNIP3P41, LINC01331, MIR4464, CRISP3, PGK2, SH2D4A, SLC7A13, CSMD3, PEBP4, FAM135B, LINC01503, KDM4C, NPFFR1, LRRC20, LINC02752, ZBED5-AS1, RPS21P8, RPS20P32, MYO16-AS1, MYO16, NXN, COL5A3, SPACA6, CELSR1, or combinations thereof.

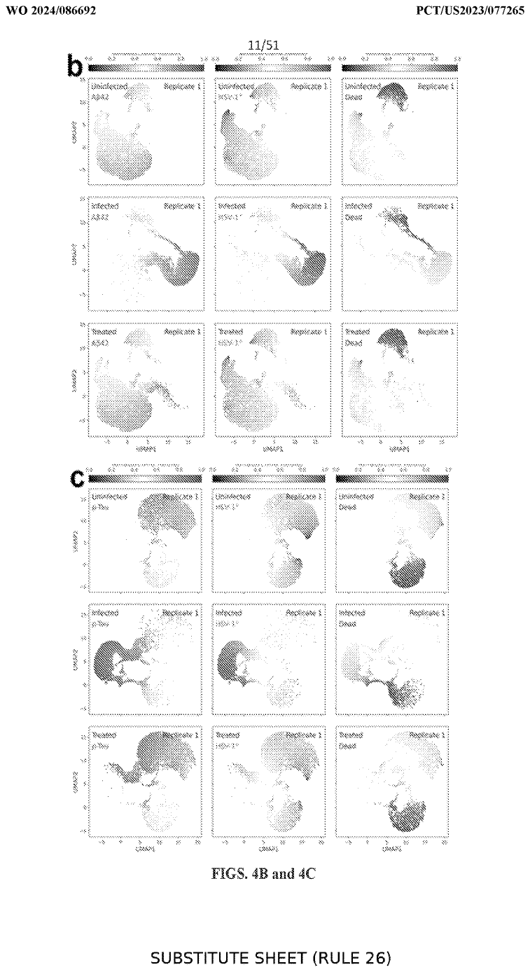

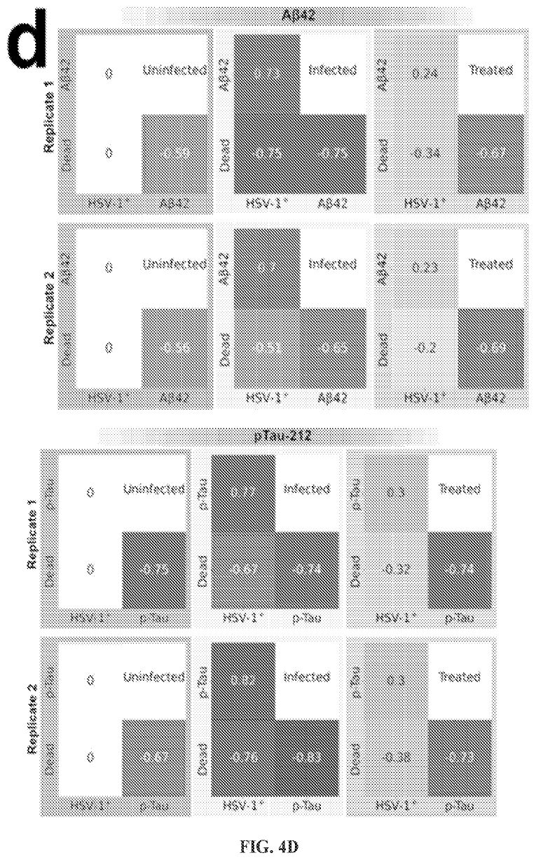

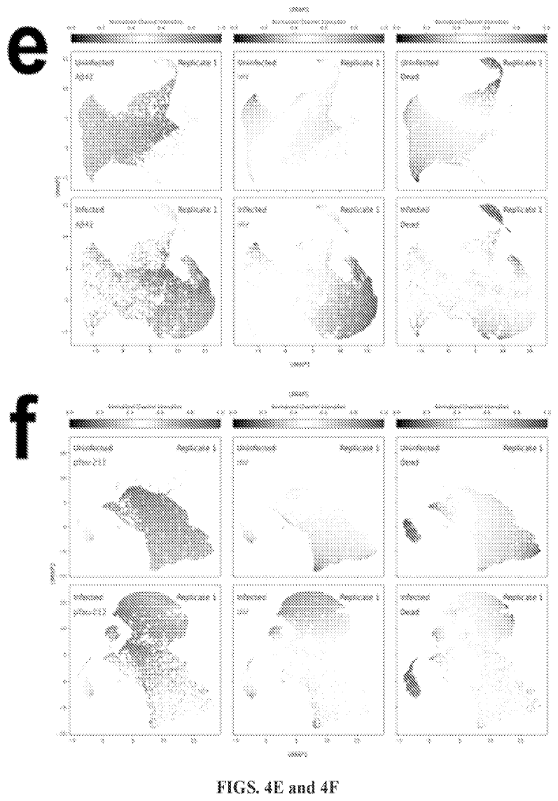

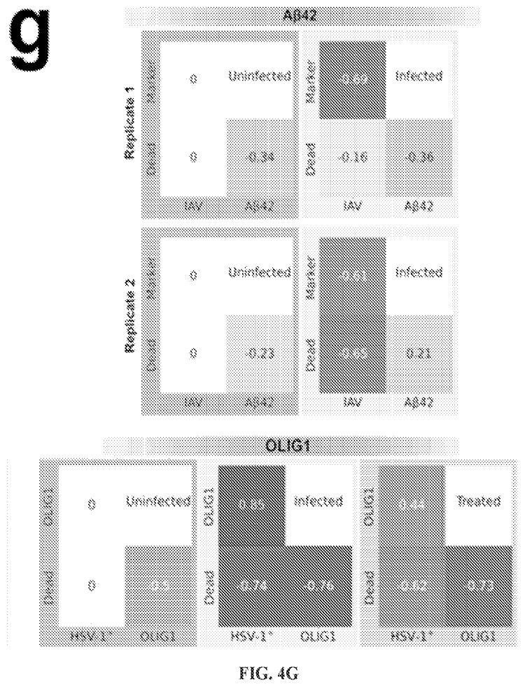

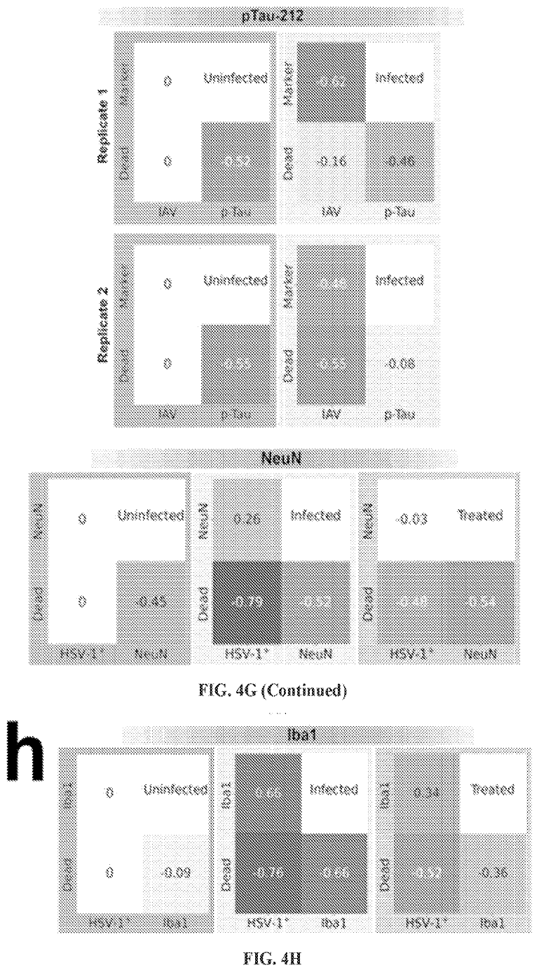

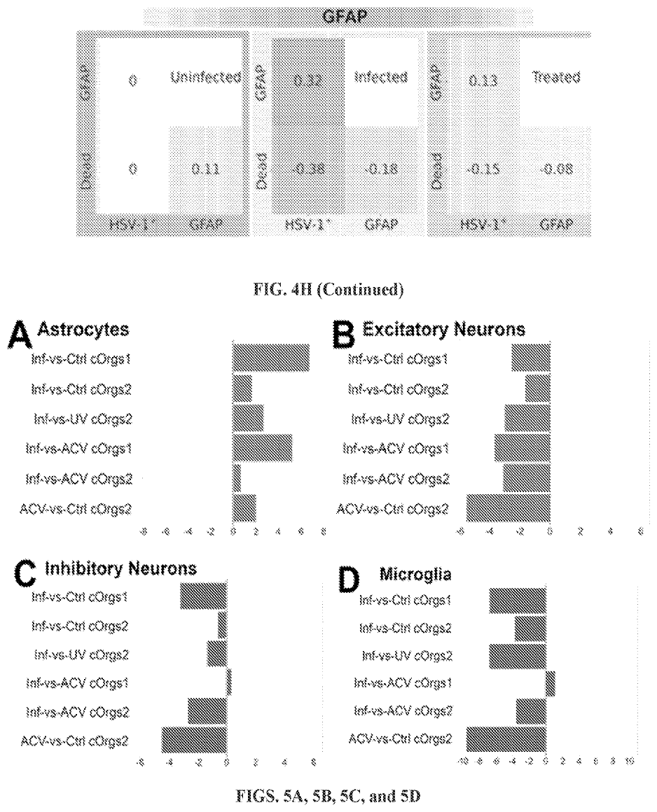

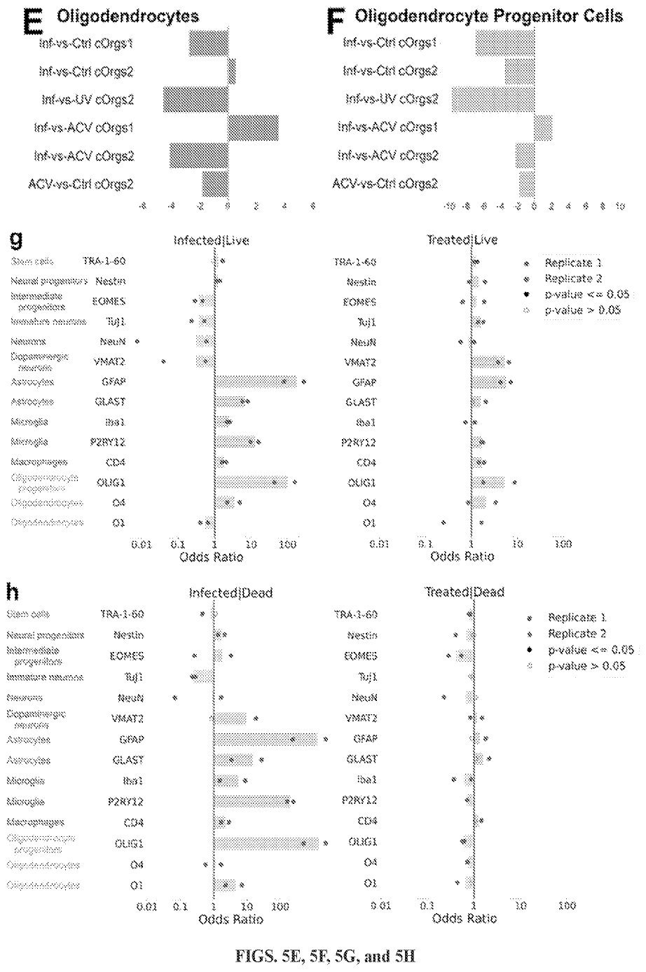

In some embodiments, the one or more genes affected by disorders affecting height comprise SKIV2L, MIR588, CENPW, LINC00476, PTCH1, HMGA2, MIR6074, ACAN, GDF5, ZNFX1, IGF2BP3, ZBTB38, MTRFILP2, CCDC26, RNA5SP56, PSMC1P12, MTMR11, RPL32P13, LCORL, HHIP, HHIP-AS1, CEP85, DCST2, ATAD2B, RPL36AP14, ADCY5, LEKR1, LINC00880, AFF1, CDKAL1, HMGA1, CYCSP55, ESR1, LINC02603, RNY4P18, MIR7702, STRBP, HKDC1, Y_RNA, ADRB1, NHLRC2, APOLD1, OBI1-AS1, LINC00446, PHF23, C20orf203, CENPM, CTBP2P6, MIR670, ADGRG6, SV2A, SF3B4, HMGB3P23, KCNB1, LINC02213, GTPBP4, TRIM48, SLC5A12, ROCR, APBB2, ADAMTS17, HSPEIP1, RN7SL680P, MSH2, RPP14, HTD2, DLEU1, PRKCH, SLC38A6, CFAP61, CDC5L, AP3D1, DYM, CCNE1, C19orf12, KNTC1, LINC01149, DSE, ARHGEF3, TASOR, SYN2, GSTM5P1, RPS23P3, FKBP9, KCNN3, RN7SKP263, CRADD, RNGTT, RNU2-61P, HFE, H4C3, DNASEIL3, MOCS2, IGF2BP2-AS1, IGF2BP2, UQCC1, FAT3, TUBBP3, MICALL2, NIPBL, GLIS3, MTND4LP25, IP6K3, UQCC2, NIPSNAP3A, LPAR3, SSX2IP, WFIKKN1, ZNF462, AGPS, DPP6, JAZF1, RPL35AP34, CPPED1, APTX, NPAS3, SP2-AS1, TCF21, TBPL1, RGS1, RGS13, RNU6-168P, LINC02227, ENPP2, MIR4460, SVEP1, PDE11A, LINC02248, GABRB3, JAKMIP3, BNIP3, SLC7A1, UBL3, SMC1B, FARS2, TBC1D5, IL17A, LINCMD1, COL11A1, DLEU7, RPS23P4, OTUD4, LRRK1, LINC02875, TBX4, GABBR1, DLG2, WNT4, MLIP, SSRIP2, CTAGE11P, FOXP2, MEIS1, CHD7, NEK7, HEBP1, CABLES1, TMEM241, GRHL1, KLF11, WWP2, CCDC82, PAX9, DIS3L2, IL33, TPTIP13, TACC3, RPL31P35, RNU6-1125P, CCDC91, RNU6-510P, SF3A3, NPR3, SPI1, MIR4487, RALGAPA2, INSMI, ABR, PRKAR2B, FAM110D, PDIKIL, CDC42EP3, PPP1R14BP5, RBL2, CCNF, LINC02508, RNU7-192P, LMCD1-AS1, GSS, ETV1, EIF4A1P1, RPSAP64, LINC01700, BCKDHB, RNY4, RN7SL569P, LINC02219, EEF1B2P2, ZNF786, EXT2, SFXN1, DRD1, PDZD2, ANAPC10, LINC02201, CCDC3, CLCN5, GPR78, CPZ, FBXW11, LINC02652, IFITM3P3, SPDEF, PPP6R3, GAL, RNU6-560P, LINC02695, LINC02620, BBS1, ZDHHC24, LINC02322, C14orf39, LINC02654, PTER, SLC4A10, GSPT1, LINC02096, CDRT7, ALDH1A2, SNX14, SYNCRIP, IGFN1, PHF20, WDR70, AKT3, ZRANB2-AS2, RGS12, N4BP2, KRT18P25, MIR5197, CXXC5, RN7SL366P, QKI, ADAM22, RNU6-1086P, MIR3622B, DOK2, RN7SL720P, RORB, NOC3L, PHF21A, SPON1, GPR176, CCDC9, RNU6-192P, VRK2, GPRIN3, ELP1, CRIM1, PRKG2, PLEKHN1, SLC25A5-AS1, RNY3P16, LINC02599, RPL29P19, ADCY9, SEPTIN8, ADCY3, KIZ, FGF18, MCC, STAβ1, CDK6, ANTXR1, EEDP1, RN7SL727P, LINC02024, IGSF11, RNU5A-2P, PTPN14, LSM3, LINC01267, NPM1P28, ZIC1, NPR2, SMIM23, MAFF, PLA2G6, UBE2C, LINC02725, DNAJB6P1, FOXO3, LINC00222, CHFR, RFLNA, PTPN9, MIR4522, RPS16P8, SCIN, DNMT3A, WWTR1, WWTR1-AS1, DLK1, HLA-DPB2, RNU6-271P, ZNF518A, DLC1, CSGALNACT1, KIFIA, AGXT, MIR4508, MKRN3, TMEM270, ELN, HDAC9, EIF4B, TNS2, ATXN7L3, CYP26B1, NOX4, PLEKHA1, MYO9B, SORBS2, PRDM12, FUBP3, LINC00624, DDX27, VGLL4, CYP19A1, MIR4713HG, ZNF346, FGFR4, MIR193BHG, GLDN, TNPO1, SEPTIN14P17, WNT3A, RFT1, SERBP1P3, LAMA2, LINC02682, E4F1, PGP, ABCF2-H2BE1, H2BE1, ZNF638, PAX1, RPL35P2, NUDT3, COL6A2, RPL31P14, IGFBP5, TENT5A, RPSAP72, DAAM2, MYEOV, LINC02747, ZKSCAN1, FRYL, SP7, HAGHL, ICAM1, IHH, CNN2P11, IPPK, BICD2, CSH2, GH2, DUSP16, CREBL2, CFAP65, CHAD, ACSF2, PDE8A, H3P24, TMED7, GLDC, CSH1, CSHL1, RNU6-508P, SHOX2, NSFP1, RDM1P2, ELMO1, ETV6, CREB3L2, DAPK1, LINC02872, PTEN, MED6P1, ZZZ3, GAB1, SMARCA5-AS1, INPP5B, KCNS3, SPRY4, NDFIP1, GHR, CCDCl52, SLIT3, RPS4XP6, CCDCl7, SFRP4, STARD3NL, STMP1, SPATA18, DUT, FBN1, OR5BDIP, CYCSP26, SPTBN1, BCL6, HOMEZ, UBE2Z, RPS26P45, LINC02373, ZNF143, LEMD3, MSRB3, ASTN2, SOCS2, TMEM263, RPL30P12, MIR4421, CDKN2C, ARHGEF12, EEF1AKMT2, ABRAXAS2, LAMC1, CDC42-AS1, LINC00339, CDC42, DUSP29, CAPRIN2, MOV10, BOC, NFE2L3, MIR148A, MIB2, ANK3, ATP7A, PGK1, PHKBP2, RPL29P3, PCSK6, FHOD3, MYOIF, SLC38A9, AK4P2, KCNJ12, RGP1, MSMP, CCN6, LINCO2527, GLI2, MEGF9, RRBP1, JPH3, ZCCHC14, EXOC6B, LINC01571, LINC01997, LINC00211, JAK2, LOXL2, ZBTB20, ZBTB20-AS5, RFC1, TH, MYMK, RPSAP49, AXIN1, ACTGIP13, PRRX1, MEF2C-AS1, REST, PLCG1, ZHX3, GLCCI1, ADAMTS5, ZNF318, CRIP3, CRELD1, IL17RC, HSD17B6, PRIMI, MED13L, MIR7-3HG, ANAPC16, SH3RF3, SLC25A37, ENTPD4, FBRSL1, ADAMTSL3, SIX4, DDAH1, RPSAP52, PCNPP3, ST3GAL4, SCN4A, DPEP1, GPAM, LINC02160, IL16, TRIP11, MORC3, PARPBP, CLPB, SYN3, NOG, C17orf67, PWWP3B, CSGALNACT2P2, LINC01423, KCNJ15, CDCl4B, GNAS-AS1, NCOR2, FOXM1, RESP18, DNPEP, COMMD1, KCNQ1, ARHGAP26, ZNF70, SLC27A3, GATAD2B, ZNF793, PLOD2, ZNF280C, RAB33A, SPAG6, CMIP, FIBIN, ACAP2, PPPIR2, SRRM2, ATP13A2, E2F1, XRCC1, C9, DAB2, KRT18P51, SPRY4-AS1, LINC02571, HLA-B, BAKIP1, NTAN1, PDXDC1, PSMG1, FKBP6, TMC3-AS1, MEX3B, PCSK5, SALRNA1, PKN2, RNA5SP267, SLC2A13P1, RNU4-17P, LINC01713, MIR4309, ANKRD9, PODN, DNAJC27, USP34, DPY19LIP1, ZNRF2P1, BABAM2, ISL1, RNU6-1296P, INSR, IER2, PIP4K2B, SPTY2D1, TRPS1, FBN2, ZNF619P1, TTC4P1, TC2N, SLC44A2, LHPP, SEPTIN9, ZMIZ1, AXDND1, ADAMTS6, ZNF652-AS1, TLE3, LINC00485, FILNC1, HAUS3, POLN, FERD3L, POLRIF, OR5D16, OR5L2, GATAD2A, RNU6-500P, BOD1, PSMC3, DPY19L3, PCDHB16, IGFBP3, DAZL, LTBP1, BCKDHA, PKIA-AS1, HIGDIAP18, RORA, SMAD3, AXIN2, BPTF, MYL6P5, GRB7, MIEN1, NFIC, CXCR3, LINC00891, CCN3, PHC1, LOXL1, FNDC3B, MAX, RAD51B, FBLN5, ARID5B, R3HDM1, BNC2, ACBD4, LINC02728, GAβ2, GACAT3, RN7SL104P, NCL, PPMIB, SDR16C5, CHCHD7, GHSR, LINC01990, BBX, TNNT1, FLI1, CATSPER1, CST6, STC2, NSD1, L3MBTL3, PVT1, ZFAT, ECPAS, ZNF483, MSI2, FAAH2, LINC00629, MIR424, PIP4K2A, ZNF438, PPIF, ZCCHC24, CCND2-AS1, U2, DELEC1, QSOX2, IL17RD, SERPINH1, RNA5SP292, B4GALNT3, THOC1, TET2, LINC02181, C16orf95, GCH1, SAMD4A, CTDSP2, ATP23, CATSPER3, PITX1, RPSAP20, CACNAIE, TGFB2, ARHGEF11, CDC42SE1, MLLT11, DDR2, ALDH2, RN7SKP208, MIR217HG, PLEC, SMPD3, SPSB1, LINC02606, ZNF76, MRPL45P2, NPEPPS, EIF2AK3, ITSN1, LINC02044, GMNC, OSTN, LIN28B, LIN28B-AS1, FILIP1, CSF1, AHCYL1, VSIG10L2, RN7SKP15, PTHLH, LPARI, SLC38A4, SLC41A2, CHST11, TMEM256P1, NEUROG3, NOCT, CCDCl62P, GCKR, MARK3, CKB, TET1, LINC01091, PPIAP76, RCN3, LINC02869, SLC12A2, ZNF713, MIR375, CRYBA2, PIK3R1, VIL1, TACR3, FOXD2-AS1, FOXD2, SDR16C6P, EFEMP1, SUPT3H, POLR3E, TSEN15, COLGALT2, HMGN2P15, EZHI, EXOC7P1, RPL34-DT, MIR34AHG, H6PD, SERPINE2, STAT1, LINC01169, SMAD6, PTP4A3, GPR20, TRA2B, SCUBE3, MECOM, CCDC80, ATP4B, TFDP1, NHEJ1, LINC01556, KRT18P1, PRKG1, QPCT, RNU6-1116P, ECM1P2, U6, WNT6, TBX15, ZBTB7C, ZBTB4, CCDC25, ADAMTS3, HNRNPAIP67, FAM205BP, FAM205A, PRKAR2A, ANKFN1, MTCO3P1, HLA-DQB1, ELF1, RPS10-NUDT3, RNA5SP124, PATZ1, FAM118A, UPK3A, TAF5, PAPPA2, DNAH2, MIRLET7BHG, BOK, FALEC, RPL23AP75, IGFBP4, MRPS18CP4, GRIN2B, GNAS, PASK, LINC02680, MKX, SSTR5-AS1, HGFAC, DOK7, COL8A1, ZNF763, GHRH, RPN2, MRPS11P1, NCLN, DNM3, CIAO3, CCDCl2, ZRANB1, NPMIP31, LOXL4, STAT6, IGFIR, HELLPAR, IGF1, LINCO2456, PLAGI, TOP1MT, TAFA4, RNU6-61P, SPRY2, AMZ1, GARSI, SLC25A21, RPGRIP1, HNRNPC, ARID3B, LINC01505, WDR72, NFATC1, ZNF521, SOBP, FEZ2, LTBP2, CAMKID, CROCC2, SNED1, CEP120, SLC22A3, MOXD1, RREB1, BMP6, SNRNP48, H2BC8, H4C5, LINC00547, LINC02464, FAM169B, IRAIN, RYK, ANAPC13, HMGB3P13, RYBP, LINC00877, DIRC3, GDPD5, CNPY2-AS1, ITPK1, PEX11A, PIEZO1, GMDS, SMOX, RPL21P2, LINC00310, KCNE2, LINC01258, CLCP2, CHAMP1, NPM1P47, C2CD4B, COG6, PPIAP23, MIR17HG, PGPEPIL, C12orf57, PTPN6, COPG1, N4BP2L2, GALR1, VGLL2, VDAC2P3, PNRC2P1, LOXL3, DOK1, FADS2, EPB41L2, MIR497HG, UNCX, ZFAND2A-DT, LINC00536, EIF3H, RPL39P33, ESCO2, CHD8, PTGR1, PDCL2, RNU6-148P, BRWD1P2, SEMA4C, ANKRD39, LMO1, PDE3A, ATP5MC2, ATF7, COPB1, ZNF493, CXCR2P1, RUFY4, HLA-DRA, HLA-DRB9, SLC23A2, BRDT, ICA1, SLIRPP1, PGR-AS1, LINC00364, CCT8LIP, IHO1, BBS9, IL20RB, SPC25, NOSTRIN, EFR3B, MMP24OS, ABHD15, ABHD15-AS1, LINC02507, TMEM184C, TMTC2, PCCB, PLCG2, STAU1, CSEIL, TCERGIL, UNC5D, COL25A1, CFAP43, RAB3IP, CDHR2, FIG. 4, SH3GL3, MYCBPAP, RSAD1, PML, SFMBT1, NCOA6, SEMA3D, TEAD1, HLA-A, HLA-U, TULP1, FKBP5, AKR1C2, TULP4, SOX5, LRRC43, THRB, WNT5A, SLC35G2, STAG1-DT, ZNF844, MYO3B-AS1, MYO3B, LINC02739, OR4D7P, CACNAID, ADCY6, TRIM38, PNPT1, LNX1, COA6-AS1, SLC35F3, LINC02570, IGHV4-39, IGHVIII-38-1, SLC25A13, RNU2-36P, FRS2, TOMM22P5, PRLHR, SGCZ, CDK5RAP2, KRT18P35, RASA2, MKRN6P, FANCE, H2BC6, LINC02588, WDR11-AS1, LINC01581, LINC01579, PIPOX, MYO18A, TEX49, OR2B2, OR2W6P, HEATR5B, H1-4, H2AC6, COPZ2, CDK5RAP3, SLC39A4, TNS1, WNK3, SUZ12, LRRC37B, BORCS8-MEF2B, BORCS8, MEF2B, ATOH7, MYPN, NOP9, CIDEB, LTB4R2, CPAMD8, TNNT2, TCP11, ANKSIA, CRB1, CBFA2T2, BACH2, HOXA3, BRINP1, CDH13, GPC5, CDCA7L, DGKH, SIN3A, KRT18P9, GNA12, CEP250, DCAF16, FAM184B, ZNF678, PRELIDIP1, RPS4XP9, SCMH1, CEP63, PRR13P4, ILRUN, ADGRD1, MAF, ZNF510, NUCKS1, TRPM3, TMED2, DDX55, C12orf60, SMCO3, ZNF192P1, YAP1, ST6GALNAC3, ASB17, ZNF879, GRM6, DAB1, FUT2, MAMSTR, LINC00471, RSRC1, PPP2R2A, H2BC7, RNU6-200P, LINC02120, PROCR, MFAP2, ATAD5, DOTIL, ZNF367, MIR3936HG, EPB41L1, NFATC4, UQCRHP1, NCR3, DLEU2, RPL12P19, PXMP4, CENPO, PTPRJ, PDS5B, TMEM176A, AOC1, DCBLD1, NXNP1, LINC02283, HOMER1, MIR9-3HG, POLG-DT, RNU6-669P, PCAT7, ANKRD60, KRT23, KRT20, C9orf152, PALM2AKAP2, NUP37, EXT1, FREM1, CATSPER4, MICA, HLA-S, TMED10, H2AC9P, DCAF12, RN7SKP114, NKX2-1-AS1, PAPPA, RAB40C, DYNC211, SPATA31C2, CORO1CP1, ADAP2, BCR, ADAMTS10, MAP3K3, COL27A1, CASC20, PPARD, TNXB, NPPC, PITX1-AS1, GOLIM4, ZDHHC7, LINC02176, PPIL6, MRTFA, TNRC6B, CYP20A1, RN7SL88P, YPEL5P3, PBX1, PBX1-AS1, FSTL5, FASTKD2, MAGI2, PIGF, PHACTR2, PAK5, RSU1, OR10D4P, OR10D5P, CEP152, RPS4XP19, BICC1, TSEN34, TMC2, UMADI, TACC2, GSGIL, TMED9, CPEB4, DSCAM, DSCAM-IT1, SORBS1, ZNF804A, SNX29, ZNF536, RNA5SP431, NAV2, TGFA, AADACL3, AADACL4, PRKCA, TMEM100, ARSL, STRADA, CYP2E1, CS, TMEM132C, MAML2, OR2I1P, TFAM, SPATA33, ATF7-NPFF, DCLK1, TTLL5, RNU7-133P, SMIM29, LYRM4, SULF1, PEAK3, RNA5SP181, DIRC3-AS1, RN7SKP43, CCDC88A, CARMAL, RN7SKP93, SEPTIN2, CD79B, PKN2-AS1, USP21P1, VTA1, POU5F1, ITPR3, ZNF341, FBLN2, HDAC11, ILRUN-AS1, SNRPC, DRAM1, KIF27, C9orf64, C9orf92, CAMKMT, ZNF142, LINC02834, RHOD, SBNO1, RPL32P3, H1-10-AS1, MSLN, ID4, OR2J3, OR2J2, H3P27, RPL26P20, FER, MIRLET7A1HG, PTPDC1, OR4A7P, OR4A5, CRIPT, LINC01118, CTU2, MY09A, ZNRD2-AS1, STAT2, DOCK3, GFPT2, GIT1, ANKRD13B, DTL, MICAL1, ZBTB24, DDX6, RNU6-376P, LIN28A, ZNF652, FLJ40194, NME2, NME1-NME2, PRKCZ, NUCB2, HTRID, SLBP, KCNJ16, FOX06, MAP3K1, SRRM1, CLIC4, CPN1, ZC3H11B, DIPKIA, WWC2, ZFAS1, DMAC2, B3GNT8, SLCO1C1, MLST8, CASKINI, SUCLG2, ADAM28, DRAIC, RNFT1P2, GIPC2, SNAP47, ZNF847P, LINC01184, ADAMTS2, ZNF354C, H2BC5, PSORSIC2, PSORSIC1, AIF1, PRRC2A, CNRIP1, PPP3R1, ECE1, EIF4E2, CAPZA1, PHGDH, CYTL1, STK32B, HABP4, AKRIC6P, U8, NUP160, RPS2P32, ZNF804B, HMGN1P19, APTR, LUZP1, ITIH3, OTUD7B, STAG1, RNA5SP77, CLOCK, BCL7A, LINC00377, ARF4P4, FANCI, POLG, PER1, HES7, SUMO2P17, NACA2, TCAMIP, ILF3, KIZ-AS1, SLC23A3, EXTL3, NCAPG, FADS1, CDK10, TMEM88, DNM2, RBL1, CTBP2, RN7SKP46, RBBP8, GDF5-AS1, CNOT9, PLCD4, TTLL4, SLC17A2, UBD, MUC22, HCG22, POLR2B, LINC01365, PDE5A, ANXA5, EXOSC9, pp 12613, NUDT6, SPATA5, JADRR, SERF1AP1, STMN1P2, LINC00616, RN7SL311P, GUSBP5, GYPA, C4orf51, ZNF827, ATP5MGP4, FAM160A1, RPS3AP18, TMEM154, TMEM131L, FGA, FGG, PDGFC, WDR1, FNIP2, PPID, RAPGEF2, AGRN, VWA1, CDK11B, SKI, ACTRT2, TASIR1, ERRFI1, LINC01714, RERE, SLC25A33, KIFIB, MTOR, PDPN, HSPB7, CROCCP3, CROCC, ARHGEF10L, RCC2, CAPZB, MICOS10-NBL1, NBL1, MUL1, RPS4XP4, EIF4G3, LINC02596, RAPIGAP, RUNX3, LINC02793, IFITM3P7, MACO1, UBXN11, DPPA2P2, HMGN2, ARIDIA, GPN2, TRNP1, YTHDF2, MATN1-AS1, COL16A1, SPOCD1, PTP4A2, KHDRBS1, TMEM39B, PHC2, SH3D21, MEAF6, FHL3, RLF, COL9A2, KCNQ4, CTPS1, HIVEP3, H4C8, H3C9P, ECEL1P3, HMGB3P2, DHFRP2, USP8P1, RPL3P2, FAM83C, PXYLP1, KDM2A, TMCC1, BZWIP1, SSC5D, SAMHD1, SUFU, SHROOM4, MIR548AZ, SIAH1, CHSY1, XYLT1, LINC01097, NKX3-2, TEFM, HEMK1, FAM155A, LINC02361, NOC4L, KRT42P, ENPP1, NPM1, DAPK2, MIR422A, BCL2L2-PABPN1, BCL2L2, LINC01572, SENP6, MFSD2B, PREX1, AEBP2, RHOBTB2, PEBP4, TNFSF10, LINC02068, RNU6-133P, AHCY, ITCH, CELF6, POMC, LINC01381, TNFSF18, DCUNID4, PCGF5, TEDC2, CRISPLD2, MOAP1, CYB5AP3, HCP5, PCBD2, CFB, PPPICA, CARNS1, TRMT61A, MAP6, NOA1, CCND2, HEXIM2, FMNL1-DT, ALPP, ARFGEF2, PNKD, CARD11, OR5V1, MCMBP, USP37, DNM3OS, FERIL4, ERGIC3, HCG18, HCG17, HLA-L, HSPG2, NUF2, RGS5, BAG6, RAPSN, TBX21, OSBPL7, PDE10A, DDX51, ACOT11, SEC16A, RPL23AP44, FAR2, PPL, FSDIL, GRK5, KCNQ3, LINC00243, BANF1P5, MC4R, TFEC, RGS3, ZNF322, GUSBP2, MAP2, LINC02506, TRAF7, TSBP1-AS1, TSBP1, PPPIR16A, GTF2H1, KRT19P4, PBLD, NFKBIL1, SOCS5, DNAJC27-AS1, FAM180B, NDUFS3, SUZ12P1, CRLF3, RILPL2, PDLIM4, P4HA2, TOP2A, RNA5SP441, ZSCAN2, LHX3, TMEM250, TMEM196, SERPINA1, CENPP, FOXP1, SLC35D2, ERVW-1, PEX1, COL15A1, RBM6, SENP2, LIPH, LINC02618, CHKA, KMT5B, SLC16A11, CLEC10A, RUNX1, RBMS3, RBMS3-AS2, DOC2A, IKZF1, NFATC2, SLC6A4, KRTAP7-1, KRTAP11-1, ZNF341-AS1, CREB5, NMBR, PKD1, COX5BP8, ABL2, CACUL1, MAPIS, TMPRSS5, NKAIN3, PAFAHIB2, SIK3, SYTL2, UTS2, SH3YL1, MLPH, COL6A3, GASKIA, ACHE, CHCHD3, RBPMS, RORA-AS1, TSPAN3, ZFHX3, PEMT, DCST1, VPS45, AIP, MAP2K2, ERC1, KDM6B, DNAJB4, DNASE2B, GNG5, COL24A1, LINC02795, ODF2L, GBP1, GBP3, LRRC8C, TGFBR3, EVI5, RPL5, CDCl4A, OLFM3, CELSR2, LINC01768, NDUFA5P10, PTPRF, ZSWIM5, MAST2, RPL21P24, FAF1, RAB3B, NRDC, ZFYVE9, SHISAL2A, SLC1A7, GLIS1, LINC02784, SSBP3, DHCR24, LINC01755, PLPP3, NFIA, PATJ, LINC00466, JAK1, PDE4B, DNAI4, GNG12-AS1, ZRANB2, ST7L, WARS2, WARS2-IT1, PDE4DIP, CHDIL, BCL9, RN7SL480P, PLEKHO1, RPRD2, ARNT, PRUNE1, ZNF687-AS1, PSMD4, GEMIN2P1, RN7SL372P, ADAR, HMGN2P18, KRTCAP2, YY1AP1, LMNA, MEF2D, PIGC, SUCO, LHX4, KIAA1614, SMG7, RGL1, Clorf21, EDEM3, HMCN1, RNU6-778P, NR5A2, IPO9-AS1, NAV1, MYOG, ZC3H11A, ZBED6, PLEKHA6, LRRN2, RNA5SP74, RAB29, PFKFB2, YOD1, MIR29B2CHG, UTP25, HHAT, ST13P19, PPP2R5A, LINC02608, HIVEP2, LINC01645, NPL, LINC01688, CMTM8, ARHGAP24, DAP, SALL4P1, ACTBL2, LINC02667, LINC02426, SOCS2-AS1, Metazoa_SRP, SIAH3, WDCP, NCOA1, DTNB, KIF3C, FOSL2, LBH, MEMO1, DPY30, BIRC6, ATP6VOE1P3, HNRNPAIP61, VIT, RMDN2, CYP1B1-AS1, CYP1B1, DHX57, MAP4K3, C2orf91, PKDCC, COX7A2L, CHORDCIP1, LINC02580, ZFP36L2, THADA, ABCG5, PDSSIP2, RNU6-566P, LINC01121, SRBD1, PRKCE, EPAS1, TTC7A, STPG4, CALM2, EPCAM-DT, FOXN2, ACYP2, PPP4R3B, BCL11A, LINC00309, MIR4433B, SPRED2, RN7SL635P, RPS15AP15, KRT18P33, LINC01812, WDR92, AAK1, PCBP1-AS1, LINC01816, TIA1, C2orf42, PAIP2B, DYSF, ITGA6, SPR, DGUOK-AS1, MIAP, VAMP5, RNF103-CHMP3, IGKVID-16, IGKVID-17, AFF3, MAP4K4, SLC9A4, AHCYP3, LINC00486, LINC01102, MRPS9-AS2, LINC01159, FHL2, ECRG4, RPS21P2, SRSF3P4, GCC2, SMIM12P1, ACOXL, MIR4435-2HG, BCL2L11, SLC35F5, RNU6-744P, PTPN4, RPL27P7, LINC01101, SDCCAG8, ZBTB18, TGIF2P1, ADSS2, PXDN, MYTIL, SOX11, LINC01248, LINC01247, LINC01824, IAH1, YWHAQ, RNU4-73P, CYS1, RRM2, RN7SL66P, ROCK2, MIR3681HG, TRIB2, OSR1, LINC01808, LINC00954, CISDIP1, MATN3, RPS16P2, RN7SL140P, RHOB, RNA5SP86, KLHL29, LLPH-DT, SETD2, ASB3, RTN4, LINC01122, LINC02831, RNA5SP99, RNF103, LINC01965, PANTR1, LRPIB, ARHGAP15, TEX41, CREB1, RBMS1, SCN2A, METAPID, CIR1, PLCL1, FTCDNL1, RN7SL717P, PARD3B, LANCL1-AS1, MEGF11, TIPIN, MAP2K5, ADPGK, LOXL1-AS1, LINGO1, RASGRF1, ANKRD34C-AS1, ABHD17C, RTN4RL1, RPA1, NUP88, NCOR1, TTC19, MAP2K3, ASIC2, GGNBP2, PRR15L, IGF2BP1, KIF2B, WBP2, RPTOR, BAIAP2, AIDAP3, PTPRM, RMC1, NPC1, LINC01915, ASXL3, PIK3C3, FGF19, DNAJB6P5, NRK, COL11A2, ADAMTS12, MIER3, LEPR, NEGR1, RPL31P12, FPGT-TNNI3K, TNNI3K, USP33, HMGB1P18, ADGRL2, TTLL7, LINC02609, BARHL2, EEF1A1P11, DPYD, PRMT6, GPR61, KCND3, NRAS, ZBTB7B, MEX3A, IQGAP3, PRRC2C, CYCSP53, PRDX6-AS1, MRPS17P3, CADM2, C3orf38, ABCF2P1, PROS2P, RNU6-712P, NSUN3, ARMC10P1, RAP1BP2, ALCAM, IFT57, SNX4, CPNE4, EPHB1, LINC02032, LINC02006, CFAP20DC, TIPARP-AS1, KRT18P34, SLC2A2, NLGN1, SOX2-OT, PLEKHA3, FKBP7, PDE1A, FRZB, MIR548AE1, FAM171B, COL5A2, GLS, SF3B1, LINC01923, RNU7-147P, TMEM18-DT, SATB2, SATB2-AS1, SPATS2L, STRADB, FZD7, KIAA2012, BMPR2, PLEKHM3, RPE, CPS1, ERBB4, FN1, LINC00607, RIT2, SYT4, SETBP1, RAB27B, TCF4, LINC01415, GRP, SEC11C, BCL2, CDH7, LINC01916, LINC01541, KLF16, SF3A2, ZBTB7A, SMARCA4, ZNF490, MAST3, PGPEP1, CRTC1, SUGP1, RNA5SP472, ZNF507, PEPD, APOE, MARK4, RPL7P4, NRM, MSL2, ITGB8, TBXAS1, RABGAPIL, ENTRIP2, MRPS14, SEC16B, LINC01741, BRINP3, IPO9, OPTC, ATP2B4, LINC01774, SYT14, SERTAD4-AS1, LYPLAL1-AS1, DNAH14, GALNT2, LINC01874, LINC01865, TMEM18, LINC01875, KCNK3, SMIM7P1, SLC8A1-AS1, LDHAP3, HNRNPA1P57, STON1-GTF2A1L, GTF2A1L, LHCGR, NRXN1, MAP3K2, UGGT1, HS6ST1, MGAT5, ZRANB3, LINC01412, MBD5, RPS29P8, IL36B, LINC01876, GPD2, CDK7P1, GALNT5, TANC1, FIGN, PRPS1P1, STK39, RN7SL813P, TLK1, METTL8, DAP3P2, RPS15P4, DYNC1I2, RPL21P38, SP3, HOXD13, HOXD12, PDE11A-AS1, GIPR, ZC3H4, PPFIA3, SYT3, LINC01872, STK35, KIF16B, RN7SKP140, NKX2-4, ENTPD6, MIR663AHG, LINC01597, HCK, PIGPP3, MAFB, LINC01370, PPIAP21, RNU6-743P, CDH22, LINC01524, LINC01440, RNA5SP487, TCEA2, BRWD1, FLNB, NMUR1, PDE6D, DIS3L2P1, ECEL1P1, EFHD1, GIGYF2, AGAP1, PER2, LINC01937, TWIST2, U3, GPC1, SNED1-AS1, HDLBP, ITPR1, LMCD1, RAD18, THUMPD3, THUMPD3-AS1, SLCO3A1, NTHL1, RBFOX1, LINC02177, RNA5SP404, GPRC5B, GPR139, SNRPEP3, PDILT, RBBP6, TNRC6A, SH2B1, TAOK2, STX4, STX1B, FTO, NFAT5, AARS1, PMFBP1, RFWD3, LINC02125, MIR4719, FGF12, LINC01878, SPHKAP, FBXO36, SETD5, PPARG, SGO1-AS1, RARB, KRT8P18, CCK, SALL4P6, SEC22C, VIPR1, MIR138-1, LARS2, IP6K2, ACTL11P, MSTIR, IQCF6, IQCF4P, STIMATE, STIMATE-MUSTN1, ERC2, FHIT, PTPRG, CADPS, SAMMSON, COX6CP6, BACE2, ADARB1, BID, DGCR8, ACO2, PLXNB2, MAPK11, SLC35E2B, TTC34, THAP3, PHF13, CAMTA1, RNU1-1, LINC01783, LACTBL1, TEX46, ADGRB2, KIAA1522, CSMD2, MACF1, NFYC-AS1, RIMS3, IPP, PDZKIIP1, TAL1, AGBL4, TMBIM1, RETREG2, ZFAND2B, OBSL1, ACSL3, KCNE4, FAM124B, CUL3, DOCK10, CCDCl95, NYAP2, RHBDD1, AGFG1, SCYGR9, SPATA3, RNU1-58P, RNU6-797P, BANK1, POGLUT1, FSTL1, ITGB5, DNAJB8, EEFSEC, PLXND1, OR7E129P, TRH, NPHP3-ACAD11, ACAD11, LINC02004, AMOTL2, KY, MRAS, HMGN2P25, GRK7, XRN1, PCOLCE2, RPL21P71, LINC02315, LRFN5, YWHAQP1, MDGA2, SYT16, GALNT16, DPF3, NRXN3, GALC, RPS6KA5, UNC79, BCL11B, LINC00523, MIR412, TRAF3, SEMA6D, TMOD2, RPSAP55, ONECUT1, LINC02490, MYO1E, PCSK1, MIR583HG, FBXL17, TUBAP15, RNU6-718P, LINC02240, JADE2, RNU6-236P, LINC01470, GRIA1, MFAP3, WWC1, RANBP17, HIVEP1, PHACTR1, MIR548A1HG, RNF144B, E2F3, ZBED9, CLIC1, LYPLA2P1, MYL12BP3, PACSIN1, MSRA, GATA4, LINC00208, TUSC3, HAS2-AS1, PTK2B, ELP3, PURG, C8orf86, FGFR1, VNIR46P, HGSNAT, PRKDC, CLVS1, LINC01414, LINC00967, RNA5SP271, KCNB2, HNF4G, RNU2-54P, LINC01109, RALYL, CNGB3, CNBD1, VIRMA, LINC02894, MROH5, MIR1302-7, TSNARE1, KDM4C, PTPRD, RNU2-47P, JKAMPP1, NFIB, CCDCl71, LINGO2, MIR873, ME2P1, UBAP2, PAX5, PHF2, ERCC6L2, NAMA, KRT8P11, TEX10, EPB41L4B, RPL35AP22, TTLL11, CRB2, LMX1B, GOLGA2, BDNF, BDNF-AS, LINC02755, ARL14EP, MIR129-2, HSD17B12, C11orf96, ALKBH3-AS1, LINC02696, SYT13, OR5W2, OR511, BAD, GPR137, EFEMP2, CCND1, GUCY2EP, TSKU, NAALAD2, SMCO4, DYNC2H1, TTC12, CADM1, VPS11, MIR100HG, RN7SL167P, SNX19, NTM, OPCML, FIBCD1, ELOCP3, MLLT10, LINC00838, LINC02628, JMJDIC, ADK, GRID1, CRTAC1, PAX2, GBF1, TCF7L2, ENO4, LINC02641, CHID1, STK33, ARNTL, PPIAP16, FLNB-AS1, ABHD6, RNU6-281P, PPP4R2, POUIF1, KRT8P25, HTRIF, DCBLD2, ST3GAL6, TRMT10C, FAM172BP, NEPRO, ATP2B2, RAF1, LINC00620, WNT7A, XPC, FGD5, RPL24P7, UBE2E2-AS1, LINC00691, NR1D2, RNA5SP126, NEK10, GADL1, SUSD5, FBXL2, CLASP2, LRRFIP2, ITGA9, ZMYM2, GPR12, MTIF3, RNU6-63P, HMGB1, UBE2L5, LINC00598, LINC00558, ZNF646P1, LINC00458, DNAJA1P1, CTAGE16P, LINC01075, PCDH9-AS2, PCDH9, CCT5P2, NIPA2P5, MOB1AP1, HS6ST3, STK24-AS1, NUS1P4, TEX29, LINC02337, RNU11-5P, LINC02326, PRKD1, AKAP6, PRORP, MOCS2-DT, FST, IL6ST, C5orf67, RPL26P19, PDE4D, TBCA, AP3B1, SCAMP1, ARSB, DMGDH, TENT2, KRT18P45, SERINC5, DBIP2, LINC01337, SSBP2, MIR4280HG, LINC02144, LINC02161, ADGRV1, LUCATI, MTCO1P24, CTBP2P4, HNRNPKP1, PRRC1, ADAMTS19, RNU7-53P, LINC01950, RAPGEF6, FNIP1, PPP2CA, RNU6-456P, LINC01843, DDX46, MTND4P12, C5orf66, LECT2, GFRA3, CDC23, UBE2D2, PSD2-AS1, HAUS1P1, CD14, ARAP3, FGF1, NR3C1, HMHB1, GRPEL2, GRPEL2-AS1, PDE6A, CSFIR, ARSI, SPARC, CYFIP2, EBF1, LINC01845, FABP6, PWWP2A, TENM2, STK10, DUSP1, ERGICI, CREBRF, FAF2, CLTB, UNC5A, RGS14, LMAN2, PDLIM7, B4GALT7, FAM153A, FAM153CP, N4BP3, RNF130, RASGEF1C, MAPK9, MGAT1, TRIM7, TRIM41, EXOC2, FOXC1, GMDS-DT, BTF3P7, RN7SL554P, TXNDC5, BLOC1S5-TXNDC5, RN7SL332P, ANKH, SEPHS2P1, HNRNPKP5, CDH6, DROSHA, GOLPH3, SPEF2, SKP2, SLC1A3, NIPBL-DT, NUP155, CPLANE1, RPSAP38, FBX04, SELENOP, ZNF131, ANXA2R, ZDHHC11B, RNU6-480P, JARID2, DTNBP1, MBOAT1, CASC15, TDP2, KIAA0319, CARMIL1, CMAHP, LEMD2, MIR1275, MIR7159, GRM4, TAF11, UHRF1BP1, PAM, STARD4-AS1, APC, DCP2, REEP5, PRR16, SRFBP1, FTMT, ZNF608, LMNB1-DT, LINC02057, CWC27, LINC02242, TMEM171, LINC02230, FOXD1, HEXB, AFAP1, SLC2A9, CLNK, ZNF518B, LINC02498, HSP90AB2P, FBXL5, MED28, KCNIP4, ANAPC4, LINC02357, SMIM20, KLF3-AS1, TLR6, FAM114A1, KLHL5, SMIM14, RHOH, BEND4, GABRG1, SLC39A8, LINC02428, RNU6-351P, TET2-AS1, PPA2, RN7SL89P, LEF1-AS1, RPSAP34, STPG2, RRH, CAMK2D, ARSJ, NDST3, SYNPO2, MYOZ2, KLHL2P1, CPE, PDCD6-AHRR, PDCD6, LINC02102, DAP-DT, HNRNPA1P20, MBNL1, LINC00881, CCNL1, PHC3, LINC00578, ZMAT3, PARL, PSMD2, NMRAL2P, LINC01991, LPP-AS2, LPP, P3H2, CCDC50, CPN2, XXYLT1, FGFRL1, NKX1-1, FGFR3, NSD2, TBC1D14, ZAR1, FIPIL1, GSX2, RPL22P13, CRACD, CEP135, SOWAHB, CFAP299, BMP3, VAMP9P, PTPN13, HSD17B13, DMP1, HSP90AB3P, MEPE, RNU6-907P, TIGD2, LINC02267, C4orf17, MTTP, C4orf54, RNU6-356P, SFRP1, KAT6A, ANK1, SMIM19, LINC00293, MAPK6P4, LINC02847, EFCAB1, ST18, BHLHE22, XKR4, SEPTIN10P1, PENK, RPL37P6, RNU6-13P, LINC01606, TOX, DNAJC5B, MYBL1, PREX2, NDUFS5P6, EYA1, JPH1, CRISPLD1, CASC9, MROHI, DGAT1, KANK1, AK3, RPS3AP54, RNF2P1, MPDZ, PRDXIP1, PES1P2, RPL3P11, ADAMTSL1, SAXO1, IFNW1, CDKN2B-AS1, MOB3B, PTENP1, TRBV25OR9-2, IL11RA, SPAAR, ANXA1, OTX2P1, RPS19P6, TLE1, MTCO3P40, FRMD3, HNRNPK, TUT7, CDK20, RNU6-86P, SPIN1, GADD45G, UNQ6494, ROR2, NFIL3, BEND3P2, PRSS47, SUSD3, CARD19, XPO7, HR, EGR3, STC1, NKX2-6, RNU1-148P, ADAMDEC1, KCTD9, BNIP3L, DPYSL2, ADRA1A, SCARA5, PHF20L1, NRG1, RN7SL621P, RNF122, LINC01605, RNF5P1, TACC1, RPL3P10, ADAM32, IDO2, LINC02866, TCIM, SIRLNT, WNK2, RNU6-829P, MIR4291, BARX1, MFSD14B, PTMAP12, FBP2, AOPEP, FANCC, SRSF1, DYNLL2-DT, TEX14, BCAS3, MED13, TLK2, PRELID3BP3, MIR633, ZHX2, ATAD2, TRIB1, LINC00861, PCATI, CASC11, MYC, CYRIB, ASAP1, TMEM71, ST3GAL1-DT, NCRNA00250, MIR30D, KCNK9, AGO2, SLC45A4, GML, GLI4, MINCR, IQANK1, LINC01111, ZFHX4, CPNE3, LRRC69, SLC26A7, C8orf37-AS1, VPS13B, RPS20P23, RNU4-83P, KLF10, RSPO2, MAL2-AS1, RN7SKP153, DEPTOR, COL14A1, TANC2, ACE3P, ACE, FTSJ3, TEX2, POLG2, SMURF2, RGS9, CEP112, SLC16A6, ARSG, FAM20A, MAP2K6, LINC01497, KCNJ2, CALM2P1, RNU7-155P, TMEM259, SBNO2, GNG7, EIF4BP3, HSD17B3, PRXL2C, TRMO, TGFBR1, RN7SL794P, NR4A3, STX17-AS1, PLPPR1, SMC2, RAD23B, LINC01509, HAUS6, TXN, MUSK, SUSD1, KIAA1958, SLC31A1, ALAD, POLE3, TNC, LINC00474, LINC01613, AHCYP2, C5, UST, RMND1, SYNE1, RGS17, RNA5SP225, TIAM2, H3P28, ARID1B, TMEM181, CACYBPP3, IGF2R, LNCDAT, DACT2, SMOC2, LINC01615, THBS2, PRKARIB, MAFK, MAD1L1, GRIFIN, LFNG, TNRC18, DAGLB, KDELR2, TMEM106B, TAS2R2P, SPDYE3, GTF3AP5, MEOX2, CRPPA, RNMTL1P2, PRPSIL1, PNPLA1, DINOL, CDKNIA, RPL12P2, COX6A1P2, PIM1, MDGA1, ZFAND3, KCNK5, KIF6, MOCS1, FOXP4-AS1, LINC01276, NPM1P51, MDFI, USP49, RRP36, RN7SL403P, LINC02537, SCIRT, RUNX2, CD2AP, EFHC1, TRAM2, GCLC, NANOGP3, HMGCLL1, BMP5, DST, BEND6, GAPDHP15, RBBP4P4, RNU7-66P, LINC01626, MY06, ELOVL4, LINC01621, LINC02542, UBE3D, SNAP91, TBX18, ZNF292, SRSF12, MIR548AI, BDH2P1, MCHR2, HACE1, RN7SKP211, AFGIL, ARMC2, SESNI, CD164, AMD1, LINC02518, LINC02880, NT5DC1, FRK, GPRC6A, RFX6, RNU2-8P, RNU4-76P, LINCO2523, HEY2, RSPO3, C6orf58, TMEM244, TMEM200A, SMLR1, HLA-C, AKAP7, MIR548AJ1, STX7, EYA4, TARID, PDE7B, ABRACL, REPS1, LINC01625, ATP5PBP6, MIR3668, HLA-DOA, ADAT2, AIG1, GRM1, SHPRH, SAMD5, LRFN2, OARD1, POLH, GTPBP2, TFAP2B, RPS17P5, FTH1P5, PKHD1, ADGRB3, EPHA7, MMS22L, MIR2113, PRDX2P4, SIM1, NKAIN2, NCOA7, OPRM1, RNU7-152P, PRKN, ALPK1, LINC02465, TERFIP3, RNU1-89P, MAML3, INPP4B, SLC10A7, APOBEC3AP1, LINC01019, NIMIK, MAST4, POC5, RNU6-727P, TMEM161B-AS1, MEF2C-AS2, POLD2P1, NR2F1-AS1, IGSF9B, LINC02706, LINC02714, WNK1, CACNAIC, SLC15A5, PLEKHA5, PDZRN4, PRKAG1, DDN-AS1, FAIM2, POU6F1, CBX5, PA2G4, RPL41, LINC02403, RPL21P103, RAB21, ANKSIB, C12orf42, GPN3, MAPKAPK5-AS1, KDM2B, ANKLE2, RNA5SP379, CTDSPL, CTNNB1, LIMD1, LRRC2, PRSS45P, PTHIR, KIF9, CDC25A, RN7SL664P, PFKFB4, GNAI2, CACNA2D2, C3orf18, DCAF1, ACY1, ABHD14A-ACY1, SMIM4, CACNA2D3, LRTMI, DENND6A-AS1, DENND6A, YEATS2, DGKG, ETV5, RNF168, UBXN7-AS1, LINC02438, PCDH7, KLF3, KIT, LINC02232, MTCO3P28, RNU6-699P, ANTXR2, PCAT4, ABCG2, UNC5C, ADHIB, PPP3CA, LINC01445, AUTS2, STAG3L2, POM121C, DTX2P1, DTX2P1-UPK3BP1-PMS2P11, AP1S1, RELN, BUB3P1, EIF3IP1, PPPIR3A, SMIM30, CADPS2, H4P1, KLF14, DGKI, ZC3HAVI, TTC26, KCNH2, RN7SL178P, NPY, PDE1C, POU6F2, ZMIZ2, FIGNL1, DDC, ATP5MF-PTCD1, PTCD1, CALNI, MLXIPL, ELN-AS1, GTF2IRD1, HIP1, PHTF2, CD36, HMGN2P11, HNRNPA1P8, ABCB1, ANKIB1, RN7SL7P, CALCR, COL1A2, DYNC1I1, RNU7-188P, SEM1, DLX6-AS1, SMURF1, KPNA7, CYP3A4, CYP3A7, AZGP1, PRCC, VANGL2, LMX1A, LRRC52-AS1, MGST3, KIFAP3, GORAB-AS1, HAUS4P1, GORAB, RC3H1, RNU2-12P, U7, MORF4LIP7, ASTN1, HAPLN2, RALGPS2, FAM20B, ABCB5, RAPGEF5, STEAPIB, KLHL7-DT, FAM126A, MALSUI, FCF1P1, CCDCl26, MPP6, RNU6-1103P, NPVF, TSEN15P3, HOXA11, PSMC1P2, HIBADH, TAXIBP1, AQP1, KBTBD2, TBX20, EPDR1, TRGC2, GLI3, GCK, OGDH, SNHG15, FTLP15, EPS15P1, MRPL42P4, JAZF1-AS1, GRB10, CCT6A, GUSB, MTND4P3, GAPDHP24, GPATCH2, SPATA17, SLC30A10, HLX-AS1, TDRD10, DISP1, WDR26, CNIH4, ACBD3, LINC01703, CDC42BPA, LINC01641, WNT9A, CICP26, HMGB1P26, URB2, TTC13, IRF2BP2, LINC00184, GGPS1, TBCE, FMN2, ZAN, RN7SL549P, RPS29P15, SERPINE1, AZGP1P2, MYL10, CUX1, MIR4467, LRWD1, DPY19L2P2, SRPK2, CAV2, COMETT, CPED1, PTPRZ1, TMEM229A, IRF5, AHCYL2, ZC3HC1, LINC-PINT, LINC00513, MKLN1, RNU6-92P, ST13P7, CALDI, TUBB3P2, CNOT4, SLC23A4P, NUP205, PARP12, MKRNI, DENND2A, CUL1, C7orf33, NOS3, CHPF2, NUB1, LINC01006, ESYT2, NCAPG2, CSMD1, PPPIR3B, LINC00681, FGL1, MTUS1, LPL, MAP3K7CL, SCAF4, EVA1C, CFAP298-TCP10L, LINC01690, OLIG2, SON, CBR3-AS1, RPS9P1, SIM2, TTC3, ERG, RPL23AP12, PCBP2P1, BRWD1-AS1, HMGN1, GET1-SH3BGR, SH3BGR, CBS, U2AF1, COL6A1, HDHD5, MICAL3, PRODH, SCARF2, MTATP6P23, LRRC20, CHST3, KCNMA1, DLG5, POLR3A, BMPRIA, ATAD1, CFLIP1, RNLS, SLC16A12, HECTD2, RPS27P1, FGFBP3, TNKS2, NIP7P1, XRCC6P1, RPL17P34, CEP55, KCNMB4, PTPRR, E2F7, PPFIA2, LINC02392, LUM, LINC01619, NACAP8, RPL41P5, METAP2, CDK17, RMST, WASHC3, PAH, C12orf73, TXNRDI, C12orf45, ALDHIL2, CASC18, NUAK1, ST13P3, CKAP4, PWP1, CMKLR1, MMAβ, UBE3B, SH2B3, ATXN2, TBX3, RN7SL865P, LINC02463, RNFT2, SPRING1, FBXW8, SIRT4, RNU4-1, RPS27P25, COX6A1, SPPL3, ANAPC5, HPD, MLXIP, HCAR2, HIPIR, TCTN2, GTF2H3, DNAH10, SCARB1, ETNK1, UBC, EP400P1, XPO4, LATS2, RPSAP54, MIPEP, LNX2, PLUT, FRY, STARD13, STARD13-AS, NBEA, SPART, SPART-AS1, SMAD9, LINC00571, CDKN2AIPNLP3, AZUIP1, MIR320D1, FOXO1, RN7SL597P, KBTBD6, SMIM2-AS1, ZC3H13, LRCHI, LINC01198, GNG5P5, POLR2KP2, ITM2B, FNDC3A, CAβ39L, PHF11, RNY4P30, KPNA3, RNY4P9, CTAGE1OP, RNASEH2B, GUCY1B2, CNMD, RNY4P29, POLR3KP2, SLC39A11, GRB2, FOXJ1, C17orf99, SYNGR2, PGS1, USP36, TIMP2, LGALS3BP, AATK, RENO1, BAHCC1, CENPX, FOXK2, FN3K, USP14, COLEC12, SMCHD1, DLGAP1, AKAIN1, IMPA2, CEP192, TTC39C, NPMIP2, GAREM1, ZNF396, INO80C, LINC01478, SKOR2, CTIF, SMAD7, CXXCI, DCC, TCF4-AS1, ZNF532, LMAN1, CCBE1, RPS3AP49, CNDP2, ZNF516, PPIL2, MIR130B, MN1, LINC02554, TTC28-AS1, ASCC2, MTMR3, SLC35E4, DEPDC5, HMGXB4, MYH9, H1-0, TRIOBP, APOBEC3A, CBX6, FUNDC2P4, PDGFB, TAβ1, ZC3H7B, MEII, CYBER3, PACSIN2, SCUBE1, RIBC2, FBLN1, ATXN10, LINC00899, WNT7B, PPARA, CERK, TBC1D22A, BRD1, LINC02669, KLF6, RPL26P28, UCN3, TASOR2, LINC00707, SFMBT2, PROSER2, PROSER2-AS1, UPF2, SEC61A2, NUDT5, FRMD4A, NMT2, CUBN, MRC1, TMEM236, AIFM1P1, ARL5B, OTUD1, PRTFDC1, ENKUR, ODAD2, WAC, RNU4ATAC6P, LYZL1, JCAD, LINC02644, ARHGAP12, RNU6-1244P, CCDC7, PARD3, RASGEFIA, ZNF485, ARHGAP22, DRGX, C10orf71, SGMS1, MIX23P2, LNCAROD, SLC16A9, LINC01553, CABCOCO1, ADO, ALDH7A1P4, SELENON, PEF1-AS1, ADAMTSL4-AS2, ADAMTSL4, LRRC58, GYG1, SEPTIN7P14, POR, RALGPS1, ANGPTL2, LRRC8A, HTRA1, PDE3B, FAM111A, LTBP3, COL2A1, CNPY2, LRP10, HERC1, HAPLN3, SPSB3, EME2, ADCY7, CLEC3A, CLEC11A, PCNA, ZDHHC20P4, LINC02342, DACH1, RPS10P21, PSMD10P3, KLF5, RNU4-10P, RNU6-66P, RNY1P8, KLF12, LMO7, EDNRB, LINC01080, LINC00351, LINC00379, STK24, DOCK9, GGACT, NALCN, NALCN-AS1, ITGBL1, PROZ, CDCl6, NDRG2, MMP14, IL11, ZNF628, NLRP9, ZNF471, ZNF460, ZNF548, VN2R19P, C20orf194, C20orf27, SPEF1, CDS2, TARDBPP1, LINC01428, LINC01751, HAO1, TMX4, SLX4IP, LINC01752, FAT1P1, LINC02871, ISM1, B4GALT5, PTGIS, SNAII, LINC01271, RN7SL636P, COX6CP2, ATP9A, MC3R, FAM210B, AURKA, MRPS16P2, PRELID3B, TAF4, MTG2, LAMA5, RPS21, OGFR, HELZ2, SLC2A4RG, MYT1, MIR99AHG, PRKACA, ZNF333, AKAP8, BRD4, KLF2-DT, SLC35E1, BST2, CCDCl94, ARRDC2, RPL39P38, LSM4, ELL, MAU2, PBX4, ZNF14, ZNF101, ZNF486, BNIP3P15, ANKRD27, HPN-AS1, HPN, LSR, UPKIA, COX6B1, ARHGAP33, ZNF585A, ACTN4, FBXO27, HIPK4, LTBP4, ITPKC, COQ8B, CEACAMP3, CEACAM4, ERF, CIC, ZNF575, KCNN4, APOCIP1, SYMPK, PNMA8C, PNMA8A, SLC1A5, HNRNPMP2, BBC3, BICRA, ZSWIM9, CARD8, RASIP1, SNRNP70, KASH5, PRMT1, GPR32P1, SPACA6, SPACA6P-AS, LILRB5, RBM39, AAR2, DLGAP4, MANBAL, SRC, BLCAP, RPL7AP14, LINC01734, ATG3P1, TOP1, PLCG1-AS1, CHD6, MYBL2, YWHAB, PABPCIL, STK4, WFDC2, PIGT, ZSWIM3, SULF2, BANF2, SLC24A3, ZNF877P, LINC01432, FOXS1, XKR7, NOL4L, COMMD7, EFCAB8, MAPRE1, RPL12P3, BPIFA1, SNTAI, RALY, DYNLRB1, PIGU, TRPC4AP, HMGB1P37, GGTA1, DAB2IP, RABGAP1, DENNDIA, LHX2, OLFML2A, MAPKAP1, MVB12B, ST6GALNAC4, DNM1, LINC00963, RAPGEF1, OBP2B, GBGT1, BRD3, RXRA, NOTCH1, C9orf163, DIPKIB, SNHG7, ANAPC2, ADARB2, PITRM1-AS1, PITRM1, LINC02754, LRP5, C11orf24, TPCN2, SMIM38, LINC01488, LINC02584, ANO1, NUMA1, PDE2A, PDE2A-AS1, POLD3, RNF169, CHRDL2, THAP12, MYO7A, AAMDC, INTS4, CCDC83, EED, AMOTL1, FAM76B, SESN3, LINC02732, SIK2, LAYN, PCSK7, TTC36-AS1, KMT2A, TRIM29, OAF, JHY, BSX, NAPILIP1, RPUSD4, DCPS, LINC02763, ETS1, ARHGAP32, ADAMTS8, SLC6A12, SLC6A13, TSPAN9, PARP11-AS1, FIGNL2, NCOA2, CEP95, DEF6, LINC00702, TGFB3, IFT43, FGF6, FGF23, NTF3, VAMP1, TAPBPL, MLF2, MANSC1, CDKNIB, FAM234B, GNAI2P1, RPL30P11, ATF7IP, GUCY2C, H4-16, SKP1P2, SLCO1B1, LDHB, ST8SIA1, RASSF8, SSPN, MRPS35, HMGB1P49, MAPK14, G6PC3, TARSI, ECD, LINC01803, RCCD1, ABCC10, EFCAB13-DT, ENTPD1-AS1, LCOR, DNMBP, HIFIAN, FBXW4, ARMH3, SH3PXD2A, MXI1, VTI1A, AFAPIL2, CASC2, FAM204A, PRDX3, BAG3, FGFR2, RPS15AP5, HMG20B, SH3GL1, TNFAIP8L1, MYDGF, UHRF1, KDM4B, SAFB2, NDUFA11, MLLT1, TRIP10, MARCHF2, OLFM2, DNMT1, KANK2, ZNF878, ZNF799, ZNF443, HOOK2, PRDM11, LARGE2, CELF1, FOLH1, OR4A16, OR4A12P, OR8J1, UBE2L6, RPS4XP13, OR5AZIP, OR5BAIP, GLYAT, GLYATL1, WARSIP1, SLC25A47P1, CBLIF, TCN1, MS4A4A, MS4A4E, PGA5, SPINDOC, POLA2, SARTI, ACTN3, RAD9A, SBF2, DKK3, RRAS2, SOX6, PIK3C2A, ABCC8, SAAL1, MIR3159, DBX1, METTL15, LINC02742, MPPED2, DNAJC24, EIF3M, CSTF3, PAMR1, ALX4, FAM53B, ADAM12, MGMT, PPP2R2D, PWWP2B, LINC02870, PSMD13, DEAF1, EPS8L2, MOB2, LSP1, LINC01219, INS-IGF2, IGF2-AS, KCNQ10T1, CARS1, OR51D1, TRIM6, TRIM6-TRIM34, DENND2B, ERGIC2, IPO8, DENND5B, ADAMTS20, EEF1A1P17, SCAF11, OR7A19P, SLC38A2, MARK3P1, PCED1B, RAPGEF3, LALBA, OR11MIP, WNT1, DDN, LIMA1, FAM186A, DAZAP2, SMAGP, SPRYD3, ITGB7, SP1, COQ10A, ANKRD52, SDR9C7, LRP1, B4GALNT1, RPL13AP23, RNU4-20P, LINC02448, PPM1H, SRGAP1, LLPH, RNA5SP362, LINC02408, RAP1B, CPSF6, MYRFL, LINC02694, BMF, INAFM2, PLCB2, KNL1, RMDN3, INO80, CIBAR1P1, MGA, EHD4, UBR1, MTND5P40, GABPB1, HDC, USP8, DNAAF4-CCPG1, CCPG1, NEDD4, RFX7, PABPN1, MYH7, NGDN, NRL, TGMI, G2E3, PSMA6, NFKBIA, RALGAPA1, FBXO33, TOGARAMI, KLHL28, ATL1, LINC02331, RPS3AP46, MIR5580, KTN1, PELI2, OTX2-AS1, LINC01500, DAAM1, RTN1, SIX6, ANPEP, ZNF710, ZNF710-AS1, IQGAP1, BLM, CHASERR, SEPHS1P2, LINC01197, ARRDC4, LUNAR1, DNMIP46, LYSMD4, PAN2, RNF41, SMARCC2, TLN2, USP3, LINC02568, ANKDDIA, SPG21, DENND4A, HMGN2P47, LINC02205, THSD4, RPL5P3, NR2E3, GRAMD2A, ARIH1, HCN4, NEO1, NPTN, CD276, STRA6, CCDC33, CYP11A1, UBL7-AS1, SCAMP2, NRG4, PSTPIP1, HMG20A, CPEB1, HDGFL3, UBE2Q2P1, SLC28A1, AKAP13, RNU6-185P, LINC01586, DET1, KRT18P47, MFGE8, TICRR, SLC39A5, MNAT1, PPP2R5E, ESR2, PLEKHG3, NUP50P1, FNTB, CHURC1-FNTB, FUT8, NCOA4P1, GPHN, SMOC1, SLC8A3, PAPLN, ZNF410, RPS2P2, SYNDIGIL, ADCK1, CEP128, ZC3H14, MPPEIP1, TTC7B, CATSPERB, SLC24A4, CCDCl97, SERPINA5, SERPINA13P, RPSAP4, DICER1, RN7SL714P, HHIPL1, CCDC85C, WDR25, RTL1, LINC02320, DIO3, AKTI, CRIP1, PWRN4, TMCOSA, LINC01852, LINC02345, SPRED1, FAM98B, LMF1, CEROX1, CACNAIH, UNKL, PTX4, RPS3AP2, TSC2, ECI1, ABCA17P, KCTD5, ELOB, RNU1-22P, IL32, TRAP1, CREBBP, TFAP4, GLIS2, ANKS3, GLYR1, USP7, C16orf72, ZFP3-DT, SLC52A1, MED31, TXNDC17, ACAP1, NEURL4, ATP1B2, STX8, GAS7, MED9, RAI1, TOMIL2, MYO15A, DHRS7B, TMEM11, NATD1, FAM27E5, FLJ36000, RPL34P31, RAB34, PHF12, RPL35AP35, RNU4-34P, SSH2, EFCAB5, NSRP1, CPD, SKAP1, TTLL6, HOXB13, NGFR, TOB1-AS1, TOB1, LINC02073, STXBP4, LRRC37BP1, RN7SL316P, NF1, RNU6ATAC7P, RNU6-1134P, UTP6, STAC2, NEUROD2, CDK12, NR1D1, MSL1, JUP, DHX8, ETV4, SOST, WHSCIL2P, SLC25A39, SMCO4P1, FZD2, ADAM11, HEXIM1, MAP3K14, RNA5SP443, LINC02210-CRHR1, GOSR2, NABP2, KCTD18, SLC30A6, KCNIP3, FAHD2A, CNNM4, RPL24, BRK1, VHL, RPS27AP8, ADAMTS19-AS1, RNA5SP214, B3GNTL1P2, CD2AP-DT, SNORA70, H2AZP5, DLG5-AS1, CXCR5, OR4A8, OR4A2P, BANF1, ACAD10, SOAT1, TGFB2-OT1, GPBP1L1, LINC01767, RNF207, MIGA1, LY75-CD302, LY75, HOXD3, ANKRD44, LINC02030, RNU6-1252P, TMEM165, ADGRL3, RNU2-40P, IFITM3P1, UBE2H, BRAF, ZNF696, ZMAT4, SPIDR, TMEM245, PHF19, ADAMTSL2, CHD9, PHBP21, LRRK2, COPZ1, CD163L1, M6PR, LINC00642, IGDCC3, SNUPN, HDAC5, ARL17B, LRRC37A, METTL2A, BCL6B, IL1RAPL2, IGSF1, BRWD3, HK2P1, PLCB1, RNF185, WWC3, COL4A6, KDM5C, KANTR, SPRYD7P1, FGF16, TBX22, ZNF570, ZNF793-AS1, SPTLC3, LPIN3, WFDC3, FCF1P6, DMRTA2, LINC01562, KYAT3, MPHOSPH10, MPHOSPH6, MLYCD, OSGIN1, USP10, GSE1, LINC02139, LINC00917, FENDRR, FLJ30679, FOXL1, LINC02188, LINC02182, ZNF469, GALNS, CDH15, SPG7, VPS53, YWHAE, CRK, WDR81, SMG6, MIR6776, CLUH, RAPIGAP2, ZZEF1, CETP, CDH11, C16orf70, KCTD19, NRNIL, PSKH1, UTP4, PDXDC2P, PDXDC2P-NPIPB14P, DDX19B, DDX19A-DT, COG4, IL34, DHX38, CTRB2, ZFP1, TMEM170A, WWOX, SCNNIG, TEKT5, MIR3180-4, ABCC1, ABCC6, SYT17, VPS35L, ACSM3, THUMPDI, EEF2K, TLCD3B, ZNF423, CYLD, LINC02127, SOD1P2, UNGP1, SALLI, CASC22, MTND5P34, KIF17, SLC4A4, LINC01094, BMP2K-DT, BMP2K, LINC01088, BPGM, RPL17P50, PLA2G12B, CFAP70, PPP3CB, CD163, GAPDHP31, ERP27, CERS5, USP44, TMEM263-DT, RPS2P48, MTCO1P40, ISCA1P3, RPE65, ELOCP18, FAM20C, RPL12P46, RNU6-735P, MIR3685, CYFIP1, GPIBA, CHRNE, GIP, CPNE1, CLK3, HMCES, RASGEF1B, MXD4, ZFYVE28, PARP6, SF3B3, POLR2A, WNT3, SNF8, RNU1-42P, SPAG4, COPS7B, MIR1471, KIF9-AS1, POCIA, ALDOAP1, NT5DC2, RTP2, SPINK2, MTHFD2P6, SLC17A1, H3C6, BTN3A3, H2BC11, RNU2-62P, ZNF204P, H4C11, H2BC14, ZSCAN16, ZNF602P, ZSCAN16-AS1, MTCO1P49, RC3H2, KRT18P67, PSMA1, FOXRED1, RAB5B, HMGA2-AS1, HVCN1, BRAP, NAA25, RPH3A, OAS3, ATXN3, RPSAP5, AP4E1, RPL32P30, VPS13C, SLFNL1-AS1, HYI-AS1, FGGY-DT, MIER1, ZRANB2-AS1, PAX8-AS1, PAX8, GAS5, GAS5-AS1, LHX9, MICOS10, SERTAD4, HP1BP3, INTS7, FAAP20, HLX, LINC02817, CNIH3, ARID4B, LINC02774, LINC01715, MATN1, THRAP3, SLC52A3, ADAMTS1, LTN1, MAPK1, GRK3, CRYBB2P1, TTC28, SFI1, LINC01399, RNU7-167P, SNU13, RNU6-30P, ARMCX3, RNU6-309P, GAPDHP77, PARVA, KCNJ1, ODF3, BETIL, HTATIP2, EIF4A2P5, RCN1, OR9Q1, LINC00375, ARHGEF40, NYNRIN, G2E3-AS1, SFTA3, NKX2-1, MIA2, SERPINA2, DACH2, SHROOM2, LARP7, MIR302CHG, BBS7, TMEM248P1, LINC02379, LINC02615, LINC00499, THAP12P9, PRDX4P1, LRRC66, NREP, LOX, RUFY2, LINC02642, ST8SIA3, NEDD4L, MIR122HG, LRRC30, ADGRL1, ZNF420, SARS2, FBXO46, MEIOSIN, HCN1, MRPS30, SETD9, TENT4A, ANKDD1B, ADCY2, MSH3, LINC02488, RPL23AP48, HMGB3P18, DPY19L2P1, TCPIP1, C7orf25, CICP11, SUMO2P3, MTCO1P57, ABCB4, TBX18-AS1, PNRC1, MET, GHRHR, ADCYAP1R1, SILC1, BMSIP23, IGKV1OR2-2, KANSL3, SENP7, GOLGB1, IQCB1, SEC22A, OSBPL11, ZIC4, TFBIM, CLDN20, CCND3, KRT19P1, RNU7-102P, LINC01603, RNU6-1151P, MATN2, CDCA4P1, DMRTA1, B4GALT1, PCM1, MIR124-2HG, VXN, ACSM1, PPP4C, MMP2, HERPUD1, SLC12A3, SERPINF1, CCDCl44B, CYB5D2, RPS6KB1, MEP1B, GALNT1, PAK3, THOC2, AIFM1, ADGRG4, RNU6-985P, RN7SKP31, MAGEA10, GABRA3, ZNF275, GDI1, SCML2, EIF1AX, APOO, NROB1, TASL, SRPX, MIR222HG, LINC02595, EBP, TBC1D25, DGKK, NBDY, SSBL2P, KRT8P17, PJA1, AADACL2-AS1, EGFEMIP, ULK4, LINC02040, MAGI1, LINC01170, RPS27AP18, DIAPH1, LPCAT1, RPL7P20, LINC01947, SDHA, NNT, RNU6-381P, RPS6P12, RMI1, SLC28A3, MIR4290HG, FAM171A1, CACNB2, NOP58, BOK-AS1, SLC8A1, LINC01794, EML4, C2CD4A, THORLNC, LINC01956, ERCC3, WBP11P2, RNA5SP106, ACVR2A, RNU6-546P, GORASP2, KRT18P19, CFLAR, MVK, RN7SKP250, ITPR2, LINC02451, DIP2B, NOSIP, PRRG2, LINC01427, LINC00261, BCL2L1, ELK3, LINC02409, CFAP97D2, LINC02782, PLPPR5, MXD3, RIOK3, ENSA, GOLPH3L, or combinations thereof.

In some embodiments, the genes of any preceding aspect are also affected by an HSV infection. In some embodiments, the genes of any preceding aspect are also affected by an HSV-1 viral infection.

In one aspect, disclosed herein is a method of treating a neurodegenerative disease in a subject in need thereof, the method comprising collecting a biological sample from the subject, detecting a gene expression signature in a nucleic acid isolated from the biological sample, and administering a therapeutically effective amount of an anti-viral agent to the subject with the gene expression signature, wherein the gene expression signature comprises at least one gene of any preceding aspect affected by the neurodegenerative disease and a herpes simplex virus 1 (HSV-1).

In one aspect, disclosed herein is a method of treating an autoimmune disease in a subject in need thereof, the method comprising collecting a biological sample from the subject, detecting a gene expression signature in a nucleic acid isolated from the biological sample, and administering a therapeutically effective amount of an anti-viral agent to the subject with the gene expression signature, wherein the gene expression signature comprises at least one gene of any preceding aspect affected by the autoimmune disease and a herpes simplex virus 1 (HSV-1).

In one aspect, disclosed herein is a method of treating a psychiatric disorder in a subject in need thereof, the method comprising collecting a biological sample from the subject, detecting a gene expression signature in a nucleic acid isolated from the biological sample, and administering a therapeutically effective amount of an anti-viral agent to the subject with the gene expression signature, wherein the gene expression signature comprises at least one gene of any preceding aspect affected by the psychiatric disorder and a herpes simplex virus 1 (HSV-1).

In one aspect, disclosed herein is a method of treating a defective human trait, including, but not limited to height and obesity, in a subject in need thereof, the method comprising collecting a biological sample from the subject, detecting a gene expression signature in a nucleic acid isolated from the biological sample, and administering a therapeutically effective amount of an anti-viral agent to the subject with the gene expression signature, wherein the gene expression signature comprises at least one gene of any preceding aspect affected by the human trait and a herpes simplex virus 1 (HSV-1).

### Kits

In one aspect, disclosed herein is a kit for detecting a panel of genes affected by a neurological disorder and a herpes simplex virus (HSV), the kit comprising a primer composition for binding at least one gene of said panel, and an enzymatic composition for amplifying the at least one gene, wherein the at least one gene comprises any one gene selected from AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, YAP1, ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, WNT7B, or fragments thereof.

In some embodiments, the primer composition comprises one, two, or more oligonucleotides. In some embodiments, the primer composition comprises a forward primer or a reverse primer. In some embodiments, the primer composition comprises a forward primer and a reverse primer.

In some embodiments, the oligonucleotides target a nucleotide sequence within the at least one gene. In some embodiments, the enzymatic composition comprises a polymerase and a buffer. In some embodiments, the polymerase comprises a DNA polymerase or an RNA polymerase. In some embodiments, the kit can be used to generate cDNA from RNA.

In some embodiments, the kit comprises a homogenous or heterogeneous mixture of nucleotides, including, but not limited to deoxynucleoside triphosphates (dNTPs) mix. In some embodiments, the kit comprises a magnesium (Mg2+) composition. In some embodiments, the kit comprises a DNAse-free water.

In some embodiments, the kit can be combined with a nucleic acid isolation method. In some embodiments, the kit can be combined with a sequencing method, including but not limited to RNA sequencing, mRNA sequencing, Targeted RNA sequencing, Total RNA sequencing, Sanger DNA sequencing, Next generation sequencing (NGS), DNA microarray, high-throughput sequencing, or combinations thereof.

In some embodiments, the neurological disorder comprises Alzheimer's disease (AD), dementia, multiple sclerosis (MS), Parkinson's disease, Huntington's disease, obsessive compulsive disorder (OCD), attention-deficit/hyperactivity disorder (ADHD), schizophrenia, or amyotrophic lateral sclerosis (ALS).

In some embodiments, the HSV comprises an HSV-1. In some embodiments, the method further comprises a reagent for extracting a nucleic acid. In some embodiments, the method further comprises a reagent for detecting the at least one gene. In some embodiments, the nucleic acid comprises an RNA molecule.

In some aspects, disclosed here is a kit for detecting a panel of genes affected by Alzheimer's disease (AD), Attention-deficit Hyperactivity Disorder (ADHD), Amyotrophic Lateral Sclerosis (ALS), Autism Spectrum Disorder (ASD), Bipolar Disorder (BD), Body-Mass Index (BMI), Inflammatory bowl disease (IBD), Major Depressive disorder (MDD), Multiple Sclerosis (MS), Obsessive compulsive disorder (OCD), Parkinson's disease (PD), Progressive supranuclear palsy (PSP), Rheumatoid arthritis (RA), schizophrenia (SCZ), Tourette syndrome (TS), Type I Diabetes mellitus, Type II Diabetes mellitus, WHR, additional brain disorders, or disorders affecting height.

In some embodiments, the panel of genes includes, but is not limited to any gene or gene combination of any preceding aspect. In some embodiments, the genes of any preceding aspect are also affected by an HSV infection. In some embodiments, the genes of any preceding aspect are also affected by an HSV-1 viral infection.

In one aspect, disclosed herein is a kit for detecting a panel of genes affected by a neurodegenerative disease and a herpes simplex virus (HSV), the kit comprising a primer composition for binding at least one gene of said panel, and an enzymatic composition for amplifying the at least one gene, wherein the panel of genes comprises at least one gene from any preceding aspect.

In one aspect, disclosed herein is a kit for detecting a panel of genes affected by an autoimmune disease and a herpes simplex virus (HSV), the kit comprising a primer composition for binding at least one gene of said panel, and an enzymatic composition for amplifying the at least one gene, wherein the panel of genes comprises at least one gene from any preceding aspect.

In one aspect, disclosed herein is a kit for detecting a panel of genes affected by a psychiatric disease and a herpes simplex virus (HSV), the kit comprising a primer composition for binding at least one gene of said panel, and an enzymatic composition for amplifying the at least one gene, wherein the panel of genes comprises at least one gene from any preceding aspect.

In one aspect, disclosed herein is a kit for detecting a panel of genes affected by a defective human trait and a herpes simplex virus (HSV), the kit comprising a primer composition for binding at least one gene of said panel, and an enzymatic composition for amplifying the at least one gene, wherein the panel of genes comprises at least one gene from any preceding aspect.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Alzheimer's Disease-Associated Neuropathology Induced by Herpes Simplex Virus 1 Infection in Human Cerebral Organoids can be Prevented by Acyclovir Treatment

It has been contemplated that there is an infectious etiology of Alzheimer's disease (AD) remains debated. Herein, this was tested using the double-stranded DNA herpes simplex virus 1 (HSV-1) and anti-herpetic drug acyclovir (ACV) on dissociated cells from human cerebral organoids (cOrgs). By performing large-scale RNA sequencing, differentially expressed transcripts were identified in HSV-1 infected cOrgs were enriched for AD-associated GWAS genes. HSV-1 infected cOrgs also had lower extracellular Aβ42/40 ratios detected in conditioned media using ELISA assays and increased intracellular Aβ42 and phosphorylated Tau (pTau-212) expression was observed, as well as neuronal cell death, astrogliosis and increased proportions of other glia, such as GFAP+ astrocytes, Iba1+ microglia and OLIG1+oligodendrocyte progenitor cells in HSV-1 infected cOrgs using flow cytometry. Aβ42 and pTau-212 expression was strongly correlated with HSV-1 expression (Pearson's r=0.81 and 0.86) and among the cell type specific markers, only OLIG1 expression was similarly strongly correlated with HSV-1 expression (Pearson's r=0.84-0.86), which indicates a causal relationship between HSV-1, Aβ42, pTau-212 and OLIG1+ oligodendrocyte progenitor cells.

ACV treatment of HSV-1 infected cOrgs rescued most of the transcriptomic and cellular perturbations in a dosage-dependent manner; however, the treatment led to an enrichment in transcripts associated with rheumatoid arthritis and Type 1 diabetes and 19-23% of transcripts had exacerbated expression due to ACV treatment, showing potential off-target effects. Finally, post-mortem brains from 25-31% of patients with late-onset AD have transcriptomic signatures similar to HSV-1 infected cOrgs, indicating that anti-viral treatment may benefit a major subset of AD patients.

In 1991, it was first proposed that herpes simplex virus 1 (HSV-1) infection was associated with increased risk for Alzheimer's Disease (AD) when HSV-1 DNA was found in amyloid plaques of AD brains. Subsequently, additional studies reported associations between herpesviruses and AD, as well as associations between HSV-1 and a major genetic risk factor for AD, apolipoprotein E (APOE) &4 allele. Epidemiological studies on large-scale population cohorts further supported the infectious hypothesis in AD through the discovery that anti-herpetic medication reduced dementia risk in patients with HSV-1 infections and that increased risk for AD is strongly associated with herpes simplex encephalitis (HSE).

A study analyzed RNA sequence data (RNA-seq) from human post-mortem brains and reported increased transcript abundance of human herpesviruses—in particular, HHV-6A, HHV-7, HSV-1 and HSV-2 in AD. However, subsequent studies reported conflicting results: re-analyses of the RNA-seq data did not detect significant associations between AD and viral load of herpesviruses such as HHV-6A and HHV-6B. Recent studies also found that HSV-1 infection in an AD mouse model (5×FAD) did not result in Aβ aggregation.

Researchers revisited this controversy using recent, rapid technological development in human induced pluripotent stem cell (hiPSC) models such as 3D brain organoids and examined if there is an association between herpesviruses and AD. These studies have uncovered exciting and causal relationships between HSV-1 infections and pathogenesis of AD and other neurodegenerative diseases. HSV-1 was found to catalyze amyloid β-peptide (Aβ42) aggregation and dysfunction of actin cytoskeleton in human brain organoids, as well as led to HSE-associated cell type specific aberrations, thus illuminating molecular mechanisms involved in the infectious etiology of AD.

The anti-herpetic drug acyclovir (ACV) was shown to be highly effective in reducing HSV-1 replication and reducing clinical symptoms. Clinical trials using the oral anti-herpetic drug valacyclovir (VCV) as a treatment for AD have also been conducted and VCV proves to be a promising AD therapeutic.

Given the exciting discoveries that support a causal role for HSV-1 in AD, and paralleling recent research that firmly identified a causal role for an infectious etiology in another neurodegenerative disease, multiple sclerosis, large-scale RNA-seq, ELISA assays and flow cytometry experiments were performed to systematically dissect the role of HSV-1 in AD by using human cerebral organoids (cOrgs). More importantly, also the efficacy and safety of

ACV treatment was evaluated on HSV-1 infected cOrgs in the context of AD-associated transcripts and neuropathologies. This study provides key insights into the molecular and cellular biomarkers of HSV-1 associated AD and paves the way to develop high-throughput therapeutic screens for a subset of AD patients using human cerebral organoids.

### Results

HSV-1 infection and treatment induced major human transcriptome perturbations in cOrg samples but not in hiPSCs. The molecular effects of HSV-1 infection were first evaluated in a complex co-culture system using human cOrgs. Several batches of 3D cOrgs were differentiated using human induced pluripotent stem cells (hiPSCs) from a control donor without AD using a previously established protocol and dissociated cells from 2-month to 4-month cOrgs for replating as 2D monolayer cell cultures.

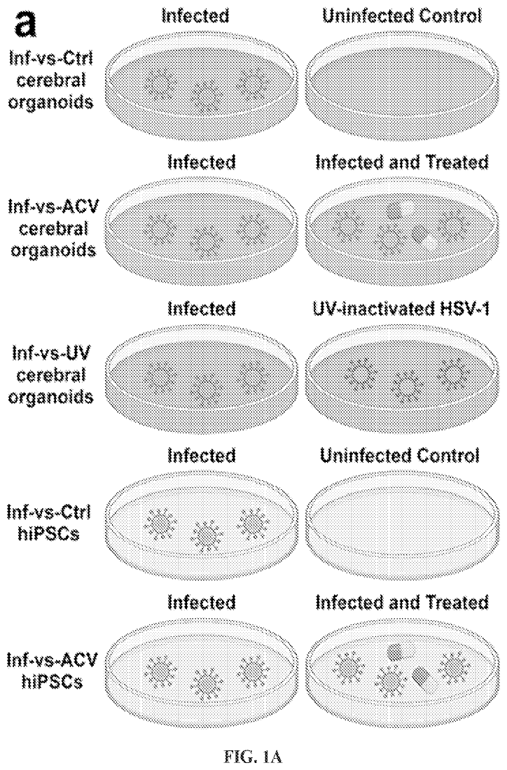

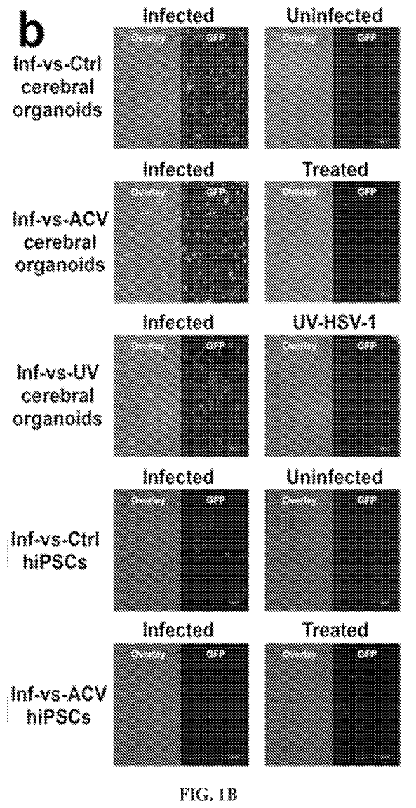

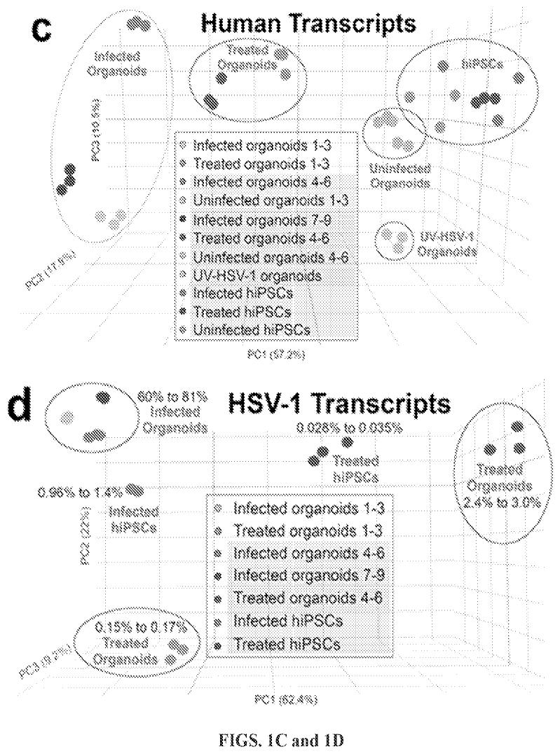

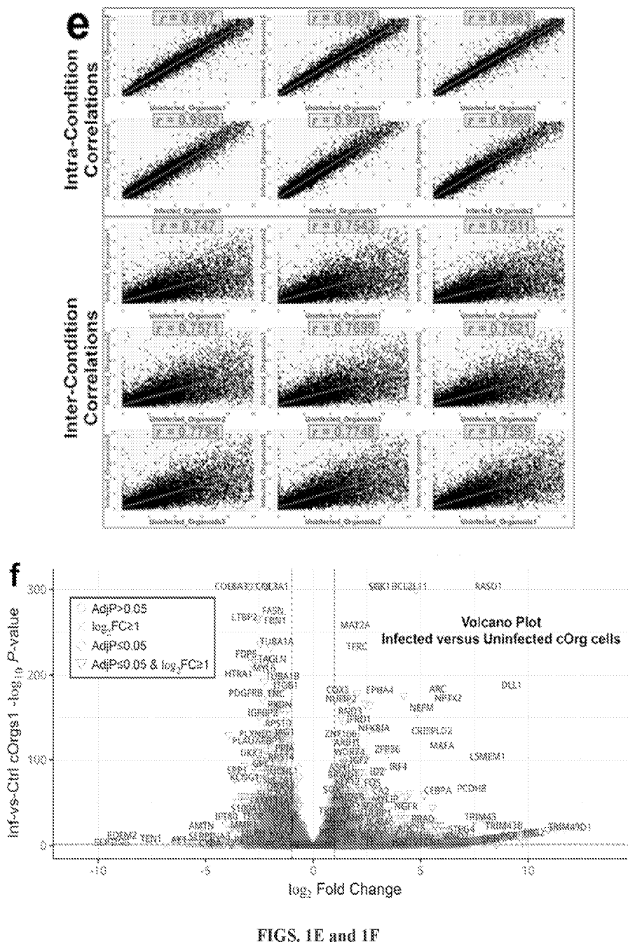

Three sets of experiments were conducted to compare HSV-1 (GFP-tagged) infected cOrg samples versus uninfected cOrg samples (Inf-vs-Ctrl cOrgs), HSV-1 infected cOrg samples versus HSV-1 infected and ACV-treated cOrg samples (Inf-vs-ACV cOrgs), and HSV-1 infected cOrg samples versus cOrg samples with UV-inactivated HSV-1 added (Inf-vs-UV cOrgs), shown in FIGS. 1A and 1B. The first 2 sets of experiments were repeated for replication. In addition, another 2 sets of experiments were conducted to compare HSV-1 infected hiPSCs from the same donor versus uninfected hiPSCs (Inf-vs-Ctrl hiPSCs) and HSV-1 infected hiPSCs versus HSV-1 infected and ACV-treated hiPSCs (Inf-vs-ACV hiPSCs). Each set of experiments was performed in triplicates, followed by total RNA extraction, ribosomal RNA depletion and RNA sequencing (RNA-seq).

3D principal components analysis (PCA) showed that most of the variation (57.2%) in the human transcripts was captured by the first principal component (PC1) (FIG. 1C and FIGS. 7A and 7B). The cOrg samples were also distinctively separated by their conditions into well-defined clusters on PC1. cOrg samples with UV-inactivated HSV-1 were clustered close to uninfected cOrg samples on PC1, whereas ACV-treated cOrg samples formed their own cluster between infected and uninfected cOrgs on PC1. All hiPSC samples clustered together into a single group regardless of the condition (infected, uninfected or ACV-treated).

To elucidate the transcriptome-wide expression patterns of HSV-1, similarly PCA was performed on the viral transcripts and found that most of the variation (62.4%) in the viral transcripts were captured by PC1 (FIG. 1D). All the infected cOrg samples clustered together and infected hiPSC samples clustered closely with the infected cOrg samples. The treated hiPSC and cOrg samples formed their own clusters, separate from the infected samples. However, both sets of ACV-treated cOrg samples showed distinctive clusters, primarily in PC1 and PC2. 0.15-0.17% of all transcripts were viral transcripts in the first set of ACV-treated cOrg samples (cOrgs1), 2.4-3% of total transcripts were viral transcripts in the second set of ACV-treated cOrg samples (cOrgs2), whereas 60-81% of all transcripts were viral transcripts in infected cOrg samples. There were 14-20 times more HSV-1 transcripts in the second set of ACV-treated cOrg samples compared to the first set of ACV-treated cOrg samples, showing that ACV treatment in the second set of cOrg samples was not as effective as in the first set of cOrg samples on suppressing viral transcript expression.

0.96-1.4% of all transcripts were viral transcripts in infected hiPSCs and 0.028-0.035% of all transcripts were viral transcripts in ACV-treated hiPSCs. There were lower viral transcript counts in the hiPSC samples than the cOrg samples, despite using a higher multiplicity of infection (MOI) for the hiPSC samples versus the cOrg samples (MOI of 4 and 2 respectively), consistent with prior reports that stem cells are highly resistant to viral infection, unlike differentiated cells.

Strong type I interferon response was observed in HSV-1 infected cOrg samples. High correlations between biological replicates of cOrg samples were observed by pooling large numbers of 3D cOrgs for the initial dissociation. Pairs of infected cOrg replicates or pairs of uninfected cOrg replicates had high correlations and low variability (Pearson's r>0.99; FIG. 1E and Table 4). The correlations between infected-uninfected cOrg pairs were lower (r=0.75-0.82). The correlations between infected-treated cOrg pairs ranged from r=0.81-0.89. On the other hand, the correlations between infected-uninfected or infected-treated hiPSC pairs were high (r=0.95-1), indicating that fewer human transcripts were perturbed by HSV-1 infection in hiPSCs. Since hiPSCs do not produce type 1 interferon in response to viral infections and respond weakly to exogenous interferon, these results were consistent with prior studies and indicated a strong interferon response in cOrg samples from HSV-1 infection.

HSV-1 infection preferentially up-regulated human transcripts in cOrg and hiPSC samples but ACV treatment did not significantly rescue the global imbalance in cOrg samples. The volcano plots for human gene expression in HSV-1 infected versus uninfected cOrgs showed that there were more up-regulated human transcripts compared to down-regulated transcripts (odds ratios, OR=1.13 and 1.14; family-wise error rate, FWER=5.9×10⁻⁴ and 1.2×10⁻⁴; FIG. 1F, FIG. 7C, and Table 5). Similarly, there were more up-regulated human transcripts in HSV-1 infected cOrgs versus cOrgs with UV-inactivated HSV-1 (OR=1.21, FWER=5.8×10⁻⁹; Extended Data FIG. 1d). In hiPSCs, a similar imbalance with more up-regulated human transcripts in HSV-1 infected versus uninfected hiPSCs was observed (OR=1.3, FWER=5.0×10⁻⁴; FIG. 7E).

There were equal proportions of up-regulated or down-regulated human transcripts in HSV-1 infected versus ACV-treated cOrgs (OR=1.02 and 1.03, FWER=0.42 and 0.36 respectively; FIGS. 7F and 7G) and in ACV-treated versus uninfected cOrgs and ACV-treated versus cOrgs with UV-inactivated HSV-1 (OR=1.1 and 1.15, FWER=0.16 and 0.051 respectively; FIGS. 7H and 7I). However, there was a significant excess of up-regulated transcripts observed in infected versus ACV-treated hiPSCs (OR-9.9, FWER=3.4×10⁻⁷; FIG. 7J). No imbalances in the proportions of up-regulated or down-regulated HSV-1 transcripts in infected versus treated cOrgs or hiPSCs were observed (FIGS. 7K, 7L, and 7M).

Collectively, these results showed that while HSV-1 infection preferentially up-regulated the expression of human transcripts in cOrgs and hiPSCs, however, ACV treatment on infected cOrgs did not significantly rescue these global imbalances (FIG. 7N).

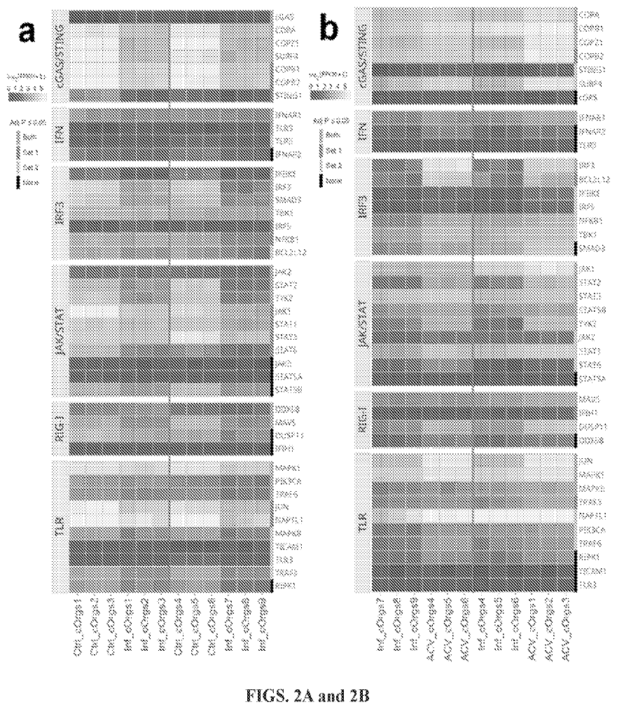

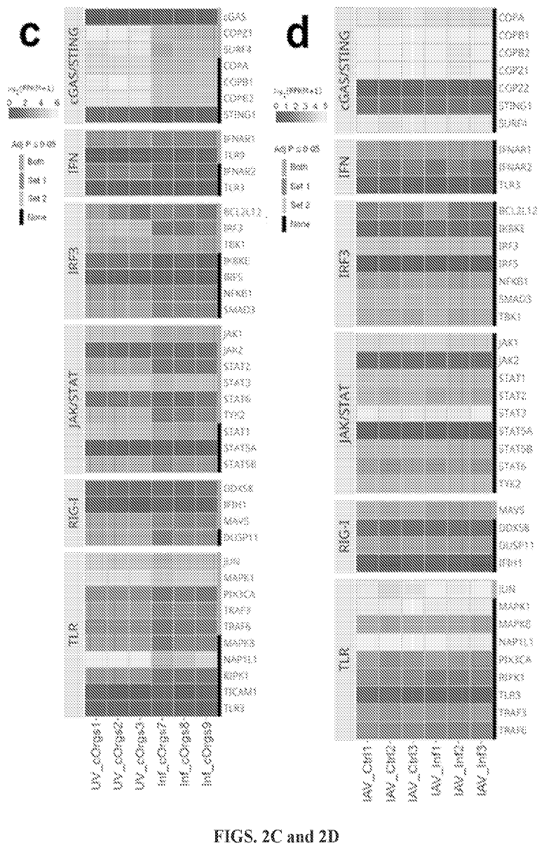

HSV-1 infection in cOrg samples perturbed the expression of transcripts in several innate immune pathways, unlike Influenza A (IAV) infection in cOrg samples. The expression of transcripts in several human immune pathways (cGAS/STING, IFN, IRF3, JAK/STAT, RIG-I and TLR) were significantly differentially perturbed in both sets of HSV-1 infected versus uninfected cOrg samples (FIG. 2A), as well as in hiPSCs (FIGS. 8A and 8B). ACV treatment rescued the expression for some of these transcripts (FIG. 2B). Infected cOrgs versus cOrgs with UV-inactivated HSV-1 showed similar differential expression as infected versus uninfected cOrg samples (FIG. 2C). To compare which DEGs in cOrg samples were specifically perturbed by the double-stranded DNA virus HSV-1, a single-stranded respiratory RNA virus (Influenza A or IAV) was used to infect cOrgs for RNA-seq (FIGS. 8B and 8C). IAV-infected versus uninfected cOrgs did not result in many significant DEGs in these human immune pathways, except in the RIG-I and TLR pathways (FIG. 2D).

HSV-1 infection induced differentially expressed genes in cOrgs that were enriched for AD-associated genes but not in hiPSCs or SH-SY5Y-differentiated neurons. Lists of genes were obtained that were identified to be associated with 21 common neurodegenerative, neuropsychiatric or autoimmune diseases, as well as human traits and related diseases through genome-wide association studies (GWAS) from the GWAS Catalog (See Detailed Description). To evaluate if the differentially expressed genes (DEGs) in infected versus uninfected cOrgs were enriched for genes associated with any of the 21 common diseases or traits, gene set enrichment analyses (GSEA) was conducted with the gene lists in the Detailed Description.

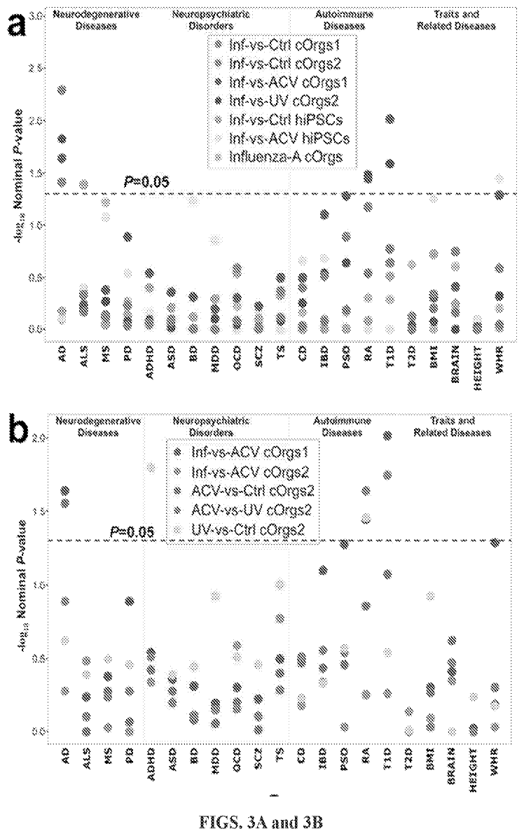

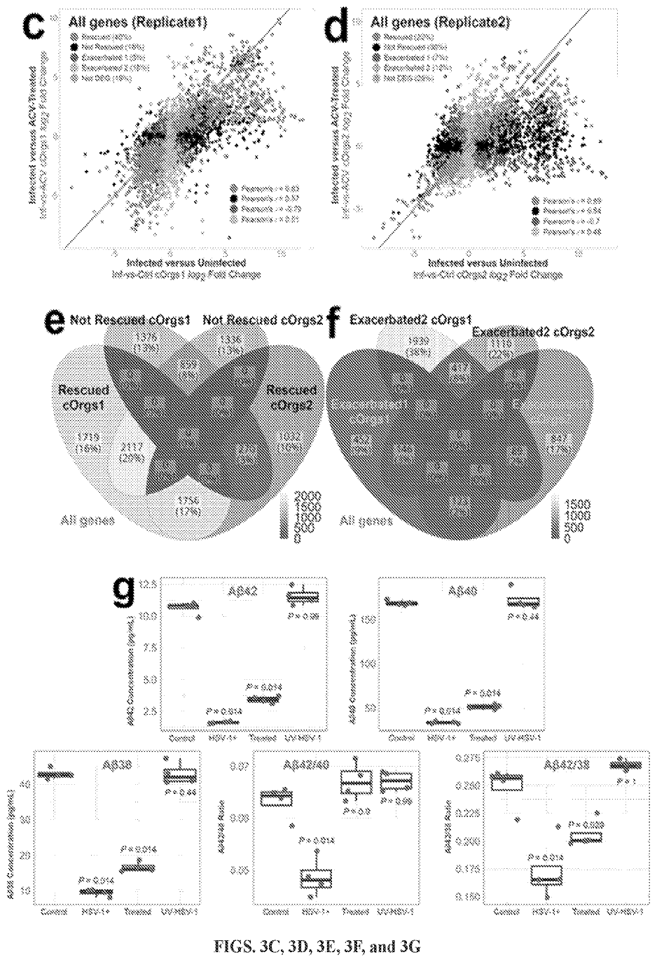

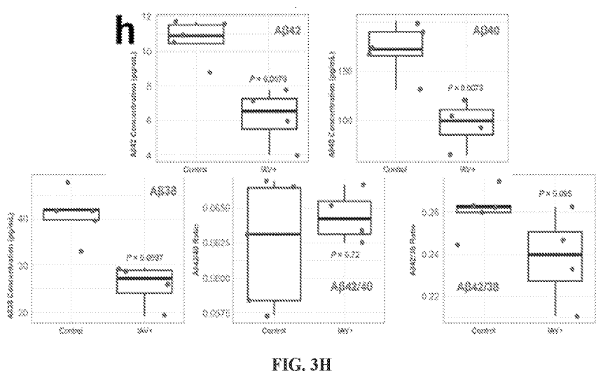

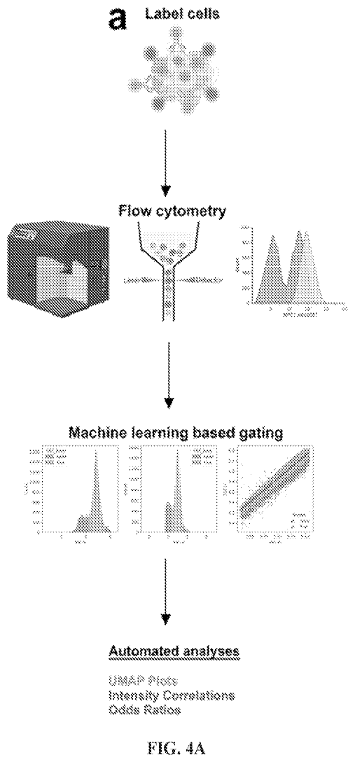

Both sets of DEGs in Inf-vs-Ctrl cOrgs1 and Inf-vs-Ctrl cOrgs2 were enriched for AD-associated genes (P=0.039 and P=5.1×10⁻³ respectively; FIG. 3A, Table 6). No enrichment was observed for the DEGs in Inf-vs-Ctrl cOrgs1 and Inf-vs-Ctrl cOrgs2 with genes associated with the other common diseases, showing that HSV-1 infection in cOrgs is preferentially perturbing the expression of AD-associated genes and gene networks. Similarly, the DEGs in HSV-1 infected cOrg cells versus cOrg cells with UV-inactivated HSV-1 (Inf-vs-UV cOrgs2) were enriched for AD-associated genes (P=0.015), indicating that the transcriptomics of cOrgs with UV-inactivated virus were largely similar to uninfected cOrgs.

It was evaluated if the DEGs in infected versus uninfected hiPSCs were enriched for AD-associated genes, but enrichment was not observed (P=0.67; FIG. 3A). GSEA was conducted using a published list of DEGs identified from HSV-1 infected versus uninfected SH-SY5Y-differentiated neurons, but any enrichment in AD-associated genes was not observed for the neuronal DEGs (P=0.38; FIG. 8D). The results indicate that several of these transcriptomic perturbations in AD-associated genes are likely to be driven by type 1 interferon response produced by cell types other than stem cells or neurons within the cOrg cells, such as glia cells.

DEGs from IAV-infected cOrgs were not enriched for AD-associated genes. To evaluate if IAV infection in cOrg cells can similarly lead to an enrichment in AD-associated genes, GSEA was performed on DEGs identified from IAV-infected versus uninfected cOrg samples. However, an enrichment for AD-associated genes was not observed (P-0.79; FIG. 3A). This showed that HSV-1 infection, but not IAV infection, could perturb the expression of many AD-associated transcripts in the cOrg cells, through the cytosolic DNA-sensing cGAS/STING/IRF3 pathways.

Dosage-dependent ACV treatment in HSV-1 infected cOrg samples rescued the expression in AD-associated genes. Next, it was evaluated if ACV treatment of HSV-1 infected cOrgs could rescue the transcriptomic perturbations in AD-associated genes observed. GSEA analyses of the discovery dataset (Inf-vs-ACV cOrgs1) showed an enrichment for AD-associated transcripts (P=0.023; FIG. 3B), indicating that ACV treatment could rescue HSV-1 induced transcriptomic perturbations in AD-associated genes. However, the replication dataset (Inf-vs-ACV cOrgs2) did not show an enrichment for AD-associated transcripts, indicating that ACV treatment in replication experiment did not rescue HSV-1 induced transcriptomic perturbations in AD-associated genes (P=0.53; FIG. 3B). As there were 14-20 times more HSV-1 viral transcripts detected in replication dataset compared to discovery dataset (FIG. 1D), it was contemplated that there were dosage-dependent differences in the amount of ACV that diffused into the cOrg cells across both experiments.

Using a previously reported annotation of viral transcripts, it was observed that there was a high correlation in the P-value ranks of differential expression for the leaky late viral transcripts (71) across both datasets (Spearman's p-0.63, P=1.6×10⁻³), but not for the true late viral transcripts (72) across both datasets (Spearman's p=0.2, P-0.47; FIGS. 9A and 9B and Table 7). The correlations in expression for leaky late and true late viral transcripts across both datasets were high (FIG. 9C, 9D, and 9E). These results are consistent with prior reports that 12 viral gene expression is perturbed by ACV treatment, but not γ₁, intermediate early or early viral gene expression.

GSEA was performed using the DEGs from ACV-vs-Ctrl cOrgs and observed an enrichment for AD-associated genes (P=0.028; FIG. 3B), which further confirmed that the ACV treatment was not as effective in inhibiting viral transcript expression in replication cOrgs2 experiments compared to discovery cOrgs1 experiments. These results indicate that ACV treatment can rescue transcriptomic perturbations in AD-associated DEGs due to HSV-1 infection in a dosage-dependent manner.

Gene ontology analyses showed that biological processes such as regulation of apoptosis, autophagy and mitochondrion organization were enriched among DEGs in both sets of Inf-vs-Ctrl cOrgs and Inf-vs-ACV cOrgs1 but not in Inf-vs-ACV cOrgs2 (Table 8). Pathways such as integrin signaling pathway, gonadotropin-releasing hormone receptor pathway and angiogenesis were enriched among DEGs in both sets of Inf-vs-Ctrl cOrgs and Inf-vs-ACV cOrgs1 but angiogenesis was not an enriched pathway in Inf-vs-ACV cOrgs2 (Table 9).

ACV treatment in HSV-1 infected cOrg cells led to enrichment of DEGs in genes associated with common autoimmune diseases. DEGs from some cOrg samples that showed enrichment in genes associated with common autoimmune diseases such as Type 1 diabetes (TID) and rheumatoid arthritis (RA). The DEGs from Inf-vs-ACV cOrgs1, Inf-vs-UV cOrgs2 and ACV-vs-UV cOrgs2 showed enrichment in genes associated with both TID and RA, whereas the DEGs from UV-vs-Ctrl cOrgs showed a similar enrichment in RA and attention-deficit hyperactivity disorder (ADHD) associated genes (FIGS. 3A and 3B). The enrichment of DEGs in TID, RA and ADHD associated genes were not observed in Inf-vs-Ctrl cOrgs. These observations intriguingly showed that there may be potential off-target transcriptomic dysregulation of genes associated with autoimmune diseases by ACV treatment, or capsid proteins found in UV-inactivated HSV-1, or both.

ACV treatment exacerbated the expression for 19-23% of human transcripts that were expressed in cOrg cells. To explore the contribution of off-target transcriptomic perturbations due to ACV treatment, two categories of off-target genes were defined. First, a “Exacerbated 1” group was defined as genes whose expression were made worse by ACV treatment. For instance, if HSV-1 infection in cOrgs down-regulated the human gene expression compared to uninfected cOrgs, but ACV treatment in infected cOrgs further down-regulated the gene expression compared to infected cOrgs, the gene would be classified into the Exacerbated 1 group. Next, a “Exacerbated 2” group was defined as genes where HSV-1 infection in cOrgs did not perturb the human gene expression but ACV treatment in infected cOrgs significantly perturbed the expression of the gene.

Collectively, both groups of exacerbated genes comprised of 19-23% among all genes that were expressed in cOrgs (FIGS. 3C and 3D). Unfortunately, exacerbated genes comprised of similarly high percentages of 22-23% among AD-associated GWAS genes (Table 10). On the other hand, the expression for most of the genes (22-40%) were rescued by ACV treatment and similarly, 26-41% of AD-associated genes were rescued by ACV treatment. These results reaffirm that ACV treatment can rescue most AD-associated transcriptomic perturbations due to HSV-1 infection, despite resulting in high percentages of exacerbated gene expression.

ACV treatment preferentially rescued AD-associated gene expression in cOrg samples. Using a 4-way Venn diagram, the overlaps between transcripts that were rescued or not rescued were explored across the cOrg replicates. The highest overlap in transcripts were rescued in cOrgs1 but were not rescued in cOrgs2 (FIG. 3E), which further supported the observations that ACV treatment was more effective in the discovery experiment than the replication experiment. The second highest overlap in transcripts were rescued in both cOrgs1 and cOrgs2, demonstrating that despite the dosage-dependent differences of ACV treatment in both sets of experiments, there were several transcripts in common that were rescued across both datasets.

Interestingly, the highest overlap in AD-associated transcripts were rescued in both cOrgs1 and cOrgs2 (FIG. 10). There was a modest excess of AD-associated transcripts that were rescued in both sets of replicate experiments, compared to the overall set of transcripts that were rescued in both sets of experiments (OR=1.33, P=0.062). These results showed that ACV treatment may have a preferential rescue for the expression of AD-associated genes in infected cOrg cells.

Similarly, there were modest excesses of RA-associated or TID-associated transcripts that were rescued in both cOrgs1 and cOrgs2, compared to the overall set of all transcripts that were rescued in both sets of experiments (OR=1.33 and 1.47, P=0.068 and 0.074 respectively). There were 7 other diseases or traits with modest enrichments of transcripts that were rescued in both cOrgs1 and cOrgs2, such as Parkinson's Disease (PD; OR=1.55, P=0.032), ADHD (OR=1.24, P=0.071), obsessive compulsive disorder (OCD; OR=1.96, P=0.0043), schizophrenia (SCZ; OR=1.21, P=0.069), inflammatory bowel disease (IBD; OR=1.29, P=0.088), height (OR=1.14, P=0.03) and waist-hip-ratio (WHR; OR=1.34, P=7.3×10⁻⁴). These results indicate that ACV treatment may preferentially rescue the expression of AD-associated genes and genes associated with several other common diseases or traits.

On the other hand, the 4-way Venn diagrams depicting both groups of exacerbated genes did not show high overlaps in any subsets that were common between both cOrg replicates (FIG. 3F and FIG. 11). This observation indicated that different sets of genes were transcriptionally exacerbated by each ACV treatment, unlike the rescued genes.

HSV-1 infection significantly lowered extracellular Aβ42/40 ratios detected in conditioned media but IAV infection did not affect extracellular Aβ42/40 ratios. To follow up on the observations that HSV-1 infection in cOrg cells led to an enrichment of AD-associated DEGs that could be rescued by ACV treatment, it was tested if HSV-1 infection could affect AD-associated neuropathology readouts and if ACV treatment could reverse AD-associated neuropathology caused by HSV-1. High-throughput ELISA assays were performed to evaluate the concentrations of extracellular Aβ42, Aβ40 and Aβ38 in conditioned media collected from cOrg cells that were uninfected, infected with HSV-1, infected and treated with ACV, as well as cOrg cells with UV-inactivated HSV-1 added. As a precaution, heat inactivation was performed on all conditioned media prior to conducting the ELISA assays.

Conditioned media from HSV-1 infected and ACV-treated cOrg cells had consistently lower concentrations of extracellular Aβ42, Aβ40 and Aβ38 compared to control conditioned media from uninfected cOrgs (Wilcoxon P=0.014; FIG. 3G, Table 11). There were significantly lower extracellular Aβ42/40 and Aβ42/38 ratios in conditioned media from HSV-1 infected cOrg cells (Wilcoxon P-0.014; FIG. 3G). ACV treatment restored extracellular Aβ42/40 ratios to similar ratios as detected in control conditioned media from uninfected cOrg cells (Wilcoxon P=0.9) but did not completely restore extracellular Aβ42/38 ratios (Wilcoxon P=0.029).

IAV infection in cOrg cells similarly led to decreased concentrations of extracellular Aβ42, Aβ40 and Aβ38 detected in the conditioned media, compared to control conditioned media from uninfected cOrg cells (Wilcoxon P=0.0097; FIG. 3H). However, extracellular Aβ42/40 and Aβ42/38 ratios in conditioned media from IAV infected cOrg cells did not differ significantly from ratios detected in control conditioned media from uninfected cOrg cells (Wilcoxon P=0.72 and 0.095 respectively; FIG. 3H). The ELISA assays were repeated using conditioned media that were subjected to UV-radiation and obtained similar results (Table 11, FIG. 12).

These results showed that HSV-1 infection in cOrg cells resulted in significantly lower extracellular Aβ42/40 ratios, but IAV infection in cOrg cells did not affect the extracellular Aβ42/40 ratios. In addition, ACV treatment of HSV-1 infected cOrg cells rescued the extracellular Aβ42/40 ratios to similar ratios as detected from uninfected cOrg cells.

HSV-1 expression in cOrg cells was highly positively correlated with intracellular expression of Aβ42 and phosphorylated Tau-Thr212 (pTau-212). Given the observations that HSV-1 infection in cOrg cells resulted in lower extracellular Aβ42/40 ratios detected in conditioned media, it was tested if HSV-1 infection in cOrg cells led to higher accumulation of cytosolic Aβ1-42 (Aβ42) or phosphorylated Tau on residue Thr212 (pTau-212). A 3-channel flow cytometry was used with 20,000-30,000 single cells for each condition, followed by machine learning based gating (FIG. 4A). A Zombie Violet dye was used to label dead cells, Alexa Fluor 647 or Allophycocyanin (APC) fluorophores conjugated antibodies were used to detect the expression of Aβ42, pTau-212 or cell type specific markers and the HSV-1 virus was tagged with green fluorescent protein (GFP). Uniform Manifold Approximation and Projection (UMAP) plots revealed that there were higher expressions of Aβ42 and pTau-212 in HSV-1 infected cOrg cells compared to uninfected cOrg cells or ACV-treated cOrg cells (FIGS. 4B and 4C, FIG. 11).

Interestingly, the adjusted Pearson's correlations in the intensities of Aβ42 and HSV-1 were high (r=0.73 and 0.7; FIG. 4D) and ACV treatment reduced the correlation in intensities of Aβ42 and HSV-1 intensities (r=0.24 and 0.23). Correlations in the intensities of pTau-212 and HSV-1 were similarly high (r=0.77 and 0.82) and ACV treatment similarly lowered the correlation in intensities of pTau-212 and HSV-1 (r=0.3 for both replicates). The correlations in intensities of HSV-1 and pTau-212 were consistently higher than the correlations in intensities of HSV-1 and Aβ42, which may indicate that hyper-phosphorylation of Tau occurred after Aβ42 aggregation, consistent with prior reports.

IAV expression in cOrg cells was not positively correlated with intracellular expression of Aβ42 and phosphorylated Tau-Thr212 (pTau-212). On the other hand, UMAP plots revealed that there were IAV infected cOrgs did not lead to higher expressions of Aβ42 and pTau-212 (FIGS. 4E and 4F). Neither were the adjusted Pearson's correlations in the intensities of Aβ42 and IAV positively correlated (r=−0.61 and −0.65; FIG. 4G). Similarly, the correlations in the intensities pTau-212 and IAV were not positively correlated (r=−0.48 and −0.6). These results support earlier observations that HSV-1 infection in cOrgs led to increased expression of Aβ42 and pTau-212 but IAV infection in cOrgs did not.

Expression of OLIG1+ oligodendrocyte progenitor cells and Iba1+ microglia were highly positively correlated with HSV-1. Correlation analyses of cell type marker intensities with HSV-1 pointed to OLIG1+ oligodendrocyte progenitor cells and Iba1+ microglia with the highest correlations (r=0.85 and 0.66 respectively; FIG. 4H). These results showed that intracellular Aβ42 and pTau-212 expression were tightly correlated with OLIG1 expression in HSV-1 infected cOrgs, consistent with prior reports that Aβ42-mediated activity led to changes in oligodendrocyte precursors in mouse and human brains and possibly due to Aβ-induced oligodendrocyte progenitor cell senescence.

Cell type enrichment analyses from RNA-seq data indicate an enrichment of astrocytes and depletion of excitatory neurons in HSV-1 infected cOrgs. Prior research reported observing reactive astrogliosis upon HSV-1 infection, however, other studies did not observe any change in the GFAP+reactive astrocyte population after HSV-1 infection. It was sought to evaluate differences in the proportions of cell types by performing cell type enrichment analyses using the Orgo-Seq method. Briefly, the Orgo-Seq method uses cell type specific transcripts identified from published single-cell RNA sequence (scRNA-seq) datasets to identify cell types enriched or depleted from RNA-seq data. A scRNA-seq dataset was utilized from human post-mortem brain samples as a reference dataset and found that astrocytes were computed to be enriched in Inf-vs-Ctrl, Inf-vs-ACV and Inf-vs-UV cOrgs (FIG. 5A), which indicates that HSV-1 infection may result in astrogliosis.

On the other hand, excitatory neurons were predicted to be depleted in Inf-vs-Ctrl, Inf-vs-ACV and Inf-vs-UV cOrgs (FIG. 5B), and similar reports of neuronal loss after HSV-1 infection had been previously observed. It was computed if inhibitory neurons, microglia, oligodendrocytes and oligodendrocyte progenitor cells may be enriched or depleted after HSV-1 infection, but found inconsistent results across cOrg datasets (FIGS. 5C, 5D, 5E, and 5F).

Flow cytometry results validated neuronal loss and astrogliosis observed from transcriptomics data, and provided insights into cellular mechanisms. To follow up on the cell type enrichment or depletion results observed from transcriptomics data, 3-channel flow cytometry experiments were performed with a Zombie Violet fluorophore to mark dead cells, an Alexa Fluor 647 or APC fluorophore conjugated to cell type specific markers, and the HSV-1 virus was tagged with GFP (FIGS. 13 and 14). The flow cytometry experiments were conducted on uninfected cOrgs, HSV-1 infected cOrgs and ACV-treated cOrgs, as well as uninfected cOrgs stained with mouse IgG isotype controls. In addition, 2 replicates of the flow cytometry experiments were repeated for each antibody marker and calculated odds ratios to quantify the results.

It was observed that decreased proportions of live neurons and neural progenitor cells: EOMES+ intermediate progenitors, Tuj1+ immature neurons, NeuN+ mature neurons and VMAT2+ dopaminergic neurons, which indicated that there was neuronal death in cOrgs due to HSV-1 infection (Infected live population in FIG. 5G). Live glia and glia progenitor cells were mostly increased in proportions, such as GFAP+ or GLAST+ astrocytes, Iba1+ or P2RY12+ microglia, OLIG1+ oligodendrocyte progenitor cells and 04+ oligodendrocytes. These results were consistent with reactive astrogliosis, activation of microglia and neuronal death previously observed in AD patients. ACV treatment of HSV-1 infected cOrgs reduced the depletion in neurons and neural progenitor cells and reduced the proportions of reactive glia cells (Treated live population in FIG. 5G). CD4+ monocytes or macrophages did not show a huge increase in proportions among infected live cells and the proportions were similar after ACV treatment, showing that the increased proportions of Iba1+ or P2RY12+ cells were likely to be driven by microglia and not monocytes nor macrophages.

One explanation for increased proportions of glia and glia progenitor cells in HSV-1 infected cOrgs is that there were decreased proportions of neurons and neural progenitor cells. Another possible explanation is that there was increased glia cell proliferation due to HSV-1 infection. It was observed that there were similar increased proportions of dead glia and glia progenitor cells (Infected dead population in FIG. 5H), showing that the more likely explanation for the results is due to neuronal and neural progenitor cell death. In addition, ACV treatment of HSV-1 infected cOrgs was able to rescue cell death across most cell types (Treated dead population in FIG. 5H).

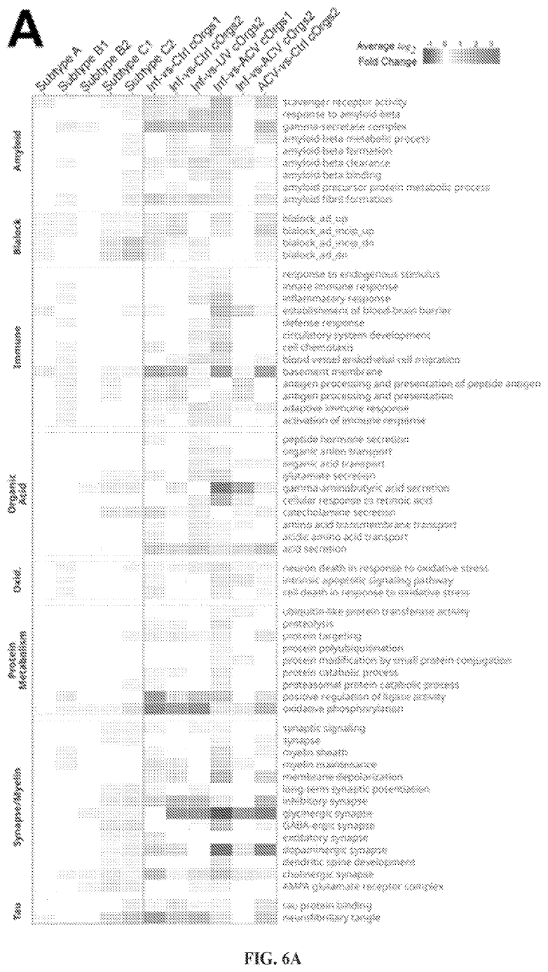

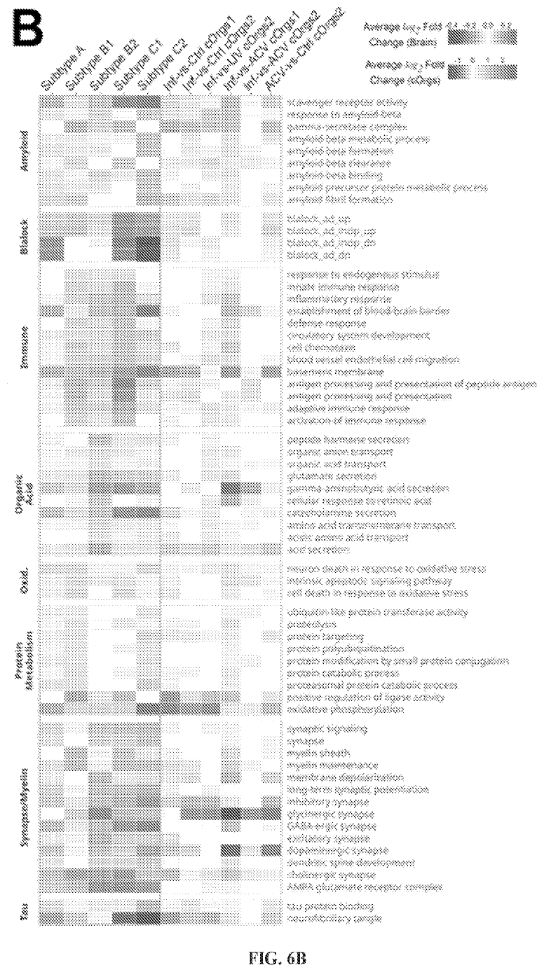

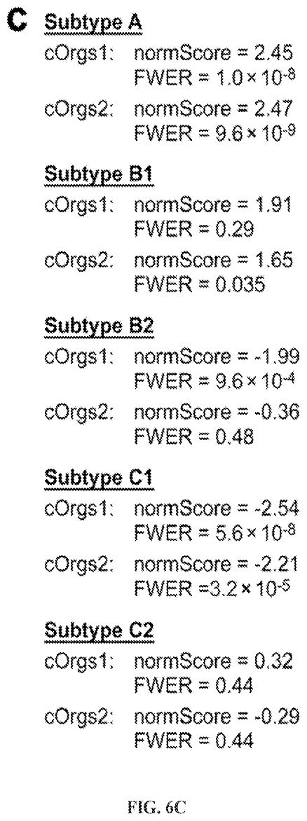

Analyses on human post-mortem brain RNA-seq revealed that 25-31% of patients with late-onset AD have transcriptomic signatures similar to HSV-1 infected cOrgs. A previous study used RNA-seq data generated using human post-mortem brain samples from patients with late-onset AD (LOAD) and performed molecular subtyping of the LOAD patients into 5 subtypes (A, B1, B2, C1 and C2). Globally, there were weaker fold changes observed in human post-mortem brains versus cOrgs (FIG. 6A). The fold changes observed were rescaled in human post-mortem brains and it was found that there were some pathways with high degrees of similarities in fold changes between the human post-mortem brains and cOrgs, such as Amyloid, Blalock, Immune, Synapse/Myelin and Tau (FIG. 6B).

A modified gene set enrichment analysis test was conducted using the DEGs from the cOrgs with the most informative DEGs from the post-mortem brains and identified a significant enrichment of the DEGs in cOrgs with subtype A (FIG. 6C and Table 9). Inf-vs-Ctrl cOrgs1, Inf-vs-Ctrl cOrgs2, Inf-vs-ACV cOrgs1 and ACV-vs-Ctrl cOrgs2 had significant positive associations with subtype A (FWER=1×10⁻⁸, 9.6×10⁻⁹, 6.4×10⁻¹⁰ and 4×10⁻⁷ respectively). Subtype A comprises of 25-31% of the LOAD patients from two cohorts, showing that a major fraction of LOAD patients may have an infectious etiology contributing to their AD pathogenesis and who may benefit from early ACV treatment.

### Discussion

Herein, it was tested whether HSV-1 infection in human cOrgs could result in in-vitro cellular and molecular readouts associated with common neurodegenerative, neuropsychiatric or autoimmune diseases. HSV-1 infected cOrgs led to AD-associated pathologies, such as transcriptomic perturbations that were enriched for AD-associated genes, decreased secreted Aβ42/40, increased intracellular Aβ42 and pTau-212 expression, neuronal death, astrogliosis, activation of microglia and increased proportions of oligodendrocyte progenitor cells. Immediate treatment with ACV on HSV-1 infected cOrgs rescued these cellular and molecular readouts.

However, it was also found that ACV treatment potentially result in off-target transcriptomic perturbations of genes associated with autoimmune diseases such as RA and TID. These off-target transcriptomic perturbations may not lead to cellular or physiological effects. Given the ongoing clinical trials for VCV as an AD therapeutics, these findings can aid in understanding the safety and efficacy of anti-HSV-1 drugs, or more broadly, anti-herpetic drugs, for AD. For instance, AD patients in clinical trials for VCV may be monitored for symptoms related to autoimmune diseases, so that side effects may be minimized.

In addition, recent evidence that the innate immune pathway cGAS/STING may be a major contributor to aging-related inflammation in neurodegenerative diseases such as AD. This study similarly pointed to neuroinflammation by the human innate immune pathways due to DNA viruses such as HSV-1 but not RNA viruses such as IAV, led to AD-associated cellular and molecular pathologies.

It was also found that post-mortem brain samples from 25-31% of LOAD patients have transcriptomic signatures that were similar to HSV-1 infected cOrgs, showing that a major fraction of AD patients benefit from anti-HSV-1 or more broadly, anti-herpetic treatment. This study can inform the selection of AD patient subtypes who may benefit from anti-viral therapeutics. The in-vitro screening platform using transcriptomics, Aβ42/40/38 ELISA assays and flow cytometry with human-derived cOrgs provides a medium-throughput or high-throughput system that can also be used to identify therapeutic targets and compounds that can increase the efficacy of cellular and molecular readouts associated with AD, while trying to reduce the off-target transcriptomic targets.

### Materials and Methods

### Standard Protocol Approval

Research performed on samples and data of human and viral origin was conducted according to protocols approved by the Institutional Review Board (IRB) and Institutional Biosafety Committee (IBC) of UMass Chan Medical School. HSV-1 and IAV are Biosafety Level (BSL) 2 pathogens.

Source of hiPSCs and Maintenance of hiPSCs

The control donor hiPSC line used (PGP1 iPSC) was a kind gift from Professor George Church's lab, which had been previously characterized and sequenced by the inventors. Flow cytometry (MACSquant VYB) was performed to confirm that >90% of the PGP1 hiPSCs are positive for TRA-1-60 (Novus Biologicals NB100-730F488). hiPSCs were plated using StemFlex media (Thermo Scientific A3349401) with 10 μM Y-27632 (Abcam ab120129) on 6-well plates coated with 0.5 mg of Matrigel Basement Membrane Matrix (Corning 354234) per plate, and subsequently dissociated using accutase (BioLegend 423201).

### HSV-1 Virus and ACV

The HSV-1 Kos strain used encoded a GFP-fused VP26 gene and was kindly provided by David Knipe's lab. A 200 mM stock solutions of ACV (Sigma PHR1254) was prepared by dissolving in UltraPure DNase/RNase-Free Distilled Water (Invitrogen 10977015) with equal molar concentrations of sodium hydroxide. Aliquots were made and kept frozen in −80 °C.

### Influena A Virus

The IAV virus (strain (A/Puerto Rico/8/1934 (H1N1))) used was purchased from Charles River Laboratories. Virus was propagated in SPF eggs in the allantoic cavity. Clarified allantoic fluid was concentrated and resuspended in Hepes-Saline and layered on a sucrose gradient. The interface band was diluted, pelleted and resuspended in a minimal volume of Hepes-Saline. Antigen was tested for protein concentrate of 2 mg of protein per mL using a Bio-Rad colorimetric protein assay. The final HA titer per 0.05 mL was 131,072 and the EID50 titer per mL was 10^{9.8}. A FITC-conjugated anti-Influenza A NP monoclonal antibody (ThermoFisher MA1-7322) at 1:20 dilution was used to detect viral proteins using flow cytometry.

### Cerebral Organoid Differentiation

A described protocol was used to generate spontaneously differentiated cOrgs from PGP1 hiPSCs. 900,000 cells were resuspended in 15 ml of StemFlex with 50 μM Y-27632 and seeded the cells across a 96-well ultra-low attachment plate (Corning CLS7007) to form embryoid bodies. After 5 days, each embryoid body was transferred to 24-well ultra-low attachment plates (Corning CLS3473) with 500 μL of neural induction media in each well. An additional 500 μL of neural induction media was added to each well on Day 8. On Day 10, each organoid was embedded in 40 μL of Matrigel (Corning 354234) on parafilm and incubated at 37 °C for 15 minutes before they were scraped into the wells containing 2 mL of differentiation media using a cell scraper. 1-2 mL of differentiation media with 10% penicillin streptomycin (ThermoFisher 15140122) per well was used to passage the organoids every 3-7 days, and the plates of organoids were placed on an orbital shaker at 90 rpm.

### Cerebral Organoid Dissociation

After 2-4 months of differentiation, 168-192 cOrgs were dissociated in each batch and cOrgs were washed twice in ice-cold 1×DPBS (ThermoFisher 14190144) for 10 minutes at 4 °C, followed by incubation in 500 μL of 0.25% Trypsin-EDTA (ThermoFisher 25200056) for 15 minutes at 37 °C and 300 rpm with repeated manual pipetting to dissociate clumps of cells. To remove cell debris, the cells were further filtered using a 30 μm MACS SmartStrainer (Miltenyi Biotec 130-098-458). Cell counts and viabilities were measured using an automated cell counter (NanoEnTek EVE). Cell viabilities ranged from 80% to 96%, and 7.8×10⁻⁶ to 4.1×10⁻⁷ live cells were obtained in total. Subsequently, the dissociated cells were passaged in differentiation media for at least a month to allow the cells to recover. The total time allowed for differentiation and recovery of the cells was recorded prior to infection as the age of the cOrg cells.

### HSV-1 and IAV Infection Experiments

The dissociated cells from 3-month cOrgs were infected using MOI of 1, 2, 4 or 10, and found that MOI of 2 resulted in >50% of infected cOrg cells that were positive for GFP expression, quantified using flow cytometry. A higher virus titer (MOI of 4) was used for hiPSCs. The number of cells needed for each experiment were counted and seeded at a density of 1×10⁶ cells/well of a 6-well tissue culture plate coated with 0.5 mg of Matrigel, using 2 mL/well of differentiation media with 10 μM Y-27632. On the following day, cells from one of the wells were counted using an automated cell counter to calculate the amount of virus needed.

Inoculation media containing 1×DPBS (ThermoFisher 14190144) with 0.5% FBS (ThermoFisher 10082147) was prepared, and aliquots of HSV-1 and ACV were kept on ice. Cells in each well were washed with 2 mL of 1×DPBS. 1 mL of inoculation media was added to each well of uninfected control cells, 1 mL of inoculation media with HSV-1 was added to each well of infected cells, and 1 mL of inoculation media with HSV-1 and 200 μM ACV was added to each well of treated cells. The cells were placed back into an incubator at 37 °C for an hour, and each well of cells were subsequently washed with 2 mL of 1×DPBS. 2 mL of differentiation media was added to each well of uninfected or infected cells, and 2 mL of differentiation media with 200 μM ACV was added to each well of treated cells. The cells were placed back into an incubator at 37 °C for 23 hours. The cells were inspected under a fluorescence microscope (Logos Biosystems CELENA S Digital Imaging System) to visually verify the presence of GFP in the infected and treated cells. A similar protocol was used for IAV infections, except that MOI of 3 was used with a 47-hour post-inoculation incubation.

### RNA Extraction and Sequencing

Cells were fixed using 4% paraformaldehyde (PFA; Fisher Scientific 50-980-487). RNA was extracted from the fixed cells using the PureLink FFPE RNA Isolation Kit (ThermoFisher K156002), and treated with Ambion DNase I (ThermoFisher AM2222) to digest genomic DNA according to the manufacturers' protocols. Total RNA was shipped overnight on dry ice to Psomagen for quantification, followed by ribosomal RNA depletion and library preparation (Illumina TruSeq Stranded Total RNA Library Prep 20020596), followed by 151 bp paired-end sequencing with a total of 40 million reads on a NovaSeq6000. RNA sequence alignment and data processing

Quality control was performed with the FastQC tool, and adapter sequences and low quality base calls were trimmed from FASTQs with Trimmomatic v0.39. Trimmed FASTQs were aligned to a concatenated GRCh37 human reference genome, human alphaherpesvirus 1 Kos strain reference genome (GenBank: JQ673480.1) and influenza A strain A/Puerto Rico/8/1934 (H1N1) reference genome (NCBI BioProject PRJNA485481) using HISAT2 v2.2.1. SAMtools v1.9 was used to extract reads aligning to the coding sequences of the virus. Expression quantification and preprocessing was performed using StringTie2 v1.3.6.

The transcript list was filtered to include only protein coding transcripts and duplicate genes in the StringTie2 output were removed, resulting in a total of 19,163 unique genes. Two approaches were compared for alignment: firstly, all transcripts were aligned to a custom reference comprised of both human and HSV-1 reference sequences and secondly, all transcripts were aligned to human and HSV-1 reference sequences individually. Pearson's r was calculated between the raw gene expression using both alignment approaches and found high correlations between both approaches (1−r2≤1×10⁻⁴). As a quality control step, lack of HSV-1 viral transcript expression was checked in all control uninfected samples. Pearson's r were calculated using the fragments per kilobase of exon per million mapped fragments (FPKM) values between sample pairs.

### Differential Expression Analyses

Differential expression analyses using the DESeq2 v1.38.2 package in R. Genes were filtered using a cutoff of counts per million (CPM) ≥1 across at least 2 samples. Raw counts were normalized using DESeq2 TMM normalization and models subsequently generated on a categorical design of infection or treatment status. The adjusted P-values from the differential expression analyses used were Benjamini-Hochberg adjusted P-values calculated from the raw P-values. Volcano plots were generated in R with EnhancedVolcano v1.11.3 and heatmaps were generated in R with ggplot2.

Ratios of Human Transcripts that were Up-Regulated Versus Down-Regulated

The ratios were calculated as the numbers of human transcripts post-quality control that were up-regulated with positive log-fold changes divided by the numbers of human transcripts post-quality control that were down-regulated with negative log-fold changes. Fisher's Exact Test P-values were calculated, compared against a null where there are equal numbers of human transcripts with positive and negative log-fold changes. To account for multiple hypotheses testing, Bonferroni correction for P-values that were 0.1 was applied to obtain the FWER values.

Gene Lists from GWAS Catalog and Gene Set Enrichment Analyses

Gene lists associated with 21 common diseases were downloaded from the NHGRI-EBI GWAS catalog in April 2022. Gene set enrichment analyses were performed using GSEA v4.3.2 to determine the enrichment of DEGs ranked by absolute log 2 fold change in decreasing order with the GWAS-associated gene lists. Analyses were performed using 10,000 permutations and a weighted enrichment statistic.

### Definitions of Rescued, not Rescued, Exacerbated 1 and 2 Transcripts

Rescued transcripts were defined as transcripts that were significantly differentially expressed (P<0.05) in both the Inf-vs-Ctrl cOrgs and Inf-vs-ACV cOrgs datasets and log 2 fold changes in both datasets were in the same direction. For instance, a positive log₂ fold change in Inf-vs-Ctrl cOrgs indicated that HSV-1 infection led to increased expression of the transcript. Similarly, a positive log 2 fold change in Inf-vs-ACV cOrgs meant that ACV treatment of HSV-1 infected cOrgs lowered the expression of the transcript, with respect to the expression in infected cOrgs.

“Not rescued” transcripts were defined as transcripts that were significantly differentially expressed (P<0.05) in Inf-vs-Ctrl cOrgs but were not significantly differentially expressed (P>0.05) in Inf-vs-ACV cOrgs.

“Exacerbated 1” transcripts were defined as transcripts that were significantly differentially expressed (P<0.05) in both the Inf-vs-Ctrl cOrgs and Inf-vs-ACV cOrgs datasets but log 2 fold changes in both datasets were in the opposite direction. For instance, a positive log 2 fold change in Inf-vs-Ctrl cOrgs indicated that HSV-1 infection led to increased expression of the transcript. However, a negative log 2 fold change in Inf-vs-ACV cOrgs meant that ACV treatment of HSV-1 infected cOrgs further increased the expression of the transcript, with respect to the expression in infected cOrgs.

“Exacerbated 2” transcripts were defined as transcripts that were not significantly perturbed in Inf-vs-Ctrl cOrgs (P>0.05) but were significantly differentially expressed in Inf-vs-ACV cOrgs (P<0.05). This indicates that ACV treatment on HSV-1 infected cOrgs significantly perturbed the expression of these transcripts while the expression of these transcripts were not perturbed by HSV-1 infection in cOrgs, with respect to the expression in uninfected cOrgs.

### Scatter Plots to Represent Rescued, not Rescued, Exacerbated 1 and 2 Transcripts

Scatter plots showed the log 2 fold changes in the Inf-vs-Ctrl cOrgs versus the log₂ fold changes in Inf-vs-ACV cOrgs for all transcripts that were in common between both datasets. The percentages of transcripts in each category (rescued, not rescued, exacerbated 1, exacerbated 2 or not DEG) were calculated as the number of transcripts in each category divided by the total number of transcripts and multiplied by 100%. Pearson's r were calculated between the log 2 fold changes in Inf-vs-Ctrl cOrgs and Inf-vs-ACV cOrgs for the transcripts in each category.

### Enrichment of Disease-Associated Rescued or Exacerbated Transcripts

Fisher's Exact Test was used to calculate the enrichment of the number of disease-associated transcripts in a group (such as the transcripts that were rescued in both cOrgs1 and cOrgs2) over the number of disease-associated transcripts that were not in the group, compared to (the total number of transcripts in the group minus the number of disease-associated transcripts in the group) over (the total number of transcripts that were not in the group minus the number of disease-associated transcripts that were not in the group).

### ELISA Assays and Analyses

After inoculation, 1 mL of fresh cOrg differentiation media was dispensed into each well and cells were incubated for 48 hours. Conditioned media from each well were collected into 2 tubes with 500 μL of media in each tube. Each set of conditions (uninfected cOrg controls for HSV-1, HSV-1 infected cOrgs, ACV-treated cOrgs, uninfected cOrg controls for IAV and IAV infected cOrgs) had 4-5 experimental replicates. One set of conditioned media was heat inactivated at 65 °C for an hour and the second set of conditioned media was UV-inactivated. Extracellular Aβ42/40/38 levels were measured using the V-PLEX Plus AB Peptide Panel 1 (6E10) Kit (MSD K15200G-1), with controls diluted in the given buffer. The Aβ peptide values were batched normalized to control in the ELISA kit. The concentrations of each AB peptide and ratios of Aβ peptides were visualized using ggplot in R. 1-tailed Wilcoxon ranked sum tests were used to evaluate the samples of interest versus matched uninfected controls.

### Antibody Labeling and Flow Cytometry Experiments

Cells were washed with 1×DPBS and pelleted into 1.5 mL microcentrifuge tubes. Each cell pellet was resuspended in 100 μL of 1×DPBS with 1:500 Zombie Violet (BioLegend 423113) and incubated for 20 mins at room temperature in the dark. The cell pellets were then washed with 500 μL of cell staining buffer (BioLegend 420201) and resuspended in either 100 μL of 4% PFA or 250 μL of Cytofix/Cytoperm fixation and permeabilization buffer (BD Biosciences 554714), and incubated at 4 °C for 20 mins at 300 rpm. For intracellular antibodies, the cells were then washed once with 500 μL of permeabilization solution. For cell surface antibodies, the cells were then washed with 500 μL of cell staining buffer, then resuspended in 105 μL of cell staining buffer with 1:21 Human TruStain FcX (BioLegend 422301), followed by incubation at 4 °C for 20 mins at 300 rpm. Each cell pellet was then resuspended in 50 μL of the respective buffer with 1:21 Human TruStain FcX and the antibody of interest, followed by incubation at 4 °C for 60 mins at 300 rpm.

The antibodies used were: Alexa Fluor 647-conjugated Aβ1-42 (Bioss Antibodies bs-0107R-BF647) at a 1:50 dilution, Alexa Fluor 647-conjugated Tau (Thr212) (Bioss Antibodies bs-5420R-BF647) at a 1:50 dilution, Alexa Fluor 647-conjugated TRA-1-60 (BioLegend 330605) at a 1:20 dilution, Alexa Fluor 647-conjugated Nestin (Novus Biologicals IC1259R-100UG) at a 1:50 dilution, Alexa Fluor 647-conjugated EOMES (Novus Biologicals IC6166R-100UG) at a 1:50 dilution, APC-conjugated TUJ1 (Biolegend 801219) at a 1:20 dilution, Alexa Fluor 647-conjugated NeuN (Novus Biologicals NBP1-92693AF647) at a 1:50 dilution, Alexa Fluor 647-conjugated Iba1 (Novus Biologicals 603102) at a 1:50 dilution, APC-conjugated P2RY12 (BioLegend 392113) at a 1:20 dilution, Alexa Fluor 647-conjugated CD4 (Biolegend 300520) at a 1:20 dilution, Alexa Fluor 647-conjugated GFAP (Novus Biologicals NBP2-33184AF647) at a 1:50 dilution, APC-conjugated GLAST (Miltenyi 130-123-555) at a 1:50 dilution, Alexa Fluor 647-conjugated OLIG1 (Bioss bs-8548R-A647) at a 1:50 dilution, Alexa Fluor 647-conjugated 04 (R&D Systems FAB1326R) at a 1:20 dilution and Alexa Fluor 647-conjugated 01 (R&D Systems FAB1327R) at a 1:20 dilution.

Isotype controls used the following antibodies: Alexa Fluor 647-conjugated rabbit IgG (Bioss Antibodies bs-0295P-A647) at a 1:50 dilution, Alexa Fluor 647-conjugated mouse IgG1 kappa (Novus Biologicals IC002R) at a 1:50 dilution, Alexa Fluor 647-conjugated mouse IgG2A kappa (Biolegend 400234F2) at a 1:20 dilution and Alexa Fluor 647-conjugated mouse IgG2B kappa (Novus Biologicals IC0041RF3) at a 1:20 dilution.

After antibody labeling, each cell pellet was washed twice with 500 μL of Cell Staining Buffer, resuspended in 300 μL of Cell Staining Buffer and transferred to a 5 mL glass tube. ABD Biosciences FACSCelesta Cell Analyzer was used at the UMass Chan FACS core facility for flow cytometry experiments. Using the FACSDiva software, a “usable cell” gate was drawn on the FSC-A/SSC-A plot to minimize the collection of debris, and 20,000-30,000 cells within the useable cell population were analyzed.

### Machine Learning Based Gating for Flow Cytometry Data

Flow cytometry data was exported from FlowJo v10 as a .csv file with the per cell values for the Alexa Fluor 488-A, Alexa Fluor 647-A, Pacific Blue-A, FSC-A, FSC-H, SSC-A, and SSC-H channel intensities. Infinite and zero values were dropped from further analyses. FSC/SSC channels were then log 10-transformed. Next, viable cells were identified by setting minimum and maximum cutoffs for the FSC-A and SSC-A values. By default, viable cells were defined as cells with intensities between the 5th and 95th percentiles in the uninfected samples.

The FSC-A and SSC-A intensities were first tested for normality based on D'Agostino and Pearson's test as implemented in the python Scipy package. If the distribution passed the normality test (P>0), the data between the 5th and 95th percentiles were kept. If the distribution failed the normality test (P=0), the data was fit to a Bayesian Gaussian Mixture model with the number of components=2. If the model divided the data into two distinct bimodal distributions, the data between the 5th and 95th percentile of the distribution with higher intensities was kept. The cutoffs determined from the uninfected samples were then applied to the infected and ACV-treated data from the same experiment.

Next, singlets were tested by fitting a linear regression model using FSC-A versus FSC-H intensities for each condition (uninfected, infected or treated). The residuals and cells with residuals greater than the 99th percentile were considered to be doublets and were removed from further analyses.

To gate the Pacific Blue-A, Alexa Fluor 488-A and Alexa Fluor 647-A intensities, the number of model distributions were first calculated in the infected samples by using a kernel density plot to identify the number of maxima. A local maximum was defined as having an intensity value of at least 15% of the global maximum of the kernel density plot. If the distribution was a unimodal distribution, the cutoff was set at the 85th percentile. If the distribution was a multi-modal distribution, the data was then fitted to a Bayesian Gaussian

Mixture model with the number of components=the number of maxima. The gates determined from the infected samples were then applied to the uninfected and ACV-treated data from the same experiment.

### UMAP Plots

UMAP was performed using the Python package umap-learn. For each flow experiment, the initial UMAP values for Alexa Fluor 488-A, Alexa Fluor 647-A, and Pacific Blue-A were normalized between 0 and 1 across all conditions (uninfected, infected and ACV-treated). The data for these three channels were then fitted to a UMAP model initialized with the number of components=2 to re-calculate UMAP1 and UMAP2 values.

### Adjusted Correlations in Alexa Fluor 488 and Alexa Fluor 647 Intensities

First, a linear correction on the Alexa Fluor 488 and Alexa Fluor 647 intensities was calculated from the uninfected samples. This enabled identification of the background correlation in the intensities. The β coefficient was obtained from the linear regression model and corrected the Alexa Fluor 647 intensities (y_{adjusted}) for the infected and ACV-treated correlations as follows:

y_{adjusted}=y_{unadjusted}−βx Equation (1)

where y_{unadjusted} were the unadjusted Alexa Fluor 647 intensities in the infected and ACV-treated data and x were the Alexa Fluor 488 intensities in the infected and ACV-treated data.
Odds Ratio Calculations from Flow Cytometry Data

Using the machine learning based gating, the cells were grouped into 4 quadrants: Q1 where the cells were negative for HSV-1 (GFP) and positive for the marker of interest (Alexa Fluor 647 or APC), Q2 where the cells were positive for both HSV-1 and the marker of interest, Q3 where the cells were positive for HSV-1 and negative for the marker of interest, and Q4 where the cells were negative for both HSV-1 and the marker of interest. ORs and Fisher's Exact Test P-values were calculated in R, where:

$$Infected\_OR ( {Marker}^{+} in HSV {1}^{+} cells ) = \frac{{Marker}_{Infected}^{+} / {Marker}_{Infected}^{-}}{{Marker}_{Uninfected}^{+} / {Marker}_{Uninfected}^{-}} \qquad Equation ( 2 )$$

$$ACV\_OR ( {Marker}^{+} in HSV {1}^{-} cells ) = \frac{{Marker}_{Treated}^{+} / {Marker}_{Treated}^{-}}{{Marker}_{Uninfected}^{+} / {Marker}_{Uninfected}^{-}} \qquad Equation ( 3 )$$

Enrichment Analyses of AD Subtype and cOrg DEGs

The results from an earlier study were used that identified subtypes using LOAD post-mortem brain samples in the Mount Sinai/JJ Peters VA Medical Center Brain Bank (MSBB-AD) and Religious Orders Study-Memory and Aging Project (ROSMAP) cohorts (See Neff, R. A. et al. Molecular subtyping of Alzheimer's disease using RNA sequencing data reveals novel mechanisms and targets. Sci Adv 7, doi: 10.1126/sciadv.abb5398 (2021)). The cumulative overlap fraction of DEGs was calculated for each of the 5 subtypes, which was defined as

$$\frac{Number of DEGs in common across at least 2 subtypes}{Total number of DEGs} .$$

The rates of change in the cumulative overlap fractions was further calculated for each subtype to identify that the points of inflection across all subtypes converge at ~200 DEGs, indicating that the top 200 DEGs across the 5 subtypes were likely to be most informative for subtype distinction.

Gene set enrichment analyses of the cOrg DEGs ranked by decreasing order of absolute log₂ fold changes and the ranked DEGs from each subtype were calculated using connectivity scores. Enrichment scores were permuted 10,000 times to determine the significance of the observed scores.

### Example 2: Acyclovir Treatment of HSV-1 Infection in Human Cerebral Organoids Rescues Alzheimer's Disease-Associated Transcriptomic and Cellular Perturbations

Innately differentiated microglia in cOrgs for interferon response. Microglia, the resident immune cells in the central nervous system, are differentiated from the mesodermal lineage in-vitro. As such, it was widely thought that cOrgs did not contain differentiated microglia. However, an earlier study had found and characterized CD11b+ microglia that differentiated innately within these cOrgs. A strong transcriptional activation of the innate immune response had been observed in the RNA-seq datasets from HSV-1 infection in dissociated cOrgs and also reported previously from HSV-1 infection in 3D cOrgs (See, Rybak-Wolf, A. et al. Modelling viral encephalitis caused by herpes simplex virus 1 infection in cerebral organoids. Nat Microbiol 8, 1252-1266, doi: 10.1038/s41564-023-01405-y (2023)). This innate immune response is produced by microglia or astrocytes within the cOrgs, although a small fraction (~3%) of neurons had been reported to produce type I interferon response. Neurons had also been reported to be activated directly by microbes to release neuropeptides and modulate the innate immune response (See, Ruhl, C. R. et al. Mycobacterium tuberculosis Sulfolipid-1 Activates Nociceptive Neurons and Induces Cough. Cell 181, 293-305 e211, doi: 10.1016/j.cell.2020.02.026 (2020) and Chiu, I. M. et al. Bacteria activate sensory neurons that modulate pain and inflammation. Nature 501, 52-57, doi: 10.1038/nature12479 (2013)). As such, the interferon response observed from the data may be driven by glia cells (microglia, astrocytes) and/or neurons that differentiated innately within these cOrgs.

Comparisons of cOrg samples with different ages may potentially point to cell type specific perturbations due to HSV-1. The correlations between uninfected cOrg samples were compared with different ages and observed high pairwise correlations between uninfected 4-month and 5-month cOrg samples (Pearson's r=0.91-0.92), although these correlations were significantly lower than the correlations of r>0.99 between uninfected cOrg samples of the same age (Wilcoxon P=4×10⁴). The lower pairwise correlations between 4-month and 5-month uninfected cOrg samples might have arisen from differences in cell type proportions or differences in temporal expression of transcripts between 4-month and 5-month cOrg samples. Significantly lower correlations between infected 4-month and 5-month cOrg samples (Pearson's r=0.86 to 0.9), versus uninfected 4-month and 5-month cOrg samples (Wilcoxon P=4.1×10⁻⁵) were observed, showing that HSV-1 is preferentially perturbing cell type specific transcripts, such as transcripts found in differentiated cell types (e.g. neurons and glia cells) that may differ in proportions between 4-month and 5-month cOrg samples. However, the 5-month cOrg samples were not aged from the 4-month cOrgs to study differences in transcript expression due to aging of the same batch of cOrg samples. Instead, independent batches of cOrg samples were differentiated with the goal of evaluating the reproducibility of HSV-1 infection and ACV-treatment in cOrgs.

Additional evidence for resistance to viral infections in hiPSCs. Comparing the infected versus uninfected cOrgs, it was observed that 62.7% of the human genes had significant differential expression (adjusted P<0.05) in the 4-month cOrg samples and 59.3% of the genes had significant differential expression in the 5-month cOrg samples. Similarly, 57.2% of the genes had significant differential expression in the cOrg samples with live HSV-1 versus UV-inactivated HSV-1. Comparing the infected versus ACV-treated cOrg samples, it was observed that 62.3% of the genes had significant differential expression in the 3-month cOrg samples and 40.3% of the genes had significant differential expression in the 5-month cOrg samples. In contrast, only 11.5% of the genes had significant differential expression in infected versus uninfected hiPSCs and 0.55% of the genes had significant differential expression in infected versus ACV-treated hiPSCs. These results again support the observations that HSV-1 infection in hiPSCs did not perturb as many human transcripts as HSV-1 infection in cOrg samples.

Transcriptomic rescue of AD-associated transcript expression was correlated with strength of down-regulation of HSV-1 true late genes mediated by ACV treatment. Given that true late (72) viral transcript expression, but not leaky late (11) viral transcript expression, had been reported to be perturbed by ACV treatment, the γ₁ versus γ₂ DEGs was compared in ACV-treated cOrgs versus infected cOrgs (FIG. 9 and Table 7). There were more γ₁ genes that were up-regulated versus down-regulated in both ACV-vs-Inf cOrg datasets, while there were more γ₂ genes that were down-regulated versus up-regulated in both datasets.

Strong correlations were observed in the differential expression of γ₁ and γ₂ genes from both sets of ACV-vs-Inf cOrgs, although differential expression of γ₁ genes was more strongly correlated (Spearman's p=0.9, P=3.4×10⁻⁶) than differential expression of γ₂ genes (Spearman's p=0.83, P=2.2×10⁻⁴). There was also a strong correlation in the P-value ranks of the differential expression in γ₁ genes was not observed across both datasets (Spearman's p=0.63, P=1.6×10⁻³), but a strong correlation in the P-value ranks of the differential expression in 12 genes across both datasets (Spearman's p=0.2, P=0.47).

There were significantly more up-regulated γ₁ genes in the replication dataset ACV-vs-Inf cOrgs2 (1-tailed binomial P=9.6×10⁻³), compared to the discovery dataset ACV-vs-Inf cOrgs1 (1-tailed binomial P=0.072). On the other hand, there were significantly more down-regulated γ₂ genes in the discovery dataset ACV-vs-Inf cOrgs1 (1-tailed binomial P=0.033), compared to the replication dataset ACV-vs-Inf cOrgs2 (1-tailed binomial P=0.21). The γ₁ and 72 genes in ACV-vs-Inf hiPSCs were not disproportionately up-regulated or down-regulated (1-tailed binomial P=0.13 and 0.5 respectively). Taken together, these results show that the higher abundance of viral transcripts (14-20 times more) in the replication dataset ACV-vs-Inf cOrgs2 compared to the discovery dataset ACV-vs-Inf cOrgs1 is due to lower amounts of ACV diffused into the cOrgs in the replication dataset and that is associated with weaker down-regulation of HSV-1 12 genes mediated by ACV treatment in the replication dataset.

Additional analyses of human transcripts that were up-regulated versus down-regulated. Unlike Inf-vs-UV cOrgs2 where it was observed an enrichment for AD-associated genes (P=0.015; FIG. 2A), an enrichment in ACV-vs-UV cOrgs2 (P=0.13; FIG. 2B) was not observed, indicating that there was rescue in HSV-1 induced transcriptomic perturbations despite the less effective ACV treatment. Both sets of Inf-vs-ACV cOrgs1 and Inf-vs-ACV cOrgs2 also showed balanced ratios of human transcripts that were up-regulated versus down-regulated (FWER-0.42 and 0.36 respectively; FIG. 7N), unlike the ratios from the Inf-vs-Ctrl cOrgs where there were more up-regulated human transcripts (FWER=1.2×10⁻⁴ and 5.9×10⁻⁴). Volcano plots showed similar differential expression of HSV-1 viral transcripts in both sets of Inf-vs-ACV cOrgs (FIG. 8).

Use of multiple cell type specific antibody markers to generate hypotheses into cellular mechanisms. The use of multiple antibody markers in the flow cytometry experiments provide additional insights into the cellular mechanisms affected by HSV-1 infection in cOrgs. Given that there were decreased proportions of neural progenitors, intermediate progenitors, immature neurons and mature neurons, the likely mechanism is neuronal cell death, as opposed to decreased neuronal differentiation. Similarly, increased proportions of GLAST+ astrocytes and P2RY12+ microglia were observed in HSV-1 infected cOrgs, showing reactive astrogliosis and activation of microglia. The lack of a significantly increased proportion of CD4+ cells in HSV-1 infected cOrgs show that the P2RY12+ cells are likely to be microglia, rather than monocytes or macrophages. There was a huge increase in the proportions of OLIG1+ cells and modestly increased proportions of 04+ and 01+ cells in HSV-1 infected cOrgs, showing that oligodendrocyte progenitor cells increased, rather than mature oligodendrocytes.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
Gene Set 1. AD-associated genes that are involved in neuroinflammation.
Gene Set 1

| | | | | | |
|---|---|---|---|---|---|
| AARS2 | CELF2 | F2RL1 | ITSN2 | PPP4R3A | SPPL2A |
| ABCA1 | CENPM | FAM83E | KCNN2 | PRKD3 | SQSTM1 |
| ALDH1A2 | COL18A1 | FAT1 | LAMA1 | PRRC2C | SUDS3 |
| ARIH1 | DDAH1 | FERMT2 | LINGO2 | RAPSN | TGFB2 |
| BMP1 | DDX18 | FOXN3 | MOBP | RBMS3 | TMEM106B |
| BMPER | DST | G3BP1 | MRC1 | RPTOR | TULP4 |
| CAMK4 | DVL2 | GPBP1 | NKAIN3 | SERPINE1 | WAC |
| CD2AP | ERBB3 | HDAC9 | PAK2 | SETD7 | WDR70 |
| CDKAL1 | ERBB4 | HRK | PEX6 | SORD | YAP1 |
| CELF1 | F2R | HYI | PLEC | SPATS1 | |

TABLE 2
Gene Set 2. Additional AD-associated genes that are
involved in neuroinflammation.
Gene Set 2

| | | | |
|---|---|---|---|
| ACER3 | CLEC3B | NECTIN2 | SYNJ1 |
| AFF1 | DCHS2 | NEPRO | TMED9 |
| AHNAK | DMXL1 | OSBPL6 | TNRC6A |
| AP4M1 | DOCK4 | PFDN1 | TOP1 |
| APOE | EPC2 | POLN | TSKU |
| BCKDK | FAM163A | PSMC3 | TSPAN14 |
| BIN1 | FNIP1 | RNF6 | TST |
| CCDC85C | FOXE1 | SLC35C1 | TXNL1 |
| CDC5L | HSPG2 | SP6 | WNT7B |
| CDR2L | MED12L | STRADA | |

TABLE 3
Off-Target Gene Set.
Gene Set 3

| | | | | |
|---|---|---|---|---|
| ABCA7 | CCDC134 | EYA4 | MTHFD1L | RAPGEF6 |
| ADAM10 | CCDC88C | FOXN2 | MTSS2 | SCARA3 |
| ADAMTS1 | CDON | FZD6 | NCS1 | SGK1 |
| ADAMTS4 | CFAP20DC | GPC6 | NDUFAF6 | SLC4A8 |
| ADARB2 | CNN2 | HERC2 | NFIC | SPRED2 |
| ADCY8 | CRY2 | HSD17B12 | NME9 | SZT2 |
| ADGRF2 | CYB561 | INSIG1 | OARD1 | TNRC6C |
| AHCYL1 | CYCS | KCNN3 | OTOGL | TOMM40 |
| ALPK2 | DAPL1 | LIMS2 | PDE7B | UBXN11 |
| ANO4 | DDX25 | LIN28B | PICALM | VAT1L |
| AP2A2 | DSG2 | LIPG | PKNOX2 | ZCCHC10 |
| APP | ELMO1 | LRAT | PLEKHG1 | ZNF224 |
| ARVCF | EN2 | MAP2K5 | PMAIP1 | ZNF225 |
| BDH1 | EPOP | MAPK7 | PNPLA7 | ZNF292 |
| C1S | EXOC4 | MROH1 | RAB3D | ZNF652 |
| | | | | ZNF804A |

TABLE 4
Overall summary of the bulk RNA sequence datasets that
were generated, with the Pearson's correlations calculated
for samples according to their conditions and batches.

| Virus | Sample_ID | Age_of_Organoids | RIN |
|---|---|---|---|
| HSV-1 | Treated_Organoids1 | 3-months | 6.5 |
| HSV-1 | Treated_Organoids2 | 3-months | 6.7 |
| HSV-1 | Treated_Organoids3 | 3-months | 6.2 |
| HSV-1 | Infected_Organoids4 | 3-months | 8 |
| HSV-1 | Infected_Organoids5 | 3-months | 9.1 |
| HSV-1 | Infected_Organoids6 | 3-months | 8.1 |
| HSV-1 | Uninfected_Organoids_1 | 4-months | 8.3 |
| HSV-1 | Uninfected_Organoids2 | 4-months | 9 |
| HSV-1 | Uninfected_Organoids3 | 4-months | 8.5 |
| HSV-1 | Infected_Organoids1 | 4-months | 6.8 |
| HSV-1 | Infected_Organoids2 | 4-months | 6.9 |
| HSV-1 | Infected_Organoids3 | 4-months | 7.3 |
| HSV-1 | Uninfected_Organoids4 | 5-months | 9.5 |
| HSV-1 | Uninfected_Organoids5 | 5-months | 9.6 |
| HSV-1 | Uninfected_Organoids6 | 5-months | 9.6 |
| HSV-1 | Infected_Organoids7 | 5-months | 9.7 |
| HSV-1 | Infected_Organoids8 | 5-months | 9.6 |
| HSV-1 | Infected_Organoids9 | 5-months | 9.5 |
| HSV-1 | Treated_Organoids4 | 5-months | 9.6 |
| HSV-1 | Treated_Organoids5 | 5-months | 9.8 |
| HSV-1 | Treated_Organoids6 | 5-months | 9.6 |
| HSV-1 | UV-HSV-1_Organoids1 | 5-months | 9.5 |
| HSV-1 | UV-HSV-1_Organoids2 | 5-months | 9.3 |
| HSV-1 | UV-HSV-1_Organoids3 | 5-months | 9.4 |
| HSV-1 | Uninfected_hiPSCs1 | None | 9.5 |
| HSV-1 | Uninfected_hiPSCs2 | None | 9.4 |
| HSV-1 | Uninfected_hiPSCs3 | None | 9 |
| HSV-1 | Infected_hiPSCs1 | None | 9.3 |
| HSV-1 | Infected_hiPSCs2 | None | 9.5 |
| HSV-1 | Infected_hiPSCs3 | None | 6.1 |
| HSV-1 | Treated_hiPSCs1 | None | 9.1 |
| HSV-1 | Treated_hiPSCs2 | None | 9.5 |
| HSV-1 | Treated_hiPSCs3 | None | 9.7 |
| IAV | IAV_Uninfected_Organoids_1 | 7-months | 8.9 |
| IAV | IAV_Uninfected_Organoids2 | 7-months | 8.5 |
| IAV | IAV_Uninfected_Organoids3 | 7-months | 8.6 |
| IAV | IAV_Infected_Organoids1 | 7-months | 8.7 |
| IAV | IAV_Infected_Organoids2 | 7-months | 8.3 |
| IAV | IAV_Infected_Organoids3 | 7-months | 8.5 |

TABLES 5A and 5B
Numbers of human and HSV-1 differentially expressed
genes with positive or negative log2(Fold Change).

| | Inf-vs-Ctrl | Inf-vs-Ctrl | Inf-vs-UV | Inf-vs-ACV | Inf-vs-ACV |
|---|---|---|---|---|---|
| A. | cOrgs1 | cOrgs2 | cOrgs2 | cOrgs1 | cOrgs2 |
| Total number of human genes | 14672 | 14333 | 14245 | 14315 | 14510 |
| Number of differential | 9205 | 8498 | 8155 | 8919 | 5849 |
| expressed genes (DEGs) | | | | | |
| Number of DEGs with | 4898 | 4510 | 4472 | 4514 | 2974 |
| log2FoldChange > 0 | | | | | |
| Number of DEGs with | 4307 | 3988 | 3683 | 4405 | 2875 |
| log2FoldChange < 0 | | | | | |
| Odds Ratio | 1.14 | 1.13 | 1.21 | 1.02 | 1.03 |
| Fisher's Exact Test | 1.36E−05 | 6.58E−05 | 6.47E−10 | 4.20E−01 | 3.60E−01 |
| P | | | | | |
| 95% lower | 1.07 | 1.06 | 1.14 | 0.97 | 0.96 |
| confidence interval | | | | | |
| 95% upper | 1.21 | 1.20 | 1.29 | 1.09 | 1.11 |
| confidence interval | | | | | |

| | ACV-vs-Ctrl | ACV-vs-UV | Inf-vs-Ctrl | Inf-vs-ACV |
|---|---|---|---|---|
| A. | cOrgs2 | cOrgs2 | hiPSCs | hiPSCs |
| Total number of human genes | 13884 | 13770 | 14714 | 13759 |
| Number of | 9869 | 9124 | 1581 | 76 |
| differential expressed | | | | |
| genes (DEGs) | | | | |
| Number of DEGs with | 5033 | 4750 | 904 | 69 |
| log2FoldChange > 0 | | | | |
| Number of DEGs with | 4836 | 4374 | 677 | 7 |
| log2FoldChange < 0 | | | | |
| Odds Ratio | 1.04 | 1.09 | 1.34 | 9.70 |
| Fisher's Exact Test P | 1.63E−01 | 5.62E−03 | 5.55E−05 | 3.73E−08 |
| 95% lower confidence | 0.98 | 1.02 | 1.16 | 3.81 |
| interval | | | | |
| 95% upper confidence | 1.10 | 1.15 | 1.54 | 28.31 |
| interval | | | | |

| | Inf-vs-ACV | Inf-vs-ACV | Inf-vs-ACV |
|---|---|---|---|
| B. | cOrgs1 | cOrgs2 | hiPSCs |
| Total number of viral | 74 | 74 | 74 |
| genes | | | |
| Number of | 59 | 68 | 37 |
| differential expressed | | | |
| genes (DEGs) | | | |
| Number of DEGs with | 30 | 34 | 17 |
| log2FoldChange > 0 | | | |
| Number of DEGs with | 29 | 34 | 20 |
| log2FoldChange < 0 | | | |
| OR | 1.03 | 1.00 | 0.85 |
| FET P | 1 | 1 | 0.82 |
| 95% lower CI | 0.47 | 0.48 | 0.31 |
| 95% upper CI | 2.27 | 2.07 | 2.35 |

TABLE 6
Nominal P-values from GSEA results for enrichment of differential gene expression results.

| Dataset | AD | ALS | MS | PD | ADHD | ASD | BD | MDD | OCD | SCZ | TS |
|---|---|---|---|---|---|---|---|---|---|---|---|
| Inf-vs-Ctrl cOrgs1 | 0.039 | 0.43 | 0.77 | 0.59 | 0.86 | 0.85 | 0.98 | 1 | 0.26 | 0.99 | 0.43 |
| Inf-vs-Ctrl cOrgs2 | 0.0051 | 0.68 | 0.73 | 0.92 | 0.89 | 0.8 | 0.99 | 1 | 0.83 | 0.96 | 0.77 |
| Inf-vs-ACV cOrgs1 | 0.023 | 0.58 | 0.42 | 0.13 | 0.29 | 0.44 | 0.49 | 0.64 | 0.5 | 0.6 | 0.32 |
| Inf-vs-UV cOrgs2 | 0.015 | 0.63 | 0.54 | 0.84 | 0.81 | 0.96 | 0.99 | 0.79 | 0.9 | 0.99 | 0.83 |
| Inf-vs-Ctrl hiPSCs | 0.67 | 0.041 | 0.9 | 0.73 | 0.4 | 0.62 | 0.76 | 0.51 | 0.29 | 0.79 | 0.47 |
| Inf-vs-ACV hiPSCs | 0.78 | 0.4 | 0.085 | 0.29 | 0.7 | 1 | 0.059 | 0.14 | 1 | 0.67 | 1 |
| Inf-vs-Ctrl Neurons | 0.38 | 0.43 | 0.35 | 0.013 | 0.0017 | 0.006 | 0.022 | 0.085 | 0.15 | 0.034 | 0.052 |
| Inf-vs-ACV cOrgs2 | 0.53 | 0.33 | 0.94 | 1 | 0.46 | 0.64 | 0.77 | 0.71 | 0.26 | 0.78 | 0.17 |
| ACV-vs-Ctrl cOrgs2 | 0.028 | 1 | 0.53 | 0.86 | 0.38 | 0.53 | 0.83 | 0.88 | 0.63 | 0.97 | 0.4 |
| ACV-vs-UV cOrgs2 | 0.13 | 0.79 | 0.58 | 0.53 | 0.31 | 0.63 | 0.79 | 0.7 | 0.7 | 0.79 | 0.52 |
| UV-vs-Ctrl cOrgs2 | 0.24 | 0.41 | 0.32 | 0.35 | 0.016 | 0.41 | 0.36 | 0.12 | 0.31 | 0.35 | 0.1 |
| IVA-Inf-vs-Ctrl cOrgs | 0.79 | 0.42 | 0.061 | 0.54 | 0.92 | 0.92 | 0.98 | 0.8 | 0.6 | 0.87 | 0.78 |

| | CD | IBD | PSO | RA | T1D | T2D | BMI | BRAIN | HEIGHT | WHR |
|---|---|---|---|---|---|---|---|---|---|---|
| Inf-vs-Ctrl cOrgs1 | 0.4 | 0.31 | 0.13 | 0.068 | 0.23 | 0.75 | 0.46 | 0.18 | 1 | 0.26 |
| Inf-vs-Ctrl cOrgs2 | 0.93 | 0.84 | 0.65 | 0.29 | 0.17 | 0.99 | 0.63 | 0.69 | 1 | 0.9 |
| Inf-vs-ACV cOrgs1 | 0.32 | 0.08 | 0.053 | 0.036 | 0.0097 | 1 | 0.5 | 0.39 | 0.95 | 0.052 |
| Inf-vs-UV cOrgs2 | 0.56 | 0.29 | 0.23 | 0.033 | 0.026 | 0.92 | 0.84 | 1 | 1 | 0.48 |
| Inf-vs-Ctrl hiPSCs | 1 | 0.99 | 0.99 | 0.83 | 0.31 | 1 | 0.19 | 0.57 | 0.99 | 0.97 |
| Inf-vs-ACV hiPSCs | 0.22 | 0.21 | 0.95 | 1 | 1 | 0.8 | 0.056 | 0.67 | 0.8 | 0.036 |
| Inf-vs-Ctrl Neurons | 0.13 | 0.05 | 0.09 | 1 | 0.025 | 0.073 | 0.0068 | 0.092 | 0.0022 | 0.004 |
| Inf-vs-ACV cOrgs2 | 0.66 | 0.46 | 0.93 | 0.56 | 0.55 | 0.73 | 0.81 | 0.45 | 1 | 0.93 |
| ACV-vs-Ctrl cOrgs2 | 0.34 | 0.37 | 0.35 | 0.14 | 0.085 | 1 | 0.54 | 0.24 | 1 | 0.65 |
| ACV-vs-UV cOrgs2 | 0.31 | 0.28 | 0.29 | 0.023 | 0.018 | 1 | 0.93 | 0.34 | 1 | 0.5 |
| UV-vs-Ctrl cOrgs2 | 0.59 | 0.47 | 0.27 | 0.036 | 0.29 | 0.98 | 0.12 | 1 | 0.58 | 0.67 |
| IVA-Inf-vs-Ctrl cOrgs | 0.69 | 0.8 | 0.69 | 0.5 | 0.52 | 0.24 | 0.99 | 0.25 | 0.88 | 0.63 |

TABLE 7
Probabilities of over-expression or under-expression of HSV-1 leaky late and true late genes.

| | LeakyLate_ACV- | LeakyLate_ACV- | TrueLate_ACV- | TrueLate_ACV- |
|---|---|---|---|---|
| | vs-Inf_cOrgs1 | vs-Inf_cOrgs2 | vs-Inf_cOrgs1 | vs-Inf_cOrgs2 |
| Spearman's rho for | 0.63 | | 0.2 | |
| adjusted P-values | | | | |
| Probability of | 1.60E−03 | | 0.47 | |
| Spearman's rho | | | | |
| | Probability of up- | Probability of | 2-tailed | 1-tailed |
| | regulation | down-regulation | P-value | P-value |
| LeakyLate_ACV- | 0.71 | 0.29 | 0.14 | 0.07 |
| vs-Inf_cOrgs1 | | | | |
| LeakyLate_ACV- | 0.79 | 0.21 | 0.02 | 0.01 |
| vs-Inf_cOrgs2 | | | | |
| LeakyLate_ACV- | 0.69 | 0.31 | 0.27 | 0.13 |
| vs-Inf hiPSCs | | | | |
| | Probability of up- | Probability of | 2-tailed | 1-tailed |
| | regulation | down-regulation | P-value | P-value |
| TrueLate ACV- | 0.18 | 0.82 | 0.07 | 0.03 |
| vs-Inf_cOrgs1 | | | | |
| TrueLate ACV- | 0.36 | 0.64 | 0.42 | 0.21 |
| vs-Inf_cOrgs2 | | | | |
| TrueLate ACV- | 0.50 | 0.50 | 1.00 | 0.50 |
| vs-Inf hiPSCs | | | | |

TABLE 8
Pathway enrichment of the significantly differentially
expressed genes and Rescued Pathways are pathways in common between
Inf-vs-Ctrl_cOrgs1, Inf-vs-Ctrl_cOrgs2 and Inf-vs-ACV_cOrgs1 but are not in Inf-vs-ACV_cOrgs2.

| | Human | Input | | | | | |
|---|---|---|---|---|---|---|---|
| PANTHER | genes- | list- | | | fold | raw P- | |
| pathways | 20589 | 8966 | expected | over/under | enrichment | value | FDR |
| Integrin | 200 | 146 | 89.82 | + | 1.63 | 1.06E−05 | 8.46E−04 |
| signaling | | | | | | | |
| pathway | | | | | | | |
| (P00034) | | | | | | | |
| Gonadotropin- | 237 | 156 | 106.44 | + | 1.47 | 2.29E−04 | 1.22E−02 |
| releasing | | | | | | | |
| hormone | | | | | | | |
| receptor | | | | | | | |
| pathway | | | | | | | |
| (P06664) | | | | | | | |
| Angiogenesis | 181 | 121 | 81.29 | + | 1.49 | 8.85E−04 | 3.54E−02 |
| (P00005) | | | | | | | |

TABLE 9
Percentages of AD GWAS genes in both datasets.

| Category | cOrgs1 | cOrgs2 |
|---|---|---|
| Rescued | 41% | 26% |
| Not Rescued | 19% | 24% |
| Exacerbated 1 | 5% | 12% |
| Exacerbated 2 | 18% | 10% |
| Not DEG | 17% | 28% |

TABLES 10A, 10B, 10C, and 10D
ELISA assay measurements for Abeta42/40/38.

| | | | | Calc. Conc. |
|---|---|---|---|---|
| | Virus | Description | Signal | Mean |
| A. SAMPLE | | | | |
| UV_CM_Ctrl_1 | HSV-1 | Conditioned media that had been | 483 | 33.23242029 |
| | | UV-inactivated from uninfected | | |
| | | control cOrg cells | | |
| UV_CM_Ctrl_2 | HSV-1 | Conditioned media that had been | 462 | 31.71287991 |
| | | UV-inactivated from uninfected | | |
| | | control cOrg cells | | |
| UV_CM_Ctrl_3 | HSV-1 | Conditioned media that had been | 466 | 32.00524962 |
| | | UV-inactivated from uninfected | | |
| | | control cOrg cells | | |
| UV_CM_Ctrl_4 | HSV-1 | Conditioned media that had been | 473 | 32.513524 |
| | | UV-inactivated from uninfected | | |
| | | control cOrg cells | | |
| UV_CM_Inf_1 | HSV-1 | Conditioned media that had been | 208 | 7.142191187 |
| | | UV-inactivated from HSV-1 | | |
| | | infected cOrg cells | | |
| UV_CM_Inf_2 | HSV-1 | Conditioned media that had been | 215 | 8.229234804 |
| | | UV-inactivated from HSV-1 | | |
| | | infected cOrg cells | | |
| UV_CM_Inf_3 | HSV-1 | Conditioned media that had been | 260 | 13.99279056 |
| | | UV-inactivated from HSV-1 | | |
| | | infected cOrg cells | | |
| UV_CM_Inf_4 | HSV-1 | Conditioned media that had been | 212 | 7.773264756 |
| | | UV-inactivated from HSV-1 | | |
| | | infected cOrg cells | | |
| UV_CM_ACV_1 | HSV-1 | Conditioned media that had been | 218 | 8.672188261 |
| | | UV-inactivated from ACV-treated | | |
| | | and HSV-1 infected cOrg cells | | |
| UV_CM_ACV_2 | HSV-1 | Conditioned media that had been | 210 | 7.461264676 |
| | | UV-inactivated from ACV-treated | | |
| | | and HSV-1 infected cOrg cells | | |
| UV_CM_ACV_3 | HSV-1 | Conditioned media that had been | 230 | 10.33749872 |
| | | UV-inactivated from ACV-treated | | |
| | | and HSV-1 infected cOrg cells | | |
| UV_CM_ACV_4 | HSV-1 | Conditioned media that had been | 238 | 11.37203986 |
| | | UV-inactivated from ACV-treated | | |
| | | and HSV-1 infected cOrg cells | | |
| UV_CM_UV- | HSV-1 | Conditioned media that had been | 592 | 40.60415864 |
| HSV1_1 | | UV-inactivated from cOrg cells with | | |
| | | UV-inactivated HSV-1 | | |
| UV_CM_UV- | HSV-1 | Conditioned media that had been | 519 | 35.7554183 |
| HSV1_2 | | UV-inactivated from cOrg cells with | | |
| | | UV-inactivated HSV-1 | | |
| UV_CM_UV- | HSV-1 | Conditioned media that had been | 494 | 34.01381076 |
| HSV1_3 | | UV-inactivated from cOrg cells with | | |
| | | UV-inactivated HSV-1 | | |
| UV_CM_UV- | HSV-1 | Conditioned media that had been | 509 | 35.06428377 |
| HSV1_4 | | UV-inactivated from cOrg cells with | | |
| | | UV-inactivated HSV-1 | | |
| HI_CM_Ctrl_1 | HSV-1 | Conditioned media that had been | 622 | 42.50903373 |
| | | heat-inactivated from uninfected | | |
| | | control cOrg cells | | |
| HI_CM_Ctrl_2 | HSV-1 | Conditioned media that had been | 606 | 41.4988623 |
| | | heat-inactivated from uninfected | | |
| | | control cOrg cells | | |
| HI_CM_Ctrl_3 | HSV-1 | Conditioned media that had been | 623 | 42.57174607 |
| | | heat-inactivated from uninfected | | |
| | | control cOrg cells | | |
| HI_CM_Ctrl_4 | HSV-1 | Conditioned media that had been | 663 | 45.0416457 |
| | | heat-inactivated from uninfected | | |
| | | control cOrg cells | | |
| HI_CM_Inf_1 | HSV-1 | Conditioned media that had been | 214 | 8.078764618 |
| | | heat-inactivated from HSV-1 | | |
| | | infected cOrg cells | | |
| HI_CM_Inf_2 | HSV-1 | Conditioned media that had been | 228 | 10.0702728 |
| | | heat-inactivated from HSV-1 | | |
| | | infected cOrg cells | | |
| HI_CM_Inf_3 | HSV-1 | Conditioned media that had been | 224 | 9.524165655 |
| | | heat-inactivated from HSV-1 | | |
| | | infected cOrg cells | | |
| HI_CM_Inf_4 | HSV-1 | Conditioned media that had been | 229 | 10.20434925 |
| | | heat-inactivated from HSV-1 | | |
| | | infected cOrg cells | | |
| HI_CM_ACV_1 | HSV-1 | Conditioned media that had been | 277 | 15.8502417 |
| | | heat-inactivated from cOrg cells | | |
| | | with UV-inactivated HSV-1 | | |
| HI_CM_ACV_2 | HSV-1 | Conditioned media that had been | 305 | 18.68225575 |
| | | heat-inactivated from cOrg cells | | |
| | | with UV-inactivated HSV-1 | | |
| HI_CM_ACV_3 | HSV-1 | Conditioned media that had been | 284 | 16.58198926 |
| | | heat-inactivated from cOrg cells | | |
| | | with UV-inactivated HSV-1 | | |
| HI_CM_ACV_4 | HSV-1 | Conditioned media that had been | 274 | 15.53112004 |
| | | heat-inactivated from cOrg cells | | |
| | | with UV-inactivated HSV-1 | | |
| HI_CM_UV- | HSV-1 | Conditioned media that had been | 701 | 47.32330021 |
| HSV1_1 | | heat-inactivated from cOrg cells | | |
| | | with UV-inactivated HSV-1 | | |
| HI_CM_UV- | HSV-1 | Conditioned media that had been | 632 | 43.13397434 |
| HSV1_2 | | heat-inactivated from cOrg cells | | |
| | | with UV-inactivated HSV-1 | | |
| HI_CM_UV- | HSV-1 | Conditioned media that had been | 592 | 40.60415864 |
| HSV1_3 | | heat-inactivated from cOrg cells | | |
| | | with UV-inactivated HSV-1 | | |
| HI_CM_UV- | HSV-1 | Conditioned media that had been | 597 | 40.92488399 |
| HSV1_4 | | heat-inactivated from cOrg cells | | |
| | | with UV-inactivated HSV-1 | | |
| UV_CM_IAV_ | IAV | Conditioned media that had been | 458 | 31.41907519 |
| Ctrl_1 | | UV-inactivated from uninfected | | |
| | | control cOrg cells | | |
| UV_CM_IAV_ | IAV | Conditioned media that had been | 574 | 39.43822701 |
| Ctrl_2 | | UV-inactivated from uninfected | | |
| | | control cOrg cells | | |
| UV_CM_IAV_ | IAV | Conditioned media that had been | 656 | 44.6146723 |
| Ctrl_3 | | UV-inactivated from uninfected | | |
| | | control cOrg cells | | |
| UV_CM_IAV_ | IAV | Conditioned media that had been | 712 | 47.97292352 |
| Ctrl_4 | | UV-inactivated from uninfected | | |
| | | control cOrg cells | | |
| UV_CM_IAV_ | IAV | Conditioned media that had been | 711 | 47.91405998 |
| Ctrl_5 | | UV-inactivated from uninfected | | |
| | | control cOrg cells | | |
| UV_CM_IAV_Inf_1 | IAV | Conditioned media that had been | 444 | 30.37904834 |
| | | UV-inactivated from IAV infected | | |
| | | cOrg cells | | |
| UV_CM_IAV_Inf_2 | IAV | Conditioned media that had been | 432 | 29.47235356 |
| | | UV-inactivated from IAV infected | | |
| | | cOrg cells | | |
| UV_CM_IAV_Inf_3 | IAV | Conditioned media that had been | 406 | 27.45493325 |
| | | UV-inactivated from IAV infected | | |
| | | cOrg cells | | |
| UV_CM_IAV_Inf_4 | IAV | Conditioned media that had been | 304 | 18.58514248 |
| | | UV-inactivated from IAV infected | | |
| | | cOrg cells | | |
| HI_CM_IAV_Ctrl_1 | IAV | Conditioned media that had been | 481 | 33.08930434 |
| | | heat-inactivated from uninfected | | |
| | | control cOrg cells | | |
| HI_CM_IAV_Ctrl_2 | IAV | Conditioned media that had been | 579 | 39.76390985 |
| | | heat-inactivated from uninfected | | |
| | | control cOrg cells | | |
| HI_CM_IAV_Ctrl_3 | IAV | Conditioned media that had been | 612 | 41.8791878 |
| | | heat-inactivated from uninfected | | |
| | | control cOrg cells | | |
| HI_CM_IAV_Ctrl_4 | IAV | Conditioned media that had been | 710 | 47.8551581 |
| | | heat-inactivated from uninfected | | |
| | | control cOrg cells | | |
| HI_CM_IAV_Ctrl_5 | IAV | Conditioned media that had been | 612 | 41.8791878 |
| | | heat-inactivated from uninfected | | |
| | | control cOrg cells | | |
| HI_CM_IAV_Inf_1 | IAV | Conditioned media that had been | 433 | 29.54847219 |
| | | heat-inactivated from IAV infected | | |
| | | cOrg cells | | |
| HI_CM_IAV_Inf_2 | IAV | Conditioned media that had been | 423 | 28.78252465 |
| | | heat-inactivated from IAV infected | | |
| | | cOrg cells | | |
| HI_CM_IAV_Inf_3 | IAV | Conditioned media that had been | 385 | 25.76633575 |
| | | heat-inactivated from IAV infected | | |
| | | cOrg cells | | |
| HI_CM_IAV_Inf_4 | IAV | Conditioned media that had been | 310 | 19.16387704 |
| | | heat-inactivated from IAV infected | | |
| | | cOrg cells | | |
| B. SAMPLE | | | | |
| UV_CM_Ctrl_1 | HSV-1 | Conditioned media that had been | 3053 | 137.4731021 |
| | | UV-inactivated from uninfected | | |
| | | control cOrg cells | | |
| UV_CM_Ctrl_2 | HSV-1 | Conditioned media that had been | 2878 | 131.5771288 |
| | | UV-inactivated from uninfected | | |
| | | control cOrg cells | | |
| UV_CM_Ctrl_3 | HSV-1 | Conditioned media that had been | 2900 | 132.3250303 |
| | | UV-inactivated from uninfected | | |
| | | control cOrg cells | | |
| UV_CM_Ctrl_4 | HSV-1 | Conditioned media that had been | 3070 | 138.0395398 |
| | | UV-inactivated from uninfected | | |
| | | control cOrg cells | | |
| UV_CM_Inf_1 | HSV-1 | Conditioned media that had been | 533 | 29.05674302 |
| | | UV-inactivated from HSV-1 | | |
| | | infected cOrg cells | | |
| UV_CM_Inf_2 | HSV-1 | Conditioned media that had been | 561 | 30.96719159 |
| | | UV-inactivated from HSV-1 | | |
| | | infected cOrg cells | | |
| UV_CM_Inf_3 | HSV-1 | Conditioned media that had been | 541 | 29.60849996 |
| | | UV-inactivated from HSV-1 | | |
| | | infected cOrg cells | | |
| UV_CM_Inf_4 | HSV-1 | Conditioned media that had been | 530 | 28.84855643 |
| | | UV-inactivated from HSV-1 | | |
| | | infected cOrg cells | | |
| UV_CM_ACV_1 | HSV-1 | Conditioned media that had been | 516 | 27.86743537 |
| | | UV-inactivated from ACV-treated | | |
| | | and HSV-1 infected cOrg cells | | |
| UV_CM_ACV_2 | HSV-1 | Conditioned media that had been | 465 | 24.14331805 |
| | | UV-inactivated from ACV-treated | | |
| | | and HSV-1 infected cOrg cells | | |
| UV_CM_ACV_3 | HSV-1 | Conditioned media that had been | 560 | 30.89993199 |
| | | UV-inactivated from ACV-treated | | |
| | | and HSV-1 infected cOrg cells | | |
| UV_CM_ACV_4 | HSV-1 | Conditioned media that had been | 605 | 33.86219748 |
| | | UV-inactivated from ACV-treated | | |
| | | and HSV-1 infected cOrg cells | | |
| UV_CM_UV- | HSV-1 | Conditioned media that had been | 3957 | 166.2683799 |
| HSV1_1 | | UV-inactivated from cOrg cells | | |
| | | with UV-inactivated HSV-1 | | |
| UV_CM_UV- | HSV-1 | Conditioned media that had been | 3194 | 142.1390356 |
| HSV1_2 | | UV-inactivated from cOrg cells | | |
| | | with UV-inactivated HSV-1 | | |
| UV_CM_UV- | HSV-1 | Conditioned media that had been | 2719 | 126.1113069 |
| HSV1_3 | | UV-inactivated from cOrg cells | | |
| | | with UV-inactivated HSV-1 | | |
| UV_CM_UV- | HSV-1 | Conditioned media that had been | 2853 | 130.7248257 |
| HSV1_4 | | UV-inactivated from cOrg cells | | |
| | | with UV-inactivated HSV-1 | | |
| HI_CM_Ctrl_1 | HSV-1 | Conditioned media that had been | 3978 | 166.9089223 |
| | | heat-inactivated from uninfected | | |
| | | control cOrg cells | | |
| HI_CM_Ctrl_2 | HSV-1 | Conditioned media that had been | 3934 | 165.5655177 |
| | | heat-inactivated from uninfected | | |
| | | control cOrg cells | | |
| HI_CM_Ctrl_3 | HSV-1 | Conditioned media that had been | 4163 | 172.5035607 |
| | | heat-inactivated from uninfected | | |
| | | control cOrg cells | | |
| HI_CM_Ctrl_4 | HSV-1 | Conditioned media that had been | 4039 | 168.7631223 |
| | | heat-inactivated from uninfected | | |
| | | control cOrg cells | | |
| HI_CM_Inf_1 | HSV-1 | Conditioned media that had been | 577 | 32.03416299 |
| | | heat-inactivated from HSV-1 | | |
| | | infected cOrg cells | | |
| HI_CM_Inf_2 | HSV-1 | Conditioned media that had been | 624 | 35.07646701 |
| | | heat-inactivated from HSV-1 | | |
| | | infected cOrg cells | | |
| HI_CM_Inf_3 | HSV-1 | Conditioned media that had been | 581 | 32.29828175 |
| | | heat-inactivated from HSV-1 | | |
| | | infected cOrg cells | | |
| HI_CM_Inf_4 | HSV-1 | Conditioned media that had been | 607 | 33.99097522 |
| | | heat-inactivated from HSV-1 | | |
| | | infected cOrg cells | | |
| HI_CM_ACV_1 | HSV-1 | Conditioned media that had been | 878 | 49.80911504 |
| | | heat-inactivated from cOrg cells | | |
| | | with UV-inactivated HSV-1 | | |
| HI_CM_ACV_2 | HSV-1 | Conditioned media that had been | 962 | 54.23021169 |
| | | heat-inactivated from cOrg cells | | |
| | | with UV-inactivated HSV-1 | | |
| HI_CM_ACV_3 | HSV-1 | Conditioned media that had been | 929 | 52.51435462 |
| | | heat-inactivated from cOrg cells | | |
| | | with UV-inactivated HSV-1 | | |
| HI_CM_ACV_4 | HSV-1 | Conditioned media that had been | 841 | 47.802396 |
| | | heat-inactivated from cOrg cells | | |
| | | with UV-inactivated HSV-1 | | |
| HI_CM_UV- | HSV-1 | Conditioned media that had been | 4736 | 189.3275429 |
| HSV1_1 | | heat-inactivated from cOrg cells | | |
| | | with UV-inactivated HSV-1 | | |
| HI_CM_UV- | HSV-1 | Conditioned media that had been | 4035 | 168.6418255 |
| HSV1_2 | | heat-inactivated from cOrg cells | | |
| | | with UV-inactivated HSV-1 | | |
| HI_CM_UV- | HSV-1 | Conditioned media that had been | 3966 | 166.543038 |
| HSV1_3 | | heat-inactivated from cOrg cells | | |
| | | with UV-inactivated HSV-1 | | |
| HI_CM_UV- | HSV-1 | Conditioned media that had been | 3882 | 163.9713079 |
| HSV1_4 | | heat-inactivated from cOrg cells | | |
| | | with UV-inactivated HSV-1 | | |
| UV_CM_IAV_Ctrl_1 | IAV | Conditioned media that had been | 2656 | 123.9149039 |
| | | UV-inactivated from uninfected | | |
| | | control cOrg cells | | |
| UV_CM_IAV_Ctrl_2 | IAV | Conditioned media that had been | 4022 | 168.2473312 |
| | | UV-inactivated from uninfected | | |
| | | control cOrg cells | | |
| UV_CM_IAV_Ctrl_3 | IAV | Conditioned media that had been | 4572 | 184.5852831 |
| | | UV-inactivated from uninfected | | |
| | | control cOrg cells | | |
| UV_CM_IAV_Ctrl_4 | IAV | Conditioned media that had been | 5127 | 200.4196613 |
| | | UV-inactivated from uninfected | | |
| | | control cOrg cells | | |
| UV_CM_IAV_Ctrl_5 | IAV | Conditioned media that had been | 5113 | 200.027444 |
| | | UV-inactivated from uninfected | | |
| | | control cOrg cells | | |
| UV_CM_IAV_Inf_1 | IAV | Conditioned media that had been | 2657 | 123.949909 |
| | | UV-inactivated from IAV infected | | |
| | | cOrg cells | | |
| UV_CM_IAV_Inf_2 | IAV | Conditioned media that had been | 2431 | 115.9161977 |
| | | UV-inactivated from IAV infected | | |
| | | cOrg cells | | |
| UV_CM_IAV_Inf_3 | IAV | Conditioned media that had been | 2082 | 102.9660975 |
| | | UV-inactivated from IAV infected | | |
| | | cOrg cells | | |
| UV_CM_IAV_Inf_4 | IAV | Conditioned media that had been | 1135 | 62.84417081 |
| | | UV-inactivated from IAV infected | | |
| | | cOrg cells | | |
| HI_CM_IAV_Ctrl_1 | IAV | Conditioned media that had been | 2865 | 131.1342537 |
| | | heat-inactivated from uninfected | | |
| | | control cOrg cells | | |
| HI_CM_IAV_Ctrl_2 | IAV | Conditioned media that had been | 3926 | 165.3207194 |
| | | heat-inactivated from uninfected | | |
| | | control cOrg cells | | |
| HI_CM_IAV_Ctrl_3 | IAV | Conditioned media that had been | 4144 | 171.9328819 |
| | | heat-inactivated from uninfected | | |
| | | control cOrg cells | | |
| HI_CM_IAV_Ctrl_4 | IAV | Conditioned media that had been | 5127 | 200.4196613 |
| | | heat-inactivated from uninfected | | |
| | | control cOrg cells | | |
| HI_CM_IAV_Ctrl_5 | IAV | Conditioned media that had been | 4749 | 189.7010988 |
| | | heat-inactivated from uninfected | | |
| | | control cOrg cells | | |
| HI_CM_IAV_Inf_1 | IAV | Conditioned media that had been | 2616 | 122.5108856 |
| | | heat-inactivated from IAV | | |
| | | infected cOrg cells | | |
| HI_CM_IAV_Inf_2 | IAV | Conditioned media that had been | 2178 | 106.6010876 |
| | | heat-inactivated from IAV | | |
| | | infected cOrg cells | | |
| HI_CM_IAV_Inf_3 | IAV | Conditioned media that had been | 1805 | 92.11838709 |
| | | heat-inactivated from IAV | | |
| | | infected cOrg cells | | |
| HI_CM_IAV_Inf_4 | IAV | Conditioned media that had been | 1170 | 64.51872713 |
| | | heat-inactivated from IAV | | |
| | | infected cOrg cells | | |
| C. SAMPLE | | | | |
| UV_CM_Ctrl_1 | HSV-1 | Conditioned media that had been | 5664 | 12.28454063 |
| | | UV-inactivated from uninfected | | |
| | | control cOrg cells | | |
| UV_CM_Ctrl_2 | HSV-1 | Conditioned media that had been | 5725 | 12.40496245 |
| | | UV-inactivated from uninfected | | |
| | | control cOrg cells | | |
| UV_CM_Ctrl_3 | HSV-1 | Conditioned media that had been | 5604 | 12.1659493 |
| | | UV-inactivated from uninfected | | |
| | | control cOrg cells | | |
| UV_CM_Ctrl_4 | HSV-1 | Conditioned media that had been | 5594 | 12.1461701 |
| | | UV-inactivated from uninfected | | |
| | | control cOrg cells | | |
| UV_CM_Inf_1 | HSV-1 | Conditioned media that had been | 1002 | 2.365934768 |
| | | UV-inactivated from HSV-1 | | |
| | | infected cOrg cells | | |
| UV_CM_Inf_2 | HSV-1 | Conditioned media that had been | 1004 | 2.370787239 |
| | | UV-inactivated from HSV-1 | | |
| | | infected cOrg cells | | |
| UV_CM_Inf_3 | HSV-1 | Conditioned media that had been | 829 | 1.941098081 |
| | | UV-inactivated from HSV-1 | | |
| | | infected cOrg cells | | |
| UV_CM_Inf_4 | HSV-1 | Conditioned media that had been | 865 | 2.030389803 |
| | | UV-inactivated from HSV-1 | | |
| | | infected cOrg cells | | |
| UV_CM_ACV_1 | HSV-1 | Conditioned media that had been | 1205 | 2.852606732 |
| | | UV-inactivated from ACV-treated | | |
| | | and HSV-1 infected cOrg cells | | |
| UV_CM_ACV_2 | HSV-1 | Conditioned media that had been | 1088 | 2.573506923 |
| | | UV-inactivated from ACV-treated | | |
| | | and HSV-1 infected cOrg cells | | |
| UV_CM_ACV_3 | HSV-1 | Conditioned media that had been | 1324 | 3.13301275 |
| | | UV-inactivated from ACV-treated | | |
| | | and HSV-1 infected cOrg cells | | |
| UV_CM_ACV_4 | HSV-1 | Conditioned media that had been | 1544 | 3.643461191 |
| | | UV-inactivated from ACV-treated | | |
| | | and HSV-1 infected cOrg cells | | |
| UV_CM_UV- | HSV-1 | Conditioned media that had been | 6742 | 14.39246649 |
| HSV1_1 | | UV-inactivated from cOrg cells | | |
| | | with UV-inactivated HSV-1 | | |
| UV_CM_UV- | HSV-1 | Conditioned media that had been | 6441 | 13.80800802 |
| HSV1_2 | | UV-inactivated from cOrg cells | | |
| | | with UV-inactivated HSV-1 | | |
| UV_CM_UV- | HSV-1 | Conditioned media that had been | 6070 | 13.08334892 |
| HSV1_3 | | UV-inactivated from cOrg cells | | |
| | | with UV-inactivated HSV-1 | | |
| UV_CM_UV- | HSV-1 | Conditioned media that had been | 6218 | 13.37301574 |
| HSV1_4 | | UV-inactivated from cOrg cells | | |
| | | with UV-inactivated HSV-1 | | |
| HI_CM_Ctrl_1 | HSV-1 | Conditioned media that had been | 4928 | 10.81940831 |
| | | heat-inactivated from uninfected | | |
| | | control cOrg cells | | |
| HI_CM_Ctrl_2 | HSV-1 | Conditioned media that had been | 4934 | 10.83144854 |
| | | heat-inactivated from uninfected | | |
| | | control cOrg cells | | |
| HI_CM_Ctrl_3 | HSV-1 | Conditioned media that had been | 5017 | 10.99783488 |
| | | heat-inactivated from uninfected | | |
| | | control cOrg cells | | |
| HI_CM_Ctrl_4 | HSV-1 | Conditioned media that had been | 4461 | 9.876958781 |
| | | heat-inactivated from uninfected | | |
| | | control cOrg cells | | |
| HI_CM_Inf_1 | HSV-1 | Conditioned media that had been | 741 | 1.720588023 |
| | | heat-inactivated from HSV-1 | | |
| | | infected cOrg cells | | |
| HI_CM_Inf_2 | HSV-1 | Conditioned media that had been | 717 | 1.659848314 |
| | | heat-inactivated from HSV-1 | | |
| | | infected cOrg cells | | |
| HI_CM_Inf_3 | HSV-1 | Conditioned media that had been | 684 | 1.575872281 |
| | | heat-inactivated from HSV-1 | | |
| | | infected cOrg cells | | |
| HI_CM_Inf_4 | HSV-1 | Conditioned media that had been | 663 | 1.522141804 |
| | | heat-inactivated from HSV-1 | | |
| | | infected cOrg cells | | |
| HI_CM_ACV_1 | HSV-1 | Conditioned media that had been | 1509 | 3.562883717 |
| | | heat-inactivated from cOrg cells | | |
| | | with UV-inactivated HSV-1 | | |
| HI_CM_ACV_2 | HSV-1 | Conditioned media that had been | 1567 | 3.696290744 |
| | | heat-inactivated from cOrg cells | | |
| | | with UV-inactivated HSV-1 | | |
| HI_CM_ACV_3 | HSV-1 | Conditioned media that had been | 1405 | 3.322079123 |
| | | heat-inactivated from cOrg cells | | |
| | | with UV-inactivated HSV-1 | | |
| HI_CM_ACV_4 | HSV-1 | Conditioned media that had been | 1318 | 3.11895195 |
| | | heat-inactivated from cOrg cells | | |
| | | with UV-inactivated HSV-1 | | |
| HI_CM_UV- | HSV-1 | Conditioned media that had been | 5754 | 12.46216104 |
| HSV1_1 | | heat-inactivated from cOrg cells | | |
| | | with UV-inactivated HSV-1 | | |
| HI_CM_UV- | HSV-1 | Conditioned media that had been | 5322 | 11.60660837 |
| HSV1_2 | | heat-inactivated from cOrg cells | | |
| | | with UV-inactivated HSV-1 | | |
| HI_CM_UV- | HSV-1 | Conditioned media that had been | 4937 | 10.83746803 |
| HSV1_3 | | heat-inactivated from cOrg cells | | |
| | | with UV-inactivated HSV-1 | | |
| HI_CM_UV- | HSV-1 | Conditioned media that had been | 5134 | 11.23184889 |
| HSV1_4 | | heat-inactivated from cOrg cells | | |
| | | with UV-inactivated HSV-1 | | |
| UV_CM_IAV_Ctrl_1 | IAV | Conditioned media that had been | 3990 | 8.914914679 |
| | | UV-inactivated from uninfected | | |
| | | control cOrg cells | | |
| UV_CM_IAV_Ctrl_2 | IAV | Conditioned media that had been | 5056 | 11.07590789 |
| | | UV-inactivated from uninfected | | |
| | | control cOrg cells | | |
| UV_CM_IAV_Ctrl_3 | IAV | Conditioned media that had been | 5326 | 11.61456544 |
| | | UV-inactivated from uninfected | | |
| | | control cOrg cells | | |
| UV_CM_IAV_Ctrl_4 | IAV | Conditioned media that had been | 5375 | 11.71198459 |
| | | UV-inactivated from uninfected | | |
| | | control cOrg cells | | |
| UV_CM_IAV_Ctrl_5 | IAV | Conditioned media that had been | 5336 | 11.63445514 |
| | | UV-inactivated from uninfected | | |
| | | control cOrg cells | | |
| UV_CM_IAV_Inf_1 | IAV | Conditioned media that had been | 3591 | 8.089713509 |
| | | UV-inactivated from IAV infected | | |
| | | cOrg cells | | |
| UV_CM_IAV_Inf_2 | IAV | Conditioned media that had been | 3402 | 7.695193421 |
| | | UV-inactivated from IAV infected | | |
| | | cOrg cells | | |
| UV_CM_IAV_Inf_3 | IAV | Conditioned media that had been | 2792 | 6.403607487 |
| | | UV-inactivated from IAV infected | | |
| | | cOrg cells | | |
| UV_CM_IAV_Inf_4 | IAV | Conditioned media that had been | 1638 | 3.858786065 |
| | | UV-inactivated from IAV infected | | |
| | | cOrg cells | | |
| HI_CM_IAV_Ctrl_1 | IAV | Conditioned media that had been | 3889 | 8.706964853 |
| | | heat-inactivated from uninfected | | |
| | | control cOrg cells | | |
| HI_CM_IAV_Ctrl_2 | IAV | Conditioned media that had been | 4736 | 10.43322595 |
| | | heat-inactivated from uninfected | | |
| | | control cOrg cells | | |
| HI_CM_IAV_Ctrl_3 | IAV | Conditioned media that had been | 5267 | 11.49712947 |
| | | heat-inactivated from uninfected | | |
| | | control cOrg cells | | |
| HI_CM_IAV_Ctrl_4 | IAV | Conditioned media that had been | 5370 | 11.7020485 |
| | | heat-inactivated from uninfected | | |
| | | control cOrg cells | | |
| HI_CM_IAV_Ctrl_5 | IAV | Conditioned media that had been | 4957 | 10.87758731 |
| | | heat-inactivated from uninfected | | |
| | | control cOrg cells | | |
| HI_CM_IAV_Inf_1 | IAV | Conditioned media that had been | 3432 | 7.757981197 |
| | | heat-inactivated from IAV infected | | |
| | | cOrg cells | | |
| HI_CM_IAV_Inf_2 | IAV | Conditioned media that had been | 3119 | 7.099650299 |
| | | heat-inactivated from IAV infected | | |
| | | cOrg cells | | |
| HI_CM_IAV_Inf_3 | IAV | Conditioned media that had been | 2603 | 5.997016733 |
| | | heat-inactivated from IAV infected | | |
| | | cOrg cells | | |
| HI_CM_IAV_Inf_4 | IAV | Conditioned media that had been | 1715 | 4.034052625 |
| | | heat-inactivated from IAV infected | | |
| | | cOrg cells | | |
| D. SAMPLE | | | | |
| UV_CM_Ctrl_1 | HSV-1 | Conditioned media that had been | 0.0894 | 0.3697 |
| | | UV-inactivated from uninfected | | |
| | | control cOrg cells | | |
| UV_CM_Ctrl_2 | HSV-1 | Conditioned media that had been | 0.0943 | 0.3912 |
| | | UV-inactivated from uninfected | | |
| | | control cOrg cells | | |
| UV_CM_Ctrl_3 | HSV-1 | Conditioned media that had been | 0.0919 | 0.3801 |
| | | UV-inactivated from uninfected | | |
| | | control cOrg cells | | |
| UV_CM_Ctrl_4 | HSV-1 | Conditioned media that had been | 0.0880 | 0.3736 |
| | | UV-inactivated from uninfected | | |
| | | control cOrg cells | | |
| UV_CM_Inf_1 | HSV-1 | Conditioned media that had been | 0.0814 | 0.3313 |
| | | UV-inactivated from HSV-1 | | |
| | | infected cOrg cells | | |
| UV_CM_Inf_2 | HSV-1 | Conditioned media that had been | 0.0766 | 0.2881 |
| | | UV-inactivated from HSV-1 | | |
| | | infected cOrg cells | | |
| UV_CM_Inf_3 | HSV-1 | Conditioned media that had been | 0.0656 | 0.1387 |
| | | UV-inactivated from HSV-1 | | |
| | | infected cOrg cells | | |
| UV_CM_Inf_4 | HSV-1 | Conditioned media that had been | 0.0704 | 0.2612 |
| | | UV-inactivated from HSV-1 | | |
| | | infected cOrg cells | | |
| UV_CM_ACV_1 | HSV-1 | Conditioned media that had been | 0.1024 | 0.3289 |
| | | UV-inactivated from ACV-treated | | |
| | | and HSV-1 infected cOrg cells | | |
| UV_CM_ACV_2 | HSV-1 | Conditioned media that had been | 0.1066 | 0.3449 |
| | | UV-inactivated from ACV-treated | | |
| | | and HSV-1 infected cOrg cells | | |
| UV_CM_ACV_3 | HSV-1 | Conditioned media that had been | 0.1014 | 0.3031 |
| | | UV-inactivated from ACV-treated | | |
| | | and HSV-1 infected cOrg cells | | |
| UV_CM_ACV_4 | HSV-1 | Conditioned media that had been | 0.1076 | 0.3204 |
| | | UV-inactivated from ACV-treated | | |
| | | and HSV-1 infected cOrg cells | | |
| UV_CM_UV- | HSV-1 | Conditioned media that had been | 0.0866 | 0.3545 |
| HSV1_1 | | UV-inactivated from cOrg cells | | |
| | | with UV-inactivated HSV-1 | | |
| UV_CM_UV- | HSV-1 | Conditioned media that had been | 0.0971 | 0.3862 |
| HSV1_2 | | UV-inactivated from cOrg cells | | |
| | | with UV-inactivated HSV-1 | | |
| UV_CM_UV- | HSV-1 | Conditioned media that had been | 0.1037 | 0.3846 |
| HSV1_3 | | UV-inactivated from cOrg cells | | |
| | | with UV-inactivated HSV-1 | | |
| UV_CM_UV- | HSV-1 | Conditioned media that had been | 0.1023 | 0.3814 |
| HSV1_4 | | UV-inactivated from cOrg cells | | |
| | | with UV-inactivated HSV-1 | | |
| HI_CM_Ctrl_1 | HSV-1 | Conditioned media that had been | 0.0648 | 0.2545 |
| | | heat-inactivated from uninfected | | |
| | | control cOrg cells | | |
| HI_CM_Ctrl_2 | HSV-1 | Conditioned media that had been | 0.0654 | 0.2610 |
| | | heat-inactivated from uninfected | | |
| | | control cOrg cells | | |
| HI_CM_Ctrl_3 | HSV-1 | Conditioned media that had been | 0.0638 | 0.2583 |
| | | heat-inactivated from uninfected | | |
| | | control cOrg cells | | |
| HI_CM_Ctrl_4 | HSV-1 | Conditioned media that had been | 0.0585 | 0.2193 |
| | | heat-inactivated from uninfected | | |
| | | control cOrg cells | | |
| HI_CM_Inf_1 | HSV-1 | Conditioned media that had been | 0.0537 | 0.2130 |
| | | heat-inactivated from HSV-1 | | |
| | | infected cOrg cells | | |
| HI_CM_Inf_2 | HSV-1 | Conditioned media that had been | 0.0473 | 0.1648 |
| | | heat-inactivated from HSV-1 | | |
| | | infected cOrg cells | | |
| HI_CM_Inf_3 | HSV-1 | Conditioned media that had been | 0.0488 | 0.1655 |
| | | heat-inactivated from HSV-1 | | |
| | | infected cOrg cells | | |
| HI_CM_Inf_4 | HSV-1 | Conditioned media that had been | 0.0448 | 0.1492 |
| | | heat-inactivated from HSV-1 | | |
| | | infected cOrg cells | | |
| HI_CM_ACV_1 | HSV-1 | Conditioned media that had been | 0.0715 | 0.2248 |
| | | heat-inactivated from cOrg cells | | |
| | | with UV-inactivated HSV-1 | | |
| HI_CM_ACV_2 | HSV-1 | Conditioned media that had been | 0.0682 | 0.1979 |
| | | heat-inactivated from cOrg cells | | |
| | | with UV-inactivated HSV-1 | | |
| HI_CM_ACV_3 | HSV-1 | Conditioned media that had been | 0.0633 | 0.2003 |
| | | heat-inactivated from cOrg cells | | |
| | | with UV-inactivated HSV-1 | | |
| HI_CM_ACV_4 | HSV-1 | Conditioned media that had been | 0.0652 | 0.2008 |
| | | heat-inactivated from cOrg cells | | |
| | | with UV-inactivated HSV-1 | | |
| HI_CM_UV- | HSV-1 | Conditioned media that had been | 0.0658 | 0.2633 |
| HSV1_1 | | heat-inactivated from cOrg cells | | |
| | | with UV-inactivated HSV-1 | | |
| HI_CM_UV- | HSV-1 | Conditioned media that had been | 0.0688 | 0.2691 |
| HSV1_2 | | heat-inactivated from cOrg cells | | |
| | | with UV-inactivated HSV-1 | | |
| HI_CM_UV- | HSV-1 | Conditioned media that had been | 0.0651 | 0.2669 |
| HSV1_3 | | heat-inactivated from cOrg cells | | |
| | | with UV-inactivated HSV-1 | | |
| HI_CM_UV- | HSV-1 | Conditioned media that had been | 0.0685 | 0.2745 |
| HSV1_4 | | heat-inactivated from cOrg cells | | |
| | | with UV-inactivated HSV-1 | | |
| UV_CM_IAV_Ctrl_1 | IAV | Conditioned media that had been | 0.0719 | 0.2837 |
| | | UV-inactivated from uninfected | | |
| | | control cOrg cells | | |
| UV_CM_IAV_Ctrl_2 | IAV | Conditioned media that had been | 0.0658 | 0.2808 |
| | | UV-inactivated from uninfected | | |
| | | control cOrg cells | | |
| UV_CM_IAV_Ctrl_3 | IAV | Conditioned media that had been | 0.0629 | 0.2603 |
| | | UV-inactivated from uninfected | | |
| | | control cOrg cells | | |
| UV_CM_IAV_Ctrl_4 | IAV | Conditioned media that had been | 0.0584 | 0.2441 |
| | | UV-inactivated from uninfected | | |
| | | control cOrg cells | | |
| UV_CM_IAV_Ctrl_5 | IAV | Conditioned media that had been | 0.0582 | 0.2428 |
| | | UV-inactivated from uninfected | | |
| | | control cOrg cells | | |
| UV_CM_IAV_Inf_1 | IAV | Conditioned media that had been | 0.0653 | 0.2663 |
| | | UV-inactivated from IAV infected | | |
| | | cOrg cells | | |
| UV_CM_IAV_Inf_2 | IAV | Conditioned media that had been | 0.0664 | 0.2611 |
| | | UV-inactivated from IAV infected | | |
| | | cOrg cells | | |
| UV_CM_IAV_Inf_3 | IAV | Conditioned media that had been | 0.0622 | 0.2332 |
| | | UV-inactivated from IAV infected | | |
| | | cOrg cells | | |
| UV_CM_IAV_Inf_4 | IAV | Conditioned media that had been | 0.0614 | 0.2076 |
| | | UV-inactivated from IAV infected | | |
| | | cOrg cells | | |
| HI_CM_IAV_Ctrl_1 | IAV | Conditioned media that had been | 0.0664 | 0.2631 |
| | | heat-inactivated from uninfected | | |
| | | control cOrg cells | | |
| HI_CM_IAV_Ctrl_2 | IAV | Conditioned media that had been | 0.0631 | 0.2624 |
| | | heat-inactivated from uninfected | | |
| | | control cOrg cells | | |
| HI_CM_IAV_Ctrl_3 | IAV | Conditioned media that had been | 0.0669 | 0.2745 |
| | | heat-inactivated from uninfected | | |
| | | control cOrg cells | | |
| HI_CM_IAV_Ctrl_4 | IAV | Conditioned media that had been | 0.0584 | 0.2445 |
| | | heat-inactivated from uninfected | | |
| | | control cOrg cells | | |
| HI_CM_IAV_Ctrl_5 | IAV | Conditioned media that had been | 0.0573 | 0.2597 |
| | | heat-inactivated from uninfected | | |
| | | control cOrg cells | | |
| HI_CM_IAV_Inf_1 | IAV | Conditioned media that had been | 0.0633 | 0.2626 |
| | | heat-inactivated from IAV infected | | |
| | | cOrg cells | | |
| HI_CM_IAV_Inf_2 | IAV | Conditioned media that had been | 0.0666 | 0.2467 |
| | | heat-inactivated from IAV infected | | |
| | | cOrg cells | | |
| HI_CM_IAV_Inf_3 | IAV | Conditioned media that had been | 0.0651 | 0.2327 |
| | | heat-inactivated from IAV infected | | |
| | | cOrg cells | | |
| HI_CM_IAV_Inf_4 | IAV | Conditioned media that had been | 0.0625 | 0.2105 |
| | | heat-inactivated from IAV infected | | |
| | | cOrg cells | | |

Table 10A shows results for Abeta38.
Table 10B shows results for Abeta40.
Table 10C shows results for Abeta42.
Table 10D shows the Abeta 42/40 and Abeta 42/38 ratios.

TABLE 11
Enrichment of DEGs from cOrgs with informative DEGs.

| Subtype | cOrg_Data | Neff_2021.score | Neff_2021.normScore | Neff_2021.pvalue | Neff_2021.adjP | FWER |
|---|---|---|---|---|---|---|
| A | Inf-vs- | 0.27 | 2.45 | 1.66544E−10 | 3.33089E−10 | 1.0E−08 |
| | Ctrl_cOrgs1 | | | | | |
| A | Inf-vs- | 0.28 | 2.47 | 1.06772E−10 | 3.20317E−10 | 9.6E−09 |
| | Ctrl_cOrgs2 | | | | | |
| A | Inf-vs- | 0.01 | 0.13 | 0.389671191 | 0.389671191 | 0.39 |
| | UV_cOrgs2 | | | | | |
| A | Inf-vs- | 0.29 | 2.59 | 3.5708E−12 | 2.14248E−11 | 6.4E−10 |
| | ACV_cOrgs1 | | | | | |
| A | Inf-vs- | −0.02 | −0.21 | 0.337329312 | 0.389671191 | 0.39 |
| | ACV_cOrgs2 | | | | | |
| A | ACV-vs- | 0.26 | 2.29 | 8.85197E−09 | 1.3278E−08 | 4.0E−07 |
| | Ctrl_cOrgs2 | | | | | |
| B1 | Inf-vs- | 0.35 | 1.91 | 0.003208297 | 0.009624892 | 0.29 |
| | Ctrl_cOrgs1 | | | | | |
| B1 | Inf-vs- | 0.30 | 1.65 | 0.017378267 | 0.034756535 | 0.035 |
| | Ctrl_cOrgs2 | | | | | |
| B1 | Inf-vs- | 0.27 | 1.48 | 0.040900054 | 0.058779074 | 0.059 |
| | UV_cOrgs2 | | | | | |
| B1 | Inf-vs- | 0.26 | 1.44 | 0.048982562 | 0.058779074 | 0.059 |
| | ACV_cOrgs1 | | | | | |
| B1 | Inf-vs- | −0.10 | −0.65 | 0.38978527 | 0.38978527 | 0.39 |
| | ACV_cOrgs2 | | | | | |
| B1 | ACV-vs- | 0.41 | 2.27 | 0.000149029 | 0.000894172 | 0.027 |
| | Ctrl_cOrgs2 | | | | | |
| B2 | Inf-vs- | −0.26 | −1.99 | 1.06984E−05 | 3.20951E−05 | 9.6E−04 |
| | Ctrl_cOrgs1 | | | | | |
| B2 | Inf-vs- | −0.05 | −0.36 | 0.29441133 | 0.477223623 | 0.48 |
| | Ctrl_cOrgs2 | | | | | |
| B2 | Inf-vs- | −0.04 | −0.28 | 0.32046546 | 0.477223623 | 0.48 |
| | UV_cOrgs2 | | | | | |
| B2 | Inf-vs- | 0.31 | 2.36 | 2.3348E−09 | 1.40088E−08 | 4.2E−07 |
| | ACV_cOrgs1 | | | | | |
| B2 | Inf-vs- | 0.01 | 0.06 | 0.45319215 | 0.477223623 | 0.48 |
| | ACV_cOrgs2 | | | | | |
| B2 | ACV-vs- | 0.00 | −0.03 | 0.477223623 | 0.477223623 | 0.48 |
| | Ctrl_cOrgs2 | | | | | |
| C1 | Inf-vs- | −0.36 | −2.54 | 3.12276E−10 | 1.87365E−09 | 5.6E−08 |
| | Ctrl_cOrgs1 | | | | | |
| C1 | Inf-VS- | −0.31 | −2.21 | 3.5968E−07 | 1.07904E−06 | 3.2E−05 |
| | Ctrl_cOrgs2 | | | | | |
| C1 | Inf-vs- | −0.11 | −0.76 | 0.216468181 | 0.259761817 | 0.26 |
| | UV_cOrgs2 | | | | | |
| C1 | Inf-vs- | 0.17 | 1.20 | 0.057508106 | 0.086262159 | 0.086 |
| | ACV_cOrgs1 | | | | | |
| C1 | Inf-vs- | 0.02 | 0.12 | 0.435380142 | 0.435380142 | 0.44 |
| | ACV_cOrgs2 | | | | | |
| C1 | ACV-vs- | −0.28 | −2.03 | 9.13019E−06 | 1.82604E−05 | 5.5E−04 |
| | Ctrl_cOrgs2 | | | | | |
| C2 | Inf-vs- | 0.04 | 0.32 | 0.333469214 | 0.444095635 | 0.44 |
| | Ctrl_cOrgs1 | | | | | |
| C2 | Inf-vs- | −0.03 | −0.29 | 0.343776819 | 0.444095635 | 0.44 |
| | Ctrl_cOrgs2 | | | | | |
| C2 | Inf-vs- | 0.06 | 0.47 | 0.293925566 | 0.444095635 | 0.44 |
| | UV_cOrgs2 | | | | | |
| C2 | Inf-vs- | 0.01 | 0.08 | 0.450305396 | 0.450305396 | 0.45 |
| | ACV_cOrgs1 | | | | | |
| C2 | Inf-vs- | −0.06 | −0.49 | 0.287978103 | 0.444095635 | 0.44 |
| | ACV_cOrgs2 | | | | | |
| C2 | ACV-vs- | 0.03 | 0.23 | 0.370079696 | 0.444095635 | 0.44 |
| | Ctrl_cOrgs2 | | | | | |

1. A method of treating a neurological disorder in a subject in need thereof, the method comprising:
collecting a biological sample from the subject,
detecting a gene expression signature from the biological sample, and
administering a therapeutically effective amount of an anti-viral agent to the subject with the gene expression signature, wherein the gene expression signature comprises at least one gene affected by the neurological disorder and a herpes simplex virus 1 (HSV-1).

2. The method of claim 1, wherein the at least one gene is selected from a first gene set comprising AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, or YAP1.

3. The method of claim 1, wherein the at least one gene is selected from a second gene set comprising ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, or WNT7B.

4. The method of claim 1, wherein the first gene set is combined with the second gene set.

5. The method of claim 1, wherein the method detects an increase or decrease in expression of AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, YAP1, ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, WNT7B, or a combination thereof, relative to a healthy control subject.

6. The method of claim 1, wherein the neurological disorder comprises Alzheimer's disease (AD), dementia, multiple sclerosis (MS), Parkinson's disease, Huntington's disease, obsessive compulsive disorder (OCD), attention-deficit/hyperactivity disorder (ADHD), schizophrenia, or amyotrophic lateral sclerosis (ALS).

7. The method of claim 1, wherein the anti-viral agent comprises acyclovir or valacyclovir.

8. The method of claim 1, wherein the anti-viral agent is administered with a therapeutic agent.

9. The method of claim 1, wherein the therapeutic agent comprises a neurological therapeutic agent.

10. The method of claim 9, wherein the neurological therapeutic agent comprises levetiracetam, gabapentin, topiramate, lamotrigine, carbidopa, levodopa, donepezil HCl, sumatriptan succinate, oxcarbazepine, amitriptyline HCl, memantine HCl, divalprox sodium, pregabalin, pramipexole, ropinirole, rotigotine, clozapine, quetiapine, olanzapine, galantamine, rivastigmine, trifluoperazine, methylphenidate, atomoxetine, amphetamine, dextroamphetamine, pemoline, perphenazine, aducanumab, lecanemab, suvorexant, brexpiprazole, or a combination thereof.

11. The method of claim 1, wherein the method decreases amyloid-β (Aβ-42) or phosphorylated Tau (pTau-212) concentrations in the subject relative to an untreated subject with the neurological disorder.

12. The method of claim 1, wherein the method prevents progression of the neurological disorder.

13. The method of claim 1, wherein the biological sample comprises a cerebrospinal fluid (CSF), blood, serum, or tissue biopsy.

14. The method of claim 1, wherein the gene expression signature from the biological sample comprises detecting or sequencing an RNA molecule.

15. A kit for detecting a panel of genes affected by a neurological disorder and a herpes simplex virus (HSV), the kit comprising:
a primer composition for binding at least one gene of said panel, and
an enzymatic composition for amplifying the at least one gene,
wherein the at least one gene is selected from AARS2, ABCA1, ALDH1A2, ARIH1, BMP1, BMPER, CAMK4, CD2AP, CDKAL1, CELF2, CENPM, COL18A1, DDAH1, DDX18, DST, DVL2, ERBB3, ERBB4, F2R, F2RL1, FAM83E, FAT1, FERMT2, FOXN3, G3BP1, HDAC9, HRK, HYI, ITSN2, KCNN2, LAMA1, LINGO2, MOBP, MRC1, NKAIN3, PAK2, PEX6, PLEC, PPP4R3A, PRKD3, PRRC2C, RAPSN, RBMS3, RPTOR, SERPINE1, SETD7, SORD, SPATS1, SPPL2A, SQSTM1, SUDS3, TGFB2, TMEM106B, TULP4, WAC, WDR70, YAP1, ACER3, AFF1, AHNAK, AP4M1, APOE, BCKDK, BIN1, CCDC85C, CDC5L, CDR2L, CLEC3B, DCHS2, DMXL1, DOCK4, EPC2, FAM163A, FNIP1, FOXE1, HSPG2, MED12L, NECTIN2, NEPRO, OSBPL6, PFDN1, POLN, PSMC3, RFN6, SLC35C1, SP6, STRADA SYNJ1, TMED9, TNRC6A, TOP1, TSKU, TSPAN14, TST, TXNL1, WNT7B, or fragments thereof.

16. (canceled)

17. (canceled)

18. (canceled)

19. (canceled)

20. The kit of claim 15, wherein the enzymatic composition comprises a polymerase and a buffer.

21. The kit of claim 15, wherein the neurological disorder comprises Alzheimer's disease (AD), dementia, multiple sclerosis (MS), Parkinson's disease, Huntington's disease, obsessive compulsive disorder (OCD), attention-deficit/hyperactivity disorder (ADHD), schizophrenia, or amyotrophic lateral sclerosis (ALS).

22. The kit of claim 15, wherein the HSV comprises an HSV-1.

23. The kit of claim 15, further comprising a reagent for extracting a nucleic acid.

24. The kit of claim 15, further comprising a reagent for detecting the at least one gene.

25. (canceled)
